(19) United States
(12) Patent Application Publication
(10) Pub. No.: US 2024/0084301 A1
(43) Pub. Date: Mar. 14, 2024

(54) RNAI CONSTRUCTS AND METHODS FOR INHIBITING FAM13A EXPRESSION

(71) Applicant: AMGEN INC., Thousand Oaks, CA (US)

(72) Inventors: Elizabeth Ann KILLION, Van Nuys, CA (US); Jerry Ryan HOLDER, Simi Valley, CA (US); Bryan MEADE, Westlake Village, CA (US); Justin K. MURRAY, Moorpark, CA (US); Oliver R. HOMANN, Berkeley, CA (US); Briana Renee GRIEGO, Oxnard, CA (US); Murielle Marie VENIANT ELLISON, Thousand Oaks, CA (US)

(73) Assignee: AMGEN INC., Thousand Oaks, CA (US)

(21) Appl. No.: 18/357,495

(22) Filed: Jul. 24, 2023

Related U.S. Application Data
(60) Provisional application No. 63/391,860, filed on Jul. 25, 2022.

(51) Int. Cl. C12N 15/113; A61P 1/16; A61P 3/04

(52) U.S. Cl. CPC C12N 15/113; A61P 1/16; A61P 3/04; C12N 2310/14; C12N 2310/315; C12N 2310/321; C12N 2310/322

(57) ABSTRACT

The present application relates to compositions and methods for modulating expression of Family with Sequence Similarity 13 Member A (FAM13A) protein. In particular, the present application relates to nucleic acid-based therapeutics for reducing FAM13A gene expression via RNA interference and methods of using such nucleic acid-based therapeutics to reduce abdominal adiposity, reduce body weight, reduce fat mass, improve metabolic parameters including insulin resistance and non-alcoholic steatohepatitis (NASH), and reduce risk of myocardial infarction.

### CROSS REFERENCE TO RELATED APPLICATIONS

This application claims priority to U.S. Provisional Patent Application No. 63/391,860, filed Jul. 25, 2022, which is incorporated herein by reference in its entirety.

### TECHNICAL FIELD

The present application relates to compositions and methods for modulating expression of Family with Sequence Similarity 13 Member A (FAM13A) protein. In particular, the present application relates to nucleic acid-based therapeutics for reducing FAM13A gene expression via RNA interference and methods of using such nucleic acid-based therapeutics.

### REFERENCE TO THE SEQUENCE LISTING

The present application is being filed along with a Sequence Listing in electronic format. The Sequence Listing is provided as an XML file entitled 10121-U502-SEC_ST26, created Jul. 14, 2023, which is 2.81 MB in size. The information in the electronic format of the Sequence Listing is incorporated herein by reference in its entirety.

### BACKGROUND

Obesity, or excess adiposity, is recognized as a disease and is established as a major risk factor for cardiovascular disease (CVD). The most common measure of adiposity, body-mass-index (BMI), results in increased odds risk of myocardial infarction (MI). However, this association is substantially reduced after adjustment for waist-to-hip ratio (WHR), a measurement that reflects a visceral body fat distribution pattern (also known as central or abdominal obesity). WHR has been shown to be more robustly related to MI risk with individuals in the highest quintile for WHR having a 2.52-fold increase in odds ratio (p<0.001), a finding that persists even after adjustment for BMI. Yusuf et al., Lancet 366:1640-1649 (2005); Cao et al., Medicine (Baltimore) 97, e11639 (2018); de Koning et al., Eur. Heart J., 28, 850-856 (2007). These data indicate that WHR is a better predictor of MI risk than BMI and that this metric overcomes some key limitations of BMI (e.g., high muscle mass).

FAM13A (also known as FAM13A1, KIAA0914, or ARHGAP48) is a cytosolic protein that has been shown to regulate AMP-activated protein kinase (AMPK) activity, and it has been linked to regulation of hepatic glucose, lipid metabolism, body fat distribution, and adipocyte function. Lin et al., iScience 23, 100928 (2020); Fathzadeh et al., Nature Communications 11, 1465 (2020). For example, human genetic evidence has linked FAM13A with HDL cholesterol, body mass index (BMI)-adjusted fasting insulin levels, and WHR adjusted for BMI. In vitro, FAM13A knockdown in human mesenchymal stem cells increases adipocyte differentiation and thermogenesis while overexpression causes apoptosis of pre-adipocytes and inhibits adipogenesis. Lundback et al., Diabetologia, 2018; Tang et al., Int. J. Obesity, 2019; Fathzadeh et al., Nat. Comm., 2020. Additionally, FAM13A KO mice are protected against diet-induced obesity (DIO), have improved hepatic insulin sensitivity, and increased hepatocyte oxygen consumption rate. Lin et al., iScience, 2020.

### SUMMARY

The present application relates, in part, to the design and generation of RNAi constructs that target the FAM13A gene and reduce its expression. The sequence-specific inhibition of FAM13A gene expression is useful for reducing abdominal adiposity, reducing body weight, reducing fat mass, improving metabolic parameters including insulin resistance and non-alcoholic steatohepatitis (NASH), and reducing risk of myocardial infarction. Accordingly, in one embodiment, the present application provides an RNAi construct comprising a sense strand and an antisense strand, wherein the antisense strand comprises a region comprising a sequence that is substantially complementary to a FAM13A mRNA sequence. In some embodiments, the RNAi construct is targeted only to the liver. In some embodiments, the antisense strand comprises a sequence that is substantially complementary to the sequence of at least 15 contiguous nucleotides of a region of the human FAM13A mRNA sequence (SEQ ID NO: 1) with no more than 1, 2, or 3 mismatches. In some embodiments, the antisense strand comprises a region comprising a sequence that is substantially complementary to at least 15 contiguous nucleotides within particular regions of the FAM13A mRNA sequence set forth in SEQ ID NO: 1, such as within nucleotides 1300-1375 or 4900-5300 of SEQ ID NO: 1. In certain embodiments, the antisense strand comprises a region comprising at least 15 contiguous nucleotides from an antisense sequence listed in Table 1 or Table 2.

In some embodiments, the sense strand of the RNAi constructs described herein comprises a sequence that is sufficiently complementary to the sequence of the antisense strand to form a duplex region of about 15 to about 30 base pairs in length, about 17 to about 24 base pairs in length, or about 19 to about 21 base pairs in length. In some embodiments, the sense and antisense strands are each independently about 19 to about 30 nucleotides in length, or about 19 to about 23 nucleotides in length. In some embodiments, the RNAi constructs comprise one or two blunt ends. In other embodiments, the RNAi constructs comprise one or two nucleotide overhangs. Such nucleotide overhangs may comprise 1 to 6 unpaired nucleotides and can be located at the 3′ end of the sense strand, the 3′ end of the antisense strand, or the 3′ end of both the sense and antisense strand. In certain embodiments, the RNAi constructs comprise an overhang of two unpaired nucleotides at the 3′ end of the sense strand and the 3′ end of the antisense strand. In other embodiments, the RNAi constructs comprise an overhang of two unpaired nucleotides at the 3′ end of the antisense strand and a blunt end at the 3′ end of the sense strand/5′ end of the antisense strand.

The disclosed RNAi constructs may comprise one or more modified nucleotides, including nucleotides having modifications to the ribose ring, nucleobase, or phosphodiester backbone. In some embodiments, the RNAi constructs comprise one or more 2′-modified nucleotides. Such 2′-modified nucleotides can include 2′-fluoro modified nucleotides, 2′-O-methyl modified nucleotides, 2′-O-methoxyethyl modified nucleotides, 2′-O-alkyl modified nucleotides, 2′-O-allyl modified nucleotides, bicyclic nucleic acids (BNA), deoxyribonucleotides, or combinations thereof. In one particular embodiment, the RNAi constructs comprise one or more 2′-fluoro modified nucleotides, 2′-O-methyl modified nucleotides, or combinations thereof. In some embodiments, all of the nucleotides in the sense and antisense strand of the RNAi construct are modified nucleotides. Abasic nucleotides may be incorporated into the disclosed RNAi constructs, for example, as the terminal nucleotide at the 3′ end, the 5′ end, or both the 3′ end and the 5′ end of the sense strand. In such embodiments, the abasic nucleotide may be inverted, e.g., linked to the adjacent nucleotide through a 3′-3′ internucleotide linkage or a 5′-5′ internucleotide linkage.

In some embodiments, the RNAi constructs comprise at least one backbone modification, such as a modified internucleotide or internucleotide linkage. In certain embodiments, the RNAi constructs described herein comprise at least one phosphorothioate internucleotide linkage. In particular embodiments, the phosphorothioate internucleotide linkages may be positioned at the 3′ or 5′ ends of the sense and/or antisense strands. For instance, in some embodiments, the antisense strand comprises two consecutive phosphorothioate internucleotide linkages between the terminal nucleotides at both the 3′ and 5′ ends. In some such embodiments, the sense strand comprises one or two phosphorothioate internucleotide linkages between the terminal nucleotides at its 3′ end.

In some embodiments, the RNAi constructs of this application may target a particular region of the human FAM13A mRNA transcript set forth in SEQ ID NO: 1. In some embodiments, the sequence of the antisense strand may be fully complementary to the sequence of at least 15 contiguous nucleotides of the specific regions of the human FAM13A transcript (SEQ ID NO: 1). In some embodiments, the sequence of the antisense strand may be substantially complementary to the sequence of at least 15 contiguous nucleotides of the specific regions of the human FAM13A transcript (SEQ ID NO: 1) with no more than 1, 2, or 3 mismatches between the sequence of the antisense strand and the sequence of the specific regions of the human FAM13A transcript. In certain embodiments, the antisense strand and/or the sense strand of the RNAi constructs may comprise or consist of a sequence from the antisense and sense sequences listed in Table 1. In some embodiments, the sense and antisense strands, respectively, comprise or consist of SEQ ID NOs: 15 and 559, SEQ ID NOs: 24 and 568, SEQ ID NOs: 125 and 669, SEQ ID NOs: 127 and 671, SEQ ID NOs: 222 and 766, SEQ ID NOs: 406 and 950, SEQ ID NOs: 448 and 992, SEQ ID NOs: 498 and 1042, SEQ ID NOs: 502 and 1046, SEQ ID NOs: 503 and 1047, SEQ ID NOs: 504 and 1048, SEQ ID NOs: 513 and 1057, SEQ ID NOs: 526 and 1070, SEQ ID NOs: 527 and 1071, SEQ ID NOs: 533 and 1077, or SEQ ID NOs: 534 and 1078.

In some embodiments, the RNAi construct comprises particular sequences with particular modification patterns, which are referred to as duplexes herein. In certain embodiments, the antisense strand and/or the sense strand of the RNAi constructs, with particular modification patterns, may comprise or consist of antisense and sense sequences listed in Table 2 as particular duplexes. In some embodiments, the RNAi construct is a duplex called D-1557, D-1597, D-1612, D-1614, D-1623, D-1650, D-1667, D-1680, D-1682, D-1685, D-1686, D-1690, D-1697, D-1698, D-1699, D-1702, D-1704, D-1705, D-1709, D-1768, D-1846, D-1849, D-1853, D-1856, D-1858, D-1861, D-1862, D-1863, D-1864, D-1865, D-1866, D-1868, D-1869, D-1870, D-1871, D-1873, D-1875, D-1876, D-1877, D-1878, D-1879, D-1880, D-1881, D-1883, D-1884, D-1885, D-1886, D-1887, D-1888, D-1899, D-1896, D-1955, D-1970, D-1972, D-1975, D-1976, D-1977, D-1979, D-1980, D-1981, D-1982, D-1983, D-1984, D-1985, D-1987, D-1988, D-1989, D-1990, D-1991, D-1992, D-1993, D-1994, D-1995, D-1996, D-1997, D-1998, D-2000, D-2001, D-2002, D-2003, D-2004, D-2005, D-2012, D-2013, D-2014, D-2017, D-2021, D-2022, D-2023, D-2040, D-2044, D-2045, D-2047, D-2049, D-2051, D-2052, D-2053, D-2054, D-2058, D-2061, D-2075, D-2077, D-2079, D-2080, D-2081, D-2083, D-2090, D-2091, or D-2093. In some embodiments, the RNAi construct is a duplex observed to knock down FAM13A expression by greater than 80%.

The disclosed RNAi constructs may comprise a ligand to facilitate delivery or uptake of the RNAi constructs to specific tissues or cells, such as liver or adipose cells. In certain embodiments, the ligand targets delivery of the RNAi constructs to hepatocytes. In these and other embodiments, the ligand may comprise galactose, galactosamine, or N-acetyl-galactosamine (GalNAc). In certain embodiments, the ligand comprises a multivalent galactose or multivalent GalNAc moiety, such as a trivalent or tetravalent galactose or GalNAc moiety. The ligand may be covalently attached to the 5′ or 3′ end of the sense strand of the RNAi construct, optionally through a linker. In some embodiments, the RNAi constructs comprise a ligand and linker comprising a structure according to any one of Formulas I to IX described herein. In certain embodiments, the RNAi constructs comprise a ligand and linker comprising a structure according to Formula VII. In other embodiments, the RNAi constructs comprise a ligand and linker comprising a structure according to Formula IV. In some embodiments, the ligand comprises a long-chain fatty acid such as lauric acid (C12), myristic acid (C14), palmitic acid (C16), stearic acid (C18), eicosapentaenoic acid (C20), or docosanoic acid (C22). In some embodiments, the ligand is attached through a phosphodiester or phosphorothioate linkage.

The present application also provides pharmaceutical compositions comprising any of the RNAi constructs described herein and a pharmaceutically acceptable carrier, excipient, or diluent. Such pharmaceutical compositions are particularly useful for reducing expression of the FAM13A gene in the cells (e.g., liver or adipose cells) of a patient in need thereof. Patients who may be administered a disclosed pharmaceutical composition include patients diagnosed with or at risk of obesity, including patients displaying a high WHR and patients diagnosed with abdominal obesity. Patients who may be administered a disclosed pharmaceutical composition also can include patients diagnosed with or at risk of metabolic conditions such as fatty liver disease (e.g., NAFLD, NASH, alcoholic fatty liver disease, or alcoholic steatohepatitis), insulin resistance and type 2 diabetes (T2D), hypertriglyceridemia, or hypercholesterolemia. The present application also provides methods of treating patients in need of reduction of expression of the FAM13A gene expression in their cells, including patients diagnosed with or at risk of obesity, abdominal obesity, fatty liver disease (e.g., NAFLD, NASH, alcoholic fatty liver disease, or alcoholic steatohepatitis), insulin resistance and type 2 diabetes (T2D), hypertriglyceridemia, or hypercholesterolemia. These methods comprise administering an RNAi construct or pharmaceutical composition described herein. In some embodiments, the RNAi construct is administered with a ligand that targets the RNAi construct to the liver or hepatocytes.

The use of FAM13A-targeting RNAi constructs in any of the methods described herein or for preparation of medicaments for administration according to the methods described herein is specifically contemplated. For instance, the present application includes a FAM13A-targeting RNAi construct for use in treating, preventing, or reducing the risk of developing obesity, abdominal obesity, fatty liver disease (e.g., NAFLD, NASH, alcoholic fatty liver disease, or alcoholic steatohepatitis), insulin resistance and type 2 diabetes (T2D), hypertriglyceridemia, or hypercholesterolemia in a patient in need thereof.

### BRIEF DESCRIPTION OF THE FIGURES

FIG. 1 shows the results of a genomic analysis performed to examine the association of three common FAM13A variants for their association with adjusted for BMI (WHRadjBMI), triglyceride levels, HDL cholesterol levels, systolic blood pressure, and FAM13A expression in subcutaneous adipose tissue eQTL data.

FIGS. 2A and 2B show the results of an in vitro dose-response study of Fam13a siRNA's effects in Renca cells and primary adipocytes.

FIGS. 3A-3D show the results of an in vivo study of Fam13a siRNA's ability to knock down murine Fam13a mRNA expression levels in the liver and white adipose tissue of mice.

FIGS. 4A-4C show the results of an in vivo study of Fam13a siRNA's effects on body weight and fat mass of mice.

FIG. 5 is a table showing the effects of C16- and GalNAc-linked Fam13a siRNA in obese mice after 60 days of treatment. Fam13a siRNAs had significant effects on body weight, fat mass, cumulative food intake, liver weight, insulin levels, total cholesterol, LDL cholesterol, and ALT levels.

FIG. 6 is a diagram compiling the locations which of a range of human FAM13A siRNA triggers target on the human FAM13A mRNA transcript. The depicted triggers were all efficacious in reducing FAM13A mRNA levels and are divided in this diagram according to whether the maximum observed knockdown for that trigger fell within the range of 40-60% knockdown, 60-80% knockdown, or greater than 80% knockdown.

FIGS. 7A-7R are depictions of different modification patterns that may be applied to siRNA trigger sequences, with each figure showing a hybridized sense (top) and antisense (bottom) strand. In these figures, the solid circles correspond to 2′-O-methyl ribonucleotides, the open circles correspond to 2′-deoxy-2′-fluoro (“2′-fluoro”) ribonucleotides, and the hatched circles correspond to inverted abasic deoxynucleotides. Bold lines indicate where a phosphorothioate bond is used in place of the standard phosphodiester bond between nucleotides. Finally, arrows represent where a ligand (e.g., GalNAc or a fatty acid) may be attached to a polynucleotide.

FIGS. 8A-8D show the results of testing FAM13A siRNA in an AAV human FAM13A mouse model. FIGS. 8A and 8B show that a range of different members of the T-4999 and T-5043 trigger families, respectively, reduced expression of FAM13A mRNA in the liver. FIGS. 8C and 8D show that C22-conjugated members of the T-4999 and T-5043 trigger families, and to a lesser extent GalNAc-conjugated members of the T-4999 and T-5043 trigger families, were able to reduce expression of FAM13A mRNA in adipose tissue. In each of FIGS. 8A-8D, the “*” denotes those duplexes that were conjugated to C22, while those without an asterisk were conjugated to GalNAc.

FIGS. 9A-9C show the results of testing human-mouse cross reactive FAM13A siRNA duplexes, with knockdown noted in liver, inguinal white adipose tissue, and epididymal white adipose tissue.

FIGS. 10A and 10B show that treating diet-induced obese (DIO) mice with human-mouse cross reactive FAM13A siRNA duplexes prevented the increases in body weight and fat mass associated with the DIO model.

FIGS. 11A-11E show the results of treating cynomolgus monkeys with a single dose of human-cynomolgus monkey cross reactive FAM13A siRNA. FIGS. 11A and 11B show that knockdown was achieved in both liver and adipose tissue. FIGS. 11C-11E show that FAM13A siRNA treatment resulted in decreases in serum cholesterol, LDL, and HDL, respectively.

### DETAILED DESCRIPTION

The present application is directed to compositions and methods for regulating the expression of the FAM13A gene in a cell or mammal. In some embodiments, compositions comprise RNAi constructs that target a mRNA transcribed from the FAM13A gene, particularly the human FAM13A gene, and reduce expression of the FAM13A protein in a cell or mammal. Such RNAi constructs are useful for treating, preventing, or reducing the risk of developing obesity, hepatosteatosis, insulin resistance and type 2 diabetes (T2D), hypertriglyceridemia, or hypercholesterolemia in a patient in need thereof.

### RNAi Constructs

As used herein, the term “RNAi construct” refers to an agent comprising an RNA molecule that is capable of downregulating expression of a target gene (e.g., the FAM13A gene) via an RNA interference mechanism when introduced into a cell. RNA interference is the process by which a nucleic acid molecule induces the cleavage and degradation of a target RNA molecule (e.g., messenger RNA or mRNA molecule) in a sequence-specific manner, e.g., through an RNA-induced silencing complex (RISC) pathway. In some embodiments, the RNAi construct comprises a double-stranded RNA molecule comprising two antiparallel strands of contiguous nucleotides that are sufficiently complementary to each other to hybridize to form a duplex region. “Hybridize” or “hybridization” refers to the pairing of complementary polynucleotides, typically via hydrogen bonding (e.g., Watson-Crick, Hoogsteen or reversed Hoogsteen hydrogen bonding) between complementary bases in the two polynucleotides. The strand comprising a region comprising a sequence that is substantially complementary to a target sequence (e.g., target mRNA) is referred to as the “antisense strand” or “guide strand.” The “sense strand” or “passenger strand” refers to the strand that includes a region that is substantially complementary to a region of the antisense strand. In some embodiments, the sense strand may comprise a region that has a sequence that is substantially identical to the target sequence.

A double-stranded RNA molecule may include chemical modifications to ribonucleotides, including modifications to the ribose sugar, base, or backbone components of the ribonucleotides, such as those described herein or known in the art. Any such modifications, as used in a double-stranded RNA molecule (e.g., siRNA, shRNA, or the like), are encompassed by the term “double-stranded RNA” for the purposes of this disclosure. Details on potential modifications to the RNAi constructs described herein are provided in the Modification and Preparation of RNAi Constructs section below.

As used herein, a first sequence is “complementary” to a second sequence if a polynucleotide comprising the first sequence can hybridize to a polynucleotide comprising the second sequence to form a duplex region under certain conditions, such as physiological conditions. Other such conditions can include moderate or stringent hybridization conditions, which are known to those of skill in the art. A first sequence is fully complementary (100% complementary) to a second sequence if a polynucleotide comprising the first sequence base pairs with a polynucleotide comprising the second sequence over the entire length of one or both nucleotide sequences without any mismatches. A sequence is “substantially complementary” to a target sequence, or has “substantial identity to” a target sequence, if the sequence is at least about 80%, 85%, 90%, 95%, 96%, 97%, 98%, or 99% complementary to a target sequence. Percent complementarity can be calculated by dividing the number of bases in a first sequence that are complementary to bases at corresponding positions in a second or target sequence by the total length of the first sequence. A sequence may also be said to be substantially complementary to another sequence if there are no more than 5, 4, 3, or 2 mismatches over a 30 base pair duplex region when the two sequences are hybridized. Generally, if any nucleotide overhangs, as defined herein, are present, the sequence of such overhangs is not considered in determining the degree of complementarity between two sequences. By way of example, a sense strand of 21 nucleotides in length and an antisense strand of 21 nucleotides in length that hybridize to form a 19 base pair duplex region with a 2-nucleotide overhang at the 3′ end of each strand would be fully complementary as the term is used herein.

In some embodiments, a region of the antisense strand comprises a sequence that is substantially or fully complementary to a region of the target RNA sequence (e.g., the FAM13A mRNA sequence). In such embodiments, the sense strand may comprise a sequence that is fully complementary to the sequence of the antisense strand. In other such embodiments, the sense strand may comprise a sequence that is substantially complementary to the sequence of the antisense strand, e.g., having 1, 2, 3, 4, or 5 mismatches in the duplex region formed by the sense and antisense strands. In certain embodiments, it is preferred that any mismatches occur within the terminal regions (e.g., within 6, 5, 4, 3, or 2 nucleotides of the 5′ and/or 3′ ends of the strands). In one embodiment, any mismatches in the duplex region formed from the sense and antisense strands occur within 6, 5, 4, 3, or 2 nucleotides of the 5′ end of the antisense strand.

In certain embodiments, the sense strand and antisense strand of the double-stranded RNA may be two separate molecules that hybridize to form a duplex region but are otherwise unconnected. Such double-stranded RNA molecules formed from two separate strands are referred to as “small interfering RNAs” or “short interfering RNAs” (siRNAs). Thus, in some embodiments, the RNAi constructs comprise an siRNA.

In other embodiments, the sense strand and the antisense strand that hybridize to form a duplex region may be part of a single RNA molecule, i.e., the sense and antisense strands are part of a self-complementary region of a single RNA molecule. In such cases, a single RNA molecule comprises a duplex region (also referred to as a stem region) and a loop region. The 3′ end of the sense strand is connected to the 5′ end of the antisense strand by a contiguous sequence of unpaired nucleotides, which will form the loop region. The loop region is typically of a sufficient length to allow the RNA molecule to fold back on itself such that the antisense strand can base pair with the sense strand to form the duplex or stem region. The loop region can comprise from about 3 to about 25, from about 5 to about 15, or from about 8 to about 12 unpaired nucleotides. Such RNA molecules with at least partially self-complementary regions are referred to as “short hairpin RNAs” (shRNAs). In certain embodiments, the RNAi constructs comprise a shRNA. The length of a single, at least partially self-complementary RNA molecule can be from about 40 nucleotides to about 100 nucleotides, from about 45 nucleotides to about 85 nucleotides, or from about 50 nucleotides to about 60 nucleotides and comprise a duplex region and loop region each having the lengths recited herein.

In some embodiments, the RNAi constructs comprise a sense strand and an antisense strand, wherein the antisense strand comprises a region having a sequence that is substantially or fully complementary to a FAM13A messenger RNA (mRNA) sequence. As used herein, a “FAM13A mRNA sequence” refers to any messenger RNA sequence, including allelic variants and splice variants, encoding a FAM13A protein, including FAM13A protein variants or isoforms from any species (e.g., non-human primate, human).

A FAM13A mRNA sequence also includes the transcript sequence expressed as its complementary DNA (cDNA) sequence. A cDNA sequence refers to the sequence of an mRNA transcript expressed as DNA bases (e.g., guanine, adenine, thymine, and cytosine) rather than RNA bases (e.g., guanine, adenine, uracil, and cytosine). Thus, the antisense strand of the RNAi constructs may comprise a region having a sequence that is substantially or fully complementary to a target FAM13A mRNA sequence or FAM13A cDNA sequence. A FAM13A mRNA or cDNA sequence can include, but is not limited to, any FAM13A mRNA or cDNA sequences in the Ensembl Genome or National Center for Biotechnology Information (NCBI) databases, including human sequences such as Ensembl transcript no. ENST00000264344.9 (SEQ ID NO: 1) and NCBI Reference sequence NM_022746.4. A FAM13A mRNA or cDNA sequence can also include cynomolgus monkey sequences, rhesus monkey sequences, chimpanzee sequences, rat sequences, and mouse sequences. In certain embodiments, the FAM13A mRNA sequence is the human transcript set forth below (SEQ ID NO: 1).

CCTTCCAGCCATGTGGGTTCAGCGGAAAGAGAAGCAAAACCACTCTTCCTAAAATGTTAGAA
GCTGCTCTTCGCTTACCTTGGGGCCTTTGCATTGGGAGCTGTTTTTCACATCAAAGAATATG
TGCTGAATGGAATTTTAGTATTTTGCTGTCGTTTTAATATTTTCGTCTGGTCTTCCTCAGTT
CTTCCAGACGCTTTCTGAGAGAATGGGGGCAGGAGCTCTAGCCATCTGTCAAAGTAAAGCAG
CGGTTCGGCTGAAAGAAGACATGAAAAAGATAGTGGCAGTGCCATTAAATGAACAGAAGGAT
TTTACCTATCAGAAGTTATTTGGAGTCAGTCTCCAAGAACTTGAACGGCAGGGGCTCACCGA
GAATGGCATTCCAGCAGTAGTGTGGAATATAGTGGAATATTTGACGCAGCATGGACTTACCC
AAGAAGGTCTTTTTAGGGTGAATGGTAACGTGAAGGTGGTGGAACAACTTCGACTGAAGTTC
GAGAGTGGAGTGCCCGTGGAGCTCGGGAAGGACGGTGATGTCTGCTCAGCAGCCAGTCTGTT
GAAGCTGTTTCTGAGGGAGCTGCCTGACAGTCTGATCACCTCAGCGTTGCAGCCTCGATTCA
TTCAACTCTTTCAGGATGGCAGAAATGATGTTCAGGAGAGTAGCTTAAGAGACTTAATAAAA
GAGCTGCCAGACACCCACTACTGCCTCCTCAAGTACCTTTGCCAGTTCTTGACAAAAGTAGC
CAAGCATCATGTGCAGAATCGCATGAATGTTCACAATCTCGCCACTGTATTTGGGCCAAATT
GCTTTCATGTGCCACCTGGGCTTGAAGGCATGAAGGAACAGGACCTGTGCAACAAGATAATG
GCTAAAATTCTAGAAAATTACAATACCCTGTTTGAAGTAGAGTATACAGAAAATGATCATCT
GAGATGTGAAAACCTGGCTAGGCTTATCATAGTAAAAGAGGTCTATTATAAGAACTCCCTGC
CCATCCTTTTAACAAGAGGCTTAGAAAGAGACATGCCAAAACCACCTCCAAAAACCAAGATC
CCAAAATCCAGGAGTGAGGGATCTATTCAGGCCCACAGAGTACTGCAACCAGAGCTATCTGA
TGGCATTCCTCAGCTCAGCTTGCGGCTAAGTTATAGAAAAGCCTGCTTGGAAGACATGAATT
CAGCAGAGGGTGCTATTAGTGCCAAGTTGGTACCCAGTTCACAGGAAGATGAAAGACCTCTG
TCACCTTTCTATTTGAGTGCTCATGTACCCCAAGTCAGCAATGTGTCTGCAACCGGAGAACT
CTTAGAAAGAACCATCCGATCAGCTGTAGAACAACATCTTTTTGATGTTAATAACTCTGGAG
GTCAAAGTTCAGAGGACTCAGAATCTGGAACACTATCAGCATCTTCTGCCACATCTGCCAGA
CAGCGCCGCCGCCAGTCCAAGGAGCAGGATGAAGTTCGACATGGGAGAGACAAGGGACTTAT
CAACAAAGAAAATACTCCTTCTGGGTTCAACCACCTTGATGATTGTATTTTGAATACTCAGG
AAGTCGAAAAGGTACACAAAAATACTTTTGGTTGTGCTGGAGAAAGGAGCAAGCCTAAACGT
CAGAAATCCAGTACTAAACTTTCTGAGCTTCATGACAATCAGGACGGTCTTGTGAATATGGA
AAGTCTCAATTCCACACGATCTCATGAGAGAACTGGACCTGATGATTTTGAATGGATGTCTG
ATGAAAGGAAAGGAAATGAAAAAGATGGTGGACACACTCAGCATTTTGAGAGCCCCACAATG
AAGATCCAGGAGCATCCCAGCCTATCTGACACCAAACAGCAGAGAAATCAAGATGCCGGTGA
CCAGGAGGAGAGCTTTGTCTCCGAAGTGCCCCAGTCGGACCTGACTGCATTGTGTGATGAAA
AGAACTGGGAAGAGCCTATCCCTGCTTTCTCCTCCTGGCAGCGGGAGAACAGTGACTCTGAT
GAAGCCCACCTCTCGCCGCAGGCTGGGCGCCTGATCCGTCAGCTGCTGGACGAAGACAGCGA
CCCCATGCTCTCTCCTCGGTTCTACGCTTATGGGCAGAGCAGGCAATACCTGGATGACACAG
AAGTGCCTCCTTCCCCACCAAACTCCCATTCTTTCATGAGGCGGCGAAGCTCCTCTCTGGGG
TCCTATGATGATGAGCAAGAGGACCTGACACCTGCCCAGCTCACACGAAGGATTCAGAGCCT
TAAAAAGAAGATCCGGAAGTTTGAAGATAGATTCGAAGAAGAGAAGAAGTACAGACCTTCCC
ACAGTGACAAAGCAGCCAATCCGGAGGTTCTGAAATGGACAAATGACCTTGCCAAATTCCGG
AGACAACTTAAAGAATCAAAACTAAAGATATCTGAAGAGGACCTAACTCCCAGGATGCGGCA
GCGAAGCAACACACTCCCCAAGAGTTTTGGTTCCCAACTTGAGAAAGAAGATGAGAAGAAGC
AAGAGCTGGTGGATAAAGCAATAAAGCCCAGTGTTGAAGCCACATTGGAATCTATTCAGAGG
AAGCTCCAGGAGAAGCGAGCGGAAAGCAGCCGCCCTGAGGACATTAAGGATATGACCAAAGA
CCAGATTGCTAATGAGAAAGTGGCTCTGCAGAAAGCTCTGTTATATTATGAAAGCATTCATG
GACGGCCGGTAACAAAGAACGAACGGCAGGTGATGAAGCCACTATACGACAGGTACCGGCTG
GTCAAACAGATCCTCTCCCGAGCTAACACCATACCCATCATTGGTTCCCCCTCCAGCAAGCG
GAGAAGCCCTTTGCTGCAGCCAATTATCGAGGGCGAAACTGCTTCCTTCTTCAAGGAGATAA
AGGAAGAAGAGGAGGGGTCAGAAGACGATAGCAATGTGAAGCCAGACTTCATGGTCACTCTG
AAAACCGATTTCAGTGCACGATGCTTTCTGGACCAATTCGAAGATGACGCTGATGGATTTAT
TTCCCCAATGGATGATAAAATACCATCAAAATGCAGCCAGGACACAGGGCTTTCAAATCTCC
ATGCTGCCTCAATACCTGAACTCCTGGAACACCTCCAGGAAATGAGAGAAGAAAAGAAAAGG
ATTCGAAAGAAACTTCGGGATTTTGAAGACAACTTTTTCAGACAGAATGGAAGAAATGTCCA
GAAGGAAGACCGCACTCCTATGGCTGAAGAATACAGTGAATATAAGCACATAAAGGCGAAAC
TGAGGCTCCTGGAGGTGCTCATCAGCAAGAGAGACACTGATTCCAAGTCCATGTGAGGGGCA
TGGCCAAGCACAGGGGGCTGGCAGCTGCGGTGAGAGTTTACTGTCCCCAGAGAAAGTGCAGC
TCTGGAAGGCAGCCTTGGGGCTGGCCCTGCAAAGCATGCAGCCCTTCTGCCTCTAGACCATT
TGGCATCGGCTCCTGTTTCCATTGCCTGCCTTAGAAACTGGCTGGAAGAAGACAATGTGACC
TGACTTAGGCATTTTGTAATTGGAAAGTCAAGACTGCAGTATGTGCACATGCGCACGCGCAT
GCACGCACACACACACACAGTAGTGGAGCTTTCCTAACACTAGCAGAGATTAATCACTACAT
TAGACAACACTCATCTACAGAGAATATACACTGTTCTTCCCTGGATAACTGAGAAACAAGAG
ACCATTCTCTGTCTAACTGTGATAAAAACAAGCTCAGGACTTTATTCTATAGAGCAAACTTG
CTGTGGAGGGCCATGCTCTCCTTGGACCCAGTTAACTGCAAACGTGCATTGGAGCCCTATTT
GCTGCCGCTGCCATTCTAGTGACCTTTCCACAGAGCTGCGCCTTCCTCACGTGTGTGAAAGG
TTTTCCCCTTCAGCCCTCAGGTAGATGGAAGCTGCATCTGCCCACGATGGCAGTGCAGTCAT
CATCTTCAGGATGTTTCTTCAGGACTTCCTCAGCTGACAAGGAATTTTGGTCCCTGCCTAGG
ACCGGGTCATCTGCAGAGGACAGAGAGATGGTAAGCAGCTGTATGAATGCTGATTTTAAAAC
CAGGTCATGGGAGAAGAGCCTGGAGATTCTTTCCTGAACACTGACTGCACTTACCAGTCTGA
TTTTATCGTCAAACACCAAGCCAGGCTAGCATGCTCATGGCAATCTGTTTGGGGCTGTTTTG
TTGTGGCACTAGCCAAACATAAAGGGGCTTAAGTCAGCCTGCATACAGAGGATCGGGGAGAG
AAGGGGCCTGTGTTCTCAGCCTCCTGAGTACTTACCAGAGTTTAATTTTTTTAAAAAAAATC
TGCACTAAAATCCCCAAACTGACAGGTAAATGTAGCCCTCAGAGCTCAGCCCAAGGCAGAAT
CTAAATCACACTATTTTCGAGATCATGTATAAAAAGAAAAAAAAGAAGTCATGCTGTGTGGC
CAATTATAATTTTTTTCAAAGACTTTGTCACAAAACTGTCTATATTAGACATTTTGGAGGGA
CCAGGAAATGTAAGACACCAAATCCTCCATCTCTTCAGTGTGCCTGATGTCACCTCATGATT
TGCTGTTACTTTTTTAACTCCTGCGCCAAGGACAGTGGGTTCTGTGTCCACCTTTGTGCTTT
GCGAGGCCGAGCCCAGGCATCTGCTCGCCTGCCACGGCTGACCAGAGAAGGTGCTTCAGGAG
CTCTGCCTTAGACGACGTGTTACAGTATGAACACACAGCAGAGGCACCCTCGTATGTTTTGA
AAGTTGCCTTCTGAAAGGGCACAGTTTTAAGGAAAAGAAAAAGAATGTAAAACTATACTGAC
CCGTTTTCAGTTTTAAAGGGTCGTGAGAAACTGGCTGGTCCAATGGGATTTACAGCAACATT
TTCCATTGCTGAAGTGAGGTAGCAGCTCTCTTCTGTCAGCTGAATGTTAAGGATGGGGAAAA
AGAATGCCTTTAAGTTTGCTCTTAATCGTATGGAAGCTTGAGCTATGTGTTGGAAGTGCCCT
GGTTTTAATCCATACACAAAGACGGTACATAATCCTACAGGTTTAAATGTACATAAAAATAT
AGTTTGGAATTCTTTGCTCTACTGTTTACATTGCAGATTGCTATAATTTCAAGGAGTGAGAT
TATAAATAAAATGATGCACTTTAGGATGTTTCCTATTTTTGAAATCTGAACATGAATCATTC
ACATGACCAAAAATTGTGTTTTTTTAAAAATACATGTCTAGTCTGTCCTTTAATAGCTCTCT
TAAATAAGCTATGATATTAATCAGATCATTACCAGTTAGCTTTTAAAGCACATTTGTTTAAG
ACTATGTTTTTGGAAAAATACGCTACAGAATTTTTTTTTAAGCTACAAATAAATGAGATGCT
ACTAATTGTTTTGGAATCTGTTGTTTCTGCCAAAGGTAAATTAACTAAAGATTTATTCAGGA
ATCCCCATTTGAATTTGTATGATTCAATAAAAGAAAACACCAAGTAAGTTATATAAAATAAA
TTGTGTATGAGATGTTGTGTTTTCCTTTGTAATTTCCACTAACTAACTAACTAACTTATATT
CTTCATGGAATGGAGCCCAGAAGAAATGAGAGGAAGCCCTTTTCACACTAGATCTTATTTGA
AGAAATGTTTGTTAGTCAGTCAGTCAGTGGTTTCTGGCTCTGCCGAGGGAGATGTGTTCCCC
AGCAACCATTTCTGCAGCCCAGAATCTCAAGGCACTAGAGGCGGTGTCTTAATTAATTGGCT
TCACAAAGACAAAATGCTCTGGACTGGGATTTTTCCTTTGCTGTGTTGGGAATATGTGTTTA
TTAATTAGCACATGCCAACAAAATAAATGTCAAGAGTTATTTCATAAGTGTAAGTAAACTTA
AGAATTAAAGAGTGCAGACTTATAATTTTC

A region of the antisense strand can be substantially complementary or fully complementary to at least 15 consecutive nucleotides of the FAM13A mRNA sequence. In certain embodiments, the region of the antisense strand comprises a sequence that is substantially complementary to the sequence of at least 15, at least 16, at least 17, at least 18, or at least 19 contiguous nucleotides of a region of the FAM13A mRNA sequence (e.g., a human FAM13A mRNA sequence (SEQ ID NO: 1)) with no more than 1, 2, or 3 mismatches. In related embodiments, the antisense strand comprises a region having a sequence that is substantially complementary to the sequence of at least 15, at least 16, at least 17, at least 18, or at least 19 contiguous nucleotides of a region of the FAM13A mRNA sequence with no more than 1 mismatch. In some embodiments, the target region of the FAM13A mRNA sequence to which the antisense strand comprises a region of complementarity can range from about 15 to about 30 consecutive nucleotides, from about 16 to about 28 consecutive nucleotides, from about 18 to about 26 consecutive nucleotides, from about 17 to about 24 consecutive nucleotides, from about 19 to about 30 consecutive nucleotides, from about 19 to about 25 consecutive nucleotides, from about 19 to about 23 consecutive nucleotides, or from about 19 to about 21 consecutive nucleotides. In certain embodiments, the region of the antisense strand comprising a sequence that is substantially or fully complementary to a FAM13A mRNA sequence may comprise at least 15 contiguous nucleotides from an antisense sequence listed in Table 1 or Table 2. In other embodiments, the sequence of the antisense strand comprises at least 16, at least 17, at least 18, or at least 19 contiguous nucleotides from an antisense sequence listed in Table 1 or Table 2.

In some embodiments, the region of the antisense strand comprising a sequence that is substantially or fully complementary to a FAM13A mRNA sequence may comprise at least 15 contiguous nucleotides from a region that is particularly susceptible to being targeted by a RNAi construct. Therefore, in some embodiments, the region of the antisense strand comprising a sequence that is substantially or fully complementary to a FAM13A mRNA sequence may comprise at least 15 contiguous nucleotides from within nucleotides 1300-1375, 1625-1700, 2075-2175, or 4900-5300 of the human FAM13A mRNA sequence set forth in SEQ ID NO: 1. In some embodiments, the region of the antisense strand comprising a sequence that is substantially or fully complementary to a FAM13A mRNA sequence may comprise at least 15 contiguous nucleotides from a sub-section of these regions. Therefore, in some embodiments, the sequence may comprise at least 15 contiguous nucleotides from nucleotides 1300-1350, 4900-5275, 4900-5250, 4900-5225, 4900-5200, 4900-5175, 4900-5150, 4900-5125, 4900-5100, 4900-5075, 4925-5300, 4925-5275, 4925-5250, 4925-5225, 4925-5200, 4925-5175, 4925-5150, 4925-5125, 4925-5100, 4925-5075, 4950-5300, 4950-5275, 4950-5250, 4950-5225, 4950-5200, 4950-5175, 4950-5150, 4950-5125, 4950-5100, 4950-5075, 4975-5300, 4975-5275, 4975-5250, 4975-5225, 4975-5200, 4975-5175, 4975-5150, 4975-5125, 4975-5100, 4975-5075, 5175-3000, 5100-5300, 5125-5300, 5150-5300, 5175-5300, 5200-5300, or 5225-5300.

The sense strand of the RNAi construct typically comprises a sequence that is sufficiently complementary to the sequence of the antisense strand such that the two strands hybridize under physiological conditions to form a duplex region. A “duplex region” refers to the region in two complementary or substantially complementary polynucleotides that form base pairs with one another, either by Watson-Crick base pairing or other hydrogen bonding interaction, to create a duplex between the two polynucleotides. The duplex region of the RNAi construct should be of sufficient length to allow the RNAi construct to enter the RNA interference pathway, e.g., by engaging the Dicer enzyme and/or the RISC complex. For instance, in some embodiments, the duplex region is about 15 to about 30 base pairs in length. Other lengths for the duplex region within this range are also suitable, such as about 15 to about 28 base pairs, about 15 to about 26 base pairs, about 15 to about 24 base pairs, about 15 to about 22 base pairs, about 17 to about 28 base pairs, about 17 to about 26 base pairs, about 17 to about 24 base pairs, about 17 to about 23 base pairs, about 17 to about 21 base pairs, about 19 to about 25 base pairs, about 19 to about 23 base pairs, or about 19 to about 21 base pairs. In certain embodiments, the duplex region is about 17 to about 24 base pairs in length. In other embodiments, the duplex region is about 19 to about 21 base pairs in length. In one embodiment, the duplex region is about 19 base pairs in length. In another embodiment, the duplex region is about 21 base pairs in length.

For embodiments in which the sense strand and antisense strand are two separate molecules (e.g., RNAi construct comprises an siRNA), the sense strand and antisense strand need not be the same length as the length of the duplex region. For instance, one or both strands may be longer than the duplex region and have one or more unpaired nucleotides or mismatches flanking the duplex region. Thus, in some embodiments, the RNAi construct comprises at least one nucleotide overhang. As used herein, a “nucleotide overhang” refers to the unpaired nucleotide or nucleotides that extend beyond the duplex region at the terminal ends of the strands. Nucleotide overhangs are typically created when the 3′ end of one strand extends beyond the 5′ end of the other strand or when the 5′ end of one strand extends beyond the 3′ end of the other strand. The length of a nucleotide overhang is generally between 1 and 6 nucleotides, 1 and 5 nucleotides, 1 and 4 nucleotides, 1 and 3 nucleotides, 2 and 6 nucleotides, 2 and 5 nucleotides, or 2 and 4 nucleotides. In some embodiments, the nucleotide overhang comprises 1, 2, 3, 4, 5, or 6 nucleotides. In some embodiments, the nucleotide overhang comprises 1 to 4 nucleotides. In certain embodiments, the nucleotide overhang comprises 2 nucleotides. In certain other embodiments, the nucleotide overhang comprises a single nucleotide.

The nucleotides in the overhang can be ribonucleotides or modified nucleotides as described herein. In some embodiments, the nucleotides in the overhang are 2′-modified nucleotides (e.g., 2′-fluoro modified nucleotides, 2′-O-methyl modified nucleotides), deoxyribonucleotides, abasic nucleotides, inverted nucleotides (e.g., inverted abasic nucleotides, inverted deoxyribonucleotides), or combinations thereof. For instance, in one embodiment, the nucleotides in the overhang are deoxyribonucleotides, e.g., deoxythymidine. In another embodiment, the nucleotides in the overhang are 2′-O-methyl modified nucleotides, 2′-fluoro modified nucleotides, 2′-methoxyethyl modified nucleotides, or combinations thereof. In other embodiments, the overhang comprises a 5′-uridine-uridine-3′ (5′-UU-3′) dinucleotide. In such embodiments, the UU dinucleotide may comprise ribonucleotides or modified nucleotides, e.g., 2′-modified nucleotides. In other embodiments, the overhang comprises a 5′-deoxythymidine-deoxythymidine-3′ (5′-dTdT-3′) dinucleotide. When a nucleotide overhang is present in the antisense strand, the nucleotides in the overhang can be complementary to the target gene sequence, form a mismatch with the target gene sequence, or comprise some other sequence (e.g., polypyrimidine or polypurine sequence, such as UU, TT, AA, GG, etc.).

The nucleotide overhang can be at the 5′ end or 3′ end of one or both strands. For example, in one embodiment, the RNAi construct comprises a nucleotide overhang at the 5′ end and the 3′ end of the antisense strand. In another embodiment, the RNAi construct comprises a nucleotide overhang at the 5′ end and the 3′ end of the sense strand. In some embodiments, the RNAi construct comprises a nucleotide overhang at the 5′ end of the sense strand and the 5′ end of the antisense strand. In other embodiments, the RNAi construct comprises a nucleotide overhang at the 3′ end of the sense strand and the 3′ end of the antisense strand.

The RNAi constructs may comprise a single nucleotide overhang at one end of the double-stranded RNA molecule and a blunt end at the other. A “blunt end” means that the sense strand and antisense strand are fully base-paired at the end of the molecule and there are no unpaired nucleotides that extend beyond the duplex region. In some embodiments, the RNAi construct comprises a nucleotide overhang at the 3′ end of the sense strand and a blunt end at the 5′ end of the sense strand and 3′ end of the antisense strand. In other embodiments, the RNAi construct comprises a nucleotide overhang at the 3′ end of the antisense strand and a blunt end at the 5′ end of the antisense strand and the 3′ end of the sense strand. In certain embodiments, the RNAi construct comprises a blunt end at both ends of the double-stranded RNA molecule. In such embodiments, the sense strand and antisense strand have the same length and the duplex region is the same length as the sense and antisense strands (i.e., the molecule is double stranded over its entire length).

The sense strand and antisense strand in the RNAi constructs can each independently be about 15 to about 30 nucleotides in length, about 19 to about 30 nucleotides in length, about 18 to about 28 nucleotides in length, about 19 to about 27 nucleotides in length, about 19 to about 25 nucleotides in length, about 19 to about 23 nucleotides in length, about 19 to about 21 nucleotides in length, about 21 to about 25 nucleotides in length, or about 21 to about 23 nucleotides in length. In certain embodiments, the sense strand and antisense strand are each independently about 18, about 19, about 20, about 21, about 22, about 23, about 24, or about 25 nucleotides in length. In some embodiments, the sense strand and antisense strand have the same length but form a duplex region that is shorter than the strands such that the RNAi construct has two nucleotide overhangs. For instance, in one embodiment, the RNAi construct comprises (i) a sense strand and an antisense strand that are each 21 nucleotides in length, (ii) a duplex region that is 19 base pairs in length, and (iii) nucleotide overhangs of 2 unpaired nucleotides at both the 3′ end of the sense strand and the 3′ end of the antisense strand. In another embodiment, the RNAi construct comprises (i) a sense strand and an antisense strand that are each 23 nucleotides in length, (ii) a duplex region that is 21 base pairs in length, and (iii) nucleotide overhangs of 2 unpaired nucleotides at both the 3′ end of the sense strand and the 3′ end of the antisense strand. In other embodiments, the sense strand and antisense strand have the same length and form a duplex region over their entire length such that there are no nucleotide overhangs on either end of the double-stranded molecule. In one such embodiment, the RNAi construct is blunt ended (e.g., has two blunt ends) and comprises (i) a sense strand and an antisense strand, each of which is 21 nucleotides in length, and (ii) a duplex region that is 21 base pairs in length. In another such embodiment, the RNAi construct is blunt ended (e.g., has two blunt ends) and comprises (i) a sense strand and an antisense strand, each of which is 23 nucleotides in length, and (ii) a duplex region that is 23 base pairs in length. In still another such embodiment, the RNAi construct is blunt ended (e.g., has two blunt ends) and comprises (i) a sense strand and an antisense strand, each of which is 19 nucleotides in length, and (ii) a duplex region that is 19 base pairs in length.

In other embodiments, the sense strand or the antisense strand is longer than the other strand and the two strands form a duplex region having a length equal to that of the shorter strand such that the RNAi construct comprises at least one nucleotide overhang. For example, in one embodiment, the RNAi construct comprises (i) a sense strand that is 19 nucleotides in length, (ii) an antisense strand that is 21 nucleotides in length, (iii) a duplex region of 19 base pairs in length, and (iv) a nucleotide overhang of 2 unpaired nucleotides at the 3′ end of the antisense strand. In another embodiment, the RNAi construct comprises (i) a sense strand that is 21 nucleotides in length, (ii) an antisense strand that is 23 nucleotides in length, (iii) a duplex region of 21 base pairs in length, and (iv) a nucleotide overhang of 2 unpaired nucleotides at the 3′ end of the antisense strand.

The antisense strand of the RNAi constructs can comprise or consist of the sequence of any one of the antisense sequences listed in Table 1 or Table 2, the sequence of nucleotides 1-19 of any of these antisense sequences, or the sequence of nucleotides 2-19 of any of these antisense sequences. Thus, in some embodiments, the antisense strand comprises or consists of a sequence selected from SEQ ID NOs: 546-1089 or 1938-2785. In other embodiments, the antisense strand comprises or consists of a sequence of nucleotides 1-19 of any one of SEQ ID NOs: 546-1089 or 1938-2785. In still other embodiments, the antisense strand comprises or consists of a sequence of nucleotides 2-19 of any one of SEQ ID NOs: 546-1089 or 1938-2785.

In these and other embodiments, the sense strand of the RNAi constructs can comprise or consist of the sequence of any one of the sense sequences listed in Table 1 or Table 2, the sequence of nucleotides 1-19 of any of these sense sequences, or the sequence of nucleotides 2-19 of any of these sense sequences. Thus, in some embodiments, the sense strand comprises or consists of a sequence selected from SEQ ID NOs: 2-545 or 1090-1937. In other embodiments, the sense strand comprises or consists of a sequence of nucleotides 1-19 of any one of SEQ ID NOs: 2-545 or 1090-1937. In still other embodiments, the sense strand comprises or consists of a sequence of nucleotides 2-19 of any one of SEQ ID NOs: 2-545 or 1090-1937.

In certain embodiments, the RNAi constructs comprise (i) a sense strand comprising or consisting of a sequence selected from 2-545 or 1090-1937 and (ii) an antisense strand comprising or consisting of a sequence selected from SEQ ID NOs: 546-1089 or 1938-2785. In some embodiments, the RNAi construct can be any of the duplex compounds listed in Table 1 or Table 2 (including the unmodified nucleotide sequences and/or modified nucleotide sequences of the compounds). In certain embodiments, the RNAi construct is D-1539, D-1544, D-1545, D-1549, D-1557, D-1559, D-1573, D-1579, D-1586, D-1597, D-1607, D-1611, D-1612, D-1614, D-1623, D-1631, D-1636, D-1639, D-1640, D-1643, D-1644, D-1645, D-1646, D-1648, D-1652, D-1661, D-1667, D-1672, or D-1694.

### Modification and Preparation of RNAi Constructs

The RNAi constructs disclosed herein may comprise one or more modified nucleotides. A “modified nucleotide” refers to a nucleotide that has one or more chemical modifications to the nucleoside, nucleobase, pentose ring, or phosphate group. As used herein, modified nucleotides do not encompass ribonucleotides containing adenosine monophosphate, guanosine monophosphate, uridine monophosphate, and cytidine monophosphate. However, the RNAi constructs may comprise combinations of modified nucleotides and ribonucleotides. Incorporation of modified nucleotides into one or both strands of double-stranded RNA molecules can improve the in vivo stability of the RNA molecules, e.g., by reducing the molecules' susceptibility to nucleases and other degradation processes. The potency of RNAi constructs for reducing expression of the target gene can also be enhanced by incorporation of modified nucleotides.

In certain embodiments, the modified nucleotides have a modification of the ribose sugar. These sugar modifications can include modifications at the 2′ and/or 5′ position of the pentose ring as well as bicyclic sugar modifications. A 2′-modified nucleotide refers to a nucleotide having a pentose ring with a substituent at the 2′ position other than OH. Such 2′-modifications include, but are not limited to, 2′-H (e.g., deoxyribonucleotides), 2′-O-alkyl (e.g., —O—C₁-C₁₀ or —O—C₁-C₁₀ substituted alkyl), 2′-O-allyl (—O—CH₂CH═CH₂), 2′-C-allyl, 2′-deoxy-2′-fluoro (also referred to as 2′-F or 2′-fluoro), 2′-O-methyl (—OCH₃), 2′-O-methoxyethyl (—O—(CH₂)₂OCH₃), 2′-OCF₃, 2′-O(CH₂)₂SCH₃, 2′-O-aminoalkyl, 2′-amino (e.g., —NH₂), 2′-O-ethylamine, and 2′-azido. Modifications at the 5′ position of the pentose ring include, but are not limited to, 5′-methyl (R or S configuration); 5′-vinyl, and 5′-methoxy.

A “bicyclic sugar modification” refers to a modification of the pentose ring where a bridge connects two atoms of the ring to form a second ring resulting in a bicyclic sugar structure. In some embodiments the bicyclic sugar modification comprises a bridge between the 4′ and 2′ carbons of the pentose ring. Nucleotides comprising a sugar moiety with a bicyclic sugar modification are referred to herein as bicyclic nucleic acids or BNAs. Exemplary bicyclic sugar modifications include, but are not limited to, α-L-Methyleneoxy (4′-CH₂—O-2′) bicyclic nucleic acid (BNA); β-D-Methyleneoxy (4′-CH₂—O-2′) BNA (also referred to as a locked nucleic acid or LNA); Ethyleneoxy (4′-(CH₂)₂—O-2′) BNA; Aminooxy (4′-CH₂—O—N(R)-2′, wherein R is H, C₁-C₁₂ alkyl, or a protecting group) BNA; Oxyamino (4′-CH₂—N(R)—O-2′, wherein R is H, C₁-C₁₂ alkyl, or a protecting group) BNA; Methyl(methyleneoxy) (4′-CH(CH₃)—O-2′) BNA (also referred to as constrained ethyl or cEt); methylene-thio (4′-CH₂—S-2′) BNA; methylene-amino (4′-CH2-N(R)-2′, wherein R is H, C₁-C₁₂ alkyl, or a protecting group) BNA; methyl carbocyclic (4′-CH₂—CH(CH₃)-2′) BNA; propylene carbocyclic (4′-(CH₂)₃-2′) BNA; and Methoxy(ethyleneoxy) (4′-CH(CH₂OMe)-O-2′) BNA (also referred to as constrained MOE or cMOE). These and other sugar-modified nucleotides that can be incorporated into the RNAi constructs are described in U.S. Pat. No. 9,181,551, U.S. Patent Publication No. 2016/0122761, and Deleavey and Damha, Chemistry and Biology, Vol. 19: 937-954, 2012, all of which are hereby incorporated by reference in their entireties.

In some embodiments, the RNAi constructs comprise one or more 2′-fluoro modified nucleotides, 2′-O-methyl modified nucleotides, 2′-O-methoxyethyl modified nucleotides, 2′-O-alkyl modified nucleotides, 2′-O-allyl modified nucleotides, bicyclic nucleic acids (BNAs), deoxyribonucleotides, or combinations thereof. In certain embodiments, the RNAi constructs comprise one or more 2′-fluoro modified nucleotides, 2′-O-methyl modified nucleotides, 2′-O-methoxyethyl modified nucleotides, or combinations thereof. In some embodiments, the RNAi constructs comprise one or more 2′-fluoro modified nucleotides, 2′-O-methyl modified nucleotides or combinations thereof.

Both the sense and antisense strands of the RNAi constructs can comprise one or multiple modified nucleotides. For instance, in some embodiments, the sense strand comprises 1, 2, 3, 4, 5, 6, 7, 8, 9, 10 or more modified nucleotides. In certain embodiments, all nucleotides in the sense strand are modified nucleotides. In some embodiments, the antisense strand comprises 1, 2, 3, 4, 5, 6, 7, 8, 9, 10 or more modified nucleotides. In other embodiments, all nucleotides in the antisense strand are modified nucleotides. In certain other embodiments, all nucleotides in the sense strand and all nucleotides in the antisense strand are modified nucleotides. In these and other embodiments, the modified nucleotides can be 2′-fluoro modified nucleotides, 2′-O-methyl modified nucleotides, or combinations thereof.

In certain embodiments, the modified nucleotides incorporated into one or both strands of the RNAi constructs have a modification of the nucleobase (also referred to herein as “base”). A “modified nucleobase” or “modified base” refers to a base other than the naturally occurring purine bases adenine (A) and guanine (G) and pyrimidine bases thymine (T), cytosine (C), and uracil (U). Modified nucleobases can be synthetic or naturally occurring modifications and include, but are not limited to, universal bases, 5-methylcytosine (5-me-C), 5-hydroxymethyl cytosine, xanthine (X), hypoxanthine (I), 2-aminoadenine, 6-methyladenine, 6-methylguanine, and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

In some embodiments, the modified base is a universal base. A “universal base” refers to a base analog that indiscriminately forms base pairs with all the natural bases in RNA and DNA without altering the double helical structure of the resulting duplex region. Universal bases are known to those of skill in the art and include, but are not limited to, inosine, C-phenyl, C-naphthyl and other aromatic derivatives, azole carboxamides, and nitroazole derivatives, such as 3-nitropyrrole, 4-nitroindole, 5-nitroindole, and 6-nitroindole.

Other suitable modified bases that can be incorporated into the RNAi constructs include those described in Herdewijn, Antisense Nucleic Acid Drug Dev., Vol. 10: 297-310, 2000 and Peacock et al., J. Org. Chem., Vol. 76: 7295-7300, 2011, both of which are hereby incorporated by reference in their entireties. The skilled person understands guanine, cytosine, adenine, thymine, and uracil may be replaced by other nucleobases, such as the modified nucleobases described above, without substantially altering the base pairing properties of a polynucleotide comprising a nucleotide bearing such replacement nucleobase.

In some embodiments, the sense and antisense strands of the RNAi constructs may comprise one or more abasic nucleotides. An “abasic nucleotide” or “abasic nucleoside” is a nucleotide or nucleoside that lacks a nucleobase at the 1′ position of the ribose sugar. In certain embodiments, the abasic nucleotides are incorporated into the terminal ends of the sense and/or antisense strands of the RNAi constructs. In one embodiment, the sense strand comprises an abasic nucleotide as the terminal nucleotide at its 3′ end, its 5′ end, or both its 3′ and 5′ ends. In another embodiment, the antisense strand comprises an abasic nucleotide as the terminal nucleotide at its 3′ end, its 5′ end, or both its 3′ and 5′ ends. In such embodiments in which the abasic nucleotide is a terminal nucleotide, it may be an inverted nucleotide—that is, linked to the adjacent nucleotide through a 3′-3′ internucleotide linkage (when on the 3′ end of a strand) or through a 5′-5′ internucleotide linkage (when on the 5′ end of a strand) rather than the natural 3′-5′ internucleotide linkage. Abasic nucleotides may also comprise a sugar modification, such as any of the sugar modifications described above. In certain embodiments, abasic nucleotides comprise a 2′-modification, such as a 2′-fluoro modification, 2′-O-methyl modification, or a 2′-H (deoxy) modification. In one embodiment, the abasic nucleotide comprises a 2′-O-methyl modification. In another embodiment, the abasic nucleotide comprises a 2′-H modification (i.e., a deoxy abasic nucleotide).

In certain embodiments, the RNAi constructs may comprise modified nucleotides incorporated into the sense and antisense strands according to a particular pattern, such as the patterns described in WIPO Publication No. WO 2020/123410, which is hereby incorporated by reference in its entirety. RNAi constructs having such chemical modification patterns have been shown to have improved gene silencing activity in vivo. In one embodiment, the RNAi construct comprises a sense strand and an antisense strand that comprise sequences that are sufficiently complementary to each other to form a duplex region of at least 15 base pairs, wherein:
nucleotides at positions 2, 7, and 14 in the antisense strand (counting from the 5′ end) are 2′-fluoro modified nucleotides;
nucleotides in the sense strand at positions paired with positions 8 to 11 and 13 in the antisense strand (counting from the 5′ end) are 2′-fluoro modified nucleotides; and
neither the sense strand nor the antisense strand each have more than 7 total 2′-fluoro modified nucleotides.

In other embodiments, the RNAi construct comprises a sense strand and an antisense strand that comprise sequences that are sufficiently complementary to each other to form a duplex region of at least 19 base pairs, wherein:
nucleotides at positions 2, 7, and 14 in the antisense strand (counting from the 5′ end) are 2′-fluoro modified nucleotides, nucleotides at positions 4, 6, 10, and 12 (counting from the 5′ end) are optionally 2′-fluoro modified nucleotides, and all other nucleotides in the antisense strand are modified nucleotides other than 2′-fluoro modified nucleotides; and
nucleotides in the sense strand at positions paired with positions 8 to 11 and 13 in the antisense strand (counting from the 5′ end) are 2′-fluoro modified nucleotides, nucleotides in the sense strand at positions paired with positions 3 and 5 in the antisense strand (counting from the 5′ end) are optionally 2′-fluoro modified nucleotides; and all other nucleotides in the sense strand are modified nucleotides other than 2′-fluoro modified nucleotides.

In such embodiments, the modified nucleotides other than 2′-fluoro modified nucleotides can be selected from 2′-O-methyl modified nucleotides, 2′-O-methoxyethyl modified nucleotides, 2′-O-alkyl modified nucleotides, 2′-O-allyl modified nucleotides, BNAs, and deoxyribonucleotides. In these and other embodiments, the terminal nucleotide at the 3′ end, the 5′ end, or both the 3′ end and the 5′ end of the sense strand can be an abasic nucleotide or a deoxyribonucleotide. In such embodiments, the abasic nucleotide or deoxyribonucleotide may be inverted—i.e., linked to the adjacent nucleotide through a 3′-3′ internucleotide linkage (when on the 3′ end of a strand) or through a 5′-5′ internucleotide linkage (when on the 5′ end of a strand) rather than the natural 3′-5′ internucleotide linkage.

In any of the above-described embodiments, nucleotides at positions 2, 7, 12, and 14 in the antisense strand (counting from the 5′ end) are 2′-fluoro modified nucleotides. In other embodiments, nucleotides at positions 2, 4, 7, 12, and 14 in the antisense strand (counting from the 5′ end) are 2′-fluoro modified nucleotides. In yet other embodiments, nucleotides at positions 2, 4, 6, 7, 12, and 14 in the antisense strand (counting from the 5′ end) are 2′-fluoro modified nucleotides. In still other embodiments, nucleotides at positions 2, 4, 6, 7, 10, 12, and 14 in the antisense strand (counting from the 5′ end) are 2′-fluoro modified nucleotides. In alternative embodiments, nucleotides at positions 2, 7, 10, 12, and 14 in the antisense strand (counting from the 5′ end) are 2′-fluoro modified nucleotides. In certain other embodiments, nucleotides at positions 2, 4, 7, 10, 12, and 14 in the antisense strand (counting from the 5′ end) are 2′-fluoro modified nucleotides.

In any of the above-described embodiments, nucleotides in the sense strand at positions paired with positions 3, 8 to 11, and 13 in the antisense strand (counting from the 5′ end) are 2′-fluoro modified nucleotides. In some embodiments, nucleotides in the sense strand at positions paired with positions 5, 8 to 11, and 13 in the antisense strand (counting from the 5′ end) are 2′-fluoro modified nucleotides. In other embodiments, nucleotides in the sense strand at positions paired with positions 3, 5, 8 to 11, and 13 in the antisense strand (counting from the 5′ end) are 2′-fluoro modified nucleotides.

In some embodiments, the RNAi construct comprises a structure represented by Formula (A):

(A)
5′-(N_{A})ₓ N_{L} N_{L} N_{L} N_{L} N_{L} N_{L} N_{F} N_{L} N_{F} N_{F} N_{F} N_{F} N_{L} N_{L} N_{M}
N_{L} N_{M} N_{L} N_{T}(n)_{y}-3′
3′-(N_{B})_{z} N_{L} N_{L} N_{L} N_{L} N_{L} N_{F} N_{L} N_{M} N_{L} N_{M} N_{L} N_{L} N_{F} N_{M} N_{L}
N_{M} N_{L} N_{F} N_{L}-5′

In Formula (A), the top strand listed in the 5′ to 3′ direction is the sense strand and the bottom strand listed in the 3′ to 5′ direction is the antisense strand; each N_{F} represents a 2′-fluoro modified nucleotide; each N_{M} independently represents a modified nucleotide selected from a 2′-fluoro modified nucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide; each N_{L} independently represents a modified nucleotide selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide; and N_{T} represents a modified nucleotide selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide. X can be an integer from 0 to 4, provided that when x is 1, 2, 3, or 4, one or more of the N_{A} nucleotides is a modified nucleotide independently selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide. One or more of the N_{A} nucleotides can be complementary to nucleotides in the antisense strand. Y can be an integer from 0 to 4, provided that when y is 1, 2, 3, or 4, one or more n nucleotides are modified or unmodified overhang nucleotides that do not base pair with nucleotides in the antisense strand. Z can be an integer from 0 to 4, provided that when z is 1, 2, 3, or 4, one or more of the NB nucleotides is a modified nucleotide independently selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide. One or more of the NB nucleotides can be complementary to N_{A} nucleotides when present in the sense strand or can be overhang nucleotides that do not base pair with nucleotides in the sense strand.

In some embodiments in which the RNAi construct comprises a structure represented by Formula (A), there is a nucleotide overhang at the 3′ end of the sense strand—i.e., y is 1, 2, 3, or 4. In one such embodiment, y is 2. In embodiments in which there is an overhang of 2 nucleotides at the 3′ end of the sense strand (i.e., y is 2), x is 0 and z is 2 or x is 1 and z is 2. In other embodiments in which the RNAi construct comprises a structure represented by Formula (A), the RNAi construct comprises a blunt end at the 3′ end of the sense strand and the 5′ end of the antisense strand (i.e., y is 0). In such embodiments where there is no nucleotide overhang at the 3′ end of the sense strand (i.e., y is 0): (i) x is 2 and z is 4, (ii) x is 3 and z is 4, (iii) x is 0 and z is 2, (iv) x is 1 and z is 2, or (v) x is 2 and z is 2. In any of the embodiments in which x is greater than 0, the N_{A} nucleotide that is the terminal nucleotide at the 5′ end of the sense strand can be an inverted nucleotide, such as an inverted abasic nucleotide or an inverted deoxyribonucleotide.

In certain embodiments in which the RNAi construct comprises a structure represented by Formula (A), the N_{M} at positions 4 and 12 in the antisense strand counting from the 5′ end are each a 2′-fluoro modified nucleotide. In other embodiments, the N_{M} at positions 4, 6, and 12 in the antisense strand counting from the 5′ end are each a 2′-fluoro modified nucleotide. In yet other embodiments, the N_{M} at positions 4, 6, 10, and 12 in the antisense strand counting from the 5′ end are each a 2′-fluoro modified nucleotide. In alternative embodiments in which the RNAi construct comprises a structure represented by Formula (A), the N_{M} at positions 10 and 12 in the antisense strand counting from the 5′ end are each a 2′-fluoro modified nucleotide. In related embodiments, the N_{M} at positions 4, 10, and 12 in the antisense strand counting from the 5′ end are each a 2′-fluoro modified nucleotide. In other alternative embodiments in which the RNAi construct comprises a structure represented by Formula (A), the N_{M} at positions 4, 6, and 10 in the antisense strand counting from the 5′ end are each a 2′-O-methyl modified nucleotide, and the N_{M} at position 12 in the antisense strand counting from the 5′ end is a 2′-fluoro modified nucleotide. In some embodiments in which the RNAi construct comprises a structure represented by Formula (A), each N_{M} in the sense strand is a 2′-O-methyl modified nucleotide. In other embodiments, each N_{M} in the sense strand is a 2′-fluoro modified nucleotide. In still other embodiments in which the RNAi construct comprises a structure represented by Formula (A), each N_{M} in both the sense and antisense strands is a 2′-O-methyl modified nucleotide.

In any of the above-described embodiments in which the RNAi construct comprises a structure represented by Formula (A), each N_{L} in both the sense and antisense strands can be a 2′-O-methyl modified nucleotide. In these embodiments and any of the embodiments described above, N_{T} in Formula (A) can be an inverted abasic nucleotide, an inverted deoxyribonucleotide, or a 2′-O-methyl modified nucleotide.

In other embodiments, the RNAi construct comprises a structure represented by Formula (B):

(B)
5′-(N_{A})ₓ N_{L} N_{L} N_{L} N_{L} N_{M} N_{L} N_{F} N_{F} N_{F} N_{F} N_{L} N_{L} N_{L} N_{L} N_{L}
N_{L} N_{L} N_{L} N_{T} (n)_{y}-3′
3′-(N_{B})_{z} N_{L} N_{L} N_{L} N_{M} N_{L} N_{F} N_{L} N_{M} N_{L} N_{L} N_{N} N_{N} N_{N} N_{N} N_{L}
N_{M} N_{L} N_{F} N_{L}-5′

In Formula (B), the top strand listed in the 5′ to 3′ direction is the sense strand and the bottom strand listed in the 3′ to 5′ direction is the antisense strand; each N_{F} represents a 2′-fluoro modified nucleotide; each N_{M} independently represents a modified nucleotide selected from a 2′-fluoro modified nucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide; each N_{L} independently represents a modified nucleotide selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide; and N_{T} represents a modified nucleotide selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide. X can be an integer from 0 to 4, provided that when x is 1, 2, 3, or 4, one or more of the N_{A} nucleotides is a modified nucleotide independently selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide. One or more of the N_{A} nucleotides can be complementary to nucleotides in the antisense strand. Y can be an integer from 0 to 4, provided that when y is 1, 2, 3, or 4, one or more n nucleotides are modified or unmodified overhang nucleotides that do not base pair with nucleotides in the antisense strand. Z can be an integer from 0 to 4, provided that when z is 1, 2, 3, or 4, one or more of the NB nucleotides is a modified nucleotide independently selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide. One or more of the NB nucleotides can be complementary to N_{A} nucleotides when present in the sense strand or can be overhang nucleotides that do not base pair with nucleotides in the sense strand.

In some embodiments in which the RNAi construct comprises a structure represented by Formula (B), there is a nucleotide overhang at the 3′ end of the sense strand—i.e., y is 1, 2, 3, or 4. In one such embodiment, y is 2. In embodiments in which there is an overhang of 2 nucleotides at the 3′ end of the sense strand (i.e., y is 2), x is 0 and z is 2 or x is 1 and z is 2. In other embodiments in which the RNAi construct comprises a structure represented by Formula (B), the RNAi construct comprises a blunt end at the 3′ end of the sense strand and the 5′ end of the antisense strand (i.e., y is 0). In such embodiments where there is no nucleotide overhang at the 3′ end of the sense strand (i.e., y is 0): (i) x is 2 and z is 4, (ii) x is 3 and z is 4, (iii) x is 0 and z is 2, (iv) x is 1 and z is 2, or (v) x is 2 and z is 2. In any of the embodiments in which x is greater than 0, the N_{A} nucleotide that is the terminal nucleotide at the 5′ end of the sense strand can be an inverted nucleotide, such as an inverted abasic nucleotide or an inverted deoxyribonucleotide.

In certain embodiments in which the RNAi construct comprises a structure represented by Formula (B), the N_{M} at positions 4, 6, 8, 9, and 16 in the antisense strand counting from the 5′ end are each a 2′-fluoro modified nucleotide and the N_{M} at positions 7 and 12 in the antisense strand counting from the 5′ end are each a 2′-O-methyl modified nucleotide. In other embodiments, the N_{M} at positions 4 and 6 in the antisense strand counting from the 5′ end are each a 2′-fluoro modified nucleotide and the N_{M} at positions 7 to 9 in the antisense strand counting from the 5′ end are each a 2′-O-methyl modified nucleotide. In still other embodiments, the N_{M} at positions 4, 6, 8, 9, and 16 in the antisense strand counting from the 5′ end are each a 2′-O-methyl modified nucleotide and the N_{M} at positions 7 and 12 in the antisense strand counting from the 5′ end are each a 2′-fluoro modified nucleotide. In alternative embodiments in which the RNAi construct comprises a structure represented by Formula (B), the N_{M} at positions 4, 6, 8, 9, and 12 in the antisense strand counting from the 5′ end are each a 2′-O-methyl modified nucleotide and the N_{M} at positions 7 and 16 in the antisense strand counting from the 5′ end are each a 2′-fluoro modified nucleotide. In certain other embodiments in which the RNAi construct comprises a structure represented by Formula (B), the N_{M} at positions 7, 8, 9, and 12 in the antisense strand counting from the 5′ end are each a 2′-O-methyl modified nucleotide and the N_{M} at positions 4, 6, and 16 in the antisense strand counting from the 5′ end are each a 2′-fluoro modified nucleotide. In these and other embodiments in which the RNAi construct comprises a structure represented by Formula (B), the N_{M} in the sense strand is a 2′-fluoro modified nucleotide. In alternative embodiments, the N_{M} in the sense strand is a 2′-O-methyl modified nucleotide.

In any of the above-described embodiments in which the RNAi construct comprises a structure represented by Formula (B), each N_{L} in both the sense and antisense strands can be a 2′-O-methyl modified nucleotide. In these embodiments and any of the embodiments described above, N_{T} in Formula (B) can be an inverted abasic nucleotide, an inverted deoxyribonucleotide, or a 2′-O-methyl modified nucleotide.

The RNAi constructs may also comprise one or more modified internucleotide linkages. As used herein, the term “modified internucleotide linkage” refers to an internucleotide linkage other than the natural 3′ to 5′ phosphodiester linkage. In some embodiments, the modified internucleotide linkage is a phosphorous-containing internucleotide linkage, such as a phosphotriester, aminoalkylphosphotriester, an alkylphosphonate (e.g., methylphosphonate, 3′-alkylene phosphonate), a phosphinate, a phosphoramidate (e.g., 3′-amino phosphoramidate and aminoalkylphosphoramidate), a phosphorothioate, a chiral phosphorothioate, a phosphorodithioate, a thionophosphoramidate, a thionoalkylphosphonate, a thionoalkylphosphotriester, and a boranophosphate. In one embodiment, a modified internucleotide linkage is a 2′ to 5′ phosphodiester linkage. In other embodiments, the modified internucleotide linkage is a non-phosphorous-containing internucleotide linkage and thus can be referred to as a modified internucleoside linkage. Such non-phosphorous-containing linkages include, but are not limited to, morpholino linkages (formed in part from the sugar portion of a nucleoside); siloxane linkages (—O—Si(H)₂—O—); sulfide, sulfoxide and sulfone linkages; formacetyl and thioformacetyl linkages; alkene containing backbones; sulfamate backbones; methylenemethylimino (—CH₂—N(CH₃)—O—CH₂—) and methylenehydrazino linkages; sulfonate and sulfonamide linkages; amide linkages; and others having mixed N, O, S and CH₂ component parts. In one embodiment, the modified internucleoside linkage is a peptide-based linkage (e.g., aminoethylglycine) to create a peptide nucleic acid or PNA, such as those described in U.S. Pat. Nos. 5,539,082; 5,714,331; and 5,719,262. Other suitable modified internucleotide and internucleoside linkages that may be employed in the RNAi constructs are described in U.S. Pat. Nos. 6,693,187, 9,181,551, U.S. Patent Publication No. 2016/0122761, and Deleavey and Damha, Chemistry and Biology, Vol. 19: 937-954, 2012, all of which are hereby incorporated by reference in their entireties.

In certain embodiments, the RNAi constructs comprise one or more phosphorothioate internucleotide linkages. The phosphorothioate internucleotide linkages may be present in the sense strand, antisense strand, or both strands of the RNAi constructs. For instance, in some embodiments, the sense strand comprises 1, 2, 3, 4, 5, 6, 7, 8, or more phosphorothioate internucleotide linkages. In other embodiments, the antisense strand comprises 1, 2, 3, 4, 5, 6, 7, 8, or more phosphorothioate internucleotide linkages. In still other embodiments, both strands comprise 1, 2, 3, 4, 5, 6, 7, 8, or more phosphorothioate internucleotide linkages. The RNAi constructs can comprise one or more phosphorothioate internucleotide linkages at the 3′-end, the 5′-end, or both the 3′- and 5′-ends of the sense strand, the antisense strand, or both strands. For instance, in certain embodiments, the RNAi construct comprises about 1 to about 6 or more (e.g., about 1, 2, 3, 4, 5, 6 or more) consecutive phosphorothioate internucleotide linkages at the 3′-end of the sense strand, the antisense strand, or both strands. In other embodiments, the RNAi construct comprises about 1 to about 6 or more (e.g., about 1, 2, 3, 4, 5, 6 or more) consecutive phosphorothioate internucleotide linkages at the 5′-end of the sense strand, the antisense strand, or both strands. In some embodiments, the antisense strand comprises at least 1 but no more than 6 phosphorothioate internucleotide linkages and the sense strand comprises at least 1 but no more than 4 phosphorothioate internucleotide linkages. In other embodiments, the antisense strand comprises at least 1 but no more than 4 phosphorothioate internucleotide linkages and the sense strand comprises at least 1 but no more than 2 phosphorothioate internucleotide linkages.

In some embodiments, the RNAi construct comprises a single phosphorothioate internucleotide linkage between the terminal nucleotides at the 3′ end of the sense strand. In other embodiments, the RNAi construct comprises two consecutive phosphorothioate internucleotide linkages between the terminal nucleotides at the 3′ end of the sense strand. In one embodiment, the RNAi construct comprises a single phosphorothioate internucleotide linkage between the terminal nucleotides at the 3′ end of the sense strand and a single phosphorothioate internucleotide linkage between the terminal nucleotides at the 3′ end of the antisense strand. In another embodiment, the RNAi construct comprises two consecutive phosphorothioate internucleotide linkages between the terminal nucleotides at the 3′ end of the antisense strand (i.e., a phosphorothioate internucleotide linkage at the first and second internucleotide linkages at the 3′ end of the antisense strand). In another embodiment, the RNAi construct comprises two consecutive phosphorothioate internucleotide linkages between the terminal nucleotides at both the 3′ and 5′ ends of the antisense strand. In yet another embodiment, the RNAi construct comprises two consecutive phosphorothioate internucleotide linkages between the terminal nucleotides at both the 3′ and 5′ ends of the antisense strand and two consecutive phosphorothioate internucleotide linkages at the 5′ end of the sense strand. In still another embodiment, the RNAi construct comprises two consecutive phosphorothioate internucleotide linkages between the terminal nucleotides at both the 3′ and 5′ ends of the antisense strand and two consecutive phosphorothioate internucleotide linkages between the terminal nucleotides at the 3′ end of the sense strand. In another embodiment, the RNAi construct comprises two consecutive phosphorothioate internucleotide linkages between the terminal nucleotides at both the 3′ and 5′ ends of the antisense strand and two consecutive phosphorothioate internucleotide linkages between the terminal nucleotides at both the 3′ and 5′ ends of the sense strand (i.e., a phosphorothioate internucleotide linkage at the first and second internucleotide linkages at both the 5′ and 3′ ends of the antisense strand and a phosphorothioate internucleotide linkage at the first and second internucleotide linkages at both the 5′ and 3′ ends of the sense strand). In yet another embodiment, the RNAi construct comprises two consecutive phosphorothioate internucleotide linkages between the terminal nucleotides at both the 3′ and 5′ ends of the antisense strand and a single phosphorothioate internucleotide linkage between the terminal nucleotides at the 3′ end of the sense strand. In any of the embodiments in which one or both strands comprise one or more phosphorothioate internucleotide linkages, the remaining internucleotide linkages within the strands can be the natural 3′ to 5′ phosphodiester linkages. For instance, in some embodiments, each internucleotide linkage of the sense and antisense strands is selected from phosphodiester and phosphorothioate, wherein at least one internucleotide linkage is a phosphorothioate.

In embodiments in which the RNAi construct comprises a nucleotide overhang, two or more of the unpaired nucleotides in the overhang can be connected by a phosphorothioate internucleotide linkage. In certain embodiments, all the unpaired nucleotides in a nucleotide overhang at the 3′ end of the antisense strand and/or the sense strand are connected by phosphorothioate internucleotide linkages. In other embodiments, all the unpaired nucleotides in a nucleotide overhang at the 5′ end of the antisense strand and/or the sense strand are connected by phosphorothioate internucleotide linkages. In still other embodiments, all the unpaired nucleotides in any nucleotide overhang are connected by phosphorothioate internucleotide linkages.

Incorporation of a phosphorothioate internucleotide linkage introduces an additional chiral center at the phosphorous atom in the oligonucleotide and therefore creates a diastereomer pair (Rp and Sp) at each phosphorothioate internucleotide linkage. Diastereomers or diastereoisomers are different configurations of a compound that have the same molecular formula and sequence of bonded atoms but differ in the three-dimensional orientations of their atoms in space. Unlike enantiomers, diastereomers are not mirror-images of each other. Each chiral phosphate atom can be in the “R” configuration (Rp) or the “S” configuration (Sp). In certain embodiments, the RNAi constructs may comprise one or more phosphorothioate internucleotide linkages where the chiral phosphates are selected to be primarily in either the Rp or Sp configuration. For instance, in some embodiments in which the RNAi constructs have one or more phosphorothioate internucleotide linkages, at least about 70%, at least about 75%, at least about 80%, at least about 85%, at least about 90%, or at least about 95% of the chiral phosphates are in the Sp configuration. In other embodiments in which the RNAi constructs have one or more phosphorothioate internucleotide linkages, at least about 70%, at least about 75%, at least about 80%, at least about 85%, at least about 90%, or at least about 95% of the chiral phosphates are in the Rp configuration. All the chiral phosphates in the RNAi construct can be either in the Sp configuration or the Rp configuration (i.e., the RNAi construct is stereopure). In some embodiments, all the chiral phosphates in the RNAi construct are in the Sp configuration. In some embodiments, all the chiral phosphates in the RNAi construct are in the Rp configuration.

In certain embodiments, the chiral phosphates in the RNAi construct may have different configurations at different positions in the sense strand or antisense strand. In one such embodiment in which the RNAi construct comprises one or two phosphorothioate internucleotide linkages at the 5′ end of the antisense strand, the chiral phosphates at the 5′ end of the antisense strand may be in the Rp configuration. In another such embodiment in which the RNAi construct comprises one or two phosphorothioate internucleotide linkages at the 3′ end of the antisense strand, the chiral phosphates at the 3′ end of the antisense strand may be in the Sp configuration. In certain embodiments, the RNAi construct comprises two consecutive phosphorothioate internucleotide linkages between the terminal nucleotides at both the 3′ and 5′ ends of the antisense strand and two consecutive phosphorothioate internucleotide linkages between the terminal nucleotides at the 3′ end of the sense strand, wherein the chiral phosphates at the 5′ end of the antisense strand are in the Rp configuration, the chiral phosphates at the 3′ end of the antisense strand are in the Sp configuration, and the chiral phosphates at the 3′ end of the sense strand can be either in the Rp or Sp configuration. In certain other embodiments, the RNAi construct comprises two consecutive phosphorothioate internucleotide linkages between the terminal nucleotides at both the 3′ and 5′ ends of the antisense strand and a single phosphorothioate internucleotide linkage between the terminal nucleotides at the 3′ end of the sense strand, wherein the chiral phosphates at the 5′ end of the antisense strand are in the Rp configuration, the chiral phosphates at the 3′ end of the antisense strand are in the Sp configuration, and the chiral phosphate at the 3′ end of the sense strand can be either in the Rp or Sp configuration. Methods of controlling the stereochemistry of phosphorothioate linkages during oligonucleotide synthesis are known to those skilled in the art and can include methods described in Nawrot and Rebowska, Curr. Protoc. Nucleic Acid Chem. 2009, Chapter 4: doi:10.1002/0471142700.nc0434s362009; Jahns et al., Nat. Commun., Vol. 6: 6317, 2015; Knouse et al., Science, Vol. 361: 1234-1238, 2018; and Sakamuri et al., ChemBioChem, Vol. 21(9): 1304-1308, 2020.

In some embodiments of the RNAi constructs, the 5′ end of the sense strand, antisense strand, or both the antisense and sense strands comprises a phosphate moiety. As used herein, the term “phosphate moiety” refers to a terminal phosphate group that includes unmodified phosphates (—O—P═O)(OH)OH) as well as modified phosphates. Modified phosphates include phosphates in which one or more of the O and OH groups are replaced with H, O, S, N(R) or alkyl (e.g., C₁ to C₁₂) where R is H, an amino protecting group or unsubstituted or substituted alkyl (e.g., C₁ to C₁₂). Exemplary phosphate moieties include, but are not limited to, 5′-monophosphate; 5′-diphosphate; 5′-triphosphate; 5′-guanosine cap (7-methylated or non-methylated); 5′-adenosine cap or any other modified or unmodified nucleotide cap structure; 5′-monothiophosphate (phosphorothioate); 5′-monodithiophosphate (phosphorodithioate); 5′-alpha-thiotriphosphate; 5′-gamma-thiotriphosphate, 5′-phosphoramidates; 5′-vinylphosphates; 5′-alkylphosphonates (e.g., alkyl=methyl, ethyl, isopropyl, propyl, etc.); and 5′-alkyletherphosphonates (e.g., alkylether=methoxymethyl, ethoxymethyl, etc.).

The modified nucleotides that can be incorporated into the RNAi constructs may have more than one chemical modification described herein. For instance, the modified nucleotide may have a modification to the ribose sugar as well as a modification to the nucleobase. By way of example, a modified nucleotide may comprise a 2′ sugar modification (e.g., 2′-fluoro or 2′-O-methyl) and comprise a modified base (e.g., 5-methyl cytosine or pseudouracil). In other embodiments, the modified nucleotide may comprise a sugar modification in combination with a modification to the 5′ phosphate that would create a modified internucleotide or internucleotide linkage when the modified nucleotide was incorporated into a polynucleotide. For instance, in some embodiments, the modified nucleotide may comprise a sugar modification, such as a 2′-fluoro modification, a 2′-O-methyl modification, or a bicyclic sugar modification, as well as a 5′ phosphorothioate group. Accordingly, in some embodiments, one or both strands of the RNAi constructs comprise a combination of 2′ modified nucleotides or BNAs and phosphorothioate internucleotide linkages. In certain embodiments, both the sense and antisense strands of the RNAi constructs comprise a combination of 2′-fluoro modified nucleotides, 2′-O-methyl modified nucleotides, and phosphorothioate internucleotide linkages. Exemplary RNAi constructs comprising modified nucleotides and internucleotide linkages are shown in Table 2.

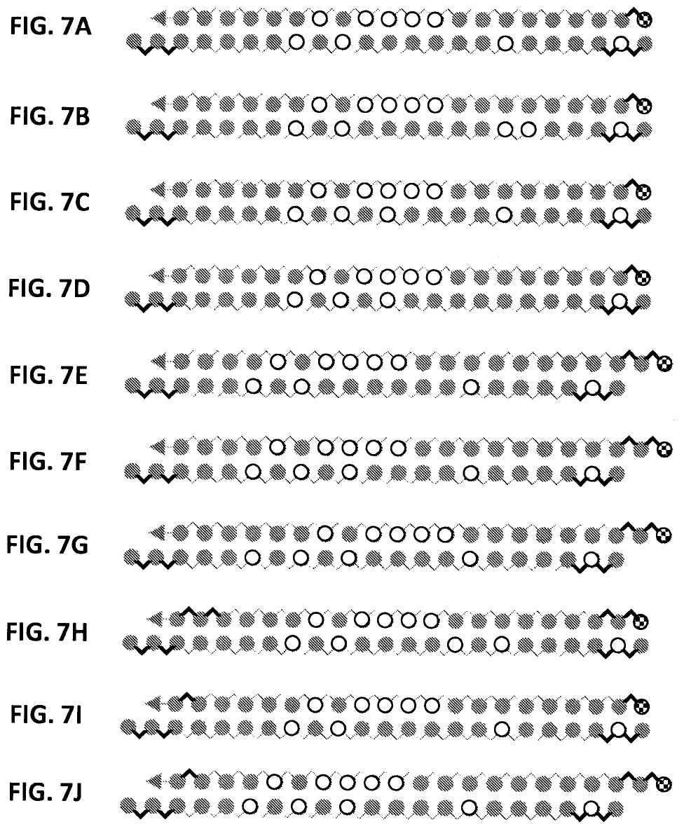

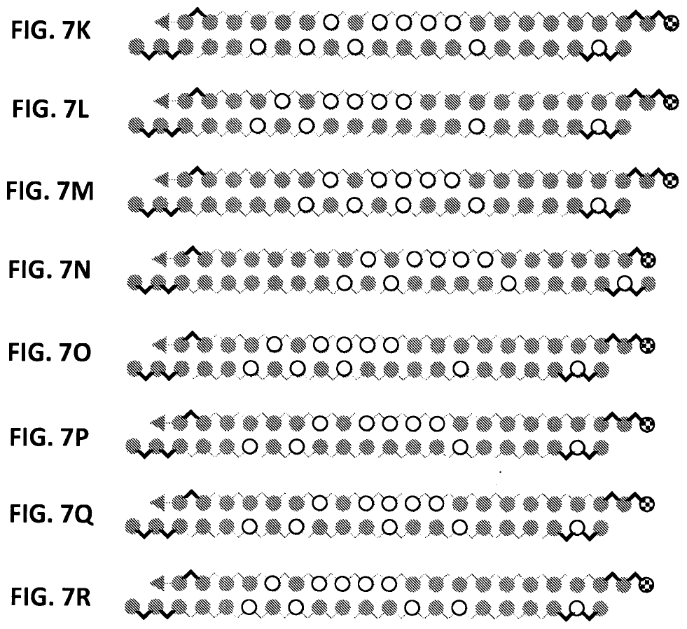

Exemplary modification patterns for RNAi constructs are shown in FIGS. 7A-7R. These patterns may be used in the context of the RNAi duplexes disclosed herein, or in the context of RNAi constructs in general. FIGS. 7A-7R each show a hybridized sense (top) and antisense (bottom) strand, in which each of the nucleotides is modified. The solid circles in FIGS. 7A-7R correspond to 2′-O-methyl ribonucleotides, while the open circles correspond to 2′-deoxy-2′-fluoro (“2′-fluoro”) ribonucleotides. The hatched circles correspond to inverted abasic deoxynucleotides. Bold lines indicate where a phosphorothioate bond is used in place of the standard phosphodiester bond between nucleotides. Finally, arrows represent where a ligand (e.g., GalNAc or a fatty acid such as C22) may be attached to the RNAi construct. As demonstrated in the Examples below, these modification patterns are effective across a range of different trigger sequences in the FAM13A sequence, indicating that they are generally applicable to RNAi constructs.

The RNAi constructs can readily be made using techniques known in the art, for example, using conventional nucleic acid solid phase synthesis. The polynucleotides of the RNAi constructs can be assembled on a suitable nucleic acid synthesizer utilizing standard nucleotide or nucleoside precursors (e.g., phosphoramidites). Automated nucleic acid synthesizers are sold commercially by several vendors, including DNA/RNA synthesizers from Applied Biosystems (Foster City, CA), MerMade synthesizers from BioAutomation (Irving, TX), and OligoPilot synthesizers from GE Healthcare Life Sciences (Pittsburgh, PA). An exemplary method for synthesizing the RNAi constructs is described in Example 3.

A 2′ silyl protecting group can be used in conjunction with acid labile dimethoxytrityl (DMT) at the 5′ position of ribonucleosides to synthesize oligonucleotides via phosphoramidite chemistry. Final deprotection conditions are known not to significantly degrade RNA products. All syntheses can be conducted in any automated or manual synthesizer on large, medium, or small scale. The syntheses may also be carried out in multiple well plates, columns, or glass slides.

The 2′-O-silyl group can be removed via exposure to fluoride ions, which can include any source of fluoride ion, e.g., those salts containing fluoride ion paired with inorganic counterions e.g., cesium fluoride and potassium fluoride or those salts containing fluoride ion paired with an organic counterion, e.g., a tetraalkylammonium fluoride. A crown ether catalyst can be utilized in combination with the inorganic fluoride in the deprotection reaction. Exemplary fluoride ion sources are tetrabutylammonium fluoride or aminohydrofluorides (e.g., combining aqueous HF with triethylamine in a dipolar aprotic solvent, e.g., dimethylformamide).

The choice of protecting groups for use on the phosphite triesters and phosphotriesters can alter the stability of the triesters towards fluoride. Methyl protection of the phosphotriester or phosphite triester can stabilize the linkage against fluoride ions and improve process yields.

Since ribonucleosides have a reactive 2′ hydroxyl substituent, it can be desirable to protect the reactive 2′ position in RNA with a protecting group that is orthogonal to a 5′-O-dimethoxytrityl protecting group, e.g., one stable to treatment with acid. Silyl protecting groups meet this criterion and can be readily removed in a final fluoride deprotection step that can result in minimal RNA degradation.

Tetrazole catalysts can be used in the standard phosphoramidite coupling reaction. Exemplary catalysts include, e.g., tetrazole, S-ethyl-tetrazole, benzylthiotetrazole, p-nitrophenyltetrazole.

As can be appreciated by the skilled artisan, further methods of synthesizing the RNAi constructs described herein will be evident to those of ordinary skill in the art. Additionally, the various synthetic steps may be performed in an alternate sequence or order to give the desired compounds. Other synthetic chemistry transformations, protecting groups (e.g., for hydroxyl, amino, etc. present on the bases) and protecting group methodologies (protection and deprotection) useful in synthesizing the RNAi constructs described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. Custom synthesis of RNAi constructs is also available from several commercial vendors, including Dharmacon, Inc. (Lafayette, CO), AxoLabs GmbH (Kulmbach, Germany), and Ambion, Inc. (Foster City, CA).

The RNAi constructs may comprise a ligand. As used herein, a “ligand” refers to any compound or molecule that is capable of interacting with another compound or molecule, directly or indirectly. The interaction of a ligand with another compound or molecule may elicit a biological response (e.g., initiate a signal transduction cascade, induce receptor-mediated endocytosis) or may just be a physical association. The ligand can modify one or more properties of the double-stranded RNA molecule to which is attached, such as the pharmacodynamic, pharmacokinetic, binding, absorption, cellular distribution, cellular uptake, charge and/or clearance properties of the RNA molecule.

The ligand may comprise a serum protein (e.g., human serum albumin, low-density lipoprotein, globulin), a cholesterol moiety, a vitamin (biotin, vitamin E, vitamin B12), a folate moiety, a steroid, a bile acid (e.g., cholic acid), a fatty acid (e.g., palmitic acid, myristic acid), a carbohydrate (e.g., a dextran, pullulan, chitin, chitosan, inulin, cyclodextrin or hyaluronic acid), a glycoside, a phospholipid, or antibody or binding fragment thereof (e.g., antibody or binding fragment that targets the RNAi construct to a specific cell type, such as liver). Other examples of ligands include dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases (e.g., EDTA), lipophilic molecules, e.g., adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, O3-(oleoyl)lithocholic acid, O3-(oleoyl)cholenic acid, dimethoxytrityl, or phenoxazine), peptides (e.g., antennapedia peptide, Tat peptide, RGD peptides), alkylating agents, polymers, such as polyethylene glycol (PEG)(e.g., PEG-40K), polyamino acids, and polyamines (e.g., spermine, spermidine).

In certain embodiments, the ligands have endosomolytic properties. The endosomolytic ligands promote the lysis of the endosome and/or transport of the RNAi construct, or its components, from the endosome to the cytoplasm of the cell. The endosomolytic ligand may be a polycationic peptide or peptidomimetic, which shows pH-dependent membrane activity and fusogenicity. In one embodiment, the endosomolytic ligand assumes its active conformation at endosomal pH. The “active” conformation is that conformation in which the endosomolytic ligand promotes lysis of the endosome and/or transport of the RNAi construct, or its components, from the endosome to the cytoplasm of the cell. Exemplary endosomolytic ligands include the GALA peptide (Subbarao et al., Biochemistry, Vol. 26: 2964-2972, 1987), the EALA peptide (Vogel et al., J. Am. Chem. Soc., Vol. 118: 1581-1586, 1996), and their derivatives (Turk et al., Biochem. Biophys. Acta, Vol. 1559: 56-68, 2002). In one embodiment, the endosomolytic component may contain a chemical group (e.g., an amino acid) which will undergo a change in charge or protonation in response to a change in pH. The endosomolytic component may be linear or branched.

In some embodiments, the ligand comprises a lipid or other hydrophobic molecule. In one embodiment, the ligand comprises a cholesterol moiety or other steroid. Cholesterol-conjugated oligonucleotides have been reported to be more active than their unconjugated counterparts (Manoharan, Antisense Nucleic Acid Drug Development, Vol. 12: 103-228, 2002). Ligands comprising cholesterol moieties and other lipids for conjugation to nucleic acid molecules have also been described in U.S. Pat. Nos. 7,851,615; 7,745,608; and 7,833,992, all of which are hereby incorporated by reference in their entireties. In another embodiment, the ligand comprises a folate moiety. Polynucleotides conjugated to folate moieties can be taken up by cells via a receptor-mediated endocytosis pathway. Such folate-polynucleotide conjugates are described in U.S. Pat. No. 8,188,247, which is hereby incorporated by reference in its entirety.

In certain embodiments, it is desirable to specifically deliver the RNAi constructs to liver cells to reduce expression of FAM13A protein specifically in the liver. Accordingly, in certain embodiments, the ligand targets delivery of the RNAi construct specifically to liver cells (e.g., hepatocytes) using various approaches as described in more detail below. In certain embodiments, the RNAi constructs are targeted to liver cells with a ligand that binds to the surface-expressed asialoglycoprotein receptor (ASGR) or component thereof (e.g., ASGR1, ASGR2).

In some embodiments, RNAi constructs can be specifically targeted to the liver by employing ligands that bind to or interact with proteins expressed on the surface of liver cells. For example, in certain embodiments, the ligands may comprise antigen binding proteins (e.g., antibodies or binding fragments thereof (e.g., Fab, scFv)) that specifically bind to a receptor expressed on hepatocytes, such as the asialoglycoprotein receptor and the LDL receptor. In some embodiments, the ligand comprises an antibody or binding fragment thereof that specifically binds to ASGR1 and/or ASGR2. In another embodiment, the ligand comprises a Fab fragment of an antibody that specifically binds to ASGR1 and/or ASGR2. A “Fab fragment” is comprised of one immunoglobulin light chain (i.e., light chain variable region (VL) and constant region (CL)) and the CH1 region and variable region (VH) of one immunoglobulin heavy chain. In another embodiment, the ligand comprises a single-chain variable antibody fragment (scFv fragment) of an antibody that specifically binds to ASGR1 and/or ASGR2. An “scFv fragment” comprises the VH and VL regions of an antibody, wherein these regions are present in a single polypeptide chain, and optionally comprising a peptide linker between the VH and VL regions that enables the FAT to form the desired structure for antigen binding. Exemplary antibodies and binding fragments thereof that specifically bind to ASGR1 that can be used as ligands for targeting the RNAi constructs to the liver are described in WIPO Publication No. WO 2017/058944, which is hereby incorporated by reference in its entirety. Other antibodies or binding fragments thereof that specifically bind to ASGR1, LDL receptor, or other liver surface-expressed proteins suitable for use as ligands in the RNAi constructs are commercially available.

In certain embodiments, it is desirable to specifically deliver the RNAi constructs to adipose tissue or adipose cells to reduce expression of FAM13A protein specifically in adipose cells. Accordingly, in certain embodiments, the ligand targets delivery of the RNAi construct specifically to adipose cells (e.g., subcutaneous white adipose tissue (scWAT) or epididymal white adipose tissue (eWAT)) using various approaches as described in more detail below. In certain embodiments, the RNAi constructs are targeted to adipose tissue or cells by conjugation to long-chain fatty acids, which are saturated or unsaturated fatty acids containing between 12 and 24 carbon atoms. In some embodiments, the long-chain fatty acid is lauric acid (C12), myristic acid (C14), palmitic acid (C16), stearic acid (C18), eicosapentaenoic acid (C20), docosanoic acid (C22), or docosahexanoic acid (C24).

In certain embodiments, it is desirable to deliver the RNAi constructs systemically to reduce expression of FAM13A protein in multiple or all cell types. Accordingly, in certain embodiments, the ligand targets delivery of the RNAi construct using methods known in the art to facilitate cellular delivery of siRNA (see, e.g., U.S. Pat. No. 10,633,653; WO 2022/016043, each of which is incorporated by reference in their entirety). In some embodiments, the RNAi constructs are targeted to cells by conjugation to cholesterol, α-tocopherol, or fatty acids. In some embodiments, the RNAi constructs are targeted to cells by conjugation to omega fatty acids. In certain embodiments, the RNAi constructs are targeted to cells by conjugation to long-chain fatty acids such as lauric acid (C12), myristic acid (C14), palmitic acid (C16), stearic acid (C18), eicosapentaenoic acid (C20), docosanoic acid (C22), or docosahexanoic acid (C24).

In certain embodiments, the ligand comprises a carbohydrate. A “carbohydrate” refers to a compound made up of one or more monosaccharide units having at least 6 carbon atoms (which can be linear, branched, or cyclic) with an oxygen, nitrogen or sulfur atom bonded to each carbon atom. Carbohydrates include, but are not limited to, the sugars (e.g., monosaccharides, disaccharides, trisaccharides, tetrasaccharides, and oligosaccharides containing from about 4, 5, 6, 7, 8, or 9 monosaccharide units), and polysaccharides, such as starches, glycogen, cellulose, and polysaccharide gums. In some embodiments, the carbohydrate incorporated into the ligand is a monosaccharide selected from a pentose, hexose, or heptose and di- and tri-saccharides including such monosaccharide units. In other embodiments, the carbohydrate incorporated into the ligand is an amino sugar, such as galactosamine, glucosamine, N-acetylgalactosamine, and N-acetylglucosamine.

In some embodiments, the ligand comprises a hexose or hexosamine. The hexose may be selected from glucose, galactose, mannose, fucose, or fructose. The hexosamine may be selected from fructosamine, galactosamine, glucosamine, or mannosamine. In certain embodiments, the ligand comprises glucose, galactose, galactosamine, or glucosamine. In one embodiment, the ligand comprises glucose, glucosamine, or N-acetylglucosamine. In another embodiment, the ligand comprises galactose, galactosamine, or N-acetyl-galactosamine. In particular embodiments, the ligand comprises N-acetyl-galactosamine. Ligands comprising glucose, galactose, and N-acetyl-galactosamine (GalNAc) are particularly effective in targeting compounds to liver cells because such ligands bind to the ASGR expressed on the surface of hepatocytes. See, e.g., D'Souza and Devarajan, J. Control Release, Vol. 203: 126-139, 2015. Examples of GalNAc- or galactose-containing ligands that can be incorporated into the RNAi constructs are described in U.S. Pat. Nos. 7,491,805; 8,106,022; and 8,877,917; U.S. Patent Publication No. 20030130186; and WIPO Publication No. WO 2013166155, all of which are hereby incorporated by reference in their entireties.

In certain embodiments, the ligand comprises a multivalent carbohydrate moiety. As used herein, a “multivalent carbohydrate moiety” refers to a moiety comprising two or more carbohydrate units capable of independently binding or interacting with other molecules. For example, a multivalent carbohydrate moiety comprises two or more binding domains comprised of carbohydrates that can bind to two or more different molecules or two or more different sites on the same molecule. The valency of the carbohydrate moiety denotes the number of individual binding domains within the carbohydrate moiety. For instance, the terms “monovalent,” “bivalent,” “trivalent,” and “tetravalent” with reference to the carbohydrate moiety refer to carbohydrate moieties with one, two, three, and four binding domains, respectively. The multivalent carbohydrate moiety may comprise a multivalent lactose moiety, a multivalent galactose moiety, a multivalent glucose moiety, a multivalent N-acetyl-galactosamine moiety, a multivalent N-acetyl-glucosamine moiety, a multivalent mannose moiety, or a multivalent fucose moiety. In some embodiments, the ligand comprises a multivalent galactose moiety. In other embodiments, the ligand comprises a multivalent N-acetyl-galactosamine moiety. In these and other embodiments, the multivalent carbohydrate moiety can be bivalent, trivalent, or tetravalent. In such embodiments, the multivalent carbohydrate moiety can be bi-antennary or tri-antennary. In some embodiments, the multivalent N-acetyl-galactosamine moiety is trivalent or tetravalent. In some embodiments, the multivalent galactose moiety is trivalent or tetravalent. Exemplary trivalent and tetravalent GalNAc-containing ligands for incorporation into the RNAi constructs are described in detail below.

The ligand can be attached or conjugated to the RNA molecule of the RNAi construct directly or indirectly. For instance, in some embodiments, the ligand is covalently attached directly to the sense or antisense strand of the RNAi construct. In other embodiments, the ligand is covalently attached via a linker to the sense or antisense strand of the RNAi construct. The ligand can be attached to nucleobases, sugar moieties, or internucleotide linkages of polynucleotides (e.g., sense strand or antisense strand) of the RNAi constructs. Conjugation or attachment to purine nucleobases or derivatives thereof can occur at any position including, endocyclic and exocyclic atoms. In certain embodiments, the 2-, 6-, 7-, or 8-positions of a purine nucleobase are attached to a ligand. Conjugation or attachment to pyrimidine nucleobases or derivatives thereof can also occur at any position. In some embodiments, the 2-, 5-, and 6-positions of a pyrimidine nucleobase can be attached to a ligand. Conjugation or attachment to sugar moieties of nucleotides can occur at any carbon atom. Exemplary carbon atoms of a sugar moiety that can be attached to a ligand include the 2′, 3′, and 5′ carbon atoms. The 1′ position can also be attached to a ligand, such as in an abasic nucleotide. Internucleotide linkages can also support ligand attachments. For phosphorus-containing linkages (e.g., phosphodiester, phosphorothioate, phosphorodithioate, phosphoroamidate, and the like), the ligand can be attached directly to the phosphorus atom or to an O, N, or S atom bound to the phosphorus atom. For amine- or amide-containing internucleotide linkages (e.g., PNA), the ligand can be attached to the nitrogen atom of the amine or amide or to an adjacent carbon atom.

In some embodiments, the ligand may be attached to the 3′ or 5′ end of either the sense or antisense strand. In certain embodiments, the ligand is covalently attached to the 5′ end of the sense strand. In such embodiments, the ligand is attached to the 5′-terminal nucleotide of the sense strand. In these and other embodiments, the ligand is attached at the 5′-position of the 5′-terminal nucleotide of the sense strand. In embodiments in which an inverted abasic nucleotide is the 5′-terminal nucleotide of the sense strand and linked to the adjacent nucleotide via a 5′-5′ internucleotide linkage, the ligand can be attached at the 3′-position of the inverted abasic nucleotide. In other embodiments, the ligand is covalently attached to the 3′ end of the sense strand. For example, in some embodiments, the ligand is attached to the 3′-terminal nucleotide of the sense strand. In certain such embodiments, the ligand is attached at the 3′-position of the 3′-terminal nucleotide of the sense strand. In embodiments in which an inverted abasic nucleotide is the 3′-terminal nucleotide of the sense strand and linked to the adjacent nucleotide via a 3′-3′ internucleotide linkage, the ligand can be attached at the 5′-position of the inverted abasic nucleotide. In alternative embodiments, the ligand is attached near the 3′ end of the sense strand, but before one or more terminal nucleotides (i.e., before 1, 2, 3, or 4 terminal nucleotides). In some embodiments, the ligand is attached at the 2′-position of the sugar of the 3′-terminal nucleotide of the sense strand. In other embodiments, the ligand is attached at the 2′-position of the sugar of the 5′-terminal nucleotide of the sense strand.

In certain embodiments, the ligand is attached to the sense or antisense strand via a linker. A “linker” is an atom or group of atoms that covalently joins a ligand to a polynucleotide component of the RNAi construct. The linker may be from about 1 to about 30 atoms in length, from about 2 to about 28 atoms in length, from about 3 to about 26 atoms in length, from about 4 to about 24 atoms in length, from about 6 to about 20 atoms in length, from about 7 to about 20 atoms in length, from about 8 to about 20 atoms in length, from about 8 to about 18 atoms in length, from about 10 to about 18 atoms in length, and from about 12 to about 18 atoms in length. In some embodiments, the linker may comprise a bifunctional linking moiety, which generally comprises an alkyl moiety with two functional groups. One of the functional groups is selected to bind to the compound of interest (e.g., sense or antisense strand of the RNAi construct) and the other is selected to bind essentially any selected group, such as a ligand as described herein. In certain embodiments, the linker comprises a chain structure or an oligomer of repeating units, such as ethylene glycol or amino acid units. Examples of functional groups that are typically employed in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like.

Linkers that may be used to attach a ligand to the sense or antisense strand in the RNAi constructs include, but are not limited to, pyrrolidine, 8-amino-3,6-dioxaoctanoic acid, succinimidyl 4-(N-maleimidomethyl)cyclohexane-1-carboxylate, 6-aminohexanoic acid, substituted C₁-C₁₀ alkyl, substituted or unsubstituted C₂-C₁₀ alkenyl or substituted or unsubstituted C₂-C₁₀ alkynyl. Suitable substituent groups for such linkers include, but are not limited to, hydroxyl, amino, alkoxy, carboxy, benzyl, phenyl, nitro, thiol, thioalkoxy, halogen, alkyl, aryl, alkenyl and alkynyl.

In certain embodiments, the linkers are cleavable. A cleavable linker is one which is sufficiently stable outside the cell, but which upon entry into a target cell is cleaved to release the two parts the linker is holding together. In some embodiments, the cleavable linker is cleaved at least 10 times, 20 times, 30 times, 40 times, 50 times, 60 times, 70 times, 80 times, 90 times, or more, or at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linkers are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linker by reduction; esterases; endosomes or agents that can create an acidic environment, e.g., those that result in a pH of five or lower; enzymes that can hydrolyze or degrade an acid cleavable linker by acting as a general acid, peptidases (which can be substrate specific), and phosphatases.

A cleavable linker may comprise a moiety that is susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable group that is cleaved at a preferred pH, thereby releasing the RNA molecule from the ligand inside the cell, or into the desired compartment of the cell.

A linker can include a cleavable group that is cleavable by a particular enzyme. The type of cleavable group incorporated into a linker can depend on the cell to be targeted. For example, liver-targeting ligands can be linked to RNA molecules through a linker that includes an ester group. Liver cells are rich in esterases, and therefore the linker will be cleaved more efficiently in liver cells than in cell types that are not esterase rich. Other types of cells rich in esterases include cells of the lung, renal cortex, and testis. Linkers that contain peptide bonds can be used when targeting cells rich in peptidases, such as liver cells and synoviocytes.

In general, the suitability of a candidate cleavable linker can be evaluated by testing the ability of a degradative agent (or condition) to cleave the candidate linker. It will also be desirable to also test the candidate cleavable linker for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In some embodiments, useful candidate linkers are cleaved at least 2, 4, 10, 20, 50, 70, or 100 times faster in the cell (or under in vitro conditions selected to mimic intracellular conditions) as compared to blood or serum (or under in vitro conditions selected to mimic extracellular conditions).

In other embodiments, redox cleavable linkers are utilized. Redox cleavable linkers are cleaved upon reduction or oxidation. An example of a reductively cleavable group is a disulfide linking group (—S—S—). To determine if a candidate cleavable linker is a suitable “reductively cleavable linker,” or for example is suitable for use with a particular RNAi construct and particular ligand, one can use one or more methods described herein. For example, a candidate linker can be evaluated by incubation with dithiothreitol (DTT), or other reducing agent known in the art, which mimics the rate of cleavage that would be observed in a cell, e.g., a target cell. The candidate linkers can also be evaluated under conditions which are selected to mimic blood or serum conditions. In a specific embodiment, candidate linkers are cleaved by at most 10% in the blood. In other embodiments, useful candidate linkers are degraded at least 2, 4, 10, 20, 50, 70, or 100 times faster in the cell (or under in vitro conditions selected to mimic intracellular conditions) as compared to blood (or under in vitro conditions selected to mimic extracellular conditions).

In yet other embodiments, phosphate-based cleavable linkers, which are cleaved by agents that degrade or hydrolyze the phosphate group, are employed to covalently attach a ligand to the sense or antisense strand of the RNAi construct. An example of an agent that hydrolyzes phosphate groups in cells are enzymes, such as phosphatases in cells. Examples of phosphate-based cleavable groups are —O—P(O)(ORk)-O—, —O—P(S)(ORk)-O—, —O—P(S)(SRk)-O—, —S—P(O) (ORk)O—, —O—P(O)(ORk)-S—, —S—P(O)(ORk)-S—, —O—P(S)(ORk)-S—, —S—P(S)(ORk)O—, —O—P(O)(Rk)O—, —O—P(S)(Rk)-O—, —S—P(O)(Rk)-O—, —S—P(S)(Rk)O—, —S—P(O)(Rk)-S—, and —O—P(S)(Rk)-S—, where Rk can be hydrogen or C₁-C₁₀ alkyl. Specific embodiments include —O—P(O)(OH)O—, —O—P(S)(OH)O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—. Another specific embodiment is —O—P(O)(OH)—O—. These candidate linkers can be evaluated using methods analogous to those described above.

In other embodiments, the linkers may comprise acid cleavable groups, which are groups that are cleaved under acidic conditions. In some embodiments, acid cleavable groups are cleaved in an acidic environment with a pH of about 6.5 or lower (e.g., about 6.0, 5.5, 5.0, or lower), or by agents, such as enzymes that can act as a general acid. In a cell, specific low pH organelles, such as endosomes and lysosomes, can provide a cleaving environment for acid cleavable groups. Examples of acid cleavable linking groups include, but are not limited to, hydrazones, esters, and esters of amino acids. Acid cleavable groups can have the general formula —C═NN—, C(O)O, or —OC(O). A specific embodiment is when the carbon attached to the oxygen of the ester (the alkoxy group) is an aryl group, substituted alkyl group, or tertiary alkyl group such as dimethyl, pentyl or t-butyl. These candidates can be evaluated using methods analogous to those described above.

In other embodiments, the linkers may comprise ester-based cleavable groups, which are cleaved by enzymes, such as esterases and amidases in cells. Examples of ester-based cleavable groups include, but are not limited to, esters of alkylene, alkenylene and alkynylene groups. Ester cleavable groups have the general formula —C(O)O—, or —OC(O)—. These candidate linkers can be evaluated using methods analogous to those described above.

In further embodiments, the linkers may comprise peptide-based cleavable groups, which are cleaved by enzymes, such as peptidases and proteases in cells. Peptide-based cleavable groups are peptide bonds formed between amino acids to yield oligopeptides (e.g., dipeptides, tripeptides) and polypeptides. Peptide-based cleavable groups include the amide group (—C(O)NH—). The amide group can be formed between any alkylene, alkenylene or alkynylene. A peptide bond is a special type of amide bond formed between amino acids to yield peptides and proteins. The peptide-based cleavage group is generally limited to the peptide bond (i.e., the amide bond) formed between amino acids yielding peptides and proteins. Peptide-based cleavable linking groups have the general formula —NHCHR^{A}C(O)NHCHR^{B}C(O)—, where R^{A} and R^{B} are the side chains of the two adjacent amino acids. These candidates can be evaluated using methods analogous to those described above.

Other types of linkers suitable for attaching ligands to the sense or antisense strands in the RNAi constructs are known in the art and can include the linkers described in U.S. Pat. Nos. 7,723,509; 8,017,762; 8,828,956; 8,877,917; and 9,181,551, all of which are hereby incorporated by reference in their entireties.

In certain embodiments, the ligand covalently attached to the sense or antisense strand of the RNAi constructs comprises a GalNAc moiety, e.g., a multivalent GalNAc moiety. In some embodiments, the multivalent GalNAc moiety is a trivalent GalNAc moiety and is attached to the 3′ end of the sense strand. In other embodiments, the multivalent GalNAc moiety is a trivalent GalNAc moiety and is attached to the 5′ end of the sense strand. In yet other embodiments, the multivalent GalNAc moiety is a tetravalent GalNAc moiety and is attached to the 3′ end of the sense strand. In still other embodiments, the multivalent GalNAc moiety is a tetravalent GalNAc moiety and is attached to the 5′ end of the sense strand.

In certain embodiments, the RNAi constructs comprise a ligand having the following structure ([Structure 1]):

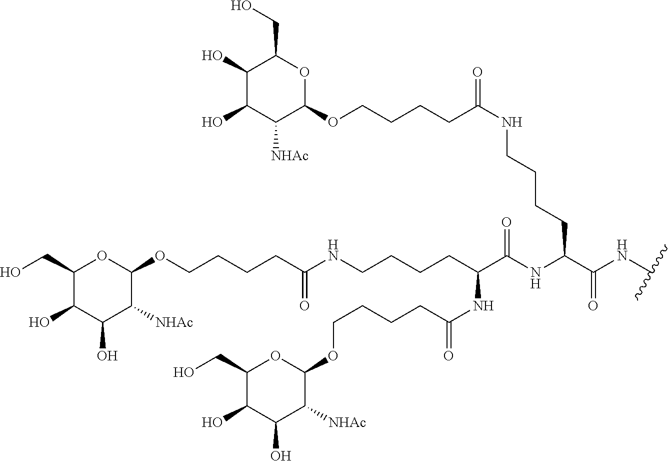 [SMILES: *NC(=O)[C@H](CCCCNC(=O)CCCCO[C@@H]1O[C@H](CO)[C@H](O)[C@H](O)[C@H]1NC(C)=O)NC(=O)[C@H](CCCCNC(=O)CCCCO[C@@H]1O[C@H](CO)[C@H](O)[C@H](O)[C@H]1NC(C)=O)NC(=O)CCCCO[C@@H]1O[C@H](CO)[C@H](O)[C@H](O)[C@H]1NC(C)=O]

In preferred embodiments, the ligand having this structure is covalently attached to the 5′ end of the sense strand (e.g., to the 5′ terminal nucleotide of the sense strand) via a linker, such as the linkers described herein. In one embodiment, the linker is an aminohexyl linker.

Exemplary trivalent and tetravalent GalNAc moieties and linkers that can be attached to the double-stranded RNA molecules in the RNAi constructs are provided in the structural formulas I-IX below. “Ac” in the formulas listed herein represents an acetyl group.

In one embodiment, the RNAi construct comprises a ligand and linker having the following structure of Formula I, wherein each n is independently 1 to 3, k is 1 to 3, m is 1 or 2, j is 1 or 2, and the ligand is attached to the 3′ end of the sense strand of the double-stranded RNA molecule (represented by the solid wavy line):

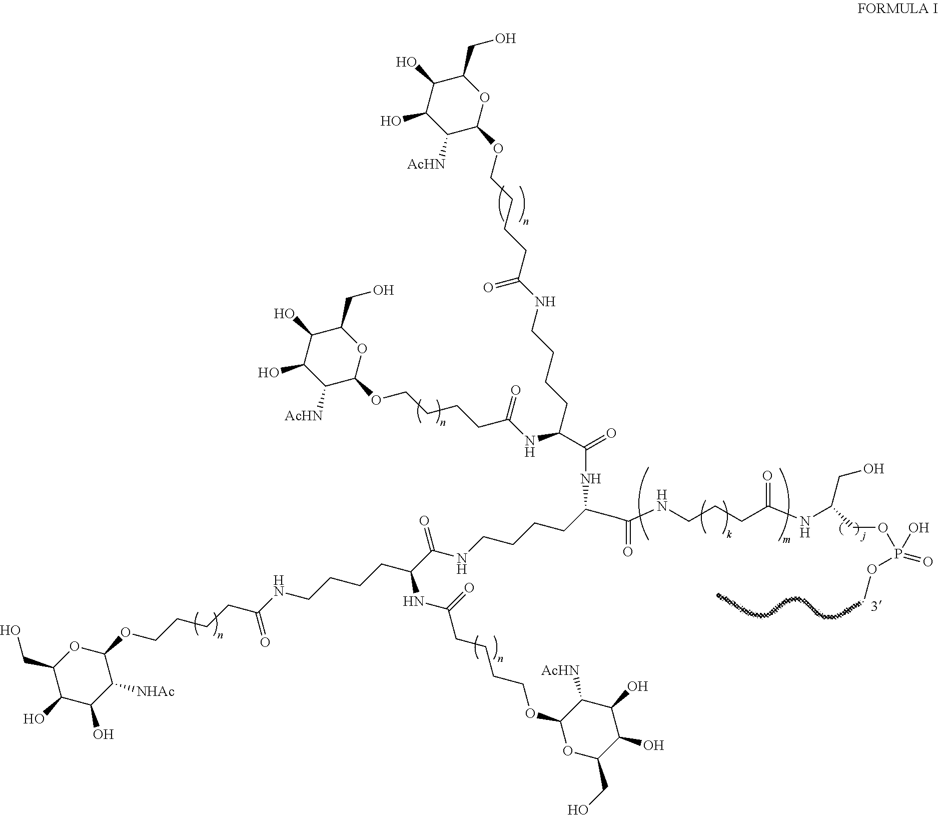

In another embodiment, the RNAi construct comprises a ligand and linker having the following structure of Formula II, wherein each n is independently 1 to 3, k is 1 to 3, m is 1 or 2, j is 1 or 2, and the ligand is attached to the 3′ end of the sense strand of the double-stranded RNA molecule (represented by the solid wavy line):

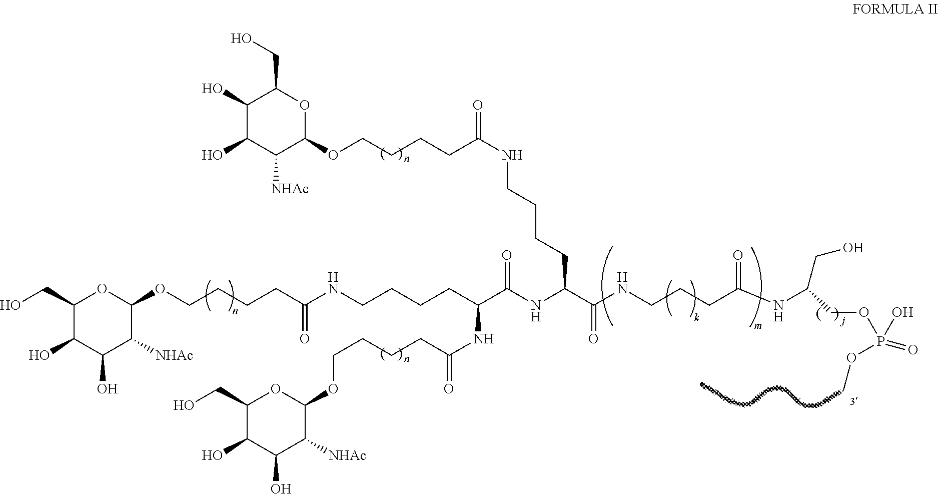

In yet another embodiment, the RNAi construct comprises a ligand and linker having the following structure of Formula III, wherein the ligand is attached to the 3′ end of the sense strand of the double-stranded RNA molecule (represented by the solid wavy line):

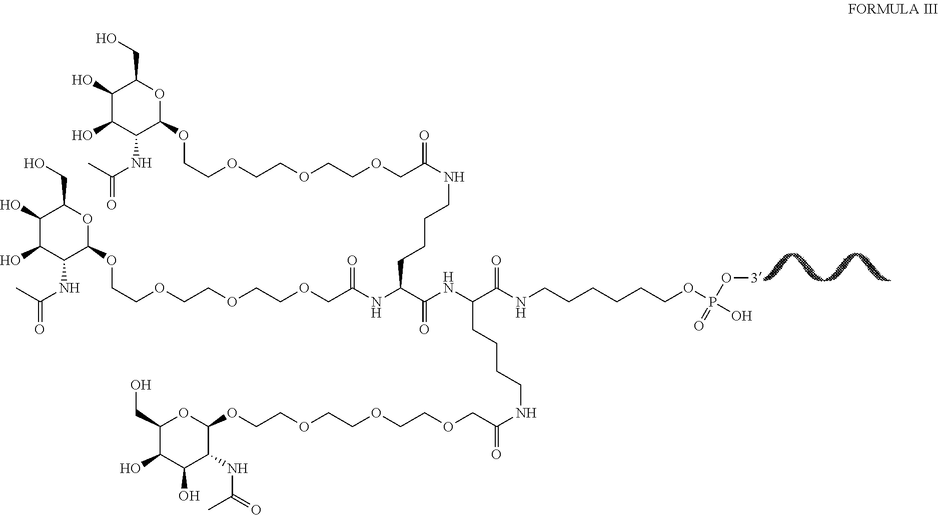

In still another embodiment, the RNAi construct comprises a ligand and linker having the following structure of Formula IV, wherein the ligand is attached to the 3′ end of the sense strand of the double-stranded RNA molecule (represented by the solid wavy line):

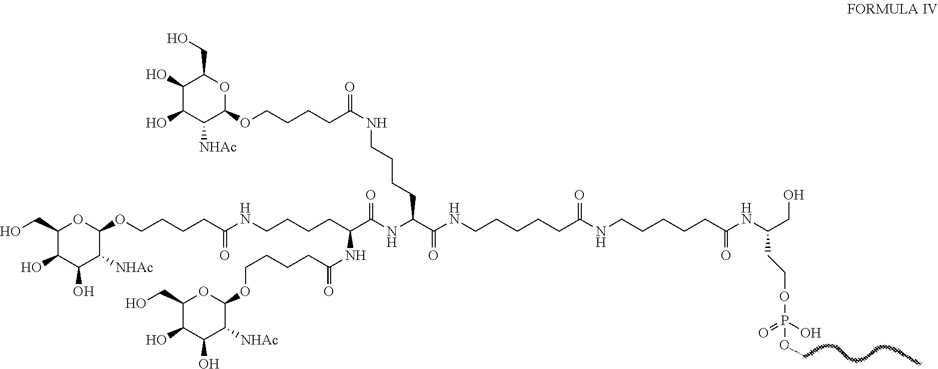 [SMILES: CC(=O)N[C@H]1[C@H](OCCCCC(=O)NCCCC[C@H](NC(=O)CCCCO[C@@H]2O[C@H](CO)[C@H](O)[C@H](O)[C@H]2NC(C)=O)C(=O)N[C@@H](CCCCNC(=O)CCCCO[C@@H]2O[C@H](CO)[C@H](O)[C@H](O)[C@H]2NC(C)=O)C(=O)NCCCCCC(=O)NCCCCCC(=O)N[C@H](CO)CCOP(=O)(O)O)O[C@H](CO)[C@H](O)[C@@H]1O]

In certain embodiments, the RNAi construct comprises a ligand and linker having the following structure of Formula V, wherein each n is independently 1 to 3, k is 1 to 3, and the ligand is attached to the 5′ end of the sense strand of the double-stranded RNA molecule (represented by the solid wavy line):

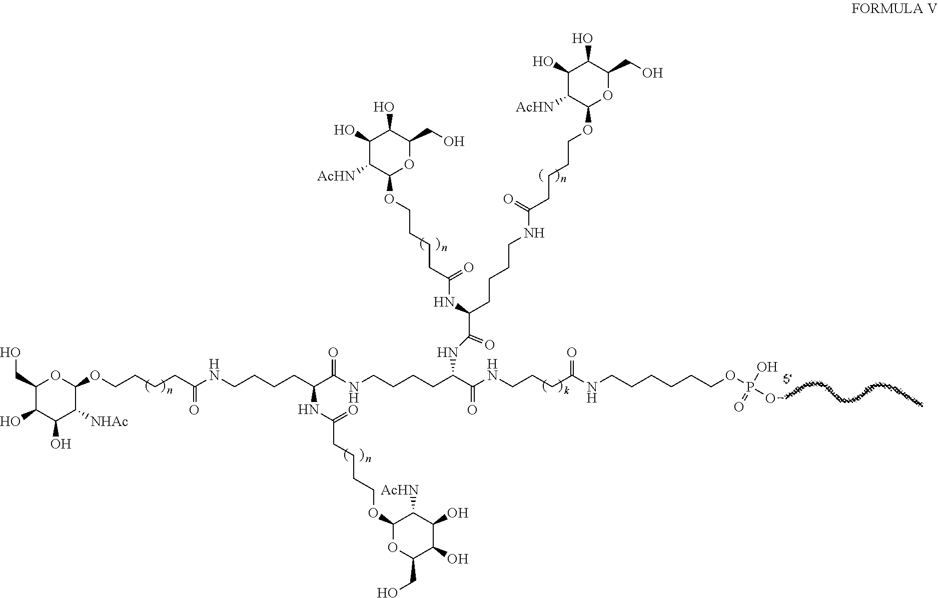

In other embodiments, the RNAi construct comprises a ligand and linker having the following structure of Formula VI, wherein each n is independently 1 to 3, k is 1 to 3, and the ligand is attached to the 5′ end of the sense strand of the double-stranded RNA molecule (represented by the solid wavy line):

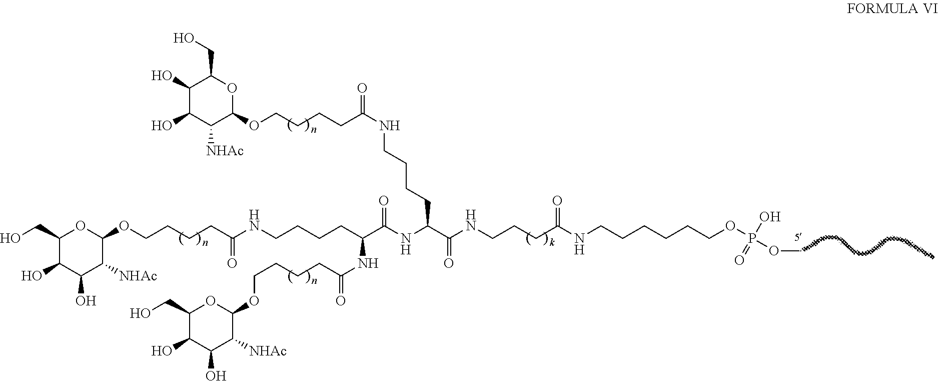

In some embodiments, the RNAi construct comprises a ligand and linker having the following structure of Formula VII, wherein X═O or S and wherein the ligand is attached to the 5′ end of the sense strand of the double-stranded RNA molecule (represented by the squiggly line):

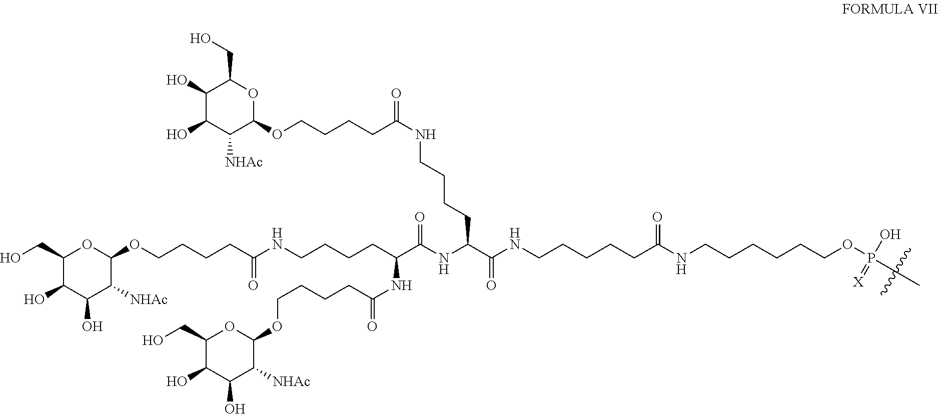

In some embodiments, the RNAi construct comprises a ligand and linker having the following structure of Formula VIII, wherein each n is independently 1 to 3 and the ligand is attached to the 5′ end of the sense strand of the double-stranded RNA molecule (represented by the solid wavy line):

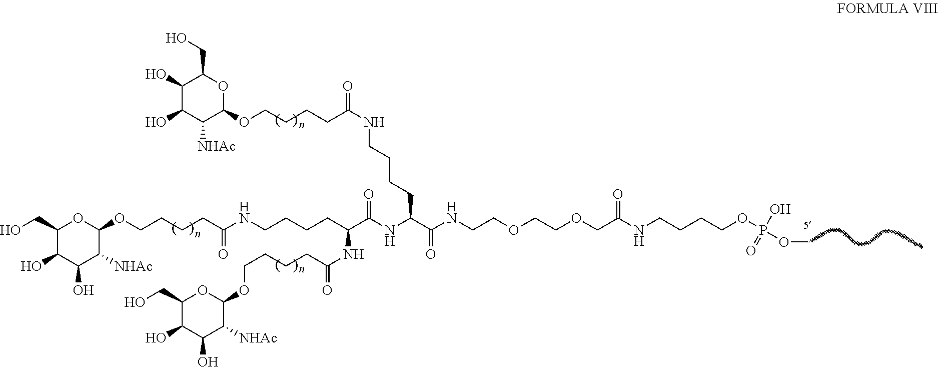

In certain embodiments, the RNAi construct comprises a ligand and linker having the following structure of Formula IX, wherein the ligand is attached to the 5′ end of the sense strand of the double-stranded RNA molecule (represented by the solid wavy line):

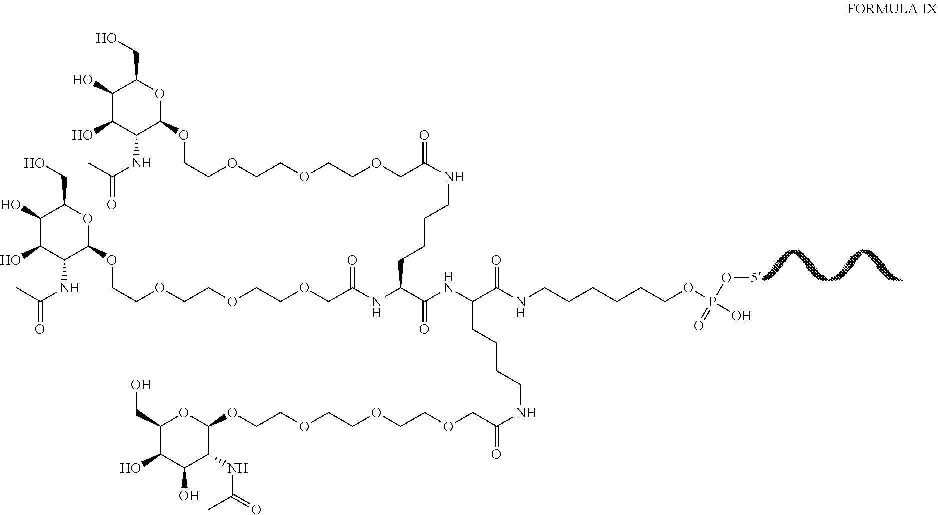

A phosphorothioate bond can be substituted for the phosphodiester bond shown in any one of Formulas I-IX to covalently attach the ligand and linker to the nucleic acid strand.

### Pharmaceutical Compositions

The present application also includes pharmaceutical compositions and formulations comprising the RNAi constructs described herein and pharmaceutically acceptable carriers, excipients, or diluents. Such compositions and formulations are useful for reducing expression of the FAM13A gene and FAM13A protein in a patient in need thereof. Where clinical applications are contemplated, pharmaceutical compositions and formulations will be prepared in a form appropriate for the intended application. Generally, this will entail preparing compositions that are essentially free of pyrogens, as well as other impurities that could be harmful to humans or animals.

The phrases “pharmaceutically acceptable” or “pharmacologically acceptable” refer to molecular entities and compositions that do not produce adverse, allergic, or other untoward reactions when administered to an animal or a human. As used herein, “pharmaceutically acceptable carrier, excipient, or diluent” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the disclosed RNAi constructs, its use in therapeutic compositions is contemplated. Supplementary active ingredients also can be incorporated into the compositions, provided they do not inactivate the RNAi constructs of the compositions.

Compositions and methods for the formulation of pharmaceutical compositions depend on a number of criteria, including, but not limited to, route of administration, type and extent of disease or disorder to be treated, or dose to be administered. In some embodiments, the pharmaceutical compositions are formulated based on the intended route of delivery. For instance, in certain embodiments, the pharmaceutical compositions are formulated for parenteral delivery. Parenteral forms of delivery include intravenous, intraarterial, subcutaneous, intrathecal, intraperitoneal, or intramuscular injection or infusion. In one embodiment, the pharmaceutical composition is formulated for intravenous delivery. In such an embodiment, the pharmaceutical composition may include a lipid-based delivery vehicle. In another embodiment, the pharmaceutical composition is formulated for subcutaneous delivery. In such an embodiment, the pharmaceutical composition may include a targeting ligand (e.g., a GalNAc-containing, fatty acid-containing, or antibody-containing ligand as described herein).

In some embodiments, the pharmaceutical compositions comprise an effective amount of an RNAi construct described herein. An “effective amount” is an amount sufficient to produce a beneficial or desired clinical result. In some embodiments, an effective amount is an amount sufficient to reduce FAM13A gene expression in a particular tissue or cell-type (e.g., liver or hepatocytes or adipose tissue) of a patient. An effective amount of an RNAi construct may be from about 0.01 mg/kg body weight to about 100 mg/kg body weight, and may be administered daily, weekly, monthly, or at longer intervals. The precise determination of what would be considered an effective amount and frequency of administration may be based on several factors, including a patient's size, age, and general condition, type of disorder to be treated (e.g., fatty liver disease, liver fibrosis, or cardiovascular disease), RNAi construct employed, and route of administration.

Administration of the disclosed pharmaceutical compositions may be via any common route so long as the target tissue is available via that route. Such routes include, but are not limited to, parenteral (e.g., subcutaneous, intramuscular, intraperitoneal, or intravenous), oral, nasal, buccal, intradermal, transdermal, and sublingual routes, or by direct injection into tissue (e.g., liver or adipose) or delivery through the hepatic portal vein. In some embodiments, the pharmaceutical composition is administered parenterally. For instance, in certain embodiments, the pharmaceutical composition is administered intravenously. In other embodiments, the pharmaceutical composition is administered subcutaneously.

Colloidal dispersion systems, such as macromolecule complexes, nanocapsules, microspheres, beads, and lipid-based systems, including oil-in-water emulsions, micelles, mixed micelles, and liposomes, may be used as delivery vehicles for the RNAi constructs. Commercially available fat emulsions that are suitable for delivering the nucleic acids include Intralipid® (Baxter International Inc.), Liposyn® (Abbott Pharmaceuticals), Liposyn® II (Hospira), Liposyn® III (Hospira), Nutrilipid (B. Braun Medical Inc.), and other similar lipid emulsions. An exemplary colloidal system for use as a delivery vehicle in vivo is a liposome (i.e., an artificial membrane vesicle). The RNAi constructs may be encapsulated within liposomes or may form complexes thereto, in particular to cationic liposomes. Alternatively, RNAi constructs may be complexed to lipids, in particular to cationic lipids. Suitable lipids and liposomes include neutral (e.g., dioleoylphosphatidyl ethanolamine (DOPE), dimyristoylphosphatidyl choline (DMPC), and dipalmitoyl phosphatidylcholine (DPPC)), distearolyphosphatidyl choline), negative (e.g., dimyristoylphosphatidyl glycerol (DMPG)), and cationic (e.g., dioleoyltetramethylaminopropyl (DOTAP) and dioleoylphosphatidyl ethanolamine (DOTMA)). The preparation and use of such colloidal dispersion systems are well known in the art. Exemplary formulations are also disclosed in U.S. Pat. Nos. 5,981,505; 6,217,900; 6,383,512; 5,783,565; 7,202,227; 6,379,965; 6,127,170; 5,837,533; 6,747,014; and WIPO Publication No. WO 03/093449.

In some embodiments, the RNAi constructs are fully encapsulated in a lipid formulation, e.g., to form a SNALP or other nucleic acid-lipid particle. As used herein, the term “SNALP” refers to a stable nucleic acid-lipid particle. SNALPs typically contain a cationic lipid, a non-cationic lipid, and a lipid that prevents aggregation of the particle (e.g., a PEG-lipid conjugate). SNALPs are exceptionally useful for systemic applications, as they exhibit extended circulation lifetimes following intravenous injection and accumulate at distal sites (e.g., sites physically separated from the administration site). The nucleic acid-lipid particles typically have a mean diameter of about 50 nm to about 150 nm, about 60 nm to about 130 nm, about 70 nm to about 110 nm, or about 70 nm to about 90 nm, and are substantially nontoxic. In addition, the nucleic acids when present in the nucleic acid-lipid particles are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; and WIPO Publication No. WO 96/40964.

The pharmaceutical compositions suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by using a coating, such as lecithin, by maintaining the required particle size in the case of dispersion and by using surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present application generally may be formulated in a neutral or salt form. Pharmaceutically acceptable salts include, for example, acid addition salts (formed with free amino groups) derived from inorganic acids (e.g., hydrochloric or phosphoric acids), or from organic acids (e.g., acetic, oxalic, tartaric, mandelic, and the like). Salts formed with the free carboxyl groups can also be derived from inorganic bases (e.g., sodium, potassium, ammonium, calcium, or ferric hydroxides) or from organic bases (e.g., isopropylamine, trimethylamine, histidine, procaine and the like). Pharmaceutically acceptable salts are described in detail in Berge et al., J. Pharmaceutical Sciences, Vol. 66: 1-19, 1977.

For parenteral administration in an aqueous solution, for example, the solution generally is suitably buffered, and the liquid diluent first rendered isotonic for example with sufficient saline or glucose. Such aqueous solutions may be used, for example, for intravenous, intramuscular, subcutaneous, and intraperitoneal administration. Preferably, sterile aqueous media are employed as is known to those of skill in the art, particularly in light of the present disclosure. By way of illustration, a single dose may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). For human administration, preparations should meet sterility, pyrogenicity, general safety and purity standards as required by FDA standards. In certain embodiments, a pharmaceutical composition comprises or consists of a sterile saline solution and an RNAi construct described herein. In other embodiments, a pharmaceutical composition comprises or consists of an RNAi construct described herein and sterile water (e.g., water for injection, WFI). In still other embodiments, a pharmaceutical composition comprises or consists of an RNAi construct described herein and phosphate-buffered saline (PBS).

In some embodiments, the pharmaceutical compositions are packaged with or stored within a device for administration. Devices for injectable formulations include, but are not limited to, injection ports, pre-filled syringes, autoinjectors, injection pumps, on-body injectors, and injection pens. Devices for aerosolized or powder formulations include, but are not limited to, inhalers, insufflators, aspirators, and the like. Thus, some embodiments comprise administration devices comprising a disclosed pharmaceutical composition for treating or preventing one or more of the diseases or disorders described herein.

### Uses for and Methods Using the Disclosed RNAi Constructs

The present application provides a method for reducing or inhibiting expression of the FAM13A gene, and thus the production of FAM13A protein, in a cell (e.g., liver cell or adipose cell) by contacting the cell with any one of the RNAi constructs described herein. The cell may be in vitro or in vivo. Any method capable of measuring FAM13A mRNA or FAM13A protein can be used to assess the efficacy of the RNAi constructs. The terms “FAM13A expression” and “expression of FAM13A,” as used herein, refer to the level of FAM13A gene transcription, amount of FAM13A mRNA present, level of FAM13A translation, and amount of FAM13A protein present. Therefore, FAM13A expression can be assessed by measuring the amount or level of FAM13A mRNA, FAM13A protein, or another biomarker linked to FAM13A expression, such as serum or plasma levels of triglycerides, cholesterol, or insulin. The phrase “reduction in FAM13A expression,” as used herein, refers to a decrease in one or more of the level of FAM13A gene transcription, amount of FAM13A mRNA present, level of FAM13A translation, and amount of FAM13A protein present.

The reduction of FAM13A expression in cells or animals treated with an RNAi construct can be determined relative to the FAM13A expression in cells or animals not treated with the RNAi construct or treated with a control RNAi construct. For instance, in some embodiments, reduction of FAM13A expression is assessed by (a) measuring the amount or level of FAM13A mRNA in cells (e.g., liver or adipose cells) treated with an RNAi construct, (b) measuring the amount or level of FAM13A mRNA in cells (e.g., liver or adipose cells) treated with a control RNAi construct (e.g., RNAi construct directed to an RNA molecule not expressed in cells or a RNAi construct having a nonsense or scrambled sequence) or no construct, and (c) comparing the measured FAM13A mRNA levels from treated cells in (a) to the measured FAM13A mRNA levels from control cells in (b). The FAM13A mRNA levels in the treated cells and controls cells can be normalized to RNA levels for a control gene (e.g., 18S ribosomal RNA or housekeeping gene) prior to comparison. FAM13A mRNA levels can be measured by a variety of methods, including Northern blot analysis, nuclease protection assays, fluorescence in situ hybridization (FISH), reverse-transcriptase (RT)-PCR, real-time RT-PCR, quantitative PCR, droplet digital PCR, and the like.

In other embodiments, reduction of FAM13A expression is assessed by (a) measuring the amount or level of FAM13A protein in cells (e.g., liver or adipose cells) treated with an RNAi construct, (b) measuring the amount or level of FAM13A protein in cells (e.g., liver or adipose cells) treated with a control RNAi construct (e.g., RNAi construct directed to an RNA molecule not expressed in cells or a RNAi construct having a nonsense or scrambled sequence) or no construct, and (c) comparing the measured FAM13A protein levels from treated cells in (a) to the measured FAM13A protein levels from control cells in (b). Methods of measuring FAM13A protein levels are known to those of skill in the art, and include Western Blots, immunoassays (e.g., ELISA), and flow cytometry.

In some embodiments, the methods to assess FAM13A expression levels are performed in vitro in cells that natively express FAM13A (e.g., liver or adipose cells) or cells that have been engineered to express FAM13A. In certain embodiments, the methods are performed in vitro in liver cells or adipose cells. Suitable liver cells include, but are not limited to, primary hepatocytes (e.g., human or non-human primate hepatocytes), HepAD38 cells, HuH-6 cells, HuH-7 cells, HuH-5-2 cells, BNLCL2 cells, Hep3B cells, or HepG2 cells. In one embodiment, the liver cells are HuH-7 cells. In another embodiment, the liver cells are human primary hepatocytes. In yet another embodiment, the liver cells are Hep3B cells. Suitable adipose cells include cells from subcutaneous white adipose tissue (scWAT), cells from epididymal white adipose tissue (eWAT), or 3T3-L1 cells.

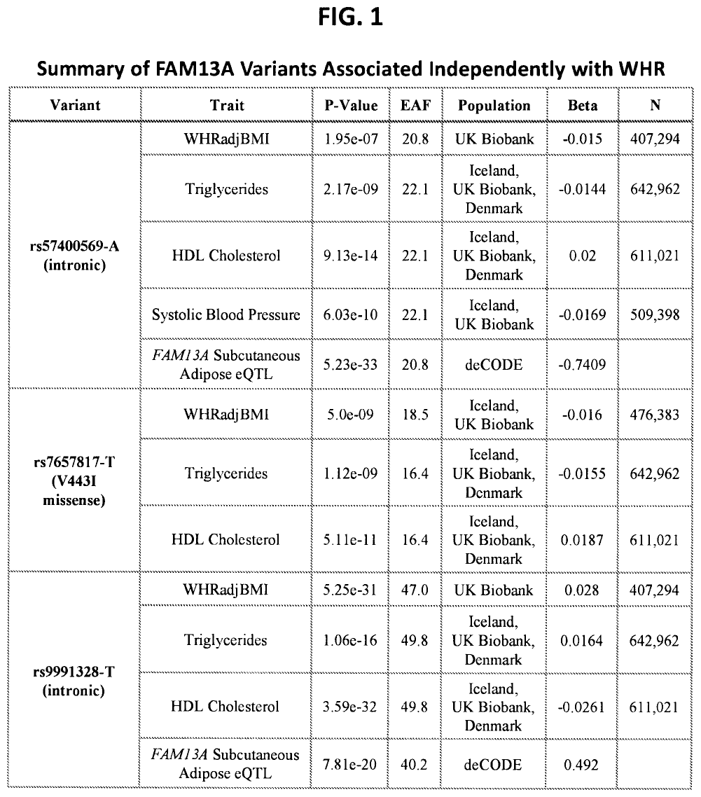

In other embodiments, the methods to assess FAM13A expression levels are performed in vivo. The RNAi constructs and any control RNAi constructs can be administered to an animal and FAM13A mRNA or FAM13A protein levels assessed in liver or adipose tissue harvested from the animal following treatment. Alternatively or additionally, a biomarker or functional phenotype associated with FAM13A expression can be assessed in the treated animals. For instance, people with FAM13A variants with reduced FAM13A expression also have reduced serum triglycerides and increased HDL cholesterol, and people with FAM13A variants with increased FAM13A expression also have increased triglycerides and decreased HDL cholesterol (FIG. 1). Additionally, FAM13A expression is significantly correlated with fasting insulin levels. Fathzadeh et al., Nature Communications 11, 1465 (2020). Thus, in some embodiments the goal and result of FAM13A knockdown is to reduce serum or plasma levels of triglycerides, cholesterol, or insulin, and such reduction can be measured in animals treated with RNAi constructs to assess the functional efficacy of reducing FAM13A expression.

In certain embodiments, expression of FAM13A mRNA or protein is reduced in liver or adipose cells by at least 40%, at least 45%, or at least 50% by an RNAi construct. In some embodiments, expression of FAM13A mRNA or protein is reduced in liver or adipose cells by at least 60%, at least 65%, at least 70%, at least 75%, at least 80%, or at least 85% by an RNAi construct. In other embodiments, the expression of FAM13A mRNA or protein is reduced in liver or adipose cells by about 90% or more, e.g., 91%, 92%, 93%, 94%, 95%, 96%, 97%, 98%, 99%, or more by an RNAi construct. The percent reduction of FAM13A expression can be measured by any of the methods described herein as well as others known in the art.

The present application provides methods for reducing or inhibiting expression of the FAM13A gene, and thus the production of FAM13A protein, in a patient in need thereof as well as methods of treating or preventing conditions, diseases, or disorders associated with FAM13A expression or activity. A “condition, disease, or disorder associated with FAM13A expression” refers to conditions, diseases, or disorders in which FAM13A expression levels are altered or where elevated expression levels of FAM13A are associated with an increased risk of developing the condition, disease, or disorder. A condition, disease, or disorder associated with FAM13A expression can also include conditions, diseases, or disorders resulting from aberrant changes in lipoprotein metabolism, such as changes resulting in abnormal or elevated levels of cholesterol, lipids, triglycerides, etc., or impaired clearance of these molecules. In certain embodiments, the RNAi constructs are particularly useful for treating or preventing abdominal adiposity, fatty liver disease (e.g., NAFLD and NASH) and cardiovascular disease (e.g., coronary artery disease and myocardial infarction), as well as reducing liver fibrosis and serum cholesterol levels.

Conditions, diseases, and disorders associated with FAM13A expression that can be treated or prevented according to the methods include, but are not limited to, fatty liver disease, such as alcoholic fatty liver disease, abdominal adiposity, alcoholic steatohepatitis, NAFLD and NASH; chronic liver disease; cirrhosis; cardiovascular disease, such as myocardial infarction, heart failure, stroke (ischemic and hemorrhagic), atherosclerosis, coronary artery disease, peripheral vascular disease (e.g., peripheral artery disease), cerebrovascular disease, vulnerable plaque, and aortic valve stenosis; familial hypercholesterolemia; venous thrombosis; hypercholesterolemia; hyperlipidemia; and dyslipidemia (manifesting, e.g., as elevated total cholesterol, elevated low-density lipoprotein (LDL), elevated very low-density lipoprotein (VLDL), elevated triglycerides, and/or low levels of high-density lipoprotein (HDL)).

In certain embodiments, the present application provides a method for reducing the expression of FAM13A protein in a patient in need thereof comprising administering to the patient any of the RNAi constructs described herein. The term “patient,” as used herein, refers to a mammal, including humans, and can be used interchangeably with the term “subject.” Preferably, the expression level of FAM13A in hepatocytes in the patient is reduced following administration of the RNAi construct as compared to the FAM13A expression level in a patient not receiving the RNAi construct or as compared to the FAM13A expression level in the patient prior to administration of the RNAi construct. In some embodiments, following administration of an RNAi construct, expression of FAM13A is reduced in the patient by at least 30%, at least 35%, at least 40%, at least 45%, at least 50%, at least 55%, at least 60%, at least 65%, at least 70%, at least 75%, at least 80%, at least 85%, or at least 90%, e.g., 91%, 92%, 93%, 94%, 95%, 96%, 97%, 98%, or 99%. The percent reduction of FAM13A expression can be measured by any of the methods described herein as well as others known in the art.

In some embodiments, a patient in need of reduction of FAM13A expression is a patient who is at risk of having a myocardial infarction. A patient who is at risk of having a myocardial infarction may be a patient who has a history of myocardial infarction (e.g., has had a previous myocardial infarction). A patient at risk of having a myocardial infarction may also be a patient who has a familial history of myocardial infarction or who has one or more risk factors of myocardial infarction. Such risk factors include, but are not limited to, hypertension, elevated levels of non-HDL cholesterol, elevated levels of triglycerides, diabetes, obesity, or history of autoimmune diseases (e.g., rheumatoid arthritis, lupus). In one embodiment, a patient who is at risk of having a myocardial infarction is a patient who has or is diagnosed with coronary artery disease. The risk of myocardial infarction in these and other patients can be reduced by administering to the patients any of the RNAi constructs described herein. Accordingly, a method for reducing the risk of myocardial infarction in a patient in need thereof comprises administering to the patient an RNAi construct described herein. In some embodiments, any of the RNAi constructs described herein may be used in the preparation of a medicament for reducing the risk of myocardial infarction in a patient in need thereof. Some embodiments comprise a FAM13A-targeting RNAi construct for use in a method for reducing the risk of myocardial infarction in a patient in need thereof.

In certain embodiments, a patient in need of reduction of FAM13A expression is a patient who is diagnosed with or at risk of cardiovascular disease. Thus, a method for treating or preventing cardiovascular disease in a patient in need thereof comprises administering any of the RNAi constructs. In some embodiments, any of the RNAi constructs described herein may be used in the preparation of a medicament for treating or preventing cardiovascular disease in a patient in need thereof. Some embodiments comprise a FAM13A-targeting RNAi construct may for use in a method for treating or preventing cardiovascular disease in a patient in need thereof. Cardiovascular disease includes, but is not limited to, myocardial infarction, heart failure, stroke (ischemic and hemorrhagic), atherosclerosis, coronary artery disease, peripheral vascular disease (e.g., peripheral artery disease), cerebrovascular disease, vulnerable plaque, and aortic valve stenosis. In some embodiments, the cardiovascular disease to be treated or prevented according to the disclosed methods is coronary artery disease. In other embodiments, the cardiovascular disease to be treated or prevented according to the disclosed methods is myocardial infarction. In yet other embodiments, the cardiovascular disease to be treated or prevented according to the disclosed methods is stroke. In still other embodiments, the cardiovascular disease to be treated or prevented according to the disclosed methods is peripheral artery disease. In certain embodiments, administration of the RNAi constructs described herein reduces the risk of non-fatal myocardial infarctions, fatal and non-fatal strokes, certain types of heart surgery (e.g., angioplasty, bypass), hospitalization for heart failure, chest pain in patients with heart disease, and/or cardiovascular events in patients with established heart disease (e.g., prior myocardial infarction, prior heart surgery, and/or chest pain with evidence of blocked arteries). In some embodiments, administration of the RNAi constructs described herein can be used to reduce the risk of recurrent cardiovascular events.

In some embodiments, a patient to be treated according to the disclosed methods is a patient who has a vulnerable plaque (also referred to as unstable plaque). Vulnerable plaques are a build-up of macrophages and lipids containing predominantly cholesterol that lie underneath the endothelial lining of the arterial wall. These vulnerable plaques can rupture resulting in the formation of a blood clot, which can potentially block blood flow through the artery and cause a myocardial infarction or stroke. Vulnerable plaques can be identified by methods known in the art, including, but not limited to, intravascular ultrasound and computed tomography (see Sahara et al., European Heart Journal, Vol. 25: 2026-2033, 2004; Budhoff, J. Am. Coll. Cardiol., Vol. 48: 319-321, 2006; Hausleiter et al., J. Am. Coll. Cardiol., Vol. 48: 312-318, 2006).

In other embodiments, a patient in need of reduction of FAM13A expression is a patient who has elevated blood levels of cholesterol (e.g., total cholesterol, non-HDL cholesterol, or LDL cholesterol). Accordingly, in some embodiments, a method for reducing blood levels (e.g., serum or plasma) of cholesterol in a patient in need thereof comprises administering to the patient any of the RNAi constructs described herein. In some embodiments, any of the RNAi constructs described herein may be used in the preparation of a medicament for reducing blood levels (e.g., serum or plasma) of cholesterol in a patient in need thereof. Some embodiments comprise a FAM13A-targeting RNAi construct for use in a method for reducing blood levels (e.g., serum or plasma) of cholesterol in a patient in need thereof. In certain embodiments, the cholesterol reduced according to the disclosed methods is LDL cholesterol. In other embodiments, the cholesterol reduced according to the disclosed methods is non-HDL cholesterol. Non-HDL cholesterol is a measure of all cholesterol-containing proatherogenic lipoproteins, including LDL cholesterol, very low-density lipoprotein, intermediate-density lipoprotein, lipoprotein(a), chylomicron, and chylomicron remnants. Non-HDL cholesterol has been reported to be a good predictor of cardiovascular risk (Rana et al., Curr. Atheroscler. Rep., Vol. 14:130-134, 2012). Non-HDL cholesterol levels can be calculated by subtracting HDL cholesterol levels from total cholesterol levels.

In some embodiments, a patient to be treated is a patient who has elevated levels of non-HDL cholesterol (e.g., elevated serum or plasma levels of non-HDL cholesterol). Ideally, levels of non-HDL cholesterol should be about 30 mg/dL above the target for LDL cholesterol levels for any given patient. In particular embodiments, a patient is administered an RNAi construct if the patient has a non-HDL cholesterol level of about 130 mg/dL or greater. In one embodiment, a patient is administered an RNAi construct if the patient has a non-HDL cholesterol level of about 160 mg/dL or greater. In another embodiment, a patient is administered an RNAi construct if the patient has a non-HDL cholesterol level of about 190 mg/dL or greater. In still another embodiment, a patient is administered an RNAi construct if the patient has a non-HDL cholesterol level of about 220 mg/dL or greater. In certain embodiments, a patient is administered an RNAi construct if the patient is at a high or very high risk of cardiovascular disease according to the 2013 ACC/AHA Guideline on the Assessment of Cardiovascular Risk (Goff et al., ACC/AHA guideline on the assessment of cardiovascular risk: a report of the American College of Cardiology/American Heart Association Task Force on Practice Guidelines. J Am Coll Cardiol, Vol. 63:2935-2959, 2014) and has a non-HDL cholesterol level of about 100 mg/dL or greater.

In certain embodiments, a patient is administered an RNAi construct described herein if they are at a moderate risk or higher for cardiovascular disease according to the 2013 ACC/AHA Guideline on the Assessment of Cardiovascular Risk (referred to herein as the “2013 Guidelines”). In certain embodiments, an RNAi construct is administered to a patient if the patient's LDL cholesterol level is greater than about 160 mg/dL. In other embodiments, an RNAi construct is administered to a patient if the patient's LDL cholesterol level is greater than about 130 mg/dL and the patient has a moderate risk of cardiovascular disease according to the 2013 Guidelines. In still other embodiments, an RNAi construct is administered to a patient if the patient's LDL cholesterol level is greater than 100 mg/dL and the patient has a high or very high risk of cardiovascular disease according to the 2013 Guidelines.

In other embodiments, a patient in need of reduction of FAM13A expression is a patient who is diagnosed with or at risk of fatty liver disease. Thus, a method for treating, preventing, or reducing the risk of developing fatty liver disease in a patient in need thereof comprises administering to the patient any of the disclosed RNAi constructs. In some embodiments, any of the RNAi constructs described herein may be used in the preparation of a medicament for treating, preventing, or reducing the risk of developing fatty liver disease in a patient in need thereof. Other embodiments comprise a FAM13A-targeting RNAi construct for use in a method for treating, preventing, or reducing the risk of developing fatty liver disease in a patient in need thereof. Fatty liver disease is a condition in which fat accumulates in the liver. There are two primary types of fatty liver disease: a first type that is associated with heavy alcohol use (alcoholic steatohepatitis) and a second type that is not related to use of alcohol (nonalcoholic fatty liver disease (NAFLD)). NAFLD is typically characterized by the presence of fat accumulation in the liver but little or no inflammation or liver cell damage. NAFLD can progress to nonalcoholic steatohepatitis (NASH), which is characterized by liver inflammation and cell damage, both of which in turn can lead to liver fibrosis and eventually cirrhosis or hepatic cancer. In certain embodiments, the fatty liver disease to be treated, prevented, or reduce the risk of developing is NAFLD. In other embodiments, the fatty liver disease to be treated, prevented, or reduce the risk of developing is NASH. In still other embodiments, the fatty liver disease to be treated, prevented, or reduce the risk of developing is alcoholic steatohepatitis. In some embodiments, a patient in need of treatment or prevention for fatty liver disease or is at risk of developing fatty liver disease has been diagnosed with type 2 diabetes, a metabolic disorder, or is obese (e.g., body mass index of ≥30.0). In other embodiments, a patient in need of treatment or prevention for fatty liver disease or is at risk of developing fatty liver disease has elevated levels of non-HDL cholesterol or triglycerides. Depending on the patient and other risk factors that patient may have, elevated levels of non-HDL cholesterol may be about 130 mg/dL or greater, about 160 mg/dL or greater, about 190 mg/dL or greater, or about 220 mg/dL or greater. Elevated triglyceride levels may be about 150 mg/dL or greater, about 175 mg/dL or greater, about 200 mg/dL or greater, or about 250 mg/dL or greater.

In certain embodiments, a patient in need of reduction of FAM13A expression is a patient who is diagnosed with or at risk of developing hepatic fibrosis or cirrhosis. Accordingly, some embodiments comprise a method for treating, preventing, or reducing liver fibrosis in a patient in need thereof comprising administering to the patient any of the disclosed RNAi constructs. Some embodiments comprise use of any of the RNAi constructs described herein in the preparation of a medicament for treating, preventing, or reducing liver fibrosis in a patient in need thereof. Some embodiments comprise a FAM13A-targeting RNAi construct for use in a method for treating, preventing, or reducing liver fibrosis in a patient in need thereof. In some embodiments, a patient at risk for developing hepatic fibrosis or cirrhosis is diagnosed with NAFLD. In other embodiments, a patient at risk for developing hepatic fibrosis or cirrhosis is diagnosed with NASH. In yet other embodiments, a patient at risk for developing hepatic fibrosis or cirrhosis is diagnosed with alcoholic steatohepatitis. In still other embodiments, a patient at risk for developing hepatic fibrosis or cirrhosis is diagnosed with hepatitis. In certain embodiments, administration of a disclosed RNAi construct prevents or delays the development of cirrhosis in the patient.

In other embodiments, a patient in need of reduction of FAM13A expression is a patient who has been diagnosed with abdominal adiposity or a high waist to hip ratio (WHR). In some embodiments, the patient in need of reduction has a waist to hip ratio in excess of 0.95, in excess of 1.0, in excess of 10.5, or in excess of 1.1. Accordingly, in some embodiments, a method for reducing abdominal adiposity or WHR in a patient in need thereof comprises administering to the patient any of the RNAi constructs described herein. In some embodiments, any of the RNAi constructs described herein may be used in the preparation of a medicament for reducing abdominal adiposity or WHR in a patient in need thereof. Some embodiments comprise a FAM13A-targeting RNAi construct for use in a method for reducing abdominal adiposity or WHR in a patient in need thereof.

In some embodiments, patients in need of reduction of FAM13A expression are treated using RNAi constructs targeted specifically to the liver. In some embodiments, the RNAi construct is targeted by conjugation to a ligand comprising N-acetyl-galactosamine (GalNAc). Accordingly, in some embodiments, a method for reducing FAM13A levels in a patient in need thereof comprises administering to the patient any of the RNAi constructs described herein that has been conjugated to GalNAc.

### Definitions of General Terms and Expressions

In order that the present disclosure can be more readily understood, certain terms are first defined. Unless defined otherwise, all technical and scientific terms used herein have the same meaning as commonly understood by one of ordinary skill in the art to which this disclosure is related. As used in this application, except as otherwise expressly provided herein, each of the following terms shall have the meaning set forth below. Additional definitions are set forth throughout the application.

Units, prefixes, and symbols are denoted in their Système International de Unites (SI) accepted form.

As used in the present disclosure and claims, the singular forms “a,” “an,” and “the” include plural forms unless the context clearly dictates otherwise. Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that wherever embodiments are described herein with the language “comprising,” otherwise analogous embodiments described in terms of “consisting of” and/or “consisting essentially of” are also provided. In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

Any compositions or methods provided herein can be combined with one or more of any of the other compositions and methods provided herein.

The following examples, including the experiments conducted and the results achieved, are provided for illustrative purposes only and are not to be construed as limiting the scope of the appended claims.

### EXAMPLES

### Example 1: Genomic and Expression Analysis of FAM13A

Genomic analysis was performed to examine the association of three common FAM13A variants for their association with adjusted for BMI (WHRadjBMI), triglyceride levels, HDL cholesterol levels, systolic blood pressure, and FAM13A expression in subcutaneous adipose tissue eQTL data. The results of this analysis are presented in FIG. 1 and show that three FAM13A variants associate independently with WHR adjusted for BMI.

First, the signal A variant rs57400569-A is an intronic SNP that is disease protective and is associated with increased HDL cholesterol and decreased WHR, triglycerides, and systolic blood pressure. rs57400569-A is associated with decreased FAM13A expression in deCODE adipose tissue eQTL data. rs57400569-A, consistent with FAM13A expression being correlated with disease state. The analysis also confirmed a reported association with blood pressure, while discovering previously unreported association with WHR, triglycerides, and HDL. rs57400569-A was also the top cis-eQTL variant in adipose.

Next, the signal B variant rs7657817-T is a protein coding missense variant that is associated with decreased WHR and triglycerides and increased HDL cholesterol. rs7657817-T is also disease protective, and the analysis confirmed previously reported literature associations.

Finally, the signal C variant rs9991328-T is an intronic SNP that is disease promoting and is associated with decreased HDL cholesterol and increased WHR, triglycerides, and FAM13A expression in deCODE adipose tissue eQTL data. Notably, rs9991328 has a strong, reproducible Genome Wide Association Study (GWAS) association with WHR in multiple studies (5-10) with a highly significant association reported in UK Biobank data (p=1×10⁻⁵¹) (5). Additionally, the rs9991328 WHR raising allele was significantly associated with increased fasting insulin levels (a measure of insulin resistance; p=5.9×10⁻²¹). rs9991328-T is disease promoting, and the analysis confirmed previously reported literature associations.

The waist-hip ratio raising alleles associate with increased triglycerides, reduced HDL cholesterol, increased systolic blood pressure, and increased FAM13A expression in subcutaneous adipose tissue.

### Example 2: siRNA-Mediated Knockdown of Murine Fam13a In Vivo

To test the hypothesis that reduced Fam13a expression is associated with reduced WHR & CVD risk factors, a series of mouse Fam13a siRNA experiments were performed. Fam13a siRNAs were conjugated to a palmitate lipid (C16) or GalNAc (attached as described in Example 3 below), and these molecules were tested for their ability to reduce Fam13a expression in cultured cells or in vivo (i.e., in adipose tissue or liver). These experiments were performed with commercially available mouse Fam13a siRNA triggers. The triggers are available from Ambion (s81721) or Dharmacon (J-041073-09), and were prepared as modified siRNA duplexes. The murine siRNA duplex sequences were:

D-0001 sense
(SEQ ID NO: 2786)
GAAAGAUUCCAGGACGAU
D-0001 antisense
(SEQ ID NO: 2787)
UAUCGUCCUGGAAUCUUUCUG
D-0002 sense
(SEQ ID NO: 2788)
GAAUCAAGAUGGUGAAGA
D-0002 antisense
(SEQ ID NO: 2789)
AUCUUCACCAUCUUGAUUCCUC
D-0003 sense
(SEQ ID NO: 2790)
AGGAAUCAAGAUGGUGAAGA
D-0003 antisense
(SEQ ID NO: 2791)
AUCUUCACCAUCUUGAUUCCUCU

These sequences were prepared as modified duplexes, as shown below. The nucleotide sequences of these modified duplexes apply the following notations: a, u, g, and c=corresponding 2′-O-methyl ribonucleotide; Af, Uf, Gf, and Cf=corresponding 2′-deoxy-2′-fluoro (“2′-fluoro”) ribonucleotide; and invAb=inverted abasic deoxynucleotide (i.e., abasic deoxynucleotide linked to adjacent nucleotide via a substituent at its 3′ position (a 3′-3′ linkage) when on the 3′ end of a strand or linked to adjacent nucleotide via a substituent at its 5′ position (a 5′-5′ internucleotide linkage) when on the 5′ end of a strand. Insertion of an “s” in the sequence indicates that the two adjacent nucleotides are connected by a phosphorothiodiester group (e.g., a phosphorothioate internucleotide linkage). Unless indicated otherwise, all other nucleotides are connected by 3′-5′ phosphodiester groups. The Fam13a siRNAs were conjugated to a palmitate lipid (C16) or GalNAc, using the methods provided in Example 3 below.

D-0004 sense
(SEQ ID NO: 2792)
gaaagaUfuCfCfAfGfgacgasus{invAb}
D-0004 antisense
(SEQ ID NO: 2793)
usAfsucguCfcuggAfaUfcuuucsusg
D-0005 sense
(SEQ ID NO: 2794)
gaaucaAfgAfUfGfGfugaagsas{invAb}
D-0005 antisense
(SEQ ID NO: 2795)
asUfscuucAfccauCfuUfgauucscsu
D-0006 sense
(SEQ ID NO: 2796)
{DCA-C6}saggaaucaAfgAfUfGfGfugaagas{invAb}
D-0006 antisense
(SEQ ID NO: 2797)
asUfscuucAfccauCfuUfgauuccuscsu

In Vitro Fam13a siRNA Treatment

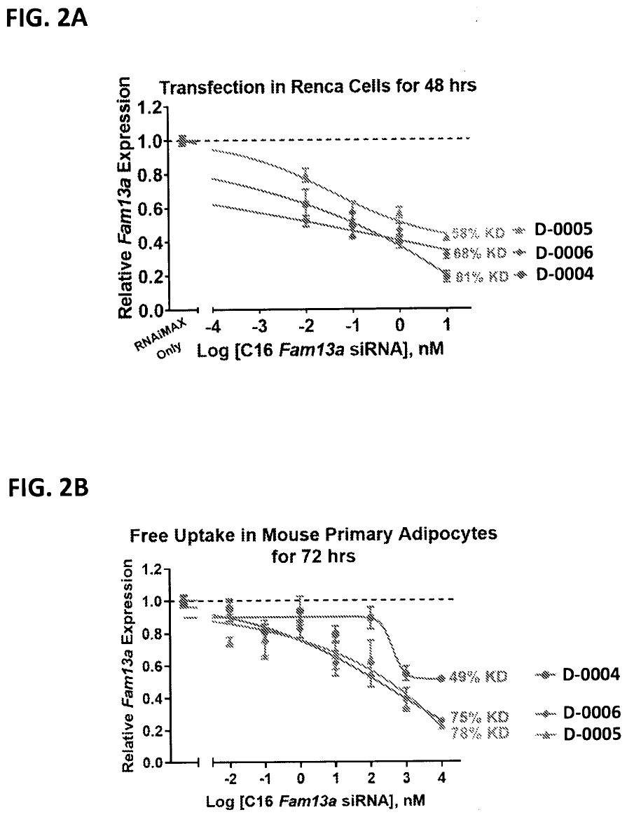

Fam13a siRNA effects on Fam13a RNA expression levels were analyzed in murine kidney-derived (Renca cell line; ATCC CRL-2947) and adipose-derived (primary adipocytes) cultured cells. FIGS. 2A and 2B show the results of this in vitro dose-response study of Fam13a siRNA's effects in Renca cells and primary adipocytes.

For experiments in Renca cells, siRNAs were transfected into cells using Lipofectamine RNAiMAX transfection reagent (Thermo Fisher Scientific). Cells were plated in 96-well plates at 12,500 cells per well in 100 μL, base medium (RPMI-1640, 10% FBS, 1% Non-essential amino acids, 1% sodium pyruvate, 2% L-glutamine, and 1% penicillin-streptomycin) and incubated overnight. For transfection, 150 μL RNAiMAX was mixed with OptiMEM (final dilution 0.3 μL RNAiMAX per well), then 1 mM siRNA was diluted to 60 μM in OptiMEM/RNAiMAx and then further diluted to 6 nM starting concentration. siRNAs were serially diluted 1:10 from 6 nM, 0.6 nM, 0.06 nM, and 0.006 nM. To the 100 μL plating media, 20 μL OptiMEM/RNAiMAX+siRNA were added for final concentrations of 1 nM, 0.1 nM, 0.01 nM, 0.001 nM, and 0 nM of each siRNA. Cells were incubated for 72 hrs. at 37° C. and 5% CO₂ then media was removed and lysed with 150 μL Buffer RLT (Qiagen). RNA was isolated using RNeasy 96 RNA isolation protocol following manufacturer's instructions (Qiagen). Real-time PCR was performed using TaqMan® RNA-to-Ct™ 1-Step Kit following manufacturer's instructions (ThermoFisher) with 4.25 μL RNA and TaqMan® gene expression assays (ThermoFisher) for Fam13a (Mm00467910) and Hprt (Mm03024075).

For experiments in primary mouse adipocytes, using the method originally described by Viswanadha and Londos (Viswanadha, S. & Londos, C. Optimized conditions for measuring lipolysis in murine primary adipocytes. J. Lipid Res. 47, 1859-1864 (2006)), the subcutaneous WAT was isolated and dissected from male DIO mice, weighed, and immediately submerged in Krebs-Ringer bicarbonate (KRB) buffer at pH 7.4 with 4% bovine serum albumin (BSA), 500 nM adenosine, and 5 mM glucose, and the stromal vascular fraction (SVF) and primary adipocytes were separated by collagenase digestion (1 mg/mL KRB) and incubated at 37° C. with shaking at 220 rpm for 1 h. After digestion, the mixture was filtered through a 250-μm gauze mesh into a 15-mL conical polypropylene tube and the infranatant containing the collagenase solution and the SVF was carefully removed using a long needle and syringe. The SVF was cultured as previously described by Hausman et al. (Hausman, D. B., Park, H. J. & Hausman, G. J. Isolation and culture of preadipocytes from rodent white adipose tissue. Methods Mol. Biol. 456, 201-219 (2008)) where the SVF containing solution was centrifuged at 200×g for 10 min to pellet the SVF cells, resuspended in 10 mL plating medium (DMEM/F12+10% FBS), then filtered through a sterile 20-μm mesh filter into a sterile 50-mL plastic centrifuge tube. SVF cells were plated in 24-well plate at 250,000 cells/well and incubated at 37° C. and 5% CO₂ overnight then the plating medium and nonadherent cells where removed, replaced with DMEM/F12 media+5% FBS, and media was replaced every two days until cells reached confluency (5-6 days after plating). Differentiation was induced by the addition of differentiation media for 48 h (DMEM/F12+5% FBS+17 nM insulin, 0.1 μM dexamethasone, 250 μM 3-Isobutyl-1-methylxanthine (IBMX), and 60 μM indomethacin). After 48 h, the differentiation media was replaced by maintenance media (DMEM/F12+10% FBS+17 nM insulin) for a total of 10 days with the maintenance media replaced every 2-3 days. On day 10 of differentiation, C16 conjugated siRNAs were diluted to 10 μM in maintenance media then 10 μM siRNA was serially diluted 1:10 from 10 μM, 1 μM, 100 nM, 10 nM, 1 nM, 0.1 nM, and 0.01 nM. Maintenance media was removed from the cells and replaced with 1.5 mL siRNA containing media and cells were incubated for 72 hrs. at 37° C. and 5% CO₂. After 72 hours, media was removed and cells were collected in 1 mL Qiazol (Qiagen) per well. RNA was isolated using RNeasy 96 universal tissue kit RNA isolation protocol following manufacturer's instructions (Qiagen). Real-time PCR was performed using TaqMan® RNA-to-Ct™ 1-Step Kit following manufacturer's instructions (ThermoFisher) with 4.25 μL RNA and TaqMan® gene expression assays (ThermoFisher) for Fam13a (Mm00467910) and Ppib (Mm00478295).

As shown in FIGS. 2A and 2B, each tested Fam13a siRNA construct reduced Fam13a expression in a dose-dependent manner. At the highest concentrations, 58%, 68%, or 81% reduction in Fam13a mRNA expression levels were observed in Renca cells. Similarly, at the highest concentrations, 49%, 75%, and 78% reduction in Fam13a mRNA levels were observed in primary adipocytes.

5-Day In Vivo Fam13a siRNA Treatment in Diet-Induced Obese Mice

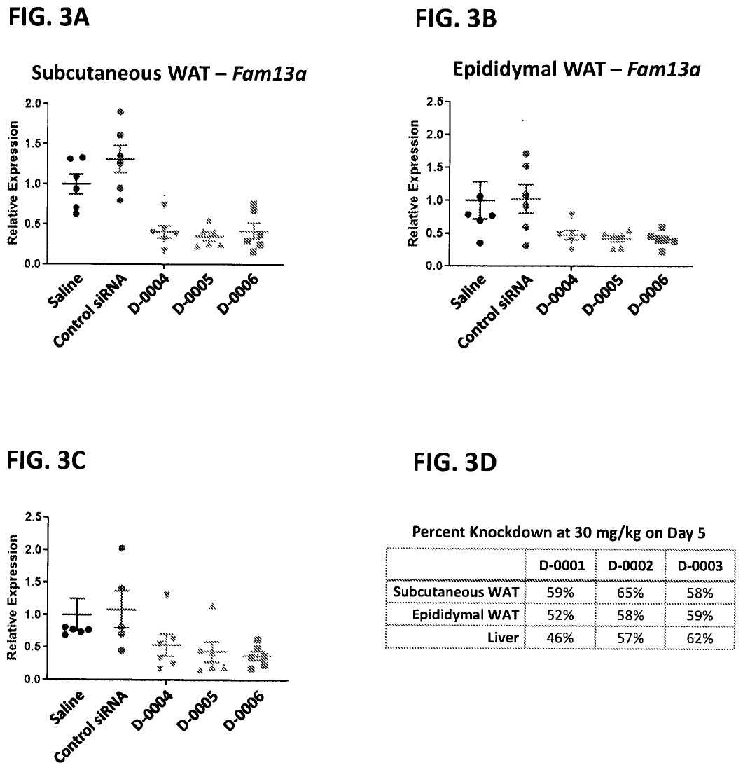

Fam13a siRNA effects on Fam13a RNA expression levels were analyzed in the high fat diet (HFD) murine model of diet-induced obesity (DIO) and insulin resistance within 5 days. Mice were placed on a HFD for 12 weeks (18 weeks old; n=6/group). Mice were then subcutaneously administered a single injection containing a 30 mg/kg dose of C16 conjugated murine Fam13a siRNA, a control C16 siRNA targeting mHprt (C16-Hprt siRNA), or vehicle control. Five days post-injection, mice were sacrificed and necropsy was performed in which subcutaneous WAT, epididymal WAT, and liver tissue were obtained. Fam13a RNA expression levels were using RNeasy 96 universal tissue kit RNA isolation protocol following manufacturer's instructions (Qiagen). Real-time PCR was performed using TaqMan® RNA-to-Ct™ 1-Step Kit following manufacturer's instructions (ThermoFisher) with 4.25 IA RNA and TaqMan® gene expression assays (ThermoFisher) for Fam13a (Mm00467910) and Ppib (Mm00478295). As shown in FIGS. 3A-3D, the Fam13a siRNA constructs reduced Fam13a RNA expression in both the liver and adipose tissue.

30-Day In Vivo Fam13a siRNA Treatment in Diet-Induced Obese Mice

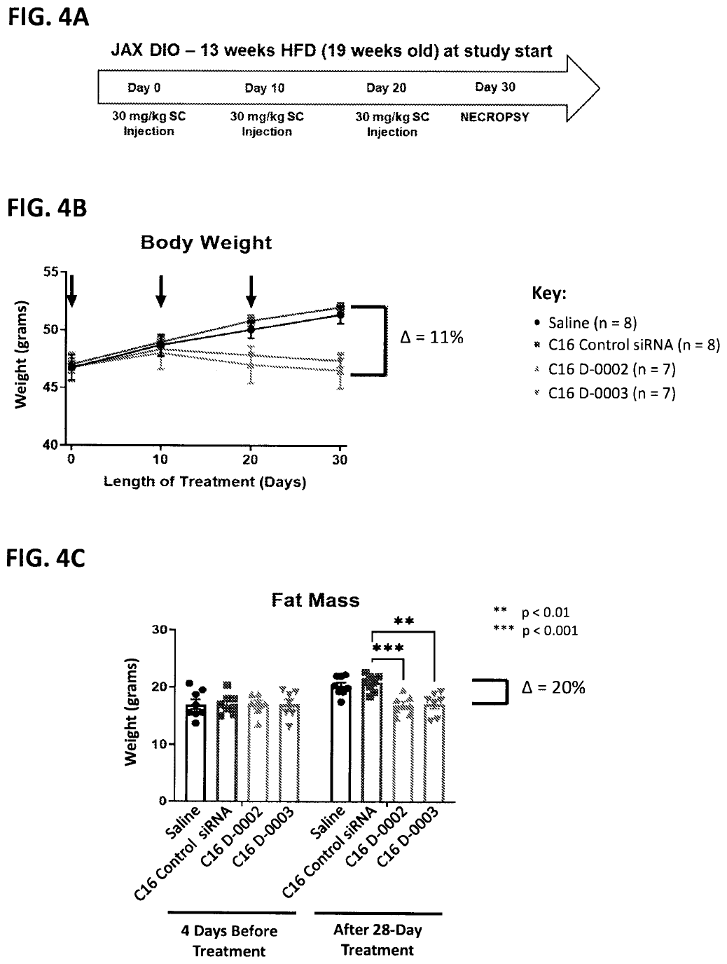

Fam13a siRNA's physiological effects were analyzed in the high fat diet (HFD) murine model of diet-induced obesity and insulin resistance after repeated siRNA injection over the course of 30 days. Mice were placed on a HFD for 12 weeks (19 weeks old; n=7 or 8 per group). Mice were then administered a 30 mg/kg dose of a C16 conjugated murine Fam13a siRNA (D-0002 or D-0003), a control C16 siRNA targeting mHprt (C16-Hprt siRNA), or vehicle control (SC) once every 10 days for a total of three doses (see FIG. 4A). Body weight was measured for each mouse at the start of treatment and every 10 days thereafter until the mice were sacrificed thirty days post-injection, when necropsy was performed. Fat mass was measured for each mouse 4 days prior to the first siRNA administration and after 28 days of treatment.

FIGS. 4B and 4C are plots showing the results of Fam13a siRNA on body weight and fat mass of mice. After 30 days of treatment, both tested Fam13a siRNAs significantly reduced body weight by 11% and fat mass by 20% compared to the controls. These data demonstrate that the C16-conjugated siRNA triggers significantly reduced Fam13a expression in vivo in adipose tissue when conjugated to C16.

Additionally, liver weight was reduced by −25%, liver triglyceride was reduced by −31%, plasma insulin was reduced by −40%, and plasma LDL was reduced by −17%. These liver-related effects indicate that the C16-conjugated siRNA triggers also were effective in liver tissue.

60-Day In Vivo Fam13a siRNA Treatment in Diet-Induced Obese Mice

An experiment was performed to compare the results using a GalNAc-conjugated Fam13a siRNA (which targets the siRNA specifically to the liver) head-to-head with the results using a C16-conjugated Fam13a siRNA (which targets the siRNA to both adipose tissue and liver). Obese mice were treated with the following molecules every 10 days for 60 days: (1) saline, (2) C16 conjugated non-targeting (NT) siRNA control (30 mg/kg), (3) C16-Fam13a siRNA (D-0002; 30 mg/kg), (4) C16-Fam13a siRNA (D-0002; 5 mg/kg), (5) GalNAc conjugated NT siRNA control (5 mg/kg), or (6) GalNAc-Fam13a siRNA (D-0002; 5 mg/kg).

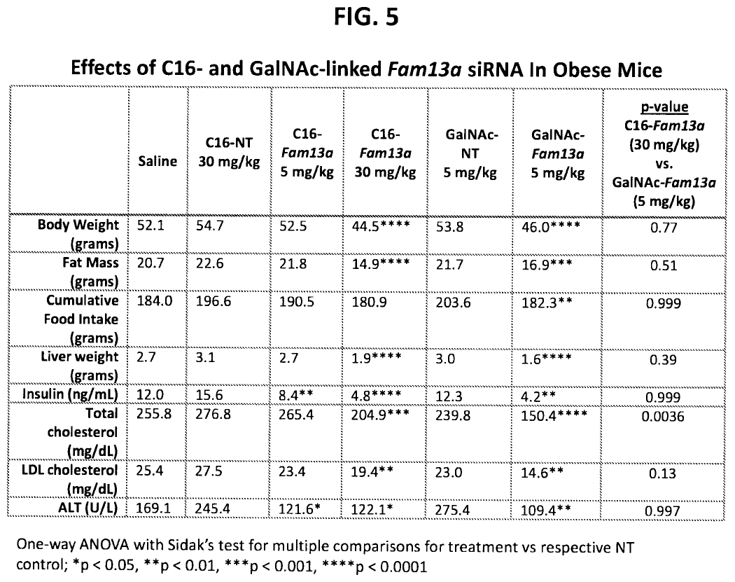

After 60 days of treatment, both C16 and GalNAc siRNA treatments significantly reduced body weight, fat mass, liver weight, insulin, total cholesterol, LDL cholesterol, and ALT compared to their respective NT siRNA controls (FIG. 5). The mouse Fam13a×GalNAc siRNA dosed at 5 mg/kg every 10 days for 60 days in obese mice significantly reduced body weight by −15%, fat mass by −22%, liver weight by −49%, insulin by −66%, total cholesterol by −37%, LDL cholesterol by −37%, and ALT by −60%. Of therapeutic importance, GalNAc-Fam13a siRNA (5 mg/kg) treatment was sufficient to significantly reduce all metabolic endpoints to at least approximately the same extent as C16-Fam13a siRNA (30 mg/kg) treatment, which demonstrates that hepatic targeting is sufficient for efficacy of Fam13a siRNA in obese mice. Additionally, GalNAc-Fam13a siRNA significantly reduced total cholesterol to a greater extent than C16-Fam13a siRNA, suggesting that hepatic specific targeting may provide enhanced therapeutic benefit beyond broad targeting by a lipid conjugate and at a 6-fold lower dose.

### Example 3: Selection, Design and Synthesis of Modified FAM13A siRNA Molecules

Candidate sequences for the design of therapeutic siRNA molecules targeting the human FAM13A gene were identified using a bioinformatics analysis of the human FAM13A transcript provided herein as SEQ ID NO: 1 (Ensembl transcript no. ENST00000264344.9). The bioinformatics analysis included performing informatic analysis of SEQ ID NO: 1, including tiling SEQ ID NO: 1 by triggers of 21 nucleotides in length. To minimize the risk of off target effects, all triggers that were complementary to human micro-RNA and with less than three base pair mismatches to any identified human gene were not prepared for functional testing. In addition, sequences were selected for their ability to cross-react with human and cynomolgus monkey FAM13A mRNA. Based on the results of the bioinformatics analysis, sequences were selected for initial synthesis and in vitro testing.

Table 1 below lists the unmodified sense and antisense sequences for duplex molecules prioritized from the bioinformatics analysis. The first nucleotide in the range of nucleotides targeted by siRNA molecules in each sequence family within the human FAM13A transcript (SEQ ID NO: 1) is also shown in Table 1.

TABLE 1
siRNA Sequences Directed to FAM13A

| Duplex | Target start in | | SEQ ID | | SEQ ID |
|---|---|---|---|---|---|
| No. | SEQ ID NO: 1 | Sense Sequence (5′-3′) | NO: | Antisense Sequence (5′-3′) | NO: |
| D-1001 | 1282 | CAUGUACCCCAAGUCAGCAAU | 2 | AUUGCUGACUUGGGGUACAUG | 546 |
| D-1002 | 1284 | UGUACCCCAAGUCAGCAAUGU | 3 | ACAUUGCUGACUUGGGGUACA | 547 |
| D-1003 | 1285 | GUACCCCAAGUCAGCAAUGUG | 4 | CACAUUGCUGACUUGGGGUAC | 548 |
| D-1004 | 1298 | GCAAUGUGUCUGCAACCGGAG | 5 | CUCCGGUUGCAGACACAUUGC | 549 |
| D-1005 | 1299 | CAAUGUGUCUGCAACCGGAGA | 6 | UCUCCGGUUGCAGACACAUUG | 550 |
| D-1006 | 1300 | AAUGUGUCUGCAACCGGAGAA | 7 | UUCUCCGGUUGCAGACACAUU | 551 |
| D-1007 | 1302 | UGUGUCUGCAACCGGAGAACU | 8 | AGUUCUCCGGUUGCAGACACA | 552 |
| D-1008 | 1303 | GUGUCUGCAACCGGAGAACUC | 9 | GAGUUCUCCGGUUGCAGACAC | 553 |
| D-1009 | 1304 | UGUCUGCAACCGGAGAACUCU | 10 | AGAGUUCUCCGGUUGCAGACA | 554 |
| D-1010 | 1305 | GUCUGCAACCGGAGAACUCUU | 11 | AAGAGUUCUCCGGUUGCAGAC | 555 |
| D-1794 | | | | | |
| D-1011 | 1306 | UCUGCAACCGGAGAACUCUUA | 12 | UAAGAGUUCUCCGGUUGCAGA | 556 |
| D-1795 | | | | | |
| D-1012 | 1307 | CUGCAACCGGAGAACUCUUAG | 13 | CUAAGAGUUCUCCGGUUGCAG | 557 |
| D-1013 | 1308 | UGCAACCGGAGAACUCUUAGA | 14 | UCUAAGAGUUCUCCGGUUGCA | 558 |
| D-1796 | | | | | |
| D-1014 | 1309 | GCAACCGGAGAACUCUUAGAA | 15 | UUCUAAGAGUUCUCCGGUUGC | 559 |
| D-1545 | | | | | |
| D-1635 | | | | | |
| D-1639 | | | | | |
| D-1640 | | | | | |
| D-1646 | | | | | |
| D-1652 | | | | | |
| D-1657 | | | | | |
| D-1662 | | | | | |
| D-1667 | | | | | |
| D-1670 | | | | | |
| D-1676 | | | | | |
| D-1681 | | | | | |
| D-1686 | | | | | |
| D-1691 | | | | | |
| D-1847 | | | | | |
| D-1849 | | | | | |
| D-1859 | | | | | |
| D-2009 | | | | | |
| D-2018 | | | | | |
| D-1015 | 1311 | AACCGGAGAACUCUUAGAAAG | 16 | CUUUCUAAGAGUUCUCCGGUU | 560 |
| D-1570 | | | | | |
| D-1016 | 1322 | UCUUAGAAAGAACCAUCCGAU | 17 | AUCGGAUGGUUCUUUCUAAGA | 561 |
| D-1017 | 1323 | CUUAGAAAGAACCAUCCGAUC | 18 | GAUCGGAUGGUUCUUUCUAAG | 562 |
| D-1018 | 1324 | UUAGAAAGAACCAUCCGAUCA | 19 | UGAUCGGAUGGUUCUUUCUAA | 563 |
| D-1019 | 1326 | AGAAAGAACCAUCCGAUCAGC | 20 | GCUGAUCGGAUGGUUCUUUCU | 564 |
| D-1817 | | | | | |
| D-1020 | 1328 | AAAGAACCAUCCGAUCAGCUG | 21 | CAGCUGAUCGGAUGGUUCUUU | 565 |
| D-1599 | | | | | |
| D-1021 | 1329 | AAGAACCAUCCGAUCAGCUGU | 22 | ACAGCUGAUCGGAUGGUUCUU | 566 |
| D-1022 | 1331 | GAACCAUCCGAUCAGCUGUAG | 23 | CUACAGCUGAUCGGAUGGUUC | 567 |
| D-1818 | | | | | |
| D-1023 | 1333 | ACCAUCCGAUCAGCUGUAGAA | 24 | UUCUACAGCUGAUCGGAUGGU | 568 |
| D-1597 | | | | | |
| D-1694 | | | | | |
| D-1700 | | | | | |
| D-1707 | | | | | |
| D-1714 | | | | | |
| D-1721 | | | | | |
| D-1728 | | | | | |
| D-1735 | | | | | |
| D-1853 | | | | | |
| D-1873 | | | | | |
| D-2006 | | | | | |
| D-2015 | | | | | |
| D-1024 | 1338 | CCGAUCAGCUGUAGAACAACA | 25 | UGUUGUUCUACAGCUGAUCGG | 569 |
| D-1569 | | | | | |
| D-1025 | 1366 | GAUGUUAAUAACUCUGGAGGU | 26 | ACCUCCAGAGUUAUUAACAUC | 570 |
| D-1543 | | | | | |
| D-1026 | 1371 | UAAUAACUCUGGAGGUCAAAG | 27 | CUUUGACCUCCAGAGUUAUUA | 571 |
| D-1027 | 1373 | AUAACUCUGGAGGUCAAAGUU | 28 | AACUUUGACCUCCAGAGUUAU | 572 |
| D-1028 | 1407 | AUCUGGAACACUAUCAGCAUC | 29 | GAUGCUGAUAGUGUUCCAGAU | 573 |
| D-1819 | | | | | |
| D-1029 | 1472 | AGGAUGAAGUUCGACAUGGGA | 30 | UCCCAUGUCGAACUUCAUCCU | 574 |
| D-1797 | | | | | |
| D-1030 | 1480 | GUUCGACAUGGGAGAGACAAG | 31 | CUUGUCUCUCCCAUGUCGAAC | 575 |
| D-1031 | 1483 | CGACAUGGGAGAGACAAGGGA | 32 | UCCCUUGUCUCUCCCAUGUCG | 576 |
| D-1032 | 1485 | ACAUGGGAGAGACAAGGGACU | 33 | AGUCCCUUGUCUCUCCCAUGU | 577 |
| D-1033 | 1487 | AUGGGAGAGACAAGGGACUUA | 34 | UAAGUCCCUUGUCUCUCCCAU | 578 |
| D-1034 | 1489 | GGGAGAGACAAGGGACUUAUC | 35 | GAUAAGUCCCUUGUCUCUCCC | 579 |
| D-1542 | | | | | |
| D-1035 | 1490 | GGAGAGACAAGGGACUUAUCA | 36 | UGAUAAGUCCCUUGUCUCUCC | 580 |
| D-1036 | 1491 | GAGAGACAAGGGACUUAUCAA | 37 | UUGAUAAGUCCCUUGUCUCUC | 581 |
| D-1037 | 1495 | GACAAGGGACUUAUCAACAAA | 38 | UUUGUUGAUAAGUCCCUUGUC | 582 |
| D-1553 | | | | | |
| D-1038 | 1496 | ACAAGGGACUUAUCAACAAAG | 39 | CUUUGUUGAUAAGUCCCUUGU | 583 |
| D-1589 | | | | | |
| D-1039 | 1500 | GGGACUUAUCAACAAAGAAAA | 40 | UUUUCUUUGUUGAUAAGUCCC | 584 |
| D-1798 | | | | | |
| D-1040 | 1514 | AAGAAAAUACUCCUUCUGGGU | 41 | ACCCAGAAGGAGUAUUUUCUU | 585 |
| D-1820 | | | | | |
| D-1933 | | | | | |
| D-1939 | | | | | |
| D-1945 | | | | | |
| D-1951 | | | | | |
| D-1957 | | | | | |
| D-1963 | | | | | |
| D-1969 | | | | | |
| D-1041 | 1520 | AUACUCCUUCUGGGUUCAACC | 42 | GGUUGAACCCAGAAGGAGUAU | 586 |
| D-1799 | | | | | |
| D-1042 | 1533 | GUUCAACCACCUUGAUGAUUG | 43 | CAAUCAUCAAGGUGGUUGAAC | 587 |
| D-1576 | | | | | |
| D-1043 | 1534 | UUCAACCACCUUGAUGAUUGU | 44 | ACAAUCAUCAAGGUGGUUGAA | 588 |
| D-1616 | | | | | |
| D-1044 | 1558 | UUGAAUACUCAGGAAGUCGAA | 45 | UUCGACUUCCUGAGUAUUCAA | 589 |
| D-1575 | | | | | |
| D-1045 | 1564 | ACUCAGGAAGUCGAAAAGGUA | 46 | UACCUUUUCGACUUCCUGAGU | 590 |
| D-1821 | | | | | |
| D-1046 | 1565 | CUCAGGAAGUCGAAAAGGUAC | 47 | GUACCUUUUCGACUUCCUGAG | 591 |
| D-1047 | 1566 | UCAGGAAGUCGAAAAGGUACA | 48 | UGUACCUUUUCGACUUCCUGA | 592 |
| D-1048 | 1568 | AGGAAGUCGAAAAGGUACACA | 49 | UGUGUACCUUUUCGACUUCCU | 593 |
| D-1049 | 1574 | UCGAAAAGGUACACAAAAAUA | 50 | UAUUUUUGUGUACCUUUUCGA | 594 |
| D-1050 | 1575 | CGAAAAGGUACACAAAAAUAC | 51 | GUAUUUUUGUGUACCUUUUCG | 595 |
| D-1051 | 1610 | GAGAAAGGAGCAAGCCUAAAC | 52 | GUUUAGGCUUGCUCCUUUCUC | 596 |
| D-1052 | 1611 | AGAAAGGAGCAAGCCUAAACG | 53 | CGUUUAGGCUUGCUCCUUUCU | 597 |
| D-1822 | | | | | |
| D-1053 | 1612 | GAAAGGAGCAAGCCUAAACGU | 54 | ACGUUUAGGCUUGCUCCUUUC | 598 |
| D-1054 | 1615 | AGGAGCAAGCCUAAACGUCAG | 55 | CUGACGUUUAGGCUUGCUCCU | 599 |
| D-1823 | | | | | |
| D-1055 | 1616 | GGAGCAAGCCUAAACGUCAGA | 56 | UCUGACGUUUAGGCUUGCUCC | 600 |
| D-1824 | | | | | |
| D-1056 | 1617 | GAGCAAGCCUAAACGUCAGAA | 57 | UUCUGACGUUUAGGCUUGCUC | 601 |
| D-1057 | 1618 | AGCAAGCCUAAACGUCAGAAA | 58 | UUUCUGACGUUUAGGCUUGCU | 602 |
| D-1825 | | | | | |
| D-1058 | 1619 | GCAAGCCUAAACGUCAGAAAU | 59 | AUUUCUGACGUUUAGGCUUGC | 603 |
| D-1574 | | | | | |
| D-1059 | 1620 | CAAGCCUAAACGUCAGAAAUC | 60 | GAUUUCUGACGUUUAGGCUUG | 604 |
| D-1800 | | | | | |
| D-1060 | 1621 | AAGCCUAAACGUCAGAAAUCC | 61 | GGAUUUCUGACGUUUAGGCUU | 605 |
| D-1801 | | | | | |
| D-1061 | 1631 | GUCAGAAAUCCAGUACUAAAC | 62 | GUUUAGUACUGGAUUUCUGAC | 606 |
| D-1610 | | | | | |
| D-1062 | 1632 | UCAGAAAUCCAGUACUAAACU | 63 | AGUUUAGUACUGGAUUUCUGA | 607 |
| D-1554 | | | | | |
| D-1063 | 1652 | UUUCUGAGCUUCAUGACAAUC | 64 | GAUUGUCAUGAAGCUCAGAAA | 608 |
| D-1064 | 1658 | AGCUUCAUGACAAUCAGGACG | 65 | CGUCCUGAUUGUCAUGAAGCU | 609 |
| D-1065 | 1661 | UUCAUGACAAUCAGGACGGUC | 66 | GACCGUCCUGAUUGUCAUGAA | 610 |
| D-1066 | 1662 | UCAUGACAAUCAGGACGGUCU | 67 | AGACCGUCCUGAUUGUCAUGA | 611 |
| D-1067 | 1663 | CAUGACAAUCAGGACGGUCUU | 68 | AAGACCGUCCUGAUUGUCAUG | 612 |
| D-1068 | 1664 | AUGACAAUCAGGACGGUCUUG | 69 | CAAGACCGUCCUGAUUGUCAU | 613 |
| D-1069 | 1665 | UGACAAUCAGGACGGUCUUGU | 70 | ACAAGACCGUCCUGAUUGUCA | 614 |
| D-1070 | 1666 | GACAAUCAGGACGGUCUUGUG | 71 | CACAAGACCGUCCUGAUUGUC | 615 |
| D-1607 | | | | | |
| D-1071 | 1667 | ACAAUCAGGACGGUCUUGUGA | 72 | UCACAAGACCGUCCUGAUUGU | 616 |
| D-1072 | 1669 | AAUCAGGACGGUCUUGUGAAU | 73 | AUUCACAAGACCGUCCUGAUU | 617 |
| D-1073 | 1670 | AUCAGGACGGUCUUGUGAAUA | 74 | UAUUCACAAGACCGUCCUGAU | 618 |
| D-1074 | 1671 | UCAGGACGGUCUUGUGAAUAU | 75 | AUAUUCACAAGACCGUCCUGA | 619 |
| D-1609 | | | | | |
| D-1075 | 1678 | GGUCUUGUGAAUAUGGAAAGU | 76 | ACUUUCCAUAUUCACAAGACC | 620 |
| D-1615 | | | | | |
| D-1695 | | | | | |
| D-1701 | | | | | |
| D-1708 | | | | | |
| D-1715 | | | | | |
| D-1722 | | | | | |
| D-1729 | | | | | |
| D-1736 | | | | | |
| D-1852 | | | | | |
| D-1867 | | | | | |
| D-2007 | | | | | |
| D-2016 | | | | | |
| D-1076 | 1693 | GAAAGUCUCAAUUCCACACGA | 77 | UCGUGUGGAAUUGAGACUUUC | 621 |
| D-1826 | | | | | |
| D-1077 | 1694 | AAAGUCUCAAUUCCACACGAU | 78 | AUCGUGUGGAAUUGAGACUUU | 622 |
| D-1078 | 1695 | AAGUCUCAAUUCCACACGAUC | 79 | GAUCGUGUGGAAUUGAGACUU | 623 |
| D-1079 | 1696 | AGUCUCAAUUCCACACGAUCU | 80 | AGAUCGUGUGGAAUUGAGACU | 624 |
| D-1080 | 1697 | GUCUCAAUUCCACACGAUCUC | 81 | GAGAUCGUGUGGAAUUGAGAC | 625 |
| D-1827 | | | | | |
| D-1081 | 1698 | UCUCAAUUCCACACGAUCUCA | 82 | UGAGAUCGUGUGGAAUUGAGA | 626 |
| D-1605 | | | | | |
| D-1082 | 1699 | CUCAAUUCCACACGAUCUCAU | 83 | AUGAGAUCGUGUGGAAUUGAG | 627 |
| D-1083 | 1700 | UCAAUUCCACACGAUCUCAUG | 84 | CAUGAGAUCGUGUGGAAUUGA | 628 |
| D-1828 | | | | | |
| D-1084 | 1701 | CAAUUCCACACGAUCUCAUGA | 85 | UCAUGAGAUCGUGUGGAAUUG | 629 |
| D-1829 | | | | | |
| D-1085 | 1703 | AUUCCACACGAUCUCAUGAGA | 86 | UCUCAUGAGAUCGUGUGGAAU | 630 |
| D-1830 | | | | | |
| D-1086 | 1704 | UUCCACACGAUCUCAUGAGAG | 87 | CUCUCAUGAGAUCGUGUGGAA | 631 |
| D-1831 | | | | | |
| D-1087 | 1705 | UCCACACGAUCUCAUGAGAGA | 88 | UCUCUCAUGAGAUCGUGUGGA | 632 |
| D-1606 | | | | | |
| D-1088 | 1709 | CACGAUCUCAUGAGAGAACUG | 89 | CAGUUCUCUCAUGAGAUCGUG | 633 |
| D-1089 | 1714 | UCUCAUGAGAGAACUGGACCU | 90 | AGGUCCAGUUCUCUCAUGAGA | 634 |
| D-1090 | 1716 | UCAUGAGAGAACUGGACCUGA | 91 | UCAGGUCCAGUUCUCUCAUGA | 635 |
| D-1832 | | | | | |
| D-1091 | 1717 | CAUGAGAGAACUGGACCUGAU | 92 | AUCAGGUCCAGUUCUCUCAUG | 636 |
| D-1833 | | | | | |
| D-1092 | 1742 | UUGAAUGGAUGUCUGAUGAAA | 93 | UUUCAUCAGACAUCCAUUCAA | 637 |
| D-1093 | 1783 | GGUGGACACACUCAGCAUUUU | 94 | AAAAUGCUGAGUGUGUCCACC | 638 |
| D-1802 | | | | | |
| D-1094 | 1801 | UUUGAGAGCCCCACAAUGAAG | 95 | CUUCAUUGUGGGGCUCUCAAA | 639 |
| D-1587 | | | | | |
| D-1095 | 1832 | AUCCCAGCCUAUCUGACACCA | 96 | UGGUGUCAGAUAGGCUGGGAU | 640 |
| D-1834 | | | | | |
| D-1096 | 1833 | UCCCAGCCUAUCUGACACCAA | 97 | UUGGUGUCAGAUAGGCUGGGA | 641 |
| D-1835 | | | | | |
| D-1097 | 1834 | CCCAGCCUAUCUGACACCAAA | 98 | UUUGGUGUCAGAUAGGCUGGG | 642 |
| D-1836 | | | | | |
| D-1098 | 1835 | CCAGCCUAUCUGACACCAAAC | 99 | GUUUGGUGUCAGAUAGGCUGG | 643 |
| D-1099 | 1836 | CAGCCUAUCUGACACCAAACA | 100 | UGUUUGGUGUCAGAUAGGCUG | 644 |
| D-1100 | 1856 | AGCAGAGAAAUCAAGAUGCCG | 101 | CGGCAUCUUGAUUUCUCUGCU | 645 |
| D-1837 | | | | | |
| D-1101 | 1890 | GAGCUUUGUCUCCGAAGUGCC | 102 | GGCACUUCGGAGACAAAGCUC | 646 |
| D-1803 | | | | | |
| D-1102 | 1896 | UGUCUCCGAAGUGCCCCAGUC | 103 | GACUGGGGCACUUCGGAGACA | 647 |
| D-1563 | | | | | |
| D-1804 | | | | | |
| D-1103 | 1899 | CUCCGAAGUGCCCCAGUCGGA | 104 | UCCGACUGGGGCACUUCGGAG | 648 |
| D-1104 | 1900 | UCCGAAGUGCCCCAGUCGGAC | 105 | GUCCGACUGGGGCACUUCGGA | 649 |
| D-1838 | | | | | |
| D-1105 | 1943 | AGAACUGGGAAGAGCCUAUCC | 106 | GGAUAGGCUCUUCCCAGUUCU | 650 |
| D-1106 | 1944 | GAACUGGGAAGAGCCUAUCCC | 107 | GGGAUAGGCUCUUCCCAGUUC | 651 |
| D-1107 | 1952 | AAGAGCCUAUCCCUGCUUUCU | 108 | AGAAAGCAGGGAUAGGCUCUU | 652 |
| D-1608 | | | | | |
| D-1108 | 2024 | AGGCUGGGCGCCUGAUCCGUC | 109 | GACGGAUCAGGCGCCCAGCCU | 653 |
| D-1109 | 2025 | GGCUGGGCGCCUGAUCCGUCA | 110 | UGACGGAUCAGGCGCCCAGCC | 654 |
| D-1110 | 2026 | GCUGGGCGCCUGAUCCGUCAG | 111 | CUGACGGAUCAGGCGCCCAGC | 655 |
| D-1111 | 2027 | CUGGGCGCCUGAUCCGUCAGC | 112 | GCUGACGGAUCAGGCGCCCAG | 656 |
| D-1112 | 2028 | UGGGCGCCUGAUCCGUCAGCU | 113 | AGCUGACGGAUCAGGCGCCCA | 657 |
| D-1113 | 2050 | CUGGACGAAGACAGCGACCCC | 114 | GGGGUCGCUGUCUUCGUCCAG | 658 |
| D-2094 | 2055 | CGAAGACAGCGACCCCAUGCU | 2807 | AGCAUGGGGUCGCUGUCUUCG | 2808 |
| D-1114 | 2066 | ACCCCAUGCUCUCUCCUCGGU | 115 | ACCGAGGAGAGAGCAUGGGGU | 659 |
| D-1568 | | | | | |
| D-1115 | 2070 | CAUGCUCUCUCCUCGGUUCUA | 116 | UAGAACCGAGGAGAGAGCAUG | 660 |
| D-1567 | | | | | |
| D-1116 | 2071 | AUGCUCUCUCCUCGGUUCUAC | 117 | GUAGAACCGAGGAGAGAGCAU | 661 |
| D-1805 | | | | | |
| D-1117 | 2073 | GCUCUCUCCUCGGUUCUACGC | 118 | GCGUAGAACCGAGGAGAGAGC | 662 |
| D-1118 | 2074 | CUCUCUCCUCGGUUCUACGCU | 119 | AGCGUAGAACCGAGGAGAGAG | 663 |
| D-1119 | 2075 | UCUCUCCUCGGUUCUACGCUU | 120 | AAGCGUAGAACCGAGGAGAGA | 664 |
| D-1601 | | | | | |
| D-1120 | 2076 | CUCUCCUCGGUUCUACGCUUA | 121 | UAAGCGUAGAACCGAGGAGAG | 665 |
| D-1121 | 2077 | UCUCCUCGGUUCUACGCUUAU | 122 | AUAAGCGUAGAACCGAGGAGA | 666 |
| D-1122 | 2078 | CUCCUCGGUUCUACGCUUAUG | 123 | CAUAAGCGUAGAACCGAGGAG | 667 |
| D-1550 | | | | | |
| D-1123 | 2079 | UCCUCGGUUCUACGCUUAUGG | 124 | CCAUAAGCGUAGAACCGAGGA | 668 |
| D-1124 | 2080 | CCUCGGUUCUACGCUUAUGGG | 125 | CCCAUAAGCGUAGAACCGAGG | 669 |
| D-1549 | | | | | |
| D-1643 | | | | | |
| D-1647 | | | | | |
| D-1651 | | | | | |
| D-1656 | | | | | |
| D-1661 | | | | | |
| D-1666 | | | | | |
| D-1671 | | | | | |
| D-1675 | | | | | |
| D-1680 | | | | | |
| D-1685 | | | | | |
| D-1690 | | | | | |
| D-1848 | | | | | |
| D-1860 | | | | | |
| D-1125 | 2081 | CUCGGUUCUACGCUUAUGGGC | 126 | GCCCAUAAGCGUAGAACCGAG | 670 |
| D-1126 | 2144 | CACCAAACUCCCAUUCUUUCA | 127 | UGAAAGAAUGGGAGUUUGGUG | 671 |
| D-1544 | | | | | |
| D-1636 | | | | | |
| D-1648 | | | | | |
| D-1653 | | | | | |
| D-1658 | | | | | |
| D-1663 | | | | | |
| D-1668 | | | | | |
| D-1672 | | | | | |
| D-1677 | | | | | |
| D-1682 | | | | | |
| D-1687 | | | | | |
| D-1692 | | | | | |
| D-1851 | | | | | |
| D-1858 | | | | | |
| D-2010 | | | | | |
| D-2019 | | | | | |
| D-1127 | 2146 | CCAAACUCCCAUUCUUUCAUG | 128 | CAUGAAAGAAUGGGAGUUUGG | 672 |
| D-1565 | | | | | |
| D-1641 | | | | | |
| D-1128 | 2151 | CUCCCAUUCUUUCAUGAGGCG | 129 | CGCCUCAUGAAAGAAUGGGAG | 673 |
| D-1539 | | | | | |
| D-1129 | 2155 | CAUUCUUUCAUGAGGCGGCGA | 130 | UCGCCGCCUCAUGAAAGAAUG | 674 |
| D-1130 | 2156 | AUUCUUUCAUGAGGCGGCGAA | 131 | UUCGCCGCCUCAUGAAAGAAU | 675 |
| D-1131 | 2157 | UUCUUUCAUGAGGCGGCGAAG | 132 | CUUCGCCGCCUCAUGAAAGAA | 676 |
| D-1132 | 2158 | UCUUUCAUGAGGCGGCGAAGC | 133 | GCUUCGCCGCCUCAUGAAAGA | 677 |
| D-1133 | 2159 | CUUUCAUGAGGCGGCGAAGCU | 134 | AGCUUCGCCGCCUCAUGAAAG | 678 |
| D-1134 | 2160 | UUUCAUGAGGCGGCGAAGCUC | 135 | GAGCUUCGCCGCCUCAUGAAA | 679 |
| D-1135 | 2182 | UCUCUGGGGUCCUAUGAUGAU | 136 | AUCAUCAUAGGACCCCAGAGA | 680 |
| D-1136 | 2218 | ACACCUGCCCAGCUCACACGA | 137 | UCGUGUGAGCUGGGCAGGUGU | 681 |
| D-1137 | 2219 | CACCUGCCCAGCUCACACGAA | 138 | UUCGUGUGAGCUGGGCAGGUG | 682 |
| D-1138 | 2221 | CCUGCCCAGCUCACACGAAGG | 139 | CCUUCGUGUGAGCUGGGCAGG | 683 |
| D-1139 | 2226 | CCAGCUCACACGAAGGAUUCA | 140 | UGAAUCCUUCGUGUGAGCUGG | 684 |
| D-1140 | 2228 | AGCUCACACGAAGGAUUCAGA | 141 | UCUGAAUCCUUCGUGUGAGCU | 685 |
| D-1806 | | | | | |
| D-1141 | 2263 | AUCCGGAAGUUUGAAGAUAGA | 142 | UCUAUCUUCAAACUUCCGGAU | 686 |
| D-1573 | | | | | |
| D-1638 | | | | | |
| D-1644 | | | | | |
| D-1645 | | | | | |
| D-2024 | | | | | |
| D-2025 | | | | | |
| D-2026 | | | | | |
| D-2027 | | | | | |
| D-2028 | | | | | |
| D-2029 | | | | | |
| D-2030 | | | | | |
| D-2031 | | | | | |
| D-2032 | | | | | |
| D-2033 | | | | | |
| D-2034 | | | | | |
| D-1142 | 2266 | CGGAAGUUUGAAGAUAGAUUC | 143 | GAAUCUAUCUUCAAACUUCCG | 687 |
| D-1547 | | | | | |
| D-1143 | 2270 | AGUUUGAAGAUAGAUUCGAAG | 144 | CUUCGAAUCUAUCUUCAAACU | 688 |
| D-1602 | | | | | |
| D-1144 | 2271 | GUUUGAAGAUAGAUUCGAAGA | 145 | UCUUCGAAUCUAUCUUCAAAC | 689 |
| D-1145 | 2275 | GAAGAUAGAUUCGAAGAAGAG | 146 | CUCUUCUUCGAAUCUAUCUUC | 690 |
| D-1839 | | | | | |
| D-1146 | 2294 | AGAAGAAGUACAGACCUUCCC | 147 | GGGAAGGUCUGUACUUCUUCU | 691 |
| D-1807 | | | | | |
| D-1147 | 2295 | GAAGAAGUACAGACCUUCCCA | 148 | UGGGAAGGUCUGUACUUCUUC | 692 |
| D-1808 | | | | | |
| D-1148 | 2296 | AAGAAGUACAGACCUUCCCAC | 149 | GUGGGAAGGUCUGUACUUCUU | 693 |
| D-1809 | | | | | |
| D-1149 | 2343 | UCUGAAAUGGACAAAUGACCU | 150 | AGGUCAUUUGUCCAUUUCAGA | 694 |
| D-1810 | | | | | |
| D-1938 | | | | | |
| D-1944 | | | | | |
| D-1950 | | | | | |
| D-1956 | | | | | |
| D-1962 | | | | | |
| D-1968 | | | | | |
| D-1974 | | | | | |
| D-2035 | | | | | |
| D-2055 | | | | | |
| D-2056 | | | | | |
| D-1150 | 2344 | CUGAAAUGGACAAAUGACCUU | 151 | AAGGUCAUUUGUCCAUUUCAG | 695 |
| D-1613 | | | | | |
| D-1151 | 2353 | ACAAAUGACCUUGCCAAAUUC | 152 | GAAUUUGGCAAGGUCAUUUGU | 696 |
| D-1598 | | | | | |
| D-1152 | 2355 | AAAUGACCUUGCCAAAUUCCG | 153 | CGGAAUUUGGCAAGGUCAUUU | 697 |
| D-1811 | | | | | |
| D-1153 | 2356 | AAUGACCUUGCCAAAUUCCGG | 154 | CCGGAAUUUGGCAAGGUCAUU | 698 |
| D-1556 | | | | | |
| D-1154 | 2358 | UGACCUUGCCAAAUUCCGGAG | 155 | CUCCGGAAUUUGGCAAGGUCA | 699 |
| D-1595 | | | | | |
| D-1155 | 2359 | GACCUUGCCAAAUUCCGGAGA | 156 | UCUCCGGAAUUUGGCAAGGUC | 700 |
| D-1156 | 2360 | ACCUUGCCAAAUUCCGGAGAC | 157 | GUCUCCGGAAUUUGGCAAGGU | 701 |
| D-1578 | | | | | |
| D-1157 | 2361 | CCUUGCCAAAUUCCGGAGACA | 158 | UGUCUCCGGAAUUUGGCAAGG | 702 |
| D-1158 | 2373 | CCGGAGACAACUUAAAGAAUC | 159 | GAUUCUUUAAGUUGUCUCCGG | 703 |
| D-1159 | 2374 | CGGAGACAACUUAAAGAAUCA | 160 | UGAUUCUUUAAGUUGUCUCCG | 704 |
| D-1160 | 2402 | AGAUAUCUGAAGAGGACCUAA | 161 | UUAGGUCCUCUUCAGAUAUCU | 705 |
| D-1161 | 2413 | GAGGACCUAACUCCCAGGAUG | 162 | CAUCCUGGGAGUUAGGUCCUC | 706 |
| D-1162 | 2416 | GACCUAACUCCCAGGAUGCGG | 163 | CCGCAUCCUGGGAGUUAGGUC | 707 |
| D-1163 | 2417 | ACCUAACUCCCAGGAUGCGGC | 164 | GCCGCAUCCUGGGAGUUAGGU | 708 |
| D-1812 | | | | | |
| D-1935 | | | | | |
| D-1941 | | | | | |
| D-1947 | | | | | |
| D-1953 | | | | | |
| D-1959 | | | | | |
| D-1965 | | | | | |
| D-1971 | | | | | |
| D-1164 | 2432 | UGCGGCAGCGAAGCAACACAC | 165 | GUGUGUUGCUUCGCUGCCGCA | 709 |
| D-1813 | | | | | |
| D-1165 | 2433 | GCGGCAGCGAAGCAACACACU | 166 | AGUGUGUUGCUUCGCUGCCGC | 710 |
| D-1166 | 2437 | CAGCGAAGCAACACACUCCCC | 167 | GGGGAGUGUGUUGCUUCGCUG | 711 |
| D-1840 | | | | | |
| D-1167 | 2439 | GCGAAGCAACACACUCCCCAA | 168 | UUGGGGAGUGUGUUGCUUCGC | 712 |
| D-1841 | | | | | |
| D-1168 | 2444 | GCAACACACUCCCCAAGAGUU | 169 | AACUCUUGGGGAGUGUGUUGC | 713 |
| D-1169 | 2457 | CAAGAGUUUUGGUUCCCAACU | 170 | AGUUGGGAACCAAAACUCUUG | 714 |
| D-1170 | 2460 | GAGUUUUGGUUCCCAACUUGA | 171 | UCAAGUUGGGAACCAAAACUC | 715 |
| D-1171 | 2462 | GUUUUGGUUCCCAACUUGAGA | 172 | UCUCAAGUUGGGAACCAAAAC | 716 |
| D-1592 | | | | | |
| D-1172 | 2534 | UUGAAGCCACAUUGGAAUCUA | 173 | UAGAUUCCAAUGUGGCUUCAA | 717 |
| D-1842 | | | | | |
| D-1173 | 2568 | CCAGGAGAAGCGAGCGGAAAG | 174 | CUUUCCGCUCGCUUCUCCUGG | 718 |
| D-1174 | 2623 | GACCAGAUUGCUAAUGAGAAA | 175 | UUUCUCAUUAGCAAUCUGGUC | 719 |
| D-1581 | | | | | |
| D-1175 | 2632 | GCUAAUGAGAAAGUGGCUCUG | 176 | CAGAGCCACUUUCUCAUUAGC | 720 |
| D-1621 | | | | | |
| D-1176 | 2677 | AGCAUUCAUGGACGGCCGGUA | 177 | UACCGGCCGUCCAUGAAUGCU | 721 |
| D-1177 | 2678 | GCAUUCAUGGACGGCCGGUAA | 178 | UUACCGGCCGUCCAUGAAUGC | 722 |
| D-1178 | 2679 | CAUUCAUGGACGGCCGGUAAC | 179 | GUUACCGGCCGUCCAUGAAUG | 723 |
| D-1179 | 2680 | AUUCAUGGACGGCCGGUAACA | 180 | UGUUACCGGCCGUCCAUGAAU | 724 |
| D-1180 | 2681 | UUCAUGGACGGCCGGUAACAA | 181 | UUGUUACCGGCCGUCCAUGAA | 725 |
| D-1181 | 2682 | UCAUGGACGGCCGGUAACAAA | 182 | UUUGUUACCGGCCGUCCAUGA | 726 |
| D-2095 | 2683 | CAUGGACGGCCGGUAACAAAG | 2809 | CUUUGUUACCGGCCGUCCAUG | 2810 |
| D-1182 | 2684 | AUGGACGGCCGGUAACAAAGA | 183 | UCUUUGUUACCGGCCGUCCAU | 727 |
| D-1183 | 2685 | UGGACGGCCGGUAACAAAGAA | 184 | UUCUUUGUUACCGGCCGUCCA | 728 |
| D-1184 | 2686 | GGACGGCCGGUAACAAAGAAC | 185 | GUUCUUUGUUACCGGCCGUCC | 729 |
| D-1185 | 2688 | ACGGCCGGUAACAAAGAACGA | 186 | UCGUUCUUUGUUACCGGCCGU | 730 |
| D-1814 | | | | | |
| D-1186 | 2689 | CGGCCGGUAACAAAGAACGAA | 187 | UUCGUUCUUUGUUACCGGCCG | 731 |
| D-1187 | 2690 | GGCCGGUAACAAAGAACGAAC | 188 | GUUCGUUCUUUGUUACCGGCC | 732 |
| D-1815 | | | | | |
| D-1188 | 2691 | GCCGGUAACAAAGAACGAACG | 189 | CGUUCGUUCUUUGUUACCGGC | 733 |
| D-1189 | 2692 | CCGGUAACAAAGAACGAACGG | 190 | CCGUUCGUUCUUUGUUACCGG | 734 |
| D-1190 | 2693 | CGGUAACAAAGAACGAACGGC | 191 | GCCGUUCGUUCUUUGUUACCG | 735 |
| D-1843 | | | | | |
| D-1191 | 2694 | GGUAACAAAGAACGAACGGCA | 192 | UGCCGUUCGUUCUUUGUUACC | 736 |
| D-1192 | 2700 | AAAGAACGAACGGCAGGUGAU | 193 | AUCACCUGCCGUUCGUUCUUU | 737 |
| D-1193 | 2719 | AUGAAGCCACUAUACGACAGG | 194 | CCUGUCGUAUAGUGGCUUCAU | 738 |
| D-1844 | | | | | |
| D-1194 | 2721 | GAAGCCACUAUACGACAGGUA | 195 | UACCUGUCGUAUAGUGGCUUC | 739 |
| D-1195 | 2722 | AAGCCACUAUACGACAGGUAC | 196 | GUACCUGUCGUAUAGUGGCUU | 740 |
| D-1196 | 2723 | AGCCACUAUACGACAGGUACC | 197 | GGUACCUGUCGUAUAGUGGCU | 741 |
| D-1197 | 2724 | GCCACUAUACGACAGGUACCG | 198 | CGGUACCUGUCGUAUAGUGGC | 742 |
| D-1198 | 2725 | CCACUAUACGACAGGUACCGG | 199 | CCGGUACCUGUCGUAUAGUGG | 743 |
| D-1199 | 2726 | CACUAUACGACAGGUACCGGC | 200 | GCCGGUACCUGUCGUAUAGUG | 744 |
| D-1845 | | | | | |
| D-1200 | 2727 | ACUAUACGACAGGUACCGGCU | 201 | AGCCGGUACCUGUCGUAUAGU | 745 |
| D-1201 | 2753 | AACAGAUCCUCUCCCGAGCUA | 202 | UAGCUCGGGAGAGGAUCUGUU | 746 |
| D-1202 | 2754 | ACAGAUCCUCUCCCGAGCUAA | 203 | UUAGCUCGGGAGAGGAUCUGU | 747 |
| D-1203 | 2756 | AGAUCCUCUCCCGAGCUAACA | 204 | UGUUAGCUCGGGAGAGGAUCU | 748 |
| D-1204 | 2759 | UCCUCUCCCGAGCUAACACCA | 205 | UGGUGUUAGCUCGGGAGAGGA | 749 |
| D-1205 | 2760 | CCUCUCCCGAGCUAACACCAU | 206 | AUGGUGUUAGCUCGGGAGAGG | 750 |
| D-1206 | 2761 | CUCUCCCGAGCUAACACCAUA | 207 | UAUGGUGUUAGCUCGGGAGAG | 751 |
| D-1207 | 2764 | UCCCGAGCUAACACCAUACCC | 208 | GGGUAUGGUGUUAGCUCGGGA | 752 |
| D-1208 | 2765 | CCCGAGCUAACACCAUACCCA | 209 | UGGGUAUGGUGUUAGCUCGGG | 753 |
| D-1209 | 2886 | GGGGUCAGAAGACGAUAGCAA | 210 | UUGCUAUCGUCUUCUGACCCC | 754 |
| D-1816 | | | | | |
| D-1210 | 2887 | GGGUCAGAAGACGAUAGCAAU | 211 | AUUGCUAUCGUCUUCUGACCC | 755 |
| D-1561 | | | | | |
| D-1211 | 2889 | GUCAGAAGACGAUAGCAAUGU | 212 | ACAUUGCUAUCGUCUUCUGAC | 756 |
| D-1620 | | | | | |
| D-1212 | 2890 | UCAGAAGACGAUAGCAAUGUG | 213 | CACAUUGCUAUCGUCUUCUGA | 757 |
| D-1560 | | | | | |
| D-1213 | 2893 | GAAGACGAUAGCAAUGUGAAG | 214 | CUUCACAUUGCUAUCGUCUUC | 758 |
| D-1559 | | | | | |
| D-1214 | 2895 | AGACGAUAGCAAUGUGAAGCC | 215 | GGCUUCACAUUGCUAUCGUCU | 759 |
| D-1558 | | | | | |
| D-1215 | 2923 | AUGGUCACUCUGAAAACCGAU | 216 | AUCGGUUUUCAGAGUGACCAU | 760 |
| D-1604 | | | | | |
| D-1216 | 2924 | UGGUCACUCUGAAAACCGAUU | 217 | AAUCGGUUUUCAGAGUGACCA | 761 |
| D-1217 | 2925 | GGUCACUCUGAAAACCGAUUU | 218 | AAAUCGGUUUUCAGAGUGACC | 762 |
| D-1218 | 2934 | GAAAACCGAUUUCAGUGCACG | 219 | CGUGCACUGAAAUCGGUUUUC | 763 |
| D-1541 | | | | | |
| D-1219 | 2937 | AACCGAUUUCAGUGCACGAUG | 220 | CAUCGUGCACUGAAAUCGGUU | 764 |
| D-1588 | | | | | |
| D-1220 | 2994 | UAUUUCCCCAAUGGAUGAUAA | 221 | UUAUCAUCCAUUGGGGAAAUA | 765 |
| D-1619 | | | | | |
| D-1221 | 3000 | CCCAAUGGAUGAUAAAAUACC | 222 | GGUAUUUUAUCAUCCAUUGGG | 766 |
| D-1557 | | | | | |
| D-1642 | | | | | |
| D-1650 | | | | | |
| D-1655 | | | | | |
| D-1660 | | | | | |
| D-1665 | | | | | |
| D-1674 | | | | | |
| D-1679 | | | | | |
| D-1684 | | | | | |
| D-1689 | | | | | |
| D-1850 | | | | | |
| D-1861 | | | | | |
| D-1222 | 3002 | CAAUGGAUGAUAAAAUACCAU | 223 | AUGGUAUUUUAUCAUCCAUUG | 767 |
| D-1579 | | | | | |
| D-1223 | 3005 | UGGAUGAUAAAAUACCAUCAA | 224 | UUGAUGGUAUUUUAUCAUCCA | 768 |
| D-1224 | 3014 | AAAUACCAUCAAAAUGCAGCC | 225 | GGCUGCAUUUUGAUGGUAUUU | 769 |
| D-1555 | | | | | |
| D-1225 | 3043 | GGGCUUUCAAAUCUCCAUGCU | 226 | AGCAUGGAGAUUUGAAAGCCC | 770 |
| D-1226 | 3044 | GGCUUUCAAAUCUCCAUGCUG | 227 | CAGCAUGGAGAUUUGAAAGCC | 771 |
| D-1227 | 3052 | AAUCUCCAUGCUGCCUCAAUA | 228 | UAUUGAGGCAGCAUGGAGAUU | 772 |
| D-1228 | 3053 | AUCUCCAUGCUGCCUCAAUAC | 229 | GUAUUGAGGCAGCAUGGAGAU | 773 |
| D-1229 | 3054 | UCUCCAUGCUGCCUCAAUACC | 230 | GGUAUUGAGGCAGCAUGGAGA | 774 |
| D-1230 | 3062 | CUGCCUCAAUACCUGAACUCC | 231 | GGAGUUCAGGUAUUGAGGCAG | 775 |
| D-1231 | 3082 | CUGGAACACCUCCAGGAAAUG | 232 | CAUUUCCUGGAGGUGUUCCAG | 776 |
| D-1232 | 3133 | CUUCGGGAUUUUGAAGACAAC | 233 | GUUGUCUUCAAAAUCCCGAAG | 777 |
| D-1586 | | | | | |
| D-1637 | | | | | |
| D-1649 | | | | | |
| D-1654 | | | | | |
| D-1659 | | | | | |
| D-1664 | | | | | |
| D-1669 | | | | | |
| D-1673 | | | | | |
| D-1678 | | | | | |
| D-1683 | | | | | |
| D-1688 | | | | | |
| D-1693 | | | | | |
| D-1233 | 3180 | CCAGAAGGAAGACCGCACUCC | 234 | GGAGUGCGGUCUUCCUUCUGG | 778 |
| D-1234 | 3183 | GAAGGAAGACCGCACUCCUAU | 235 | AUAGGAGUGCGGUCUUCCUUC | 779 |
| D-1235 | 3184 | AAGGAAGACCGCACUCCUAUG | 236 | CAUAGGAGUGCGGUCUUCCUU | 780 |
| D-1540 | | | | | |
| D-1236 | 3185 | AGGAAGACCGCACUCCUAUGG | 237 | CCAUAGGAGUGCGGUCUUCCU | 781 |
| D-1237 | 3186 | GGAAGACCGCACUCCUAUGGC | 238 | GCCAUAGGAGUGCGGUCUUCC | 782 |
| D-1238 | 3187 | GAAGACCGCACUCCUAUGGCU | 239 | AGCCAUAGGAGUGCGGUCUUC | 783 |
| D-1552 | | | | | |
| D-1239 | 3189 | AGACCGCACUCCUAUGGCUGA | 240 | UCAGCCAUAGGAGUGCGGUCU | 784 |
| D-1618 | | | | | |
| D-1240 | 3192 | CCGCACUCCUAUGGCUGAAGA | 241 | UCUUCAGCCAUAGGAGUGCGG | 785 |
| D-1585 | | | | | |
| D-1241 | 3225 | UAAGCACAUAAAGGCGAAACU | 242 | AGUUUCGCCUUUAUGUGCUUA | 786 |
| D-1242 | 3226 | AAGCACAUAAAGGCGAAACUG | 243 | CAGUUUCGCCUUUAUGUGCUU | 787 |
| D-1243 | 3228 | GCACAUAAAGGCGAAACUGAG | 244 | CUCAGUUUCGCCUUUAUGUGC | 788 |
| D-1244 | 3283 | GAUUCCAAGUCCAUGUGAGGG | 245 | CCCUCACAUGGACUUGGAAUC | 789 |
| D-1584 | | | | | |
| D-1245 | 3284 | AUUCCAAGUCCAUGUGAGGGG | 246 | CCCCUCACAUGGACUUGGAAU | 790 |
| D-1246 | 3287 | CCAAGUCCAUGUGAGGGGCAU | 247 | AUGCCCCUCACAUGGACUUGG | 791 |
| D-1247 | 3288 | CAAGUCCAUGUGAGGGGCAUG | 248 | CAUGCCCCUCACAUGGACUUG | 792 |
| D-1248 | 3291 | GUCCAUGUGAGGGGCAUGGCC | 249 | GGCCAUGCCCCUCACAUGGAC | 793 |
| D-1249 | 3327 | GCAGCUGCGGUGAGAGUUUAC | 250 | GUAAACUCUCACCGCAGCUGC | 794 |
| D-1250 | 3329 | AGCUGCGGUGAGAGUUUACUG | 251 | CAGUAAACUCUCACCGCAGCU | 795 |
| D-1251 | 3352 | CCCAGAGAAAGUGCAGCUCUG | 252 | CAGAGCUGCACUUUCUCUGGG | 796 |
| D-1252 | 3398 | CAAAGCAUGCAGCCCUUCUGC | 253 | GCAGAAGGGCUGCAUGCUUUG | 797 |
| D-1253 | 3411 | CCUUCUGCCUCUAGACCAUUU | 254 | AAAUGGUCUAGAGGCAGAAGG | 798 |
| D-1254 | 3414 | UCUGCCUCUAGACCAUUUGGC | 255 | GCCAAAUGGUCUAGAGGCAGA | 799 |
| D-1255 | 3420 | UCUAGACCAUUUGGCAUCGGC | 256 | GCCGAUGCCAAAUGGUCUAGA | 800 |
| D-1256 | 3421 | CUAGACCAUUUGGCAUCGGCU | 257 | AGCCGAUGCCAAAUGGUCUAG | 801 |
| D-1257 | 3422 | UAGACCAUUUGGCAUCGGCUC | 258 | GAGCCGAUGCCAAAUGGUCUA | 802 |
| D-1258 | 3423 | AGACCAUUUGGCAUCGGCUCC | 259 | GGAGCCGAUGCCAAAUGGUCU | 803 |
| D-1259 | 3429 | UUUGGCAUCGGCUCCUGUUUC | 260 | GAAACAGGAGCCGAUGCCAAA | 804 |
| D-1632 | | | | | |
| D-1260 | 3432 | GGCAUCGGCUCCUGUUUCCAU | 261 | AUGGAAACAGGAGCCGAUGCC | 805 |
| D-1261 | 3436 | UCGGCUCCUGUUUCCAUUGCC | 262 | GGCAAUGGAAACAGGAGCCGA | 806 |
| D-1262 | 3438 | GGCUCCUGUUUCCAUUGCCUG | 263 | CAGGCAAUGGAAACAGGAGCC | 807 |
| D-1580 | | | | | |
| D-1263 | 3498 | UAGGCAUUUUGUAAUUGGAAA | 264 | UUUCCAAUUACAAAAUGCCUA | 808 |
| D-1583 | | | | | |
| D-1264 | 3499 | AGGCAUUUUGUAAUUGGAAAG | 265 | CUUUCCAAUUACAAAAUGCCU | 809 |
| D-1582 | | | | | |
| D-1265 | 3503 | AUUUUGUAAUUGGAAAGUCAA | 266 | UUGACUUUCCAAUUACAAAAU | 810 |
| D-1266 | 3505 | UUUGUAAUUGGAAAGUCAAGA | 267 | UCUUGACUUUCCAAUUACAAA | 811 |
| D-1267 | 3510 | AAUUGGAAAGUCAAGACUGCA | 268 | UGCAGUCUUGACUUUCCAAUU | 812 |
| D-1268 | 3514 | GGAAAGUCAAGACUGCAGUAU | 269 | AUACUGCAGUCUUGACUUUCC | 813 |
| D-1269 | 3519 | GUCAAGACUGCAGUAUGUGCA | 270 | UGCACAUACUGCAGUCUUGAC | 814 |
| D-1270 | 3520 | UCAAGACUGCAGUAUGUGCAC | 271 | GUGCACAUACUGCAGUCUUGA | 815 |
| D-2096 | 3536 | UGCACAUGCGCACGCGCAUGC | 2811 | GCAUGCGCGUGCGCAUGUGCA | 2812 |
| D-2097 | 3537 | GCACAUGCGCACGCGCAUGCA | 2813 | UGCAUGCGCGUGCGCAUGUGC | 2814 |
| D-2098 | 3538 | CACAUGCGCACGCGCAUGCAC | 2815 | GUGCAUGCGCGUGCGCAUGUG | 2816 |
| D-2099 | 3539 | ACAUGCGCACGCGCAUGCACG | 2817 | CGUGCAUGCGCGUGCGCAUGU | 2818 |
| D-1271 | 3565 | ACACACACAGUAGUGGAGCUU | 272 | AAGCUCCACUACUGUGUGUGU | 816 |
| D-1272 | 3568 | CACACAGUAGUGGAGCUUUCC | 273 | GGAAAGCUCCACUACUGUGUG | 817 |
| D-1273 | 3569 | ACACAGUAGUGGAGCUUUCCU | 274 | AGGAAAGCUCCACUACUGUGU | 818 |
| D-1571 | | | | | |
| D- | 3571 | ACAGUAGUGGAGCUUUCCUAA | 275 | UUAGGAAAGCUCCACUACUGU | 819 |
| 1273B | | | | | |
| D-1274 | 3582 | GCUUUCCUAACACUAGCAGAG | 276 | CUCUGCUAGUGUUAGGAAAGC | 820 |
| D-1275 | 3589 | UAACACUAGCAGAGAUUAAUC | 277 | GAUUAAUCUCUGCUAGUGUUA | 821 |
| D-1276 | 3590 | AACACUAGCAGAGAUUAAUCA | 278 | UGAUUAAUCUCUGCUAGUGUU | 822 |
| D-1277 | 3591 | ACACUAGCAGAGAUUAAUCAC | 279 | GUGAUUAAUCUCUGCUAGUGU | 823 |
| D-1278 | 3594 | CUAGCAGAGAUUAAUCACUAC | 280 | GUAGUGAUUAAUCUCUGCUAG | 824 |
| D-1279 | 3599 | AGAGAUUAAUCACUACAUUAG | 281 | CUAAUGUAGUGAUUAAUCUCU | 825 |
| D-1280 | 3611 | CUACAUUAGACAACACUCAUC | 282 | GAUGAGUGUUGUCUAAUGUAG | 826 |
| D-1281 | 3612 | UACAUUAGACAACACUCAUCU | 283 | AGAUGAGUGUUGUCUAAUGUA | 827 |
| D-1282 | 3614 | CAUUAGACAACACUCAUCUAC | 284 | GUAGAUGAGUGUUGUCUAAUG | 828 |
| D-1283 | 3659 | GGAUAACUGAGAAACAAGAGA | 285 | UCUCUUGUUUCUCAGUUAUCC | 829 |
| D-1284 | 3676 | GAGACCAUUCUCUGUCUAACU | 286 | AGUUAGACAGAGAAUGGUCUC | 830 |
| D- | 3687 | CUGUCUAACUGUGAUAAAAAC | 287 | GUUUUUAUCACAGUUAGACAG | 831 |
| 1284B | | | | | |
| D-1285 | 3712 | UCAGGACUUUAUUCUAUAGAG | 288 | CUCUAUAGAAUAAAGUCCUGA | 832 |
| D-1286 | 3717 | ACUUUAUUCUAUAGAGCAAAC | 289 | GUUUGCUCUAUAGAAUAAAGU | 833 |
| D-1617 | | | | | |
| D-1287 | 3720 | UUAUUCUAUAGAGCAAACUUG | 290 | CAAGUUUGCUCUAUAGAAUAA | 834 |
| D-1626 | | | | | |
| D-1288 | 3721 | UAUUCUAUAGAGCAAACUUGC | 291 | GCAAGUUUGCUCUAUAGAAUA | 835 |
| D-1289 | 3723 | UUCUAUAGAGCAAACUUGCUG | 292 | CAGCAAGUUUGCUCUAUAGAA | 836 |
| D-1290 | 3741 | CUGUGGAGGGCCAUGCUCUCC | 293 | GGAGAGCAUGGCCCUCCACAG | 837 |
| D-1291 | 3755 | GCUCUCCUUGGACCCAGUUAA | 294 | UUAACUGGGUCCAAGGAGAGC | 838 |
| D-1292 | 3757 | UCUCCUUGGACCCAGUUAACU | 295 | AGUUAACUGGGUCCAAGGAGA | 839 |
| D-1293 | 3758 | CUCCUUGGACCCAGUUAACUG | 296 | CAGUUAACUGGGUCCAAGGAG | 840 |
| D-1294 | 3760 | CCUUGGACCCAGUUAACUGCA | 297 | UGCAGUUAACUGGGUCCAAGG | 841 |
| D-1295 | 3761 | CUUGGACCCAGUUAACUGCAA | 298 | UUGCAGUUAACUGGGUCCAAG | 842 |
| D-1296 | 3764 | GGACCCAGUUAACUGCAAACG | 299 | CGUUUGCAGUUAACUGGGUCC | 843 |
| D-1297 | 3765 | GACCCAGUUAACUGCAAACGU | 300 | ACGUUUGCAGUUAACUGGGUC | 844 |
| D-1298 | 3766 | ACCCAGUUAACUGCAAACGUG | 301 | CACGUUUGCAGUUAACUGGGU | 845 |
| D-1299 | 3767 | CCCAGUUAACUGCAAACGUGC | 302 | GCACGUUUGCAGUUAACUGGG | 846 |
| D-1300 | 3768 | CCAGUUAACUGCAAACGUGCA | 303 | UGCACGUUUGCAGUUAACUGG | 847 |
| D-1301 | 3769 | CAGUUAACUGCAAACGUGCAU | 304 | AUGCACGUUUGCAGUUAACUG | 848 |
| D-1302 | 3772 | UUAACUGCAAACGUGCAUUGG | 305 | CCAAUGCACGUUUGCAGUUAA | 849 |
| D-1303 | 3776 | CUGCAAACGUGCAUUGGAGCC | 306 | GGCUCCAAUGCACGUUUGCAG | 850 |
| D-1304 | 3777 | UGCAAACGUGCAUUGGAGCCC | 307 | GGGCUCCAAUGCACGUUUGCA | 851 |
| D-1551 | | | | | |
| D-1305 | 3781 | AACGUGCAUUGGAGCCCUAUU | 308 | AAUAGGGCUCCAAUGCACGUU | 852 |
| D-1306 | 3782 | ACGUGCAUUGGAGCCCUAUUU | 309 | AAAUAGGGCUCCAAUGCACGU | 853 |
| D-1307 | 3784 | GUGCAUUGGAGCCCUAUUUGC | 310 | GCAAAUAGGGCUCCAAUGCAC | 854 |
| D-1308 | 3785 | UGCAUUGGAGCCCUAUUUGCU | 311 | AGCAAAUAGGGCUCCAAUGCA | 855 |
| D-1309 | 3790 | UGGAGCCCUAUUUGCUGCCGC | 312 | GCGGCAGCAAAUAGGGCUCCA | 856 |
| D-1310 | 3791 | GGAGCCCUAUUUGCUGCCGCU | 313 | AGCGGCAGCAAAUAGGGCUCC | 857 |
| D-1311 | 3792 | GAGCCCUAUUUGCUGCCGCUG | 314 | CAGCGGCAGCAAAUAGGGCUC | 858 |
| D-1312 | 3793 | AGCCCUAUUUGCUGCCGCUGC | 315 | GCAGCGGCAGCAAAUAGGGCU | 859 |
| D-1313 | 3807 | CCGCUGCCAUUCUAGUGACCU | 316 | AGGUCACUAGAAUGGCAGCGG | 860 |
| D-1314 | 3811 | UGCCAUUCUAGUGACCUUUCC | 317 | GGAAAGGUCACUAGAAUGGCA | 861 |
| D-1315 | 3812 | GCCAUUCUAGUGACCUUUCCA | 318 | UGGAAAGGUCACUAGAAUGGC | 862 |
| D-1316 | 3818 | CUAGUGACCUUUCCACAGAGC | 319 | GCUCUGUGGAAAGGUCACUAG | 863 |
| D-1317 | 3834 | AGAGCUGCGCCUUCCUCACGU | 320 | ACGUGAGGAAGGCGCAGCUCU | 864 |
| D-1318 | 3840 | GCGCCUUCCUCACGUGUGUGA | 321 | UCACACACGUGAGGAAGGCGC | 865 |
| D-1319 | 3847 | CCUCACGUGUGUGAAAGGUUU | 322 | AAACCUUUCACACACGUGAGG | 866 |
| D-1320 | 3848 | CUCACGUGUGUGAAAGGUUUU | 323 | AAAACCUUUCACACACGUGAG | 867 |
| D-1321 | 3873 | UUCAGCCCUCAGGUAGAUGGA | 324 | UCCAUCUACCUGAGGGCUGAA | 868 |
| D-1322 | 3874 | UCAGCCCUCAGGUAGAUGGAA | 325 | UUCCAUCUACCUGAGGGCUGA | 869 |
| D-1323 | 3876 | AGCCCUCAGGUAGAUGGAAGC | 326 | GCUUCCAUCUACCUGAGGGCU | 870 |
| D- | 3907 | CACGAUGGCAGUGCAGUCAUC | 327 | GAUGACUGCACUGCCAUCGUG | 871 |
| 1323B | | | | | |
| D-1324 | 3932 | UCAGGAUGUUUCUUCAGGACU | 328 | AGUCCUGAAGAAACAUCCUGA | 872 |
| D-1325 | 3952 | UUCCUCAGCUGACAAGGAAUU | 329 | AAUUCCUUGUCAGCUGAGGAA | 873 |
| D-1326 | 3958 | AGCUGACAAGGAAUUUUGGUC | 330 | GACCAAAAUUCCUUGUCAGCU | 874 |
| D-1327 | 3968 | GAAUUUUGGUCCCUGCCUAGG | 331 | CCUAGGCAGGGACCAAAAUUC | 875 |
| D-1328 | 3969 | AAUUUUGGUCCCUGCCUAGGA | 332 | UCCUAGGCAGGGACCAAAAUU | 876 |
| D-1328 | 3971 | UUUUGGUCCCUGCCUAGGACC | 333 | GGUCCUAGGCAGGGACCAAAA | 877 |
| D-1329 | 3972 | UUUGGUCCCUGCCUAGGACCG | 334 | CGGUCCUAGGCAGGGACCAAA | 878 |
| D-1330 | 3980 | CUGCCUAGGACCGGGUCAUCU | 335 | AGAUGACCCGGUCCUAGGCAG | 879 |
| D-1331 | 4008 | ACAGAGAGAUGGUAAGCAGCU | 336 | AGCUGCUUACCAUCUCUCUGU | 880 |
| D-1548 | | | | | |
| D-1332 | 4011 | GAGAGAUGGUAAGCAGCUGUA | 337 | UACAGCUGCUUACCAUCUCUC | 881 |
| D-1333 | 4012 | AGAGAUGGUAAGCAGCUGUAU | 338 | AUACAGCUGCUUACCAUCUCU | 882 |
| D-1334 | 4013 | GAGAUGGUAAGCAGCUGUAUG | 339 | CAUACAGCUGCUUACCAUCUC | 883 |
| D-1335 | 4019 | GUAAGCAGCUGUAUGAAUGCU | 340 | AGCAUUCAUACAGCUGCUUAC | 884 |
| D-1336 | 4022 | AGCAGCUGUAUGAAUGCUGAU | 341 | AUCAGCAUUCAUACAGCUGCU | 885 |
| D-1337 | 4040 | GAUUUUAAAACCAGGUCAUGG | 342 | CCAUGACCUGGUUUUAAAAUC | 886 |
| D-1338 | 4042 | UUUUAAAACCAGGUCAUGGGA | 343 | UCCCAUGACCUGGUUUUAAAA | 887 |
| D-1339 | 4084 | CUGAACACUGACUGCACUUAC | 344 | GUAAGUGCAGUCAGUGUUCAG | 888 |
| D-1340 | 4085 | UGAACACUGACUGCACUUACC | 345 | GGUAAGUGCAGUCAGUGUUCA | 889 |
| D-1341 | 4098 | CACUUACCAGUCUGAUUUUAU | 346 | AUAAAAUCAGACUGGUAAGUG | 890 |
| D-1342 | 4103 | ACCAGUCUGAUUUUAUCGUCA | 347 | UGACGAUAAAAUCAGACUGGU | 891 |
| D-1343 | 4104 | CCAGUCUGAUUUUAUCGUCAA | 348 | UUGACGAUAAAAUCAGACUGG | 892 |
| D-1344 | 4108 | UCUGAUUUUAUCGUCAAACAC | 349 | GUGUUUGACGAUAAAAUCAGA | 893 |
| D-1345 | 4109 | CUGAUUUUAUCGUCAAACACC | 350 | GGUGUUUGACGAUAAAAUCAG | 894 |
| D-1600 | | | | | |
| D-1346 | 4110 | UGAUUUUAUCGUCAAACACCA | 351 | UGGUGUUUGACGAUAAAAUCA | 895 |
| D-1347 | 4111 | GAUUUUAUCGUCAAACACCAA | 352 | UUGGUGUUUGACGAUAAAAUC | 896 |
| D-1348 | 4112 | AUUUUAUCGUCAAACACCAAG | 353 | CUUGGUGUUUGACGAUAAAAU | 897 |
| D-1349 | 4115 | UUAUCGUCAAACACCAAGCCA | 354 | UGGCUUGGUGUUUGACGAUAA | 898 |
| D-1350 | 4116 | UAUCGUCAAACACCAAGCCAG | 355 | CUGGCUUGGUGUUUGACGAUA | 899 |
| D-1351 | 4142 | CAUGCUCAUGGCAAUCUGUUU | 356 | AAACAGAUUGCCAUGAGCAUG | 900 |
| D-1352 | 4147 | UCAUGGCAAUCUGUUUGGGGC | 357 | GCCCCAAACAGAUUGCCAUGA | 901 |
| D-1353 | 4169 | GUUUUGUUGUGGCACUAGCCA | 358 | UGGCUAGUGCCACAACAAAAC | 902 |
| D-1354 | 4170 | UUUUGUUGUGGCACUAGCCAA | 359 | UUGGCUAGUGCCACAACAAAA | 903 |
| D-1355 | 4171 | UUUGUUGUGGCACUAGCCAAA | 360 | UUUGGCUAGUGCCACAACAAA | 904 |
| D-1356 | 4172 | UUGUUGUGGCACUAGCCAAAC | 361 | GUUUGGCUAGUGCCACAACAA | 905 |
| D-1357 | 4174 | GUUGUGGCACUAGCCAAACAU | 362 | AUGUUUGGCUAGUGCCACAAC | 906 |
| D-1358 | 4175 | UUGUGGCACUAGCCAAACAUA | 363 | UAUGUUUGGCUAGUGCCACAA | 907 |
| D-1359 | 4176 | UGUGGCACUAGCCAAACAUAA | 364 | UUAUGUUUGGCUAGUGCCACA | 908 |
| D-1360 | 4177 | GUGGCACUAGCCAAACAUAAA | 365 | UUUAUGUUUGGCUAGUGCCAC | 909 |
| D-1361 | 4178 | UGGCACUAGCCAAACAUAAAG | 366 | CUUUAUGUUUGGCUAGUGCCA | 910 |
| D-1362 | 4179 | GGCACUAGCCAAACAUAAAGG | 367 | CCUUUAUGUUUGGCUAGUGCC | 911 |
| D-1363 | 4180 | GCACUAGCCAAACAUAAAGGG | 368 | CCCUUUAUGUUUGGCUAGUGC | 912 |
| D-1364 | 4185 | AGCCAAACAUAAAGGGGCUUA | 369 | UAAGCCCCUUUAUGUUUGGCU | 913 |
| D-1365 | 4186 | GCCAAACAUAAAGGGGCUUAA | 370 | UUAAGCCCCUUUAUGUUUGGC | 914 |
| D-1366 | 4187 | CCAAACAUAAAGGGGCUUAAG | 371 | CUUAAGCCCCUUUAUGUUUGG | 915 |
| D-1367 | 4188 | CAAACAUAAAGGGGCUUAAGU | 372 | ACUUAAGCCCCUUUAUGUUUG | 916 |
| D-1368 | 4189 | AAACAUAAAGGGGCUUAAGUC | 373 | GACUUAAGCCCCUUUAUGUUU | 917 |
| D-1369 | 4190 | AACAUAAAGGGGCUUAAGUCA | 374 | UGACUUAAGCCCCUUUAUGUU | 918 |
| D-1370 | 4191 | ACAUAAAGGGGCUUAAGUCAG | 375 | CUGACUUAAGCCCCUUUAUGU | 919 |
| D-1371 | 4193 | AUAAAGGGGCUUAAGUCAGCC | 376 | GGCUGACUUAAGCCCCUUUAU | 920 |
| D-1372 | 4194 | UAAAGGGGCUUAAGUCAGCCU | 377 | AGGCUGACUUAAGCCCCUUUA | 921 |
| D-1373 | 4197 | AGGGGCUUAAGUCAGCCUGCA | 378 | UGCAGGCUGACUUAAGCCCCU | 922 |
| D-1374 | 4205 | AAGUCAGCCUGCAUACAGAGG | 379 | CCUCUGUAUGCAGGCUGACUU | 923 |
| D-1375 | 4206 | AGUCAGCCUGCAUACAGAGGA | 380 | UCCUCUGUAUGCAGGCUGACU | 924 |
| D-1376 | 4210 | AGCCUGCAUACAGAGGAUCGG | 381 | CCGAUCCUCUGUAUGCAGGCU | 925 |
| D-1377 | 4211 | GCCUGCAUACAGAGGAUCGGG | 382 | CCCGAUCCUCUGUAUGCAGGC | 926 |
| D-1378 | 4212 | CCUGCAUACAGAGGAUCGGGG | 383 | CCCCGAUCCUCUGUAUGCAGG | 927 |
| D-1379 | 4219 | ACAGAGGAUCGGGGAGAGAAG | 384 | CUUCUCUCCCCGAUCCUCUGU | 928 |
| D-1380 | 4262 | GAGUACUUACCAGAGUUUAAU | 385 | AUUAAACUCUGGUAAGUACUC | 929 |
| D-2100 | 4297 | UCUGCACUAAAAUCCCCAAAC | 2819 | GUUUGGGGAUUUUAGUGCAGA | 2820 |
| D-1381 | 4298 | CUGCACUAAAAUCCCCAAACU | 386 | AGUUUGGGGAUUUUAGUGCAG | 930 |
| D-1382 | 4299 | UGCACUAAAAUCCCCAAACUG | 387 | CAGUUUGGGGAUUUUAGUGCA | 931 |
| D-1383 | 4301 | CACUAAAAUCCCCAAACUGAC | 388 | GUCAGUUUGGGGAUUUUAGUG | 932 |
| D-1384 | 4307 | AAUCCCCAAACUGACAGGUAA | 389 | UUACCUGUCAGUUUGGGGAUU | 933 |
| D-1385 | 4312 | CCAAACUGACAGGUAAAUGUA | 390 | UACAUUUACCUGUCAGUUUGG | 934 |
| D-1386 | 4313 | CAAACUGACAGGUAAAUGUAG | 391 | CUACAUUUACCUGUCAGUUUG | 935 |
| D-1387 | 4314 | AAACUGACAGGUAAAUGUAGC | 392 | GCUACAUUUACCUGUCAGUUU | 936 |
| D-1388 | 4316 | ACUGACAGGUAAAUGUAGCCC | 393 | GGGCUACAUUUACCUGUCAGU | 937 |
| D-1389 | 4359 | AUCUAAAUCACACUAUUUUCG | 394 | CGAAAAUAGUGUGAUUUAGAU | 938 |
| D-1390 | 4361 | CUAAAUCACACUAUUUUCGAG | 395 | CUCGAAAAUAGUGUGAUUUAG | 939 |
| D-1391 | 4362 | UAAAUCACACUAUUUUCGAGA | 396 | UCUCGAAAAUAGUGUGAUUUA | 940 |
| D-1392 | 4364 | AAUCACACUAUUUUCGAGAUC | 397 | GAUCUCGAAAAUAGUGUGAUU | 941 |
| D-1393 | 4366 | UCACACUAUUUUCGAGAUCAU | 398 | AUGAUCUCGAAAAUAGUGUGA | 942 |
| D-1394 | 4367 | CACACUAUUUUCGAGAUCAUG | 399 | CAUGAUCUCGAAAAUAGUGUG | 943 |
| D-1395 | 4368 | ACACUAUUUUCGAGAUCAUGU | 400 | ACAUGAUCUCGAAAAUAGUGU | 944 |
| D-1396 | 4370 | ACUAUUUUCGAGAUCAUGUAU | 401 | AUACAUGAUCUCGAAAAUAGU | 945 |
| D-1397 | 4371 | CUAUUUUCGAGAUCAUGUAUA | 402 | UAUACAUGAUCUCGAAAAUAG | 946 |
| D-1398 | 4373 | AUUUUCGAGAUCAUGUAUAAA | 403 | UUUAUACAUGAUCUCGAAAAU | 947 |
| D-1399 | 4376 | UUCGAGAUCAUGUAUAAAAAG | 404 | CUUUUUAUACAUGAUCUCGAA | 948 |
| D-1400 | 4399 | AAAAAAGAAGUCAUGCUGUGU | 405 | ACACAGCAUGACUUCUUUUUU | 949 |
| D-1401 | 4412 | UGCUGUGUGGCCAAUUAUAAU | 406 | AUUAUAAUUGGCCACACAGCA | 950 |
| D-1777 | | | | | |
| D-1937 | | | | | |
| D-1943 | | | | | |
| D-1949 | | | | | |
| D-1955 | | | | | |
| D-1961 | | | | | |
| D-1967 | | | | | |
| D-1973 | | | | | |
| D-2041 | | | | | |
| D-2045 | | | | | |
| D-2050 | | | | | |
| D-2057 | | | | | |
| D-2058 | | | | | |
| D-2059 | | | | | |
| D-2060 | | | | | |
| D-2061 | | | | | |
| D-2085 | | | | | |
| D-2091 | | | | | |
| D-1402 | 4476 | UUGGAGGGACCAGGAAAUGUA | 407 | UACAUUUCCUGGUCCCUCCAA | 951 |
| D-1403 | 4484 | ACCAGGAAAUGUAAGACACCA | 408 | UGGUGUCUUACAUUUCCUGGU | 952 |
| D-1742 | | | | | |
| D-1404 | 4485 | CCAGGAAAUGUAAGACACCAA | 409 | UUGGUGUCUUACAUUUCCUGG | 953 |
| D-1743 | | | | | |
| D-1405 | 4522 | GUGUGCCUGAUGUCACCUCAU | 410 | AUGAGGUGACAUCAGGCACAC | 954 |
| D-1406 | 4523 | UGUGCCUGAUGUCACCUCAUG | 411 | CAUGAGGUGACAUCAGGCACA | 955 |
| D-1407 | 4524 | GUGCCUGAUGUCACCUCAUGA | 412 | UCAUGAGGUGACAUCAGGCAC | 956 |
| D-1408 | 4526 | GCCUGAUGUCACCUCAUGAUU | 413 | AAUCAUGAGGUGACAUCAGGC | 957 |
| D-1409 | 4556 | UUUUUUAACUCCUGCGCCAAG | 414 | CUUGGCGCAGGAGUUAAAAAA | 958 |
| D-1410 | 4560 | UUAACUCCUGCGCCAAGGACA | 415 | UGUCCUUGGCGCAGGAGUUAA | 959 |
| D-1411 | 4562 | AACUCCUGCGCCAAGGACAGU | 416 | ACUGUCCUUGGCGCAGGAGUU | 960 |
| D-1412 | 4563 | ACUCCUGCGCCAAGGACAGUG | 417 | CACUGUCCUUGGCGCAGGAGU | 961 |
| D-1413 | 4591 | UGUCCACCUUUGUGCUUUGCG | 418 | CGCAAAGCACAAAGGUGGACA | 962 |
| D-1414 | 4593 | UCCACCUUUGUGCUUUGCGAG | 419 | CUCGCAAAGCACAAAGGUGGA | 963 |
| D-1415 | 4595 | CACCUUUGUGCUUUGCGAGGC | 420 | GCCUCGCAAAGCACAAAGGUG | 964 |
| D-1416 | 4597 | CCUUUGUGCUUUGCGAGGCCG | 421 | CGGCCUCGCAAAGCACAAAGG | 965 |
| D-1417 | 4617 | GAGCCCAGGCAUCUGCUCGCC | 422 | GGCGAGCAGAUGCCUGGGCUC | 966 |
| D-1418 | 4620 | CCCAGGCAUCUGCUCGCCUGC | 423 | GCAGGCGAGCAGAUGCCUGGG | 967 |
| D-1419 | 4622 | CAGGCAUCUGCUCGCCUGCCA | 424 | UGGCAGGCGAGCAGAUGCCUG | 968 |
| D-1420 | 4625 | GCAUCUGCUCGCCUGCCACGG | 425 | CCGUGGCAGGCGAGCAGAUGC | 969 |
| D-1421 | 4638 | UGCCACGGCUGACCAGAGAAG | 426 | CUUCUCUGGUCAGCCGUGGCA | 970 |
| D-1422 | 4668 | GAGCUCUGCCUUAGACGACGU | 427 | ACGUCGUCUAAGGCAGAGCUC | 971 |
| D-1423 | 4669 | AGCUCUGCCUUAGACGACGUG | 428 | CACGUCGUCUAAGGCAGAGCU | 972 |
| D-1424 | 4670 | GCUCUGCCUUAGACGACGUGU | 429 | ACACGUCGUCUAAGGCAGAGC | 973 |
| D-1425 | 4671 | CUCUGCCUUAGACGACGUGUU | 430 | AACACGUCGUCUAAGGCAGAG | 974 |
| D-1426 | 4672 | UCUGCCUUAGACGACGUGUUA | 431 | UAACACGUCGUCUAAGGCAGA | 975 |
| D-1427 | 4673 | CUGCCUUAGACGACGUGUUAC | 432 | GUAACACGUCGUCUAAGGCAG | 976 |
| D-1428 | 4675 | GCCUUAGACGACGUGUUACAG | 433 | CUGUAACACGUCGUCUAAGGC | 977 |
| D-1429 | 4676 | CCUUAGACGACGUGUUACAGU | 434 | ACUGUAACACGUCGUCUAAGG | 978 |
| D-1430 | 4677 | CUUAGACGACGUGUUACAGUA | 435 | UACUGUAACACGUCGUCUAAG | 979 |
| D-1431 | 4679 | UAGACGACGUGUUACAGUAUG | 436 | CAUACUGUAACACGUCGUCUA | 980 |
| D-1432 | 4682 | ACGACGUGUUACAGUAUGAAC | 437 | GUUCAUACUGUAACACGUCGU | 981 |
| D-1433 | 4703 | ACACAGCAGAGGCACCCUCGU | 438 | ACGAGGGUGCCUCUGCUGUGU | 982 |
| D-1434 | 4704 | CACAGCAGAGGCACCCUCGUA | 439 | UACGAGGGUGCCUCUGCUGUG | 983 |
| D-1435 | 4705 | ACAGCAGAGGCACCCUCGUAU | 440 | AUACGAGGGUGCCUCUGCUGU | 984 |
| D-1436 | 4706 | CAGCAGAGGCACCCUCGUAUG | 441 | CAUACGAGGGUGCCUCUGCUG | 985 |
| D-1437 | 4707 | AGCAGAGGCACCCUCGUAUGU | 442 | ACAUACGAGGGUGCCUCUGCU | 986 |
| D-1438 | 4708 | GCAGAGGCACCCUCGUAUGUU | 443 | AACAUACGAGGGUGCCUCUGC | 987 |
| D-1439 | 4709 | CAGAGGCACCCUCGUAUGUUU | 444 | AAACAUACGAGGGUGCCUCUG | 988 |
| D-1440 | 4710 | AGAGGCACCCUCGUAUGUUUU | 445 | AAAACAUACGAGGGUGCCUCU | 989 |
| D-1441 | 4711 | GAGGCACCCUCGUAUGUUUUG | 446 | CAAAACAUACGAGGGUGCCUC | 990 |
| D-1442 | 4713 | GGCACCCUCGUAUGUUUUGAA | 447 | UUCAAAACAUACGAGGGUGCC | 991 |
| D-1443 | 4717 | CCCUCGUAUGUUUUGAAAGUU | 448 | AACUUUCAAAACAUACGAGGG | 992 |
| D-1744 | | | | | |
| D-1896 | | | | | |
| D-1902 | | | | | |
| D-1908 | | | | | |
| D-1914 | | | | | |
| D-1920 | | | | | |
| D-1926 | | | | | |
| D-1932 | | | | | |
| D-2062 | | | | | |
| D-2063 | | | | | |
| D-2089 | | | | | |
| D-1444 | 4777 | AUGUAAAACUAUACUGACCCG | 449 | CGGGUCAGUAUAGUUUUACAU | 993 |
| D-1778 | | | | | |
| D-1445 | 4778 | UGUAAAACUAUACUGACCCGU | 450 | ACGGGUCAGUAUAGUUUUACA | 994 |
| D-1446 | 4779 | GUAAAACUAUACUGACCCGUU | 451 | AACGGGUCAGUAUAGUUUUAC | 995 |
| D-1590 | | | | | |
| D-1447 | 4780 | UAAAACUAUACUGACCCGUUU | 452 | AAACGGGUCAGUAUAGUUUUA | 996 |
| D-1779 | | | | | |
| D-1448 | 4784 | ACUAUACUGACCCGUUUUCAG | 453 | CUGAAAACGGGUCAGUAUAGU | 997 |
| D-1449 | 4787 | AUACUGACCCGUUUUCAGUUU | 454 | AAACUGAAAACGGGUCAGUAU | 998 |
| D-1450 | 4799 | UUUCAGUUUUAAAGGGUCGUG | 455 | CACGACCCUUUAAAACUGAAA | 999 |
| D-1745 | | | | | |
| D-1451 | 4800 | UUCAGUUUUAAAGGGUCGUGA | 456 | UCACGACCCUUUAAAACUGAA | 1000 |
| D-1452 | 4801 | UCAGUUUUAAAGGGUCGUGAG | 457 | CUCACGACCCUUUAAAACUGA | 1001 |
| D-1746 | | | | | |
| D-1453 | 4802 | CAGUUUUAAAGGGUCGUGAGA | 458 | UCUCACGACCCUUUAAAACUG | 1002 |
| D-1747 | | | | | |
| D-1454 | 4804 | GUUUUAAAGGGUCGUGAGAAA | 459 | UUUCUCACGACCCUUUAAAAC | 1003 |
| D-1630 | | | | | |
| D-1454 | 4805 | UUUUAAAGGGUCGUGAGAAAC | 460 | GUUUCUCACGACCCUUUAAAA | 1004 |
| D-1455 | 4806 | UUUAAAGGGUCGUGAGAAACU | 461 | AGUUUCUCACGACCCUUUAAA | 1005 |
| D-1748 | | | | | |
| D-1456 | 4808 | UAAAGGGUCGUGAGAAACUGG | 462 | CCAGUUUCUCACGACCCUUUA | 1006 |
| D-1457 | 4809 | AAAGGGUCGUGAGAAACUGGC | 463 | GCCAGUUUCUCACGACCCUUU | 1007 |
| D-1458 | 4819 | GAGAAACUGGCUGGUCCAAUG | 464 | CAUUGGACCAGCCAGUUUCUC | 1008 |
| D-1780 | | | | | |
| D-1459 | 4830 | UGGUCCAAUGGGAUUUACAGC | 465 | GCUGUAAAUCCCAUUGGACCA | 1009 |
| D-1460 | 4834 | CCAAUGGGAUUUACAGCAACA | 466 | UGUUGCUGUAAAUCCCAUUGG | 1010 |
| D-1781 | | | | | |
| D-1894 | | | | | |
| D-1900 | | | | | |
| D-1906 | | | | | |
| D-1912 | | | | | |
| D-1918 | | | | | |
| D-1924 | | | | | |
| D-1930 | | | | | |
| D-2064 | | | | | |
| D-2065 | | | | | |
| D-2066 | | | | | |
| D-2067 | | | | | |
| D-2068 | | | | | |
| D-2092 | | | | | |
| D-1461 | 4927 | UUUAAGUUUGCUCUUAAUCGU | 467 | ACGAUUAAGAGCAAACUUAAA | 1011 |
| D-1596 | | | | | |
| D-1462 | 4928 | UUAAGUUUGCUCUUAAUCGUA | 468 | UACGAUUAAGAGCAAACUUAA | 1012 |
| D-1594 | | | | | |
| D-1463 | 4931 | AGUUUGCUCUUAAUCGUAUGG | 469 | CCAUACGAUUAAGAGCAAACU | 1013 |
| D-1782 | | | | | |
| D-1464 | 4932 | GUUUGCUCUUAAUCGUAUGGA | 470 | UCCAUACGAUUAAGAGCAAAC | 1014 |
| D-1783 | | | | | |
| D-1895 | | | | | |
| D-1901 | | | | | |
| D-1907 | | | | | |
| D-1913 | | | | | |
| D-1919 | | | | | |
| D-1925 | | | | | |
| D-1931 | | | | | |
| D-2069 | | | | | |
| D-2070 | | | | | |
| D-2071 | | | | | |
| D-2072 | | | | | |
| D-2073 | | | | | |
| D-2074 | | | | | |
| D-2088 | | | | | |
| D-1465 | 4933 | UUUGCUCUUAAUCGUAUGGAA | 471 | UUCCAUACGAUUAAGAGCAAA | 1015 |
| D-1784 | | | | | |
| D-1466 | 4935 | UGCUCUUAAUCGUAUGGAAGC | 472 | GCUUCCAUACGAUUAAGAGCA | 1016 |
| D-1785 | | | | | |
| D-1467 | 4939 | CUUAAUCGUAUGGAAGCUUGA | 473 | UCAAGCUUCCAUACGAUUAAG | 1017 |
| D-1786 | | | | | |
| D-1468 | 4940 | UUAAUCGUAUGGAAGCUUGAG | 474 | CUCAAGCUUCCAUACGAUUAA | 1018 |
| D-1787 | | | | | |
| D-1469 | 4950 | GGAAGCUUGAGCUAUGUGUUG | 475 | CAACACAUAGCUCAAGCUUCC | 1019 |
| D-1749 | | | | | |
| D-1470 | 4951 | GAAGCUUGAGCUAUGUGUUGG | 476 | CCAACACAUAGCUCAAGCUUC | 1020 |
| D-1750 | | | | | |
| D-1471 | 4953 | AGCUUGAGCUAUGUGUUGGAA | 477 | UUCCAACACAUAGCUCAAGCU | 1021 |
| D-1751 | | | | | |
| D-1472 | 4954 | GCUUGAGCUAUGUGUUGGAAG | 478 | CUUCCAACACAUAGCUCAAGC | 1022 |
| D-1752 | | | | | |
| D-1473 | 4955 | CUUGAGCUAUGUGUUGGAAGU | 479 | ACUUCCAACACAUAGCUCAAG | 1023 |
| D-1753 | | | | | |
| D-1474 | 4956 | UUGAGCUAUGUGUUGGAAGUG | 480 | CACUUCCAACACAUAGCUCAA | 1024 |
| D-1593 | | | | | |
| D-1475 | 4957 | UGAGCUAUGUGUUGGAAGUGC | 481 | GCACUUCCAACACAUAGCUCA | 1025 |
| D-1631 | | | | | |
| D-1696 | | | | | |
| D-1703 | | | | | |
| D-1710 | | | | | |
| D-1717 | | | | | |
| D-1724 | | | | | |
| D-1731 | | | | | |
| D-1738 | | | | | |
| D-1857 | | | | | |
| D-2011 | | | | | |
| D-2020 | | | | | |
| D-1476 | 4958 | GAGCUAUGUGUUGGAAGUGCC | 482 | GGCACUUCCAACACAUAGCUC | 1026 |
| D-1754 | | | | | |
| D-1477 | 4965 | GUGUUGGAAGUGCCCUGGUUU | 483 | AAACCAGGGCACUUCCAACAC | 1027 |
| D-1755 | | | | | |
| D-1478 | 4970 | GGAAGUGCCCUGGUUUUAAUC | 484 | GAUUAAAACCAGGGCACUUCC | 1028 |
| D-1756 | | | | | |
| D-1479 | 4979 | CUGGUUUUAAUCCAUACACAA | 485 | UUGUGUAUGGAUUAAAACCAG | 1029 |
| D-1480 | 4985 | UUAAUCCAUACACAAAGACGG | 486 | CCGUCUUUGUGUAUGGAUUAA | 1030 |
| D-1481 | 4986 | UAAUCCAUACACAAAGACGGU | 487 | ACCGUCUUUGUGUAUGGAUUA | 1031 |
| D-1482 | 4987 | AAUCCAUACACAAAGACGGUA | 488 | UACCGUCUUUGUGUAUGGAUU | 1032 |
| D-1483 | 4988 | AUCCAUACACAAAGACGGUAC | 489 | GUACCGUCUUUGUGUAUGGAU | 1033 |
| D-1484 | 4989 | UCCAUACACAAAGACGGUACA | 490 | UGUACCGUCUUUGUGUAUGGA | 1034 |
| D-1788 | | | | | |
| D-1485 | 4991 | CAUACACAAAGACGGUACAUA | 491 | UAUGUACCGUCUUUGUGUAUG | 1035 |
| D-1789 | | | | | |
| D-1486 | 4993 | UACACAAAGACGGUACAUAAU | 492 | AUUAUGUACCGUCUUUGUGUA | 1036 |
| D-1634 | | | | | |
| D-1487 | 4994 | ACACAAAGACGGUACAUAAUC | 493 | GAUUAUGUACCGUCUUUGUGU | 1037 |
| D-1488 | 4995 | CACAAAGACGGUACAUAAUCC | 494 | GGAUUAUGUACCGUCUUUGUG | 1038 |
| D-1546 | | | | | |
| D-2036 | | | | | |
| D-1489 | 4996 | ACAAAGACGGUACAUAAUCCU | 495 | AGGAUUAUGUACCGUCUUUGU | 1039 |
| D-1757 | | | | | |
| D-2037 | | | | | |
| D-1490 | 4997 | CAAAGACGGUACAUAAUCCUA | 496 | UAGGAUUAUGUACCGUCUUUG | 1040 |
| D-1758 | | | | | |
| D-2038 | | | | | |
| D-1491 | 4998 | AAAGACGGUACAUAAUCCUAC | 497 | GUAGGAUUAUGUACCGUCUUU | 1041 |
| D-1562 | | | | | |
| D-2039 | | | | | |
| D-1492 | 4999 | AAGACGGUACAUAAUCCUACA | 498 | UGUAGGAUUAUGUACCGUCUU | 1042 |
| D-1614 | | | | | |
| D-1697 | | | | | |
| D-1702 | | | | | |
| D-1709 | | | | | |
| D-1716 | | | | | |
| D-1723 | | | | | |
| D-1730 | | | | | |
| D-1737 | | | | | |
| D-1856 | | | | | |
| D-1863 | | | | | |
| D-1865 | | | | | |
| D-1866 | | | | | |
| D-1869 | | | | | |
| D-1872 | | | | | |
| D-1877 | | | | | |
| D-1878 | | | | | |
| D-1879 | | | | | |
| D-1880 | | | | | |
| D-1881 | | | | | |
| D-1884 | | | | | |
| D-1887 | | | | | |
| D-1978 | | | | | |
| D-1987 | | | | | |
| D-1992 | | | | | |
| D-1997 | | | | | |
| D-2002 | | | | | |
| D-2008 | | | | | |
| D-2017 | | | | | |
| D-2049 | | | | | |
| D-2054 | | | | | |
| D-2090 | | | | | |
| D-1493 | 5005 | GUACAUAAUCCUACAGGUUUA | 499 | UAAACCUGUAGGAUUAUGUAC | 1043 |
| D-1566 | | | | | |
| D-1494 | 5008 | CAUAAUCCUACAGGUUUAAAU | 500 | AUUUAAACCUGUAGGAUUAUG | 1044 |
| D-1759 | | | | | |
| D-1495 | 5012 | AUCCUACAGGUUUAAAUGUAC | 501 | GUACAUUUAAACCUGUAGGAU | 1045 |
| D-1633 | | | | | |
| D-1496 | 5042 | UAGUUUGGAAUUCUUUGCUCU | 502 | AGAGCAAAGAAUUCCAAACUA | 1046 |
| D-1564 | | | | | |
| D-2040 | | | | | |
| D-1497 | 5043 | AGUUUGGAAUUCUUUGCUCUA | 503 | UAGAGCAAAGAAUUCCAAACU | 1047 |
| D-1611 | | | | | |
| D-1698 | | | | | |
| D-1705 | | | | | |
| D-1712 | | | | | |
| D-1719 | | | | | |
| D-1726 | | | | | |
| D-1733 | | | | | |
| D-1740 | | | | | |
| D-1855 | | | | | |
| D-1864 | | | | | |
| D-1870 | | | | | |
| D-1875 | | | | | |
| D-1883 | | | | | |
| D-1886 | | | | | |
| D-1980 | | | | | |
| D-1984 | | | | | |
| D-1989 | | | | | |
| D-1994 | | | | | |
| D-1999 | | | | | |
| D-2004 | | | | | |
| D-2013 | | | | | |
| D-2022 | | | | | |
| D-2044 | | | | | |
| D-2048 | | | | | |
| D-2053 | | | | | |
| D-1498 | 5045 | UUUGGAAUUCUUUGCUCUACU | 504 | AGUAGAGCAAAGAAUUCCAAA | 1048 |
| D-1612 | | | | | |
| D-1699 | | | | | |
| D-1704 | | | | | |
| D-1711 | | | | | |
| D-1718 | | | | | |
| D-1725 | | | | | |
| D-1732 | | | | | |
| D-1739 | | | | | |
| D-1854 | | | | | |
| D-1868 | | | | | |
| D-1871 | | | | | |
| D-1876 | | | | | |
| D-1882 | | | | | |
| D-1885 | | | | | |
| D-1888 | | | | | |
| D-1979 | | | | | |
| D-1983 | | | | | |
| D-1988 | | | | | |
| D-1993 | | | | | |
| D-1998 | | | | | |
| D-2003 | | | | | |
| D-2012 | | | | | |
| D-2021 | | | | | |
| D-2043 | | | | | |
| D-2047 | | | | | |
| D-2052 | | | | | |
| D-1499 | 5056 | UUGCUCUACUGUUUACAUUGC | 505 | GCAAUGUAAACAGUAGAGCAA | 1049 |
| D-1760 | | | | | |
| D-1500 | 5060 | UCUACUGUUUACAUUGCAGAU | 506 | AUCUGCAAUGUAAACAGUAGA | 1050 |
| D-1591 | | | | | |
| D-1624 | | | | | |
| D-1501 | 5062 | UACUGUUUACAUUGCAGAUUG | 507 | CAAUCUGCAAUGUAAACAGUA | 1051 |
| D-1577 | | | | | |
| D-1502 | 5063 | ACUGUUUACAUUGCAGAUUGC | 508 | GCAAUCUGCAAUGUAAACAGU | 1052 |
| D-1503 | 5067 | UUUACAUUGCAGAUUGCUAUA | 509 | UAUAGCAAUCUGCAAUGUAAA | 1053 |
| D-1603 | | | | | |
| D-1504 | 5068 | UUACAUUGCAGAUUGCUAUAA | 510 | UUAUAGCAAUCUGCAAUGUAA | 1054 |
| D-1629 | | | | | |
| D-1505 | 5069 | UACAUUGCAGAUUGCUAUAAU | 511 | AUUAUAGCAAUCUGCAAUGUA | 1055 |
| D-1628 | | | | | |
| D-1506 | 5079 | AUUGCUAUAAUUUCAAGGAGU | 512 | ACUCCUUGAAAUUAUAGCAAU | 1056 |
| D-1507 | 5080 | UUGCUAUAAUUUCAAGGAGUG | 513 | CACUCCUUGAAAUUAUAGCAA | 1057 |
| D-1623 | | | | | |
| D-1846 | | | | | |
| D-1706 | | | | | |
| D-1713 | | | | | |
| D-1720 | | | | | |
| D-1727 | | | | | |
| D-1734 | | | | | |
| D-1741 | | | | | |
| D-1761 | | | | | |
| D-1846 | | | | | |
| D-1862 | | | | | |
| D-1874 | | | | | |
| D-1981 | | | | | |
| D-1985 | | | | | |
| D-1990 | | | | | |
| D-1995 | | | | | |
| D-2000 | | | | | |
| D-2005 | | | | | |
| D-2014 | | | | | |
| D-2023 | | | | | |
| D-1508 | 5114 | AAUGAUGCACUUUAGGAUGUU | 514 | AACAUCCUAAAGUGCAUCAUU | 1058 |
| D-1762 | | | | | |
| D-1509 | 5115 | AUGAUGCACUUUAGGAUGUUU | 515 | AAACAUCCUAAAGUGCAUCAU | 1059 |
| D-1627 | | | | | |
| D-1763 | | | | | |
| D-1510 | 5154 | ACAUGAAUCAUUCACAUGACC | 516 | GGUCAUGUGAAUGAUUCAUGU | 1060 |
| D-1764 | | | | | |
| D-1511 | 5155 | CAUGAAUCAUUCACAUGACCA | 517 | UGGUCAUGUGAAUGAUUCAUG | 1061 |
| D-1765 | | | | | |
| D-1512 | 5194 | AAAUACAUGUCUAGUCUGUCC | 518 | GGACAGACUAGACAUGUAUUU | 1062 |
| D-1572 | | | | | |
| D- | 5195 | AAUACAUGUCUAGUCUGUCCU | 519 | AGGACAGACUAGACAUGUAUU | 1063 |
| 1512B | | | | | |
| D-1766 | | | | | |
| D-1513 | 5200 | AUGUCUAGUCUGUCCUUUAAU | 520 | AUUAAAGGACAGACUAGACAU | 1064 |
| D-1767 | | | | | |
| D-1514 | 5201 | UGUCUAGUCUGUCCUUUAAUA | 521 | UAUUAAAGGACAGACUAGACA | 1065 |
| D-1790 | | | | | |
| D-1515 | 5203 | UCUAGUCUGUCCUUUAAUAGC | 522 | GCUAUUAAAGGACAGACUAGA | 1066 |
| D-1791 | | | | | |
| D-1516 | 5204 | CUAGUCUGUCCUUUAAUAGCU | 523 | AGCUAUUAAAGGACAGACUAG | 1067 |
| D-1792 | | | | | |
| D-1892 | | | | | |
| D-1898 | | | | | |
| D-1904 | | | | | |
| D-1910 | | | | | |
| D-1916 | | | | | |
| D-1922 | | | | | |
| D-1928 | | | | | |
| D-1517 | 5205 | UAGUCUGUCCUUUAAUAGCUC | 524 | GAGCUAUUAAAGGACAGACUA | 1068 |
| D-1518 | 5207 | GUCUGUCCUUUAAUAGCUCUC | 525 | GAGAGCUAUUAAAGGACAGAC | 1069 |
| D-1793 | | | | | |
| D-1519 | 5247 | AAUCAGAUCAUUACCAGUUAG | 526 | CUAACUGGUAAUGAUCUGAUU | 1070 |
| D-1768 | | | | | |
| D-1891 | | | | | |
| D-1897 | | | | | |
| D-1903 | | | | | |
| D-1909 | | | | | |
| D-1915 | | | | | |
| D-1921 | | | | | |
| D-1927 | | | | | |
| D-2075 | | | | | |
| D-2077 | | | | | |
| D-1520 | 5249 | UCAGAUCAUUACCAGUUAGCU | 527 | AGCUAACUGGUAAUGAUCUGA | 1071 |
| D-1769 | | | | | |
| D-1934 | | | | | |
| D-1940 | | | | | |
| D-1946 | | | | | |
| D-1952 | | | | | |
| D-1958 | | | | | |
| D-1964 | | | | | |
| D-1970 | | | | | |
| D-2076 | | | | | |
| D-2078 | | | | | |
| D-1521 | 5250 | CAGAUCAUUACCAGUUAGCUU | 528 | AAGCUAACUGGUAAUGAUCUG | 1072 |
| D-1522 | 5251 | AGAUCAUUACCAGUUAGCUUU | 529 | AAAGCUAACUGGUAAUGAUCU | 1073 |
| D-1770 | | | | | |
| D-1523 | 5254 | UCAUUACCAGUUAGCUUUUAA | 530 | UUAAAAGCUAACUGGUAAUGA | 1074 |
| D-1771 | | | | | |
| D-1524 | 5255 | CAUUACCAGUUAGCUUUUAAA | 531 | UUUAAAAGCUAACUGGUAAUG | 1075 |
| D-1622 | | | | | |
| D-1525 | 5259 | ACCAGUUAGCUUUUAAAGCAC | 532 | GUGCUUUAAAAGCUAACUGGU | 1076 |
| D-1772 | | | | | |
| D-1526 | 5274 | AAGCACAUUUGUUUAAGACUA | 533 | UAGUCUUAAACAAAUGUGCUU | 1077 |
| D-1773 | | | | | |
| D-1936 | | | | | |
| D-1942 | | | | | |
| D-1948 | | | | | |
| D-1954 | | | | | |
| D-1960 | | | | | |
| D-1966 | | | | | |
| D-1972 | | | | | |
| D-2042 | | | | | |
| D-2046 | | | | | |
| D-2051 | | | | | |
| D-2079 | | | | | |
| D-2080 | | | | | |
| D-2081 | | | | | |
| D-2082 | | | | | |
| D-2083 | | | | | |
| D-2093 | | | | | |
| D-1527 | 5276 | GCACAUUUGUUUAAGACUAUG | 534 | CAUAGUCUUAAACAAAUGUGC | 1078 |
| D-1774 | | | | | |
| D-1893 | | | | | |
| D-1899 | | | | | |
| D-1905 | | | | | |
| D-1911 | | | | | |
| D-1917 | | | | | |
| D-1923 | | | | | |
| D-1929 | | | | | |
| D-1975 | | | | | |
| D-1976 | | | | | |
| D-1977 | | | | | |
| D-1982 | | | | | |
| D-1986 | | | | | |
| D-1991 | | | | | |
| D-1996 | | | | | |
| D-2001 | | | | | |
| D-2084 | | | | | |
| D-1528 | 5292 | CUAUGUUUUUGGAAAAAUACG | 535 | CGUAUUUUUCCAAAAACAUAG | 1079 |
| D-1529 | 5296 | GUUUUUGGAAAAAUACGCUAC | 536 | GUAGCGUAUUUUUCCAAAAAC | 1080 |
| D-1530 | 5300 | UUGGAAAAAUACGCUACAGAA | 537 | UUCUGUAGCGUAUUUUUCCAA | 1081 |
| D-1531 | 5338 | AAUAAAUGAGAUGCUACUAAU | 538 | AUUAGUAGCAUCUCAUUUAUU | 1082 |
| D-1625 | | | | | |
| D-1532 | 5344 | UGAGAUGCUACUAAUUGUUUU | 539 | AAAACAAUUAGUAGCAUCUCA | 1083 |
| D-1775 | | | | | |
| D-1533 | 5362 | UUUGGAAUCUGUUGUUUCUGC | 540 | GCAGAAACAACAGAUUCCAAA | 1084 |
| D-1534 | 5377 | UUCUGCCAAAGGUAAAUUAAC | 541 | GUUAAUUUACCUUUGGCAGAA | 1085 |
| D-1535 | 5378 | UCUGCCAAAGGUAAAUUAACU | 542 | AGUUAAUUUACCUUUGGCAGA | 1086 |
| D-1536 | 5402 | GAUUUAUUCAGGAAUCCCCAU | 543 | AUGGGGAUUCCUGAAUAAAUC | 1087 |
| D-1776 | | | | | |
| D-1537 | 5407 | AUUCAGGAAUCCCCAUUUGAA | 544 | UUCAAAUGGGGAUUCCUGAAU | 1088 |
| D-1538 | 5412 | GGAAUCCCCAUUUGAAUUUGU | 545 | ACAAAUUCAAAUGGGGAUUCC | 1089 |

Table 2 below provides the sequences of exemplary sense and antisense strands with chemical modifications od duplexes used in experiments disclosed herein. In Table 2, the nucleotide sequences are listed according to the following notations: a, u, g, and c=corresponding 2′-O-methyl ribonucleotide; Af, Uf, Gf, and Cf=corresponding 2′-deoxy-2′-fluoro (“2′-fluoro”) ribonucleotide; and invAb=inverted abasic deoxynucleotide (i.e., abasic deoxynucleotide linked to adjacent nucleotide via a substituent at its 3′ position (a 3′-3′ linkage) when on the 3′ end of a strand or linked to adjacent nucleotide via a substituent at its 5′ position (a 5′-5′ internucleotide linkage) when on the 5′ end of a strand. Insertion of an “s” in the sequence indicates that the two adjacent nucleotides are connected by a phosphorothiodiester group (e.g., a phosphorothioate internucleotide linkage). Unless indicated otherwise, all other nucleotides are connected by 3′-5′ phosphodiester groups. [DCA-C6] represents a conjugated docosanoic acid (C22). [GalNAc3] represents the GalNAc moiety shown in Formula VII. The [DCA-C6] and [GalNAc] ligands are covalently attached to the 5′ terminal nucleotide at the 5′ end of the sense strand via a phosphodiester bond, or a phosphorothioate bond when an “s” follows the [GalNAc3] or [DCA-C6] notation. When an invAb nucleotide was the 5′ terminal nucleotide at the 5′ end of the sense strand, it was linked to the adjacent nucleotide via a 5′-5′ linkage and the GalNAc or C22 moiety was covalently attached to the 3′ carbon of the invAb nucleotide. Otherwise, the moiety was covalently attached to the 5′ carbon of the 5′ terminal nucleotide of the sense strand.

TABLE 2
Modified FAM13A siRNA Sequences

| Duplex | | SEQ ID | | SEQ ID |
|---|---|---|---|---|
| No. | Sense Sequence (5′-3′) | NO: | Antisense Sequence (5′-3′) | NO: |
| D-1001 | csasuguaCfcCfCfAfAfgucagcaas | 1090 | asUfsugcuGfacuuggGfgUfacaugs | 1938 |
| | {invAb} | | usu | |
| D-1002 | usgsuaccCfcAfAfGfUfcagcaaugs | 1091 | asCfsauugCfugacuuGfgGfguacas | 1939 |
| | {invAb} | | usu | |
| D-1003 | gsusacccCfaAfGfUfCfagcaaugus | 1092 | asAfscauuGfcugacuUfgGfgguacs | 1940 |
| | {invAb} | | usu | |
| D-1004 | gscsaaugUfgUfCfUfGfcaaccggas | 1093 | asUfsccggUfugcagaCfaCfauugcs | 1941 |
| | {invAb} | | usu | |
| D-1005 | csasauguGfuCfUfGfCfaaccggags | 1094 | usCfsuccgGfuugcagAfcAfcauugs | 1942 |
| | {invAb} | | usu | |
| D-1006 | asasugugUfcUfGfCfAfaccggagas | 1095 | usUfscuccGfguugcaGfaCfacauus | 1943 |
| | {invAb} | | usu | |
| D-1007 | usgsugucUfgCfAfAfCfcggagaacs | 1096 | asGfsuucuCfcgguugCfaGfacacas | 1944 |
| | {invAb} | | usu | |
| D-1008 | gsusgucuGfcAfAfCfCfggagaacus | 1097 | asAfsguucUfccgguuGfcAfgacacs | 1945 |
| | {invAb} | | usu | |
| D-1009 | usgsucugCfaAfCfCfGfgagaacucs | 1098 | asGfsaguuCfuccgguUfgCfagacas | 1946 |
| | {invAb} | | usu | |
| D-1010 | gsuscugcAfaCfCfGfGfagaacucus | 1099 | asAfsgaguUfcuccggUfuGfcagacs | 1947 |
| | {invAb} | | usu | |
| D-1011 | uscsugcaAfcCfGfGfAfgaacucuus | 1100 | usAfsagagUfucuccgGfuUfgcagas | 1948 |
| | {invAb} | | usu | |
| D-1012 | csusgcaaCfcGfGfAfGfaacucuuas | 1101 | asUfsaagaGfuucuccGfgUfugcags | 1949 |
| | {invAb} | | usu | |
| D-1013 | usgscaacCfgGfAfGfAfacucuuags | 1102 | usCfsuaagAfguucucCfgGfuugcas | 1950 |
| | {invAb} | | usu | |
| D-1014 | gscsaaccGfgAfGfAfAfcucuuagas | 1103 | usUfscuaaGfaguucuCfcGfguugcs | 1951 |
| | {invAb} | | usu | |
| D-1015 | asasccggAfgAfAfCfUfcuuagaaas | 1104 | asUfsuucuAfagaguuCfuCfcgguus | 1952 |
| | {invAb} | | usu | |
| D-1016 | uscsuuagAfaAfGfAfAfccauccgas | 1105 | asUfscggaUfgguucuUfuCfuaagas | 1953 |
| | {invAb} | | usu | |
| D-1017 | csusuagaAfaGfAfAfCfcauccgaus | 1106 | asAfsucggAfugguucUfuUfcuaags | 1954 |
| | {invAb} | | usu | |
| D-1018 | ususagaaAfgAfAfCfCfauccgaucs | 1107 | usGfsaucgGfaugguuCfuUfucuaas | 1955 |
| | {invAb} | | usu | |
| D-1019 | asgsaaagAfaCfCfAfUfccgaucags | 1108 | asCfsugauCfggauggUfuCfuuucus | 1956 |
| | {invAb} | | usu | |
| D-1020 | asasagaaCfcAfUfCfCfgaucagcus | 1109 | asAfsgcugAfucggauGfgUfucuuus | 1957 |
| | {invAb} | | usu | |
| D-1021 | asasgaacCfaUfCfCfGfaucagcugs | 1110 | asCfsagcuGfaucggaUfgGfuucuus | 1958 |
| | {invAb} | | usu | |
| D-1022 | gsasaccaUfcCfGfAfUfcagcuguas | 1111 | asUfsacagCfugaucgGfaUfgguucs | 1959 |
| | {invAb} | | usu | |
| D-1023 | ascscaucCfgAfUfCfAfgcuguagas | 1112 | usUfscuacAfgcugauCfgGfauggus | 1960 |
| | {invAb} | | usu | |
| D-1024 | cscsgaucAfgCfUfGfUfagaacaacs | 1113 | usGfsuuguUfcuacagCfuGfaucggs | 1961 |
| | {invAb} | | usu | |
| D-1025 | gsasuguuAfaUfAfAfCfucuggaggs | 1114 | asCfscuccAfgaguuaUfuAfacaucs | 1962 |
| | {invAb} | | usu | |
| D-1026 | usasauaaCfuCfUfGfGfaggucaaas | 1115 | asUfsuugaCfcuccagAfgUfuauuas | 1963 |
| | {invAb} | | usu | |
| D-1027 | asusaacuCfuGfGfAfGfgucaaagus | 1116 | asAfscuuuGfaccuccAfgAfguuaus | 1964 |
| | {invAb} | | usu | |
| D-1028 | asuscuggAfaCfAfCfUfaucagcaus | 1117 | asAfsugcuGfauagugUfuCfcagaus | 1965 |
| | {invAb} | | usu | |
| D-1029 | asgsgaugAfaGfUfUfCfgacaugggs | 1118 | usCfsccauGfucgaacUfuCfauccus | 1966 |
| | {invAb} | | usu | |
| D-1030 | gsusucgaCfaUfGfGGfagagacaas | 1119 | asUfsugucUfcucccaUfgUfcgaacs | 1967 |
| | {invAb} | | usu | |
| D-1031 | csgsacauGfgGfAfGfAfgacaagggs | 1120 | usCfsccuuGfucucucCfcAfugucgs | 1968 |
| | {invAb} | | usu | |
| D-1032 | ascsauggGfaGfAfGfAfcaagggacs | 1121 | asGfsucccUfugucucUfcCfcaugus | 1969 |
| | {invAb} | | usu | |
| D-1033 | asusgggaGfaGfAfCfAfagggacuus | 1122 | usAfsagucCfcuugucUfcUfcccaus | 1970 |
| | {invAb} | | usu | |
| D-1034 | gsgsgagaGfaCfAfAfGfggacuuaus | 1123 | asAfsuaagUfcccuugUfcUfcucccs | 1971 |
| | {invAb} | | usu | |
| D-1035 | gsgsagagAfcAfAfGfGfgacuuaucs | 1124 | usGfsauaaGfucccuuGfuCfucuccs | 1972 |
| | {invAb} | | usu | |
| D-1036 | gsasgagaCfaAfGfGfGfacuuaucas | 1125 | usUfsgauaAfgucccuUfgUfcucucs | 1973 |
| | {invAb} | | usu | |
| D-1037 | gsascaagGfgAfCfUfUfaucaacaas | 1126 | usUfsuguuGfauaaguCfcCfuugucs | 1974 |
| | {invAb} | | usu | |
| D-1038 | ascsaaggGfaCfUfUfAfucaacaaas | 1127 | asUfsuuguUfgauaagUfcCfcuugus | 1975 |
| | {invAb} | | usu | |
| D-1039 | gsgsgacuUfaUfCfAfAfcaaagaaas | 1128 | usUfsuucuUfuguugaUfaAfgucccs | 1976 |
| | {invAb} | | usu | |
| D-1040 | asasgaaaAfuAfCfUfCfcuucugggs | 1129 | asCfsccagAfaggaguAfuUfuucuus | 1977 |
| | {invAb} | | usu | |
| D-1041 | asusacucCfuUfCfUfGfgguucaacs | 1130 | asGfsuugaAfcccagaAfgGfaguaus | 1978 |
| | {invAb} | | usu | |
| D-1042 | gsusucaaCfcAfCfCfUfugaugauus | 1131 | asAfsaucaUfcaagguGfgUfugaacs | 1979 |
| | {invAb} | | usu | |
| D-1043 | ususcaacCfaCfCfUfUfgaugauugs | 1132 | asCfsaaucAfucaaggUfgGfuugaas | 1980 |
| | {invAb} | | usu | |
| D-1044 | ususgaauAfcUfCfAfGfgaagucgas | 1133 | usUfscgacUfuccugaGfuAfuucaas | 1981 |
| | {invAb} | | usu | |
| D-1045 | ascsucagGfaAfGfUfCfgaaaaggus | 1134 | usAfsccuuUfucgacuUfcCfugagus | 1982 |
| | {invAb} | | usu | |
| D-1046 | csuscaggAfaGfUfCfGfaaaagguas | 1135 | asUfsaccuUfuucgacUfuCfcugags | 1983 |
| | {invAb} | | usu | |
| D-1047 | uscsaggaAfgUfCfGfAfaaagguacs | 1136 | usGfsuaccUfuuucgaCfuUfccugas | 1984 |
| | {invAb} | | usu | |
| D-1048 | asgsgaagUfcGfAfAfAfagguacacs | 1137 | usGfsuguaCfcuuuucGfaCfuuccus | 1985 |
| | {invAb} | | usu | |
| D-1049 | uscsgaaaAfgGfUfAfCfacaaaaaus | 1138 | usAfsuuuuUfguguacCfuUfuucgas | 1986 |
| | {invAb} | | usu | |
| D-1050 | csgsaaaaGfgUfAfCfAfcaaaaauas | 1139 | asUfsauuuUfuguguaCfcUfuuucgs | 1987 |
| | {invAb} | | usu | |
| D-1051 | gsasgaaaGfgAfGfCfAfagccuaaas | 1140 | asUfsuuagGfcuugcuCfcUfuucucs | 1988 |
| | {invAb} | | usu | |
| D-1052 | asgsaaagGfaGfCfAfAfgccuaaacs | 1141 | asGfsuuuaGfgcuugcUfcCfuuucus | 1989 |
| | {invAb} | | usu | |
| D-1053 | gsasaaggAfgCfAfAfGfccuaaacgs | 1142 | asCfsguuuAfggcuugCfuCfcuuucs | 1990 |
| | {invAb} | | usu | |
| D-1054 | asgsgagcAfaGfCfCfUfaaacgucas | 1143 | asUfsgacgUfuuaggcUfuGfcuccus | 1991 |
| | {invAb} | | usu | |
| D-1055 | gsgsagcaAfgCfCfUfAfaacgucags | 1144 | usCfsugacGfuuuaggCfuUfgcuccs | 1992 |
| | {invAb} | | usu | |
| D-1056 | gsasgcaaGfcCfUfAfAfacgucagas | 1145 | usUfscugaCfguuuagGfcUfugcucs | 1993 |
| | {invAb} | | usu | |
| D-1057 | asgscaagCfcUfAfAfAfcgucagaas | 1146 | usUfsucugAfcguuuaGfgCfuugcus | 1994 |
| | {invAb} | | usu | |
| D-1058 | gscsaagcCfuAfAfAfCfgucagaaas | 1147 | asUfsuucuGfacguuuAfgGfcuugcs | 1995 |
| | {invAb} | | usu | |
| D-1059 | csasagccUfaAfAfCfGfucagaaaus | 1148 | asAfsuuucUfgacguuUfaGfgcuugs | 1996 |
| | {invAb} | | usu | |
| D-1060 | asasgccuAfaAfCfGfUfcagaaaucs | 1149 | asGfsauuuCfugacguUfuAfggcuus | 1997 |
| | {invAb} | | usu | |
| D-1061 | gsuscagaAfaUfCfCfAfguacuaaas | 1150 | asUfsuuagUfacuggaUfuUfcugacs | 1998 |
| | {invAb} | | usu | |
| D-1062 | uscsagaaAfuCfCfAfGfuacuaaacs | 1151 | asGfsuuuaGfuacuggAfuUfucugas | 1999 |
| | {invAb} | | usu | |
| D-1063 | ususucugAfgCfUfUfCfaugacaaus | 1152 | asAfsuuguCfaugaagCfuCfagaaas | 2000 |
| | {invAb} | | usu | |
| D-1064 | asgscuucAfuGfAfCfAfaucaggacs | 1153 | asGfsuccuGfauugucAfuGfaagcus | 2001 |
| | {invAb} | | usu | |
| D-1065 | ususcaugAfcAfAfUfCfaggacggus | 1154 | asAfsccguCfcugauuGfuCfaugaas | 2002 |
| | (invAb} | | usu | |
| D-1066 | uscsaugaCfaAfUfCfAfggacggucs | 1155 | asGfsaccgUfccugauUfgUfcaugas | 2003 |
| | {invAb} | | usu | |
| D-1067 | csasugacAfaUfCfAfGfgacggucus | 1156 | asAfsgaccGfuccugaUfuGfucaugs | 2004 |
| | {invAb} | | usu | |
| D-1068 | asusgacaAfuCfAfGfGfacggucuus | 1157 | asAfsagacCfguccugAfuUfgucaus | 2005 |
| | {invAb} | | usu | |
| D-1069 | usgsacaaUfcAfGfGfAfcggucuugs | 1158 | asCfsaagaCfcguccuGfaUfugucas | 2006 |
| | {invAb} | | usu | |
| D-1070 | gsascaauCfaGfGfAfCfggucuugus | 1159 | asAfscaagAfccguccUfgAfuugucs | 2007 |
| | {invAb} | | usu | |
| D-1071 | ascsaaucAfgGfAfCfGfgucuugugs | 1160 | usCfsacaaGfaccgucCfuGfauugus | 2008 |
| | {invAb} | | usu | |
| D-1072 | asasucagGfaCfGfGfUfcuugugaas | 1161 | asUfsucacAfagaccgUfcCfugauus | 2009 |
| | {invAb} | | usu | |
| D-1073 | asuscaggAfcGfGfUfCfuugugaaus | 1162 | usAfsuucaCfaagaccGfuCfcugaus | 2010 |
| | {invAb} | | usu | |
| D-1074 | uscsaggaCfgGfUfCfUfugugaauas | 1163 | asUfsauucAfcaagacCfgUfccugas | 2011 |
| | {invAb} | | usu | |
| D-1075 | gsgsucuuGfuGfAfAfUfauggaaags | 1164 | asCfsuuucCfauauucAfcAfagaccs | 2012 |
| | {invAb} | | usu | |
| D-1076 | gsasaaguCfuCfAfAfUfuccacacgs | 1165 | usCfsguguGfgaauugAfgAfcuuucs | 2013 |
| | {invAb} | | usu | |
| D-1077 | asasagucUfcAfAfUfUfccacacgas | 1166 | asUfscgugUfggaauuGfaGfacuuus | 2014 |
| | {invAb} | | usu | |
| D-1078 | asasgucuCfaAfUfUfCfcacacgaus | 1167 | asAfsucguGfuggaauUfgAfgacuus | 2015 |
| | {invAb} | | usu | |
| D-1079 | asgsucucAfaUfUfCfCfacacgaucs | 1168 | asGfsaucgUfguggaaUfuGfagacus | 2016 |
| | {invAb} | | usu | |
| D-1080 | gsuscucaAfuUfCfCfAfcacgaucus | 1169 | asAfsgaucGfuguggaAfuUfgagacs | 2017 |
| | {invAb} | | usu | |
| D-1081 | uscsucaaUfuCfCfAfCfacgaucucs | 1170 | usGfsagauCfguguggAfaUfugagas | 2018 |
| | {invAb} | | usu | |
| D-1082 | csuscaauUfcCfAfCfAfcgaucucas | 1171 | asUfsgagaUfcgugugGfaAfuugags | 2019 |
| | {invAb} | | usu | |
| D-1083 | uscsaauuCfcAfCfAfCfgaucucaus | 1172 | asAfsugagAfucguguGfgAfauugas | 2020 |
| | {invAb} | | usu | |
| D-1084 | csasauucCfaCfAfCfGfaucucaugs | 1173 | usCfsaugaGfaucgugUfgGfaauugs | 2021 |
| | {invAb} | | usu | |
| D-1085 | asusuccaCfaCfGfAfUfcucaugags | 1174 | usCfsucauGfagaucgUfgUfggaaus | 2022 |
| | {invAb} | | usu | |
| D-1086 | ususccacAfcGfAfUfCfucaugagas | 1175 | asUfscucaUfgagaucGfuGfuggaas | 2023 |
| | {invAb} | | usu | |
| D-1087 | uscscacaCfgAfUfCfUfcaugagags | 1176 | usCfsucucAfugagauCfgUfguggas | 2024 |
| | {invAb} | | usu | |
| D-1088 | csascgauCfuCfAfUfGfagagaacus | 1177 | asAfsguucUfcucaugAfgAfucgugs | 2025 |
| | {invAb} | | usu | |
| D-1089 | uscsucauGfaGfAfGfAfacuggaccs | 1178 | asGfsguccAfguucucUfcAfugagas | 2026 |
| | {invAb} | | usu | |
| D-1090 | uscsaugaGfaGfAfAfCfuggaccugs | 1179 | usCfsagguCfcaguucUfcUfcaugas | 2027 |
| | {invAb} | | usu | |
| D-1091 | csasugagAfgAfAfCfUfggaccugas | 1180 | asUfscaggUfccaguuCfuCfucaugs | 2028 |
| | {invAb} | | usu | |
| D-1092 | ususgaauGfgAfUfGfUfcugaugaas | 1181 | usUfsucauCfagacauCfcAfuucaas | 2029 |
| | {invAb} | | usu | |
| D-1093 | gsgsuggaCfaCfAfCfUfcagcauuus | 1182 | asAfsaaugCfugagugUfgUfccaccs | 2030 |
| | {invAb} | | usu | |
| D-1094 | ususugagAfgCfCfCfCfacaaugaas | 1183 | asUfsucauUfguggggCfuCfucaaas | 2031 |
| | {invAb} | | usu | |
| D-1095 | asuscccaGfcCfUfAfUfcugacaccs | 1184 | usGfsguguCfagauagGfcUfgggaus | 2032 |
| | {invAb} | | usu | |
| D-1096 | uscsccagCfcUfAfUfCfugacaccas | 1185 | usUfsggugUfcagauaGfgCfugggas | 2033 |
| | {invAb} | | usu | |
| D-1097 | cscscagcCfuAfUfCfUfgacaccaas | 1186 | usUfsugguGfucagauAfgGfcugggs | 2034 |
| | {invAb} | | usu | |
| D-1098 | cscsagccUfaUfCfUfGfacaccaaas | 1187 | asUfsuuggUfgucagaUfaGfgcuggs | 2035 |
| | {invAb} | | usu | |
| D-1099 | csasgccuAfuCfUfGfAfcaccaaacs | 1188 | usGfsuuugGfugucagAfuAfggcugs | 2036 |
| | {invAb} | | usu | |
| D-1100 | asgscagaGfaAfAfUfCfaagaugccs | 1189 | asGfsgcauCfuugauuUfcUfcugcus | 2037 |
| | {invAb} | | usu | |
| D-1101 | gsasgcuuUfgUfCfUfCfcgaagugcs | 1190 | asGfscacuUfcggagaCfaAfagcucs | 2038 |
| | {invAb} | | usu | |
| D-1102 | usgsucucCfgAfAfGfUfgccccagus | 1191 | asAfscuggGfgcacuuCfgGfagacas | 2039 |
| | {invAb} | | usu | |
| D-1103 | csusccgaAfgUfGfCfCfccagucggs | 1192 | usCfscgacUfggggcaCfuUfcggags | 2040 |
| | {invAb} | | usu | |
| D-1104 | uscscgaaGfuGfCfCfCfcagucggas | 1193 | asUfsccgaCfuggggcAfcUfucggas | 2041 |
| | {invAb} | | usu | |
| D-1105 | asgsaacuGfgGfAfAfGfagccuaucs | 1194 | asGfsauagGfcucuucCfcAfguucus | 2042 |
| | {invAb} | | usu | |
| D-1106 | gsasacugGfgAfAfGfAfgccuauccs | 1195 | asGfsgauaGfgcucuuCfcCfaguucs | 2043 |
| | {invAb} | | usu | |
| D-1107 | asasgagcCfuAfUfCfCfcugcuuucs | 1196 | asGfsaaagCfagggauAfgGfcucuus | 2044 |
| | {invAb} | | usu | |
| D-1108 | asgsgcugGfgCfGfCfCfugauccgus | 1197 | asAfscggaUfcaggcgCfcCfagccus | 2045 |
| | {invAb} | | usu | |
| D-1109 | gsgscuggGfcGfCfCfUfgauccgucs | 1198 | usGfsacggAfucaggcGfcCfcagccs | 2046 |
| | {invAb} | | usu | |
| D-1110 | gscsugggCfgCfCfUfGfauccgucas | 1199 | asUfsgacgGfaucaggCfgCfccagcs | 2047 |
| | {invAb} | | usu | |
| D-1111 | csusgggcGfcCfUfGfAfuccgucags | 1200 | asCfsugacGfgaucagGfcGfcccags | 2048 |
| | {invAb} | | usu | |
| D-1112 | usgsggcgCfcUfGfAfUfccgucagcs | 1201 | asGfscugaCfggaucaGfgCfgcccas | 2049 |
| | {invAb} | | usu | |
| D-1113 | csusggacGfaAfGfAfCfagcgacccs | 1202 | asGfsggucGfcugucuUfcGfuccags | 2050 |
| | {invAb} | | usu | |
| D-1114 | ascscccaUfgCfUfCfUfcuccucggs | 1203 | asCfscgagGfagagagCfaUfggggus | 2051 |
| | {invAb} | | usu | |
| D-1115 | csasugcuCfuCfUfCfCfucgguucus | 1204 | usAfsgaacCfgaggagAfgAfgcaugs | 2052 |
| | {invAb} | | usu | |
| D-1116 | asusgcucUfcUfCfCfUfcgguucuas | 1205 | asUfsagaaCfcgaggaGfaGfagcaus | 2053 |
| | {invAb} | | usu | |
| D-1117 | gscsucucUfcCfUfCfGfguucuacgs | 1206 | asCfsguagAfaccgagGfaGfagagcs | 2054 |
| | {invAb} | | usu | |
| 0-1118 | csuscucuCfcUfCfGfGfuucuacgcs | 1207 | asGfscguaGfaaccgaGfgAfgagags | 2055 |
| | {invAb} | | usu | |
| D-1119 | uscsucucCfuCfGfGfUfucuacgcus | 1208 | asAfsgcguAfgaaccgAfgGfagagas | 2056 |
| | {invAb} | | usu | |
| D-1120 | csuscuccUfcGfGfUfUfcuacgcuus | 1209 | usAfsagcgUfagaaccGfaGfgagags | 2057 |
| | {invAb} | | usu | |
| D-1121 | uscsuccuCfgGfUfUfCfuacgcuuas | 1210 | asUfsaagcGfuagaacCfgAfggagas | 2058 |
| | {invAb} | | usu | |
| D-1122 | csusccucGfgUfUfCfUfacgcuuaus | 1211 | asAfsuaagCfguagaaCfcGfaggags | 2059 |
| | {invAb} | | usu | |
| D-1123 | uscscucgGfuUfCfUfAfcgcuuaugs | 1212 | asCfsauaaGfcguagaAfcCfgaggas | 2060 |
| | {invAb} | | usu | |
| D-1124 | cscsucggUfuCfUfAfCfgcuuauggs | 1213 | asCfscauaAfgcguagAfaCfcgaggs | 2061 |
| | {invAb} | | usu | |
| D-1125 | csuscgguUfcUfAfCfGfcuuaugggs | 1214 | asCfsccauAfagcguaGfaAfccgags | 2062 |
| | {invAb} | | usu | |
| D-1126 | csasccaaAfcUfCfCfCfauucuuucs | 1215 | usGfsaaagAfaugggaGfuUfuggugs | 2063 |
| | {invAb} | | usu | |
| D-1127 | cscsaaacUfcCfCfAfUfucuuucaus | 1216 | asAfsugaaAfgaauggGfaGfuuuggs | 2064 |
| | {invAb} | | usu | |
| D-1128 | csuscccaUfuCfUfUfUfcaugaggcs | 1217 | asGfsccucAfugaaagAfaUfgggags | 2065 |
| | {invAb} | | usu | |
| D-1129 | csasuucuUfuCfAfUfGfaggcggcgs | 1218 | usCfsgccgCfcucaugAfaAfgaaugs | 2066 |
| | {invAb} | | usu | |
| D-1130 | asusucuuUfcAfUfGfAfggcggcgas | 1219 | usUfscgccGfccucauGfaAfagaaus | 2067 |
| | {invAb} | | usu | |
| D-1131 | ususcuuuCfaUfGfAfGfgcggcgaas | 1220 | asUfsucgcCfgccucaUfgAfaagaas | 2068 |
| | {invAb} | | usu | |
| D-1132 | uscsuuucAfuGfAfGfGfcggcgaags | 1221 | asCfsuucgCfcgccucAfuGfaaagas | 2069 |
| | {invAb} | | usu | |
| D-1133 | csusuucaUfgAfGfGfCfggcgaagcs | 1222 | asGfscuucGfccgccuCfaUfgaaags | 2070 |
| | {invAb} | | usu | |
| D-1134 | ususucauGfaGfGfCfGfgcgaagcus | 1223 | asAfsgcuuCfgccgccUfcAfugaaas | 2071 |
| | {invAb} | | usu | |
| D-1135 | uscsucugGfgGfUfCfCfuaugaugas | 1224 | asUfscaucAfuaggacCfcCfagagas | 2072 |
| | {invAb} | | usu | |
| D-1136 | ascsaccuGfcCfCfAfGfcucacacgs | 1225 | usCfsguguGfagcuggGfcAfggugus | 2073 |
| | {invAb} | | usu | |
| D-1137 | csasccugCfcCfAfGfCfucacacgas | 1226 | usUfscgugUfgagcugGfgCfaggugs | 2074 |
| | {invAb} | | usu | |
| D-1138 | cscsugccCfaGfCfUfCfacacgaags | 1227 | asCfsuucgUfgugagcUfgGfgcaggs | 2075 |
| | {invAb} | | usu | |
| D-1139 | cscsagcuCfaCfAfCfGfaaggauucs | 1228 | usGfsaaucCfuucgugUfgAfgcuggs | 2076 |
| | {invAb} | | usu | |
| D-1140 | asgscucaCfaCfGfAfAfggauucags | 1229 | usCfsugaaUfccuucgUfgUfgagcus | 2077 |
| | {invAb} | | usu | |
| D-1141 | asusccggAfaGfUfUfUfgaagauags | 1230 | usCfsuaucUfucaaacUfuCfcggaus | 2078 |
| | {invAb} | | usu | |
| D-1142 | csgsgaagUfuUfGfAfAfgauagauus | 1231 | asAfsaucuAfucuucaAfaCfuuccgs | 2079 |
| | {invAb} | | usu | |
| D-1143 | asgsuuugAfaGfAfUfAfgauucgaas | 1232 | asUfsucgaAfucuaucUfuCfaaacus | 2080 |
| | {invAb} | | usu | |
| D-1144 | gsusuugaAfgAfUfAfGfauucgaags | 1233 | usCfsuucgAfaucuauCfuUfcaaacs | 2081 |
| | {invAb} | | usu | |
| D-1145 | gsasagauAfgAfUfUfCfgaagaagas | 1234 | asUfscuucUfucgaauCfuAfucuucs | 2082 |
| | (invAb} | | usu | |
| D-1146 | asgsaagaAfgUfAfCfAfgaccuuccs | 1235 | asGfsgaagGfucuguaCfuUfcuucus | 2083 |
| | {invAb} | | usu | |
| D-1147 | gsasagaaGfuAfCfAfGfaccuucccs | 1236 | usGfsggaaGfgucuguAfcUfucuucs | 2084 |
| | {invAb} | | usu | |
| D-1148 | asasgaagUfaCfAfGfAfccuucccas | 1237 | asUfsgggaAfggucugUfaCfuucuus | 2085 |
| | {invAb} | | usu | |
| D-1149 | uscsugaaAfuGfGfAfCfaaaugaccs | 1238 | asGfsgucaUfuuguccAfuUfucagas | 2086 |
| | {invAb} | | usu | |
| D-1150 | csusgaaaUfgGfAfCfAfaaugaccus | 1239 | asAfsggucAfuuugucCfaUfuucags | 2087 |
| | {invAb} | | usu | |
| D-1151 | ascsaaauGfaCfCfUfUfgccaaauus | 1240 | asAfsauuuGfgcaaggUfcAfuuugus | 2088 |
| | {invAb} | | usu | |
| D-1152 | asasaugaCfcUfUfGfCfcaaauuccs | 1241 | asGfsgaauUfuggcaaGfgUfcauuus | 2089 |
| | {invAb} | | usu | |
| D-1153 | asasugacCfuUfGECECfaaauuccgs | 1242 | asCfsggaaUfuuggcaAfgGfucauus | 2090 |
| | {invAb} | | usu | |
| D-1154 | usgsaccuUfgCfCfAfAfauuccggas | 1243 | asUfsccggAfauuuggCfaAfggucas | 2091 |
| | {invAb} | | usu | |
| D-1155 | gsasccuuGfcCfAfAfAfuuccggags | 1244 | usCfsuccgGfaauuugGfcAfaggucs | 2092 |
| | {invAb} | | usu | |
| D-1156 | ascscuugCfcAfAfAfUfuccggagas | 1245 | asUfscuccGfgaauuuGfgCfaaggus | 2093 |
| | {invAb} | | usu | |
| D-1157 | cscsuugcCfaAfAfUfUfccggagacs | 1246 | usGfsucucCfggaauuUfgGfcaaggs | 2094 |
| | {invAb} | | usu | |
| D-1158 | cscsggagAfcAfAfCfUfuaaagaaus | 1247 | asAfsuucuUfuaaguuGfuCfuccggs | 2095 |
| | {invAb} | | usu | |
| D-1159 | csgsgagaCfaAfCfUfUfaaagaaucs | 1248 | usGfsauucUfuuaaguUfgUfcuccgs | 2096 |
| | (invAb} | | usu | |
| D-1160 | asgsauauCfuGfAfAfGfaggaccuas | 1249 | usUfsagguCfcucuucAfgAfuaucus | 2097 |
| | {invAb} | | usu | |
| D-1161 | gsasggacCfuAfAfCfUfcccaggaus | 1250 | asAfsuccuGfggaguuAfgGfuccucs | 2098 |
| | {invAb} | | usu | |
| D-1162 | gsasccuaAfcUfCfCfCfaggaugcgs | 1251 | asCfsgcauCfcugggaGfuUfaggucs | 2099 |
| | {invAb} | | usu | |
| D-1163 | ascscuaaCfuCfCfCfAfggaugcggs | 1252 | asCfscgcaUfccugggAfgUfuaggus | 2100 |
| | {invAb} | | usu | |
| D-1164 | usgscggcAfgCfGfAfAfgcaacacas | 1253 | asUfsguguUfgcuucgCfuGfccgcas | 2101 |
| | {invAb} | | usu | |
| D-1165 | gscsggcaGfcGfAfAfGfcaacacacs | 1254 | asGfsugugUfugcuucGfcUfgccgcs | 2102 |
| | {invAb} | | usu | |
| D-1166 | csasgcgaAfgCfAfAfCfacacucccs | 1255 | asGfsggagUfguguugCfuUfcgcugs | 2103 |
| | {invAb} | | usu | |
| D-1167 | gscsgaagCfaAfCfAfCfacuccccas | 1256 | usUfsggggAfguguguUfgCfuucgcs | 2104 |
| | {invAb} | | usu | |
| D-1168 | gscsaacaCfaCfUfCfCfccaagagus | 1257 | asAfscucuUfggggagUfgUfguugcs | 2105 |
| | {invAb} | | usu | |
| D-1169 | csasagagUfuUfUfGfGfuucccaacs | 1258 | asGfsuuggGfaaccaaAfaCfucuugs | 2106 |
| | {invAb} | | usu | |
| D-1170 | gsasguuuUfgGfUfUfCfccaacuugs | 1259 | usCfsaaguUfgggaacCfaAfaacucs | 2107 |
| | {invAb} | | usu | |
| D-1171 | gsusuuugGfuUfCfCfCfaacuugags | 1260 | usCfsucaaGfuugggaAfcCfaaaacs | 2108 |
| | {invAb} | | usu | |
| D-1172 | ususgaagCfcAfCfAfUfuggaaucus | 1261 | usAfsgauuCfcaauguGfgCfuucaas | 2109 |
| | {invAb} | | usu | |
| D-1173 | cscsaggaGfaAfGfCfGfagcggaaas | 1262 | asUfsuuccGfcucgcuUfcUfccuggs | 2110 |
| | {invAb} | | usu | |
| D-1174 | gsasccagAfuUfGfCfUfaaugagaas | 1263 | usUfsucucAfuuagcaAfuCfuggucs | 2111 |
| | {invAb} | | usu | |
| D-1175 | gscsuaauGfaGfAfAfAfguggcucus | 1264 | asAfsgagcCfacuuucUfcAfuuagcs | 2112 |
| | {invAb} | | usu | |
| D-1176 | asgscauuCfaUfGfGfAfcggccggus | 1265 | usAfsccggCfcguccaUfgAfaugcus | 2113 |
| | {invAb] | | usu | |
| D-1177 | gscsauucAfuGfGfAfCfggccgguas | 1266 | usUfsaccgGfccguccAfuGfaaugcs | 2114 |
| | {invAb} | | usu | |
| D-1178 | csasuucaUfgGfAfCfGfgccgguaas | 1267 | asUfsuaccGfgccgucCfaUfgaaugs | 2115 |
| | {invAb} | | usu | |
| D-1179 | asusucauGfgAfCfGfGfccgguaacs | 1268 | usGfsuuacCfggccguCfcAfugaaus | 2116 |
| | {invAb} | | usu | |
| D-1180 | ususcaugGfaCfGfGfCfcgguaacas | 1269 | usUfsguuaCfcggccgUfcCfaugaas | 2117 |
| | {invAb} | | usu | |
| D-1181 | uscsauggAfcGfGfCfCfgguaacaas | 1270 | usUfsuguuAfccggccGfuCfcaugas | 2118 |
| | {invAb} | | usu | |
| D-1182 | asusggacGfgCfCfGfGfuaacaaags | 1271 | usCfsuuugUfuaccggCfcGfuccaus | 2119 |
| | {invAb} | | usu | |
| D-1183 | usgsgacgGfcCfGfGfUfaacaaagas | 1272 | usUfscuuuGfuuaccgGfcCfguccas | 2120 |
| | {invAb} | | usu | |
| D-1184 | gsgsacggCfcGfGfUfAfacaaagaas | 1273 | asUfsucuuUfguuaccGfgCfcguccs | 2121 |
| | {invAb] | | usu | |
| D-1185 | ascsggccGfgUfAfAfCfaaagaacgs | 1274 | usCfsguucUfuuguuaCfcGfgccgus | 2122 |
| | {invAb} | | usu | |
| D-1186 | csgsgccgGfuAfAfCfAfaagaacgas | 1275 | usUfscguuCfuuuguuAfcCfggccgs | 2123 |
| | {invAb} | | usu | |
| D-1187 | gsgsccggUfaAfCfAfAfagaacgaas | 1276 | asUfsucguUfcuuuguUfaCfcggccs | 2124 |
| | {invAb | | usu | |
| D-1188 | gscscgguAfaCfAfAfAfgaacgaacs | 1277 | asGfsuucgUfucuuugUfuAfccggcs | 2125 |
| | {invAb} | | usu | |
| D-1189 | cscsgguaAfcAfAfAfGfaacgaacgs | 1278 | asCfsguucGfuucuuuGfuUfaccggs | 2126 |
| | {invAb} | | usu | |
| D-1190 | csgsguaaCfaAfAfGfAfacgaacggs | 1279 | asCfscguuCfguucuuUfgUfuaccgs | 2127 |
| | {invAb} | | usu | |
| D-1191 | gsgsuaacAfaAfGAfAfcgaacggcs | 1280 | usGfsccguUfcguucuUfuGfuuaccs | 2128 |
| | {invAb} | | usu | |
| D-1192 | asasagaaCfgAfAfCfGfgcaggugas | 1281 | asUfscaccUfgccguuCfgUfucuuus | 2129 |
| | {invAb} | | usu | |
| D-1193 | asusgaagCfcAfCfUfAfuacgacags | 1282 | asCfsugucGfuauaguGfgCfuucaus | 2130 |
| | {invAb} | | usu | |
| D-1194 | gsasagccAfcUfAfUfAfcgacaggus | 1283 | usAfsccugUfcguauaGfuGfgcuucs | 2131 |
| | {invAb} | | usu | |
| D-1195 | asasgccaCfuAfUfAfCfgacagguas | 1284 | asUfsaccuGfucguauAfgUfggcuus | 2132 |
| | {invAb} | | usu | |
| D-1196 | asgsccacUfaUfAfCfGfacagguacs | 1285 | asGfsuaccUfgucguaUfaGfuggcus | 2133 |
| | {invAb} | | usu | |
| D-1197 | gscscacuAfuAfCfGfAfcagguaccs | 1286 | asGfsguacCfugucguAfuAfguggcs | 2134 |
| | {invAb} | | usu | |
| D-1198 | cscsacuaUfaCfGfAfCfagguaccgs | 1287 | asCfsgguaCfcugucgUfaUfaguggs | 2135 |
| | {invAb} | | usu | |
| D-1199 | csascuauAfcGfAfCfAfgguaccggs | 1288 | asCfscgguAfccugucGfuAfuagugs | 2136 |
| | {invAb} | | usu | |
| D-1200 | ascsuauaCfgAfCfAfGfguaccggcs | 1289 | asGfsccggUfaccuguCfgUfauagus | 2137 |
| | {invAb} | | usu | |
| D-1201 | asascagaUfcCfUfCfUfcccgagcus | 1290 | usAfsgcucGfggagagGfaUfcuguus | 2138 |
| | {invAb} | | usu | |
| D-1202 | ascsagauCfcUfCfUfCfccgagcuas | 1291 | usUfsagcuCfgggagaGfgAfucugus | 2139 |
| | {invAb} | | usu | |
| D-1203 | asgsauccUfcUfCfCfCfgagcuaacs | 1292 | usGfsuuagCfucgggaGfaGfgaucus | 2140 |
| | {invAb} | | usu | |
| D-1204 | uscscucuCfcCfGfAfGfcuaacaccs | 1293 | usGfsguguUfagcucgGfgAfgaggas | 2141 |
| | {invAb} | | usu | |
| D-1205 | cscsucucCfcGfAfGfCfuaacaccas | 1294 | asUfsggugUfuagcucGfgGfagaggs | 2142 |
| | {invAb} | | usu | |
| D-1206 | csuscuccCfgAfGfCfUfaacaccaus | 1295 | usAfsugguGfuuagcuCfgGfgagags | 2143 |
| | {invAb} | | usu | |
| D-1207 | uscsccgaGfcUfAfAfCfaccauaccs | 1296 | asGfsguauGfguguuaGfcUfcgggas | 2144 |
| | {invAb} | | usu | |
| D-1208 | cscscgagCfuAfAfCfAfccauacccs | 1297 | usGfsgguaUfgguguuAfgCfucgggs | 2145 |
| | {invAb} | | usu | |
| D-1209 | gsgsggucAfgAfAfGfAfcgauagcas | 1298 | usUfsgcuaUfcgucuuCfuGfaccccs | 2146 |
| | {invAb} | | usu | |
| D-1210 | gsgsgucaGfaAfGfAfCfgauagcaas | 1299 | asUfsugcuAfucgucuUfcUfgacccs | 2147 |
| | {invAb} | | usu | |
| D-1211 | gsuscagaAfgAfCfGfAfuagcaaugs | 1300 | asCfsauugCfuaucguCfuUfcugacs | 2148 |
| | {invAb} | | usu | |
| D-1212 | uscsagaaGfaCfGfAfUfagcaaugus | 1301 | asAfscauuGfcuaucgUfcUfucugas | 2149 |
| | {invAb} | | usu | |
| D-1213 | gsasagacGfaUfAfGfCfaaugugaas | 1302 | asUfsucacAfuugcuaUfcGfucuucs | 2150 |
| | {invAb} | | usu | |
| D-1214 | asgsacgaUfaGfCfAfAfugugaagcs | 1303 | asGfscuucAfcauugcUfaUfcgucus | 2151 |
| | {invAb} | | usu | |
| D-1215 | asusggucAfcUfCfUfGfaaaaccgas | 1304 | asUfscgguUfuucagaGfuGfaccaus | 2152 |
| | {invAb} | | usu | |
| D-1216 | usgsgucaCfuCfUfGfAfaaaccgaus | 1305 | asAfsucggUfuuucagAfgUfgaccas | 2153 |
| | {invAb} | | usu | |
| D-1217 | gsgsucacUfcUfGfAfAfaaccgauus | 1306 | asAfsaucgGfuuuucaGfaGfugaccs | 2154 |
| | {invAb} | | usu | |
| D-1218 | gsasaaacCfgAfUfUfUfcagugcacs | 1307 | asGfsugcaCfugaaauCfgGfuuuucs | 2155 |
| | {invAb} | | usu | |
| D-1219 | asasccgaUfuUfCfAfGfugcacgaus | 1308 | asAfsucguGfcacugaAfaUfcgguus | 2156 |
| | {invAb} | | usu | |
| D-1220 | usasuuucCfcCfAfAfUfggaugauas | 1309 | usUfsaucaUfccauugGfgGfaaauas | 2157 |
| | {invAb} | | usu | |
| D-1221 | cscscaauGfgAfUfGfAfuaaaauacs | 1310 | asGfsuauuUfuaucauCfcAfuugggs | 2158 |
| | {invAb} | | usu | |
| D-1222 | csasauggAfuGfAfUfAfaaauaccas | 1311 | asUfsgguaUfuuuaucAfuCfcauugs | 2159 |
| | {invAb} | | usu | |
| D-1223 | usgsgaugAfuAfAfAfAfuaccaucas | 1312 | usUfsgaugGfuauuuuAfuCfauccas | 2160 |
| | {invAb} | | usu | |
| D-1224 | asasauacCfaUfCfAfAfaaugcagcs | 1313 | asGfscugcAfuuuugaUfgGfuauuus | 2161 |
| | {invAb} | | usu | |
| D-1225 | gsgsgcuuUfcAfAfAfUfcuccaugcs | 1314 | asGfscaugGfagauuuGfaAfagcccs | 2162 |
| | {invAb} | | usu | |
| D-1226 | gsgscuuuCfaAfAfUfCfuccaugcus | 1315 | asAfsgcauGfgagauuUfgAfaagccs | 2163 |
| | {invAb} | | usu | |
| D-1227 | asasucucCfaUfGfCfUfgccucaaus | 1316 | usAfsuugaGfgcagcaUfgGfagauus | 2164 |
| | {invAb} | | usu | |
| D-1228 | asuscuccAfuGfCfUfGfccucaauas | 1317 | asUfsauugAfggcagcAfuGfgagaus | 2165 |
| | {invAb} | | usu | |
| D-1229 | uscsuccaUfgCfUfGfCfcucaauacs | 1318 | asGfsuauuGfaggcagCfaUfggagas | 2166 |
| | {invAb} | | usu | |
| D-1230 | csusgccuCfaAfUfAfCfcugaacucs | 1319 | asGfsaguuCfagguauUfgAfggcags | 2167 |
| | {invAb} | | usu | |
| D-1231 | csusggaaCfaCfCfUfCfcaggaaaus | 1320 | asAfsuuucCfuggaggUfgUfuccags | 2168 |
| | {invAb} | | usu | |
| D-1232 | csusucggGfaUfUfUfUfgaagacaas | 1321 | asUfsugucUfucaaaaUfcCfcgaags | 2169 |
| | {invAb} | | usu | |
| D-1233 | cscsagaaGfgAfAfGfAfccgcacucs | 1322 | asGfsagugCfggucuuCfcUfucuggs | 2170 |
| | {invAb} | | usu | |
| D-1234 | gsasaggaAfgAfCfCfGfcacuccuas | 1323 | asUfsaggaGfugcgguCfuUfccuucs | 2171 |
| | {invAb} | | usu | |
| D-1235 | asasggaaGfaCfCfGfCfacuccuaus | 1324 | asAfsuaggAfgugcggUfcUfuccuus | 2172 |
| | {invAb} | | usu | |
| D-1236 | asgsgaagAfcCfGfCfAfcuccuaugs | 1325 | asCfsauagGfagugcgGfuCfuuccus | 2173 |
| | {invAb} | | usu | |
| D-1237 | gsgsaagaCfcGfCfAfCfuccuauggs | 1326 | asCfscauaGfgagugcGfgUfcuuccs | 2174 |
| | {invAb} | | usu | |
| D-1238 | gsasagacCfgCfAfCfUfccuauggcs | 1327 | asGfsccauAfggagugCfgGfucuucs | 2175 |
| | {invAb} | | usu | |
| D-1239 | asgsaccgCfaCfUfCfCfuauggcugs | 1328 | usCfsagccAfuaggagUfgCfggucus | 2176 |
| | {invAb} | | usu | |
| D-1240 | cscsgcacUfcCfUfAfUfggcugaags | 1329 | usCfsuucaGfccauagGfaGfugcggs | 2177 |
| | {invAb} | | usu | |
| D-1241 | usasagcaCfaUfAfAfAfggcgaaacs | 1330 | asGfsuuucGfccuuuaUfgUfgcuuas | 2178 |
| | {invAb} | | usu | |
| D-1242 | asasgcacAfuAfAfAfGfgcgaaacus | 1331 | asAfsguuuCfgccuuuAfuGfugcuus | 2179 |
| | {invAb} | | usu | |
| D-1243 | gscsacauAfaAfGfGfCfgaaacugas | 1332 | asUfscaguUfucgccuUfuAfugugcs | 2180 |
| | {invAb} | | usu | |
| D-1244 | gsasuuccAfaGfUfCfCfaugugaggs | 1333 | asCfscucaCfauggacUfuGfgaaucs | 2181 |
| | {invAb} | | usu | |
| D-1245 | asusuccaAfgUfCfCfAfugugagggs | 1334 | asCfsccucAfcauggaCfuUfggaaus | 2182 |
| | {invAb} | | usu | |
| D-1246 | cscsaaguCfcAfUfGfUfgaggggcas | 1335 | asUfsgcccCfucacauGfgAfcuuggs | 2183 |
| | {invAb} | | usu | |
| D-1247 | csasagucCfaUfGfUfGfaggggcaus | 1336 | asAfsugccCfcucacaUfgGfacuugs | 2184 |
| | {invAb} | | usu | |
| D-1248 | gsusccauGfuGfAfGfGfggcauggcs | 1337 | asGfsccauGfccccucAfcAfuggacs | 2185 |
| | {invAb} | | usu | |
| D-1249 | gscsagcuGfcGfGfUfGfagaguuuas | 1338 | asUfsaaacUfcucaccGfcAfgcugcs | 2186 |
| | {invAb} | | usu | |
| D-1250 | asgscugcGfgUfGfAfGfaguuuacus | 1339 | asAfsguaaAfcucucaCfcGfcagcus | 2187 |
| | {invAb} | | usu | |
| D-1251 | cscscagaGfaAfAfGfUfgcagcucus | 1340 | asAfsgagcUfgcacuuUfcUfcugggs | 2188 |
| | {invAb} | | usu | |
| D-1252 | csasaagcAfuGfCfAfGfcccuucugs | 1341 | asCfsagaaGfggcugcAfuGfcuuugs | 2189 |
| | {invAb} | | usu | |
| D-1253 | cscsuucuGfcCfUfCfUfagaccauus | 1342 | asAfsauggUfcuagagGfcAfgaaggs | 2190 |
| | {invAb} | | usu | |
| D-1254 | uscsugccUfcUfAfGfAfccauuuggs | 1343 | asCfscaaaUfggucuaGfaGfgcagas | 2191 |
| | {invAb} | | usu | |
| D-1255 | uscsuagaCfcAfUfUfUfggcaucggs | 1344 | asCfscgauGfccaaauGfgUfcuagas | 2192 |
| | {invAb} | | usu | |
| D-1256 | csusagacCfaUfUfUfGfgcaucggcs | 1345 | asGfsccgaUfgccaaaUfgGfucuags | 2193 |
| | {invAb} | | usu | |
| D-1257 | usasgaccAfuUfUfGfGfcaucggcus | 1346 | asAfsgccgAfugccaaAfuGfgucuas | 2194 |
| | {invAb} | | usu | |
| D-1258 | asgsaccaUfuUfGfGfCfaucggcucs | 1347 | asGfsagccGfaugccaAfaUfggucus | 2195 |
| | {invAb} | | usu | |
| D-1259 | ususuggcAfuCfGfGfCfuccuguuus | 1348 | asAfsaacaGfgagccgAfuGfccaaas | 2196 |
| | {invAb} | | usu | |
| D-1260 | gsgscaucGfgCfUfCfCfuguuuccas | 1349 | asUfsggaaAfcaggagCfcGfaugccs | 2197 |
| | {invAb} | | usu | |
| D-1261 | uscsggcuCfcUfGfUfUfuccauugcs | 1350 | asGfscaauGfgaaacaGfgAfgccgas | 2198 |
| | {invAb} | | usu | |
| D-1262 | gsgscuccUfgUfUfUfCfcauugccus | 1351 | asAfsggcaAfuggaaaCfaGfgagccs | 2199 |
| | {invAb} | | usu | |
| D-1263 | usasggcaUfuUfUfGfUfaauuggaas | 1352 | usUfsuccaAfuuacaaAfaUfgccuas | 2200 |
| | {invAb} | | usu | |
| D-1264 | asgsgcauUfuUfGfUfAfauuggaaas | 1353 | asUfsuuccAfauuacaAfaAfugccus | 2201 |
| | {invAb} | | usu | |
| D-1265 | asusuuugUfaAfUfUfGfgaaagucas | 1354 | usUfsgacuUfuccaauUfaCfaaaaus | 2202 |
| | {invAb} | | usu | |
| D-1266 | ususuguaAfuUfGfGfAfaagucaags | 1355 | usCfsuugaCfuuuccaAfuUfacaaas | 2203 |
| | {invAb} | | usu | |
| D-1267 | asasuuggAfaAfGfUfCfaagacugcs | 1356 | usGfscaguCfuugacuUfuCfcaauus | 2204 |
| | {invAb} | | usu | |
| D-1268 | gsgsaaagUfcAfAfGfAfcugcaguas | 1357 | asUfsacugCfagucuuGfaCfuuuccs | 2205 |
| | {invAb} | | usu | |
| D-1269 | gsuscaagAfcUfGfCfAfguaugugcs | 1358 | usGfscacaUfacugcaGfuCfuugacs | 2206 |
| | {invAb} | | usu | |
| D-1270 | uscsaagaCfuGfCfAfGfuaugugcas | 1359 | asUfsgcacAfuacugcAfgUfcuugas | 2207 |
| | {invAb} | | usu | |
| D-1271 | ascsacacAfcAfGfUfAfguggagcus | 1360 | asAfsgcucCfacuacuGfuGfugugus | 2208 |
| | {invAb} | | usu | |
| D-1272 | csascacaGfuAfGfUfGfgagcuuucs | 1361 | asGfsaaagCfuccacuAfcUfgugugs | 2209 |
| | {invAb} | | usu | |
| D-1273 | ascsacagUfaGfUfGfGfagcuuuccs | 1362 | asGfsgaaaGfcuccacUfaCfugugus | 2210 |
| | {invAb} | | usu | |
| D- | ascsaguaGfuGfGfAfGfcuuuccuas | 2821 | usUfsaggaAfagcuccAfcUfacugus | 2822 |
| 1273B | {invAb} | | usu | |
| D-1274 | gscsuuucCfuAfAfCfAfcuagcagas | 1363 | asUfscugcUfaguguuAfgGfaaagcs | 2211 |
| | {invAb} | | usu | |
| D-1275 | usasacacUfaGfCfAfGfagauuaaus | 1364 | asAfsuuaaUfcucugcUfaGfuguuas | 2212 |
| | {invAb} | | usu | |
| D-1276 | asascacuAfgCfAfGfAfgauuaaucs | 1365 | usGfsauuaAfucucugCfuAfguguus | 2213 |
| | {invAb} | | usu | |
| D-1277 | ascsacuaGfcAfGfAfGfauuaaucas | 1366 | asUfsgauuAfaucucuGfcUfagugus | 2214 |
| | {invAb} | | usu | |
| D-1278 | csusagcaGfaGfAfUfUfaaucacuas | 1367 | asUfsagugAfuuaaucUfcUfgcuags | 2215 |
| | {invAb} | | usu | |
| D-1279 | asgsagauUfaAfUfCfAfcuacauuas | 1368 | asUfsaaugUfagugauUfaAfucucus | 2216 |
| | {invAb} | | usu | |
| D-1280 | csusacauUfaGfAfCfAfacacucaus | 1369 | asAfsugagUfguugucUfaAfuguags | 2217 |
| | {invAb} | | usu | |
| D-1281 | usascauuAfgAfCfAfAfcacucaucs | 1370 | asGfsaugaGfuguuguCfuAfauguas | 2218 |
| | {invAb} | | usu | |
| D-1282 | csasuuagAfcAfAfCfAfcucaucuas | 1371 | asUfsagauGfaguguuGfuCfuaaugs | 2219 |
| | {invAb} | | usu | |
| D-1283 | gsgsauaaCfuGfAfGfAfaacaagags | 1372 | usCfsucuuGfuuucucAfgUfuauccs | 2220 |
| | {invAb} | | usu | |
| D-1284 | gsasgaccAfuUfCfUfCfugucuaacs | 1373 | asGfsuuagAfcagagaAfuGfgucucs | 2221 |
| | {invAb} | | usu | |
| D- | csusgucuAfaCfUfGfUfgauaaaaas | 1374 | asUfsuuuuAfucacagUfuAfgacags | 2222 |
| 1284B | {invAb} | | usu | |
| D-1285 | uscsaggaCfuUfUfAfUfucuauagas | 1375 | asUfscuauAfgaauaaAfgUfccugas | 2223 |
| | {invAb} | | usu | |
| D-1286 | ascsuuuaUfuCfUfAfUfagagcaaas | 1376 | asUfsuugcUfcuauagAfaUfaaagus | 2224 |
| | {invAb} | | usu | |
| D-1287 | ususauucUfaUfAfGfAfgcaaacuus | 1377 | asAfsaguuUfgcucuaUfaGfaauaas | 2225 |
| | {invAb} | | usu | |
| D-1288 | usasuucuAfuAfGfAfGfcaaacuugs | 1378 | asCfsaaguUfugcucuAfuAfgaauas | 2226 |
| | {invAb} | | usu | |
| D-1289 | ususcuauAfgAfGfCfAfaacuugcus | 1379 | asAfsgcaaGfuuugcuCfuAfuagaas | 2227 |
| | {invAb} | | usu | |
| D-1290 | csusguggAfgGfGfCfCfaugcucucs | 1380 | asGfsagagCfauggccCfuCfcacags | 2228 |
| | {invAb} | | usu | |
| D-1291 | gscsucucCfuUfGfGfAfcccaguuas | 1381 | usUfsaacuGfgguccaAfgGfagagcs | 2229 |
| | {invAb} | | usu | |
| D-1292 | uscsuccuUfgGfAfCfCfcaguuaacs | 1382 | asGfsuuaaCfugggucCfaAfggagas | 2230 |
| | {invAb} | | usu | |
| D-1293 | csusccuuGfgAfCfCfCfaguuaacus | 1383 | asAfsguuaAfcuggguCfcAfaggags | 2231 |
| | {invAb} | | usu | |
| D-1294 | cscsuuggAfcCfCfAfGfuuaacugcs | 1384 | usGfscaguUfaacuggGfuCfcaaggs | 2232 |
| | {invAb} | | usu | |
| D-1295 | csusuggaCfcCfAfGfUfuaacugcas | 1385 | usUfsgcagUfuaacugGfgUfccaags | 2233 |
| | {invAb} | | usu | |
| D-1296 | gsgsacccAfgUfUfAfAfcugcaaacs | 1386 | asGfsuuugCfaguuaaCfuGfgguccs | 2234 |
| | {invAb} | | usu | |
| D-1297 | gsascccaGfuUfAfAfCfugcaaacgs | 1387 | asCfsguuuGfcaguuaAfcUfgggucs | 2235 |
| | {invAb} | | usu | |
| D-1298 | ascsccagUfuAfAfCfUfgcaaacgus | 1388 | asAfscguuUfgcaguuAfaCfugggus | 2236 |
| | {invAb} | | usu | |
| D-1299 | cscscaguUfaAfCfUfGfcaaacgugs | 1389 | asCfsacguUfugcaguUfaAfcugggs | 2237 |
| | {invAb} | | usu | |
| D-1300 | cscsaguuAfaCfUfGfCfaaacgugcs | 1390 | usGfscacgUfuugcagUfuAfacuggs | 2238 |
| | {invAb} | | usu | |
| D-1301 | csasguuaAfcUfGfCfAfaacgugcas | 1391 | asUfsgcacGfuuugcaGfuUfaacugs | 2239 |
| | {invAb} | | usu | |
| D-1302 | ususaacuGfcAfAfAfCfgugcauugs | 1392 | asCfsaaugCfacguuuGfcAfguuaas | 2240 |
| | {invAb} | | usu | |
| D-1303 | csusgcaaAfcGfUfGfCfauuggagcs | 1393 | asGfscuccAfaugcacGfuUfugcags | 2241 |
| | {invAb} | | usu | |
| D-1304 | usgscaaaCfgUfGfCfAfuuggagccs | 1394 | asGfsgcucCfaaugcaCfgUfuugcas | 2242 |
| | {invAb} | | usu | |
| D-1305 | asascgugCfaUfUfGfGfagcccuaus | 1395 | asAfsuaggGfcuccaaUfgCfacguus | 2243 |
| | {invAb} | | usu | |
| D-1306 | ascsgugcAfuUfGfGfAfgcccuauus | 1396 | asAfsauagGfgcuccaAfuGfcacgus | 2244 |
| | {invAb} | | usu | |
| D-1307 | gsusgcauUfgGfAfGfCfccuauuugs | 1397 | asCfsaaauAfgggcucCfaAfugcacs | 2245 |
| | {invAb} | | usu | |
| D-1308 | usgscauuGfgAfGfCfCfcuauuugcs | 1398 | asGfscaaaUfagggcuCfcAfaugcas | 2246 |
| | {invAb} | | usu | |
| D-1309 | usgsgagcCfcUfAfUfUfugcugccgs | 1399 | asCfsggcaGfcaaauaGfgGfcuccas | 2247 |
| | {invAb} | | usu | |
| D-1310 | gsgsagccCfuAfUfUfUfgcugccgcs | 1400 | asGfscggcAfgcaaauAfgGfgcuccs | 2248 |
| | {invAb} | | usu | |
| D-1311 | gsasgcccUfaUfUfUfGfcugccgcus | 1401 | asAfsgcggCfagcaaaUfaGfggcucs | 2249 |
| | {invAb} | | usu | |
| D-1312 | asgscccuAfuUfUfGfCfugccgcugs | 1402 | asCfsagcgGfcagcaaAfuAfgggcus | 2250 |
| | {invAb} | | usu | |
| D-1313 | cscsgcugCfcAfUfUfCfuagugaccs | 1403 | asGfsgucaCfuagaauGfgCfagcggs | 2251 |
| | {invAb} | | usu | |
| D-1314 | usgsccauUfcUfAfGfUfgaccuuucs | 1404 | asGfsaaagGfucacuaGfaAfuggcas | 2252 |
| | {invAb} | | usu | |
| D-1315 | gscscauuCfuAfGfUfGfaccuuuccs | 1405 | usGfsgaaaGfgucacuAfgAfauggcs | 2253 |
| | {invAb} | | usu | |
| D-1316 | csusagugAfcCfUfUfUfccacagags | 1406 | asCfsucugUfggaaagGfuCfacuags | 2254 |
| | {invAb} | | usu | |
| D-1317 | asgsagcuGfcGfCfCfUfuccucacgs | 1407 | asCfsgugaGfgaaggcGfcAfgcucus | 2255 |
| | {invAb} | | usu | |
| D-1318 | gscsgccuUfcCfUfCfAfcgugugugs | 1408 | usCfsacacAfcgugagGfaAfggcgcs | 2256 |
| | {invAb} | | usu | |
| D-1319 | cscsucacGfuGfUfGfUfgaaagguus | 1409 | asAfsaccuUfucacacAfcGfugaggs | 2257 |
| | {invAb} | | usu | |
| D-1320 | csuscacgUfgUfGfUfGfaaagguuus | 1410 | asAfsaaccUfuucacaCfaCfgugags | 2258 |
| | {invAb} | | usu | |
| D-1321 | ususcagcCfcUfCfAfGfguagauggs | 1411 | usCfscaucUfaccugaGfgGfcugaas | 2259 |
| | {invAb} | | usu | |
| D-1322 | uscsagccCfuCfAfGfGfuagauggas | 1412 | usUfsccauCfuaccugAfgGfgcugas | 2260 |
| | {invAb} | | usu | |
| D-1323 | asgscccuCfaGfGfUfAfgauggaags | 1413 | asCfsuuccAfucuaccUfgAfgggcus | 2261 |
| | {invAb} | | usu | |
| D- | csascgauGfgCfAfGfUfgcagucaus | 2823 | asAfsugacUfgcacugCfcAfucgugs | 2824 |
| 1323B | {invAb} | | usu | |
| D-1324 | uscsaggaUfgUfUfUfCfuucaggacs | 1414 | asGfsuccuGfaagaaaCfaUfccugas | 2262 |
| | {invAb} | | usu | |
| D-1325 | ususccucAfgCfUfGfAfcaaggaaus | 1415 | asAfsuuccUfugucagCfuGfaggaas | 2263 |
| | {invAb} | | usu | |
| D-1326 | asgscugaCfaAfGfGfAfauuuuggus | 1416 | asAfsccaaAfauuccuUfgUfcagcus | 2264 |
| | {invAb} | | usu | |
| D-1327 | gsasauuuUfgGfUfCfCfcugccuags | 1417 | asCfsuaggCfagggacCfaAfaauucs | 2265 |
| | {invAb} | | usu | |
| D-1328 | asasuuuuGfgUfCfCfCfugccuaggs | 1418 | usCfscuagGfcagggaCfcAfaaauus | 2266 |
| | {invAb} | | usu | |
| D- | ususuuggUfcCfCfUfGfccuaggacs | 2825 | asGfsuccuAfggcaggGfaCfcaaaas | 2826 |
| 1328B | {invAb} | | usu | |
| D-1329 | ususugguCfcCfUfGfCfcuaggaccs | 1419 | asGfsguccUfaggcagGfgAfccaaas | 2267 |
| | {invAb} | | usu | |
| D-1330 | csusgccuAfgGfAfCfCfgggucaucs | 1420 | asGfsaugaCfccggucCfuAfggcags | 2268 |
| | {invAb} | | usu | |
| D-1331 | ascsagagAfgAfUfGfGfuaagcagcs | 1421 | asGfscugcUfuaccauCfuCfucugus | 2269 |
| | {invAb} | | usu | |
| D-1332 | gsasgagaUfgGfUfAfAfgcagcugus | 1422 | usAfscagcUfgcuuacCfaUfcucucs | 2270 |
| | {invAb} | | usu | |
| D-1333 | asgsagauGfgUfAfAfGfcagcuguas | 1423 | asUfsacagCfugcuuaCfcAfucucus | 2271 |
| | {invAb} | | usu | |
| D-1334 | gsasgaugGfuAfAfGfCfagcuguaus | 1424 | asAfsuacaGfcugcuuAfcCfaucucs | 2272 |
| | {invAb} | | usu | |
| D-1335 | gsusaagcAfgCfUfGfUfaugaaugcs | 1425 | asGfscauuCfauacagCfuGfcuuacs | 2273 |
| | {invAb} | | usu | |
| D-1336 | asgscagcUfgUfAfUfGfaaugcugas | 1426 | asUfscagcAfuucauaCfaGfcugcus | 2274 |
| | {invAb} | | usu | |
| D-1337 | gsasuuuuAfaAfAfCfCfaggucaugs | 1427 | asCfsaugaCfcugguuUfuAfaaaucs | 2275 |
| | {invAb} | | usu | |
| D-1338 | ususuuaaAfaCfCfAfGfgucaugggs | 1428 | usCfsccauGfaccuggUfuUfuaaaas | 2276 |
| | {invAb} | | usu | |
| D-1339 | csusgaacAfcUfGfAfCfugcacuuas | 1429 | asUfsaaguGfcagucaGfuGfuucags | 2277 |
| | {invAb} | | usu | |
| D-1340 | usgsaacaCfuGfAfCfUfgcacuuacs | 1430 | asGfsuaagUfgcagucAfgUfguucas | 2278 |
| | {invAb} | | usu | |
| D-1341 | csascuuaCfcAfGfUfCfugauuuuas | 1431 | asUfsaaaaUfcagacuGfgUfaagugs | 2279 |
| | {invAb} | | usu | |
| D-1342 | ascscaguCfuGfAfUfUfuuaucgucs | 1432 | usGfsacgaUfaaaaucAfgAfcuggus | 2280 |
| | {invAb} | | usu | |
| D-1343 | cscsagucUfgAfUfUfUfuaucgucas | 1433 | usUfsgacgAfuaaaauCfaGfacuggs | 2281 |
| | {invAb} | | usu | |
| D-1344 | uscsugauUfuUfAfUfCfgucaaacas | 1434 | asUfsguuuGfacgauaAfaAfucagas | 2282 |
| | {invAb} | | usu | |
| D-1345 | csusgauuUfuAfUfCfGfucaaacacs | 1435 | asGfsuguuUfgacgauAfaAfaucags | 2283 |
| | {invAb} | | usu | |
| D-1346 | usgsauuuUfaUfCfGfUfcaaacaccs | 1436 | usGfsguguUfugacgaUfaAfaaucas | 2284 |
| | {invAb} | | usu | |
| D-1347 | gsasuuuuAfuCfGfUfCfaaacaccas | 1437 | usUfsggugUfuugacgAfuAfaaaucs | 2285 |
| | {invAb} | | usu | |
| D-1348 | asusuuuaUfcGfUfCfAfaacaccaas | 1438 | asUfsugguGfuuugacGfaUfaaaaus | 2286 |
| | {invAb} | | usu | |
| D-1349 | ususaucgUfcAfAfAfCfaccaagccs | 1439 | usGfsgcuuGfguguuuGfaCfgauaas | 2287 |
| | {invAb} | | usu | |
| D-1350 | usasucguCfaAfAfCfAfccaagccas | 1440 | asUfsggcuUfgguguuUfgAfcgauas | 2288 |
| | {invAb} | | usu | |
| D-1351 | csasugcuCfaUfGfGfCfaaucuguus | 1441 | asAfsacagAfuugccaUfgAfgcaugs | 2289 |
| | {invAb} | | usu | |
| D-1352 | uscsauggCfaAfUfCfUfguuuggggs | 1442 | asCfscccaAfacagauUfgCfcaugas | 2290 |
| | {invAb} | | usu | |
| D-1353 | gsusuuugUfuGfUfGfGfcacuagccs | 1443 | usGfsgcuaGfugccacAfaCfaaaacs | 2291 |
| | {invAb} | | usu | |
| D-1354 | ususuuguUfgUfGfGfCfacuagccas | 1444 | usUfsggcuAfgugccaCfaAfcaaaas | 2292 |
| | {invAb} | | usu | |
| D-1355 | ususuguuGfuGfGfCfAfcuagccaas | 1445 | usUfsuggcUfagugccAfcAfacaaas | 2293 |
| | {invAb} | | usu | |
| D-1356 | ususguugUfgGfCfAfCfuagccaaas | 1446 | asUfsuuggCfuagugcCfaCfaacaas | 2294 |
| | {invAb} | | usu | |
| D-1357 | gsusugugGfcAfCfUfAfgccaaacas | 1447 | asUfsguuuGfgcuaguGfcCfacaacs | 2295 |
| | {invAb} | | usu | |
| D-1358 | ususguggCfaCfUfAfGfccaaacaus | 1448 | usAfsuguuUfggcuagUfgCfcacaas | 2296 |
| | {invAb} | | usu | |
| D-1359 | usgsuggcAfcUfAfGfCfcaaacauas | 1449 | usUfsauguUfuggcuaGfuGfccacas | 2297 |
| | {invAb} | | usu | |
| D-1360 | gsusggcaCfuAfGfCfCfaaacauaas | 1450 | usUfsuaugUfuuggcuAfgUfgccacs | 2298 |
| | {invAb} | | usu | |
| D-1361 | usgsgcacUfaGfCfCfAfaacauaaas | 1451 | asUfsuuauGfuuuggcUfaGfugccas | 2299 |
| | {invAb} | | usu | |
| D-1362 | gsgscacuAfgCfCfAfAfacauaaags | 1452 | asCfsuuuaUfguuuggCfuAfgugccs | 2300 |
| | {invAb} | | usu | |
| D-1363 | gscsacuaGfcCfAfAfAfcauaaaggs | 1453 | asCfscuuuAfuguuugGfcUfagugcs | 2301 |
| | {invAb} | | usu | |
| D-1364 | asgsccaaAfcAfUfAfAfaggggcuus | 1454 | usAfsagccCfcuuuauGfuUfuggcus | 2302 |
| | {invAb} | | usu | |
| D-1365 | gscscaaaCfaUfAfAfAfggggcuuas | 1455 | usUfsaagcCfccuuuaUfgUfuuggcs | 2303 |
| | {invAb} | | usu | |
| D-1366 | cscsaaacAfuAfAfAfGfgggcuuaas | 1456 | asUfsuaagCfcccuuuAfuGfuuuggs | 2304 |
| | {invAb} | | usu | |
| D-1367 | csasaacaUfaAfAfGfGfggcuuaags | 1457 | asCfsuuaaGfccccuuUfaUfguuugs | 2305 |
| | {invAb} | | usu | |
| D-1368 | asasacauAfaAfGfGfGfgcuuaagus | 1458 | asAfscuuaAfgccccuUfuAfuguuus | 2306 |
| | {invAb} | | usu | |
| D-1369 | asascauaAfaGfGfGfGfcuuaagucs | 1459 | usGfsacuuAfagccccUfuUfauguus | 2307 |
| | {invAb} | | usu | |
| D-1370 | ascsauaaAfgGfGfGfCfuuaagucas | 1460 | asUfsgacuUfaagcccCfuUfuaugus | 2308 |
| | {invAb} | | usu | |
| D-1371 | asusaaagGfgGfCfUfUfaagucagcs | 1461 | asGfscugaCfuuaagcCfcCfuuuaus | 2309 |
| | {invAb} | | usu | |
| D-1372 | usasaaggGfgCfUfUfAfagucagccs | 1462 | asGfsgcugAfcuuaagCfcCfcuuuas | 2310 |
| | {invAb} | | usu | |
| D-1373 | asgsgggcUfuAfAfGfUfcagccugcs | 1463 | usGfscaggCfugacuuAfaGfccccus | 2311 |
| | {invAb} | | usu | |
| D-1374 | asasgucaGfcCfUfGfCfauacagags | 1464 | asCfsucugUfaugcagGfcUfgacuus | 2312 |
| | {invAb} | | usu | |
| D-1375 | asgsucagCfcUfGfCfAfuacagaggs | 1465 | usCfscucuGfuaugcaGfgCfugacus | 2313 |
| | {invAb} | | usu | |
| D-1376 | asgsccugCfaUfAfCfAfgaggaucgs | 1466 | asCfsgaucCfucuguaUfgCfaggcus | 2314 |
| | {invAb} | | usu | |
| D-1377 | gscscugcAfuAfCfAfGfaggaucggs | 1467 | asCfscgauCfcucuguAfuGfcaggcs | 2315 |
| | {invAb} | | usu | |
| D-1378 | cscsugcaUfaCfAfGfAfggaucgggs | 1468 | asCfsccgaUfccucugUfaUfgcaggs | 2316 |
| | {invAb} | | usu | |
| D-1379 | ascsagagGfaUfCfGfGfggagagaas | 1469 | asUfsucucUfccccgaUfcCfucugus | 2317 |
| | {invAb} | | usu | |
| D-1380 | gsasguacUfuAfCfCfAfgaguuuaas | 1470 | asUfsuaaaCfucugguAfaGfuacucs | 2318 |
| | {invAb} | | usu | |
| D-1381 | csusgcacUfaAfAfAfUfccccaaacs | 1471 | asGfsuuugGfggauuuUfaGfugcags | 2319 |
| | {invAb} | | usu | |
| D-1382 | usgscacuAfaAfAfUfCfcccaaacus | 1472 | asAfsguuuGfgggauuUfuAfgugcas | 2320 |
| | {invAb} | | usu | |
| D-1383 | csascuaaAfaUfCfCfCfcaaacugas | 1473 | asUfscaguUfuggggaUfuUfuagugs | 2321 |
| | {invAb} | | usu | |
| D-1384 | asasucccCfaAfAfCfUfgacagguas | 1474 | usUfsaccuGfucaguuUfgGfggauus | 2322 |
| | {invAb} | | usu | |
| D-1385 | cscsaaacUfgAfCfAfGfguaaaugus | 1475 | usAfscauuUfaccuguCfaGfuuuggs | 2323 |
| | {invAb} | | usu | |
| D-1386 | csasaacuGfaCfAfGfGfuaaauguas | 1476 | asUfsacauUfuaccugUfcAfguuugs | 2324 |
| | {invAb} | | usu | |
| D-1387 | asasacugAfcAfGfGfUfaaauguags | 1477 | asCfsuacaUfuuaccuGfuCfaguuus | 2325 |
| | {invAb} | | usu | |
| D-1388 | ascsugacAfgGfUfAfAfauguagccs | 1478 | asGfsgcuaCfauuuacCfuGfucagus | 2326 |
| | {invAb} | | usu | |
| D-1389 | asuscuaaAfuCfAfCfAfcuauuuucs | 1479 | asGfsaaaaUfagugugAfuUfuagaus | 2327 |
| | {invAb} | | usu | |
| D-1390 | csusaaauCfaCfAfCfUfauuuucgas | 1480 | asUfscgaaAfauagugUfgAfuuuags | 2328 |
| | {invAb} | | usu | |
| D-1391 | usasaaucAfcAfCfUfAfuuuucgags | 1481 | usCfsucgaAfaauaguGfuGfauuuas | 2329 |
| | {invAb} | | usu | |
| D-1392 | asasucacAfcUfAfUfUfuucgagaus | 1482 | asAfsucucGfaaaauaGfuGfugauus | 2330 |
| | {invAb} | | usu | |
| D-1393 | uscsacacUfaUfUfUfUfcgagaucas | 1483 | asUfsgaucUfcgaaaaUfaGfugugas | 2331 |
| | {invAb} | | usu | |
| D-1394 | csascacuAfuUfUfUfCfgagaucaus | 1484 | asAfsugauCfucgaaaAfuAfgugugs | 2332 |
| | {invAb} | | usu | |
| D-1395 | ascsacuaUfuUfUfCfGfagaucaugs | 1485 | asCfsaugaUfcucgaaAfaUfagugus | 2333 |
| | {invAb} | | usu | |
| D-1396 | ascsuauuUfuCfGfAfGfaucauguas | 1486 | asUfsacauGfaucucgAfaAfauagus | 2334 |
| | {invAb} | | usu | |
| D-1397 | csusauuuUfcGfAfGfAfucauguaus | 1487 | usAfsuacaUfgaucucGfaAfaauags | 2335 |
| | {invAb} | | usu | |
| D-1398 | asusuuucGfaGfAfUfCfauguauaas | 1488 | usUfsuauaCfaugaucUfcGfaaaaus | 2336 |
| | {invAb} | | usu | |
| D-1399 | ususcgagAfuCfAfUfGfuauaaaaas | 1489 | asUfsuuuuAfuacaugAfuCfucgaas | 2337 |
| | {invAb} | | usu | |
| D-1400 | asasaaaaGfaAfGfUfCfaugcugugs | 1490 | asCfsacagCfaugacuUfcUfuuuuus | 2338 |
| | {invAb} | | usu | |
| D-1401 | usgscuguGfuGfGfCfCfaauuauaas | 1491 | asUfsuauaAfuuggccAfcAfcagcas | 2339 |
| | {invAb} | | usu | |
| D-1402 | ususggagGfgAfCfCfAfggaaaugus | 1492 | usAfscauuUfccugguCfcCfuccaas | 2340 |
| | {invAb} | | usu | |
| D-1403 | ascscaggAfaAfUfGfUfaagacaccs | 1493 | usGfsguguCfuuacauUfuCfcuggus | 2341 |
| | {invAb} | | usu | |
| D-1404 | cscsaggaAfaUfGfUfAfagacaccas | 1494 | usUfsggugUfcuuacaUfuUfccuggs | 2342 |
| | {invAb} | | usu | |
| D-1405 | gsusgugcCfuGfAfUfGfucaccucas | 1495 | asUfsgaggUfgacaucAfgGfcacacs | 2343 |
| | {invAb} | | usu | |
| D-1406 | usgsugccUfgAfUfGfUfcaccucaus | 1496 | asAfsugagGfugacauCfaGfgcacas | 2344 |
| | {invAb} | | usu | |
| D-1407 | gsusgccuGfaUfGfUfCfaccucaugs | 1497 | usCfsaugaGfgugacaUfcAfggcacs | 2345 |
| | {invAb} | | usu | |
| D-1408 | gscscugaUfgUfCfAfCfcucaugaus | 1498 | asAfsucauGfaggugaCfaUfcaggcs | 2346 |
| | {invAb} | | usu | |
| D-1409 | ususuuuuAfaCfUfCfCfugcgccaas | 1499 | asUfsuggcGfcaggagUfuAfaaaaas | 2347 |
| | {invAb} | | usu | |
| D-1410 | ususaacuCfcUfGfCfGfccaaggacs | 1500 | usGfsuccuUfggcgcaGfgAfguuaas | 2348 |
| | {invAb} | | usu | |
| D-1411 | asascuccUfgCfGfCfCfaaggacags | 1501 | asCfsugucCfuuggcgCfaGfgaguus | 2349 |
| | {invAb} | | usu | |
| D-1412 | ascsuccuGfcGfCfCfAfaggacagus | 1502 | asAfscuguCfcuuggcGfcAfggagus | 2350 |
| | {invAb} | | usu | |
| D-1413 | usgsuccaCfcUfUfUfGfugcuuugcs | 1503 | asGfscaaaGfcacaaaGfgUfggacas | 2351 |
| | {invAb} | | usu | |
| D-1414 | uscscaccUfuUfGfUfGfcuuugcgas | 1504 | asUfscgcaAfagcacaAfaGfguggas | 2352 |
| | {invAb} | | usu | |
| D-1415 | csasccuuUfgUfGfCfUfuugcgaggs | 1505 | asCfscucgCfaaagcaCfaAfaggugs | 2353 |
| | {invAb} | | usu | |
| D-1416 | cscsuuugUfgCfUfUfUfgcgaggccs | 1506 | asGfsgccuCfgcaaagCfaCfaaaggs | 2354 |
| | {invAb} | | usu | |
| D-1416 | cscsuuugUfgCfUfUfUfgcgaggccs | 1507 | asGfsgccuCfgcaaagCfaCfaaaggs | 2355 |
| | {invAb} | | usu | |
| D-1417 | gsasgcccAfgGfCfAfUfcugcucgcs | 1508 | asGfscgagCfagaugcCfuGfggcucs | 2356 |
| | {invAb} | | usu | |
| D-1418 | cscscaggCfaUfCfUfGfcucgccugs | 1509 | asCfsaggcGfagcagaUfgCfcugggs | 2357 |
| | {invAb} | | usu | |
| D-1419 | csasggcaUfcUfGfCfUfcgccugccs | 1510 | usGfsgcagGfcgagcaGfaUfgccugs | 2358 |
| | {invAb} | | usu | |
| D-1420 | gscsaucuGfcUfCfGfCfcugccacgs | 1511 | asCfsguggCfaggcgaGfcAfgaugcs | 2359 |
| | {invAb} | | usu | |
| D-1421 | usgsccacGfgCfUfGfAfccagagaas | 1512 | asUfsucucUfggucagCfcGfuggcas | 2360 |
| | {invAb} | | usu | |
| D-1422 | gsasgcucUfgCfCfUfUfagacgacgs | 1513 | asCfsgucgUfcuaaggCfaGfagcucs | 2361 |
| | {invAb} | | usu | |
| D-1423 | asgscucuGfcCfUfUfAfgacgacgus | 1514 | asAfscgucGfucuaagGfcAfgagcus | 2362 |
| | {invAb} | | usu | |
| D-1424 | gscsucugCfcUfUfAfGfacgacgugs | 1515 | asCfsacguCfgucuaaGfgCfagagcs | 2363 |
| | {invAb} | | usu | |
| D-1425 | csuscugcCfuUfAfGfAfcgacgugus | 1516 | asAfscacgUfcgucuaAfgGfcagags | 2364 |
| | {invAb} | | usu | |
| D-1426 | uscsugccUfuAfGfAfCfgacguguus | 1517 | usAfsacacGfucgucuAfaGfgcagas | 2365 |
| | {invAb} | | usu | |
| D-1427 | csusgccuUfaGfAfCfGfacguguuas | 1518 | asUfsaacaCfgucgucUfaAfggcags | 2366 |
| | {invAb} | | usu | |
| D-1428 | gscscuuaGfaCfGfAfCfguguuacas | 1519 | asUfsguaaCfacgucgUfcUfaaggcs | 2367 |
| | {invAb} | | usu | |
| D-1429 | cscsuuagAfcGfAfCfGfuguuacags | 1520 | asCfsuguaAfcacgucGfuCfuaaggs | 2368 |
| | {invAb} | | usu | |
| D-1430 | csusuagaCfgAfCfGfUfguuacagus | 1521 | usAfscuguAfacacguCfgUfcuaags | 2369 |
| | {invAb} | | usu | |
| D-1431 | usasgacgAfcGfUfGfUfuacaguaus | 1522 | asAfsuacuGfuaacacGfuCfgucuas | 2370 |
| | {invAb} | | usu | |
| D-1432 | ascsgacgUfgUfUfAfCfaguaugaas | 1523 | asUfsucauAfcuguaaCfaCfgucgus | 2371 |
| | {invAb} | | usu | |
| D-1433 | ascsacagCfaGfAfGfGfcacccucgs | 1524 | asCfsgaggGfugccucUfgCfugugus | 2372 |
| | {invAb} | | usu | |
| D-1434 | csascagcAfgAfGfGfCfacccucgus | 1525 | usAfscgagGfgugccuCfuGfcugugs | 2373 |
| | {invAb} | | usu | |
| D-1435 | ascsagcaGfaGfGfCfAfcccucguas | 1526 | asUfsacgaGfggugccUfcUfgcugus | 2374 |
| | {invAb} | | usu | |
| D-1436 | csasgcagAfgGfCfAfCfccucguaus | 1527 | asAfsuacgAfgggugcCfuCfugcugs | 2375 |
| | {invAb} | | usu | |
| D-1437 | asgscagaGfgCfAfCfCfcucguaugs | 1528 | asCfsauacGfagggugCfcUfcugcus | 2376 |
| | {invAb} | | usu | |
| D-1438 | gscsagagGfcAfCfCfCfucguaugus | 1529 | asAfscauaCfgaggguGfcCfucugcs | 2377 |
| | {invAb} | | usu | |
| D-1439 | csasgaggCfaCfCfCfUfcguauguus | 1530 | asAfsacauAfcgagggUfgCfcucugs | 2378 |
| | {invAb} | | usu | |
| D-1440 | asgsaggcAfcCfCfUfCfguauguuus | 1531 | asAfsaacaUfacgaggGfuGfccucus | 2379 |
| | {invAb} | | usu | |
| D-1441 | gsasggcaCfcCfUfCfGfuauguuuus | 1532 | asAfsaaacAfuacgagGfgUfgccucs | 2380 |
| | {invAb} | | usu | |
| D-1442 | gsgscaccCfuCfGfUfAfuguuuugas | 1533 | usUfscaaaAfcauacgAfgGfgugccs | 2381 |
| | {invAb} | | usu | |
| D-1443 | cscscucgUfaUfGfUfUfuugaaagus | 1534 | asAfscuuuCfaaaacaUfaCfgagggs | 2382 |
| | {invAb} | | usu | |
| D-1444 | asusguaaAfaCfUfAfUfacugacccs | 1535 | asGfsggucAfguauagUfuUfuacaus | 2383 |
| | {invAb} | | usu | |
| D-1445 | usgsuaaaAfcUfAfUfAfcugacccgs | 1536 | asCfsggguCfaguauaGfuUfuuacas | 2384 |
| | {invAb} | | usu | |
| D-1446 | gsusaaaaCfuAfUfAfCfugacccgus | 1537 | asAfscgggUfcaguauAfgUfuuuacs | 2385 |
| | {invAb} | | usu | |
| D-1447 | usasaaacUfaUfAfCfUfgacccguus | 1538 | asAfsacggGfucaguaUfaGfuuuuas | 2386 |
| | {invAb} | | usu | |
| D-1448 | ascsuauaCfuGfAfCfCfcguuuucas | 1539 | asUfsgaaaAfcgggucAfgUfauagus | 2387 |
| | {invAb} | | usu | |
| D-1449 | asusacugAfcCfCfGfUfuuucaguus | 1540 | asAfsacugAfaaacggGfuCfaguaus | 2388 |
| | {invAb} | | usu | |
| D-1450 | ususucagUfuUfUfAfAfagggucgus | 1541 | asAfscgacCfcuuuaaAfaCfugaaas | 2389 |
| | {invAb} | | usu | |
| D-1451 | ususcaguUfuUfAfAfAfgggucgugs | 1542 | usCfsacgaCfccuuuaAfaAfcugaas | 2390 |
| | {invAb} | | usu | |
| D-1452 | uscsaguuUfuAfAfAfGfggucgugas | 1543 | asUfscacgAfcccuuuAfaAfacugas | 2391 |
| | {invAb} | | usu | |
| D-1453 | csasguuuUfaAfAfGfGfgucgugags | 1544 | usCfsucacGfacccuuUfaAfaacugs | 2392 |
| | {invAb} | | usu | |
| D-1454 | gsusuuuaAfaGfGfGfUfcgugagaas | 1545 | usUfsucucAfcgacccUfuUfaaaacs | 2393 |
| | {invAb} | | usu | |
| D- | ususuuaaAfgGfGfUfCfgugagaaas | 2827 | asUfsuucuCfacgaccCfuUfuaaaas | 2828 |
| 1454B | {invAb} | | usu | |
| D-1455 | ususuaaaGfgGfUfCfGfugagaaacs | 1546 | asGfsuuucUfcacgacCfcUfuuaaas | 2394 |
| | {invAb} | | usu | |
| D-1456 | usasaaggGfuCfGfUfGfagaaacugs | 1547 | asCfsaguuUfcucacgAfcCfcuuuas | 2395 |
| | {invAb} | | usu | |
| D-1457 | asasagggUfcGfUfGfAfgaaacuggs | 1548 | asCfscaguUfucucacGfaCfccuuus | 2396 |
| | {invAb} | | usu | |
| D-1458 | gsasgaaaCfuGfGfCfUfgguccaaus | 1549 | asAfsuuggAfccagccAfgUfuucucs | 2397 |
| | {invAb} | | usu | |
| D-1459 | usgsguccAfaUfGfGfGfauuuacags | 1550 | asCfsuguaAfaucccaUfuGfgaccas | 2398 |
| | {invAb} | | usu | |
| D-1460 | cscsaaugGfgAfUfUfUfacagcaacs | 1551 | usGfsuugcUfguaaauCfcCfauuggs | 2399 |
| | {invAb} | | usu | |
| D-1461 | ususuaagUfuUfGfCfUfcuuaaucgs | 1552 | asCfsgauuAfagagcaAfaCfuuaaas | 2400 |
| | {invAb} | | usu | |
| D-1462 | ususaaguUfuGfCfUfCfuuaaucgus | 1553 | usAfscgauUfaagagcAfaAfcuuaas | 2401 |
| | {invAb} | | usu | |
| D-1463 | asgsuuugCfuCfUfUfAfaucguaugs | 1554 | asCfsauacGfauuaagAfgCfaaacus | 2402 |
| | {invAb} | | usu | |
| D-1464 | gsusuugcUfcUfUfAfAfucguauggs | 1555 | usCfscauaCfgauuaaGfaGfcaaacs | 2403 |
| | {invAb} | | usu | |
| D-1465 | ususugcuCfuUfAfAfUfcguauggas | 1556 | usUfsccauAfcgauuaAfgAfgcaaas | 2404 |
| | {invAb} | | usu | |
| D-1466 | usgscucuUfaAfUfCfGfuauggaags | 1557 | asCfsuuccAfuacgauUfaAfgagcas | 2405 |
| | {invAb} | | usu | |
| D-1467 | csusuaauCfgUfAfUfGfgaagcuugs | 1558 | usCfsaagcUfuccauaCfgAfuuaags | 2406 |
| | {invAb} | | usu | |
| D-1468 | ususaaucGfuAfUfGfGfaagcuugas | 1559 | asUfscaagCfuuccauAfcGfauuaas | 2407 |
| | {invAb} | | usu | |
| D-1469 | gsgsaagcUfuGfAfGfCfuauguguus | 1560 | asAfsacacAfuagcucAfaGfcuuccs | 2408 |
| | {invAb} | | usu | |
| D-1470 | gsasagcuUfgAfGfCfUfauguguugs | 1561 | asCfsaacaCfauagcuCfaAfgcuucs | 2409 |
| | {invAb} | | usu | |
| D-1471 | asgscuugAfgCfUfAfUfguguuggas | 1562 | usUfsccaaCfacauagCfuCfaagcus | 2410 |
| | {invAb} | | usu | |
| D-1472 | gscsuugaGfcUfAfUfGfuguuggaas | 1563 | asUfsuccaAfcacauaGfcUfcaagcs | 2411 |
| | {invAb} | | usu | |
| D-1473 | csusugagCfuAfUfGfUfguuggaags | 1564 | asCfsuuccAfacacauAfgCfucaags | 2412 |
| | {invAb} | | usu | |
| D-1474 | ususgagcUfaUfGfUfGfuuggaagus | 1565 | asAfscuucCfaacacaUfaGfcucaas | 2413 |
| | {invAb} | | usu | |
| D-1475 | usgsagcuAfuGfUfGfUfuggaagugs | 1566 | asCfsacuuCfcaacacAfuAfgcucas | 2414 |
| | {invAb} | | usu | |
| D-1476 | gsasgcuaUfgUfGfUfUfggaagugcs | 1567 | asGfscacuUfccaacaCfaUfagcucs | 2415 |
| | {invAb} | | usu | |
| D-1477 | gsusguugGfaAfGfUfGfcccugguus | 1568 | asAfsaccaGfggcacuUfcCfaacacs | 2416 |
| | {invAb} | | usu | |
| D-1478 | gsgsaaguGfcCfCfUfGfguuuuaaus | 1569 | asAfsuuaaAfaccaggGfcAfcuuccs | 2417 |
| | {invAb} | | usu | |
| D-1479 | csusgguuUfuAfAfUfCfcauacacas | 1570 | usUfsguguAfuggauuAfaAfaccags | 2418 |
| | {invAb} | | usu | |
| D-1480 | ususaaucCfaUfAfCfAfcaaagacgs | 1571 | asCfsgucuUfuguguaUfgGfauuaas | 2419 |
| | {invAb} | | usu | |
| D-1481 | usasauccAfuAfCfAfCfaaagacggs | 1572 | asCfscgucUfuuguguAfuGfgauuas | 2420 |
| | {invAb} | | usu | |
| D-1482 | asasuccaUfaCfAfCfAfaagacggus | 1573 | usAfsccguCfuuugugUfaUfggauus | 2421 |
| | {invAb} | | usu | |
| D-1483 | asusccauAfcAfCfAfAfagacgguas | 1574 | asUfsaccgUfcuuuguGfuAfuggaus | 2422 |
| | {invAb} | | usu | |
| D-1484 | uscscauaCfaCfAfAfAfgacgguacs | 1575 | usGfsuaccGfucuuugUfgUfauggas | 2423 |
| | {invAb} | | usu | |
| D-1485 | csasuacaCfaAfAfGfAfcgguacaus | 1576 | usAfsuguaCfcgucuuUfgUfguaugs | 2424 |
| | {invAb} | | usu | |
| D-1486 | usascacaAfaGfAfCfGfguacauaas | 1577 | asUfsuaugUfaccgucUfuUfguguas | 2425 |
| | {invAb} | | usu | |
| D-1487 | ascsacaaAfgAfCfGfGfuacauaaus | 1578 | asAfsuuauGfuaccguCfuUfugugus | 2426 |
| | {invAb} | | usu | |
| D-1488 | csascaaaGfaCfGfGfUfacauaaucs | 1579 | asGfsauuaUfguaccgUfcUfuugugs | 2427 |
| | {invAb} | | usu | |
| D-1489 | ascsaaagAfcGfGfUfAfcauaauccs | 1580 | asGfsgauuAfuguaccGfuCfuuugus | 2428 |
| | {invAb} | | usu | |
| D-1490 | csasaagaCfgGfUfAfCfauaauccus | 1581 | usAfsggauUfauguacCfgUfcuuugs | 2429 |
| | {invAb} | | usu | |
| D-1491 | asasagacGfgUfAfCfAfuaauccuas | 1582 | asUfsaggaUfuauguaCfcGfucuuus | 2430 |
| | {invAb} | | usu | |
| D-1492 | asasgacgGfuAfCfAfUfaauccuacs | 1583 | usGfsuaggAfuuauguAfcCfgucuus | 2431 |
| | {invAb} | | usu | |
| D-1493 | gsusacauAfaUfCfCfUfacagguuus | 1584 | usAfsaaccUfguaggaUfuAfuguacs | 2432 |
| | {invAb} | | usu | |
| D-1494 | csasuaauCfcUfAfCfAfgguuuaaas | 1585 | asUfsuuaaAfccuguaGfgAfuuaugs | 2433 |
| | {invAb} | | usu | |
| D-1495 | asusccuaCfaGfGfUfUfuaaauguas | 1586 | asUfsacauUfuaaaccUfgUfaggaus | 2434 |
| | {invAb} | | usu | |
| D-1496 | usasguuuGfgAfAfUfUfcuuugcucs | 1587 | asGfsagcaAfagaauuCfcAfaacuas | 2435 |
| | {invAb} | | usu | |
| D-1497 | asgsuuugGfaAfUfUfCfuuugcucus | 1588 | usAfsgagcAfaagaauUfcCfaaacus | 2436 |
| | {invAb} | | usu | |
| D-1498 | ususuggaAfuUfCfUfUfugcucuacs | 1589 | asGfsuagaGfcaaagaAfuUfccaaas | 2437 |
| | {invAb} | | usu | |
| D-1499 | ususgcucUfaCfUfGfUfuuacauugs | 1590 | asCfsaaugUfaaacagUfaGfagcaas | 2438 |
| | {invAb} | | usu | |
| D-1500 | uscsuacuGfuUfUfAfCfauugcagas | 1591 | asUfscugcAfauguaaAfcAfguagas | 2439 |
| | {invAb} | | usu | |
| D-1501 | usascuguUfuAfCfAfUfugcagauus | 1592 | asAfsaucuGfcaauguAfaAfcaguas | 2440 |
| | {invAb} | | usu | |
| D-1502 | ascsuguuUfaCfAfUfUfgcagauugs | 1593 | asCfsaaucUfgcaaugUfaAfacagus | 2441 |
| | {invAb} | | usu | |
| D-1503 | ususuacaUfuGfCfAfGfauugcuaus | 1594 | usAfsuagcAfaucugcAfaUfguaaas | 2442 |
| | {invAb} | | usu | |
| D-1504 | ususacauUfgCfAfGfAfuugcuauas | 1595 | usUfsauagCfaaucugCfaAfuguaas | 2443 |
| | ( invAb} | | usu | |
| D-1505 | usascauuGfcAfGfAfUfugcuauaas | 1596 | asUfsuauaGfcaaucuGfcAfauguas | 2444 |
| | {invAb} | | usu | |
| D-1506 | asusugcuAfuAfAfUfUfucaaggags | 1597 | asCfsuccuUfgaaauuAfuAfgcaaus | 2445 |
| | {invAb} | | usu | |
| D-1507 | ususgcuaUfaAfUfUfUfcaaggagus | 1598 | asAfscuccUfugaaauUfaUfagcaas | 2446 |
| | {invAb} | | usu | |
| D-1508 | asasugauGfcAfCfUfUfuaggaugus | 1599 | asAfscaucCfuaaaguGfcAfucauus | 2447 |
| | {invAb} | | usu | |
| D-1509 | asusgaugCfaCfUfUfUfaggauguus | 1600 | asAfsacauCfcuaaagUfgCfaucaus | 2448 |
| | {invAb} | | usu | |
| D-1510 | ascsaugaAfuCfAfUfUfcacaugacs | 1601 | asGfsucauGfugaaugAfuUfcaugus | 2449 |
| | {invAb} | | usu | |
| D-1511 | csasugaaUfcAfUfUfCfacaugaccs | 1602 | usGfsgucaUfgugaauGfaUfucaugs | 2450 |
| | {invAb} | | usu | |
| D-1512 | asasauacAfuGfUfCfUfagucugucs | 1603 | asGfsacagAfcuagacAfuGfuauuus | 2451 |
| | {invAb} | | usu | |
| D- | asasuacaUfgUfCfUfAfgucuguccs | 2829 | asGfsgacaGfacuagaCfaUfguauus | 2830 |
| 1512B | {invAb} | | usu | |
| D-1513 | asusgucuAfgUfCfUfGfuccuuuaas | 1604 | asUfsuaaaGfgacagaCfuAfgacaus | 2452 |
| | {invAb} | | usu | |
| D-1514 | usgsucuaGfuCfUfGfUfccuuuaaus | 1605 | usAfsuuaaAfggacagAfcUfagacas | 2453 |
| | {invAb} | | usu | |
| D-1515 | uscsuaguCfuGfUfCfCfuuuaauags | 1606 | asCfsuauuAfaaggacAfgAfcuagas | 2454 |
| | {invAb} | | usu | |
| D-1516 | csusagucUfgUfCfCfUfuuaauagcs | 1607 | asGfscuauUfaaaggaCfaGfacuags | 2455 |
| | {invAb} | | usu | |
| D-1517 | usasgucuGfuCfCfUfUfuaauagcus | 1608 | asAfsgcuaUfuaaaggAfcAfgacuas | 2456 |
| | {invAb} | | usu | |
| D-1518 | gsuscuguCfcUfUfUfAfauagcucus | 1609 | asAfsgagcUfauuaaaGfgAfcagacs | 2457 |
| | {invAb} | | usu | |
| D-1519 | asasucagAfuCfAfUfUfaccaguuas | 1610 | asUfsaacuGfguaaugAfuCfugauus | 2458 |
| | {invAb} | | usu | |
| D-1520 | uscsagauCfaUfUfAfCfcaguuagcs | 1611 | asGfscuaaCfugguaaUfgAfucugas | 2459 |
| | {invAb} | | usu | |
| D-1521 | csasgaucAfuUfAfCfCfaguuagcus | 1612 | asAfsgcuaAfcugguaAfuGfaucugs | 2460 |
| | {invAb} | | usu | |
| D-1522 | asgsaucaUfuAfCfCfAfguuagcuus | 1613 | asAfsagcuAfacugguAfaUfgaucus | 2461 |
| | {invAb} | | usu | |
| D-1523 | uscsauuaCfcAfGfUfUfagcuuuuas | 1614 | usUfsaaaaGfcuaacuGfgUfaaugas | 2462 |
| | {invAb} | | usu | |
| D-1524 | csasuuacCfaGfUfUfAfgcuuuuaas | 1615 | usUfsuaaaAfgcuaacUfgGfuaaugs | 2463 |
| | {invAb} | | usu | |
| D-1525 | ascscaguUfaGfCfUfUfuuaaagcas | 1616 | asUfsgcuuUfaaaagcUfaAfcuggus | 2464 |
| | {invAb} | | usu | |
| D-1526 | asasgcacAfuUfUfGfUfuuaagacus | 1617 | usAfsgucuUfaaacaaAfuGfugcuus | 2465 |
| | {invAb} | | usu | |
| D-1527 | gscsacauUfuGfUfUfUfaagacuaus | 1618 | asAfsuaguCfuuaaacAfaAfugugcs | 2466 |
| | {invAb} | | usu | |
| D-1528 | csusauguUfuUfUfGEGfaaaaauacs | 1619 | asGfsuauuUfuuccaaAfaAfcauags | 2467 |
| | {invAb} | | usu | |
| D-1529 | gsusuuuuGfgAfAfAfAfauacgcuas | 1620 | asUfsagcgUfauuuuuCfcAfaaaacs | 2468 |
| | {invAb} | | usu | |
| D-1530 | ususggaaAfaAfUfAfCfgcuacagas | 1621 | usUfscuguAfgcguauUfuUfuccaas | 2469 |
| | {invAb} | | usu | |
| D-1531 | asasuaaaUfgAfGfAfUfgcuacuaas | 1622 | asUfsuaguAfgcaucuCfaUfuuauus | 2470 |
| | {invAb | | usu | |
| D-1532 | usgsagauGfcUfAfCfUfaauuguuus | 1623 | asAfsaacaAfuuaguaGfcAfucucas | 2471 |
| | {invAb} | | usu | |
| D-1533 | ususuggaAfuCfUfGfUfuguuucugs | 1624 | asCfsagaaAfcaacagAfuUfccaaas | 2472 |
| | {invAb} | | usu | |
| D-1534 | ususcugcCfaAfAfGfGfuaaauuaas | 1625 | asUfsuaauUfuaccuuUfgGfcagaas | 2473 |
| | {invAb} | | usu | |
| D-1535 | uscsugccAfaAfGfGfUfaaauuaacs | 1626 | asGfsuuaaUfuuaccuUfuGfgcagas | 2474 |
| | {invAb} | | usu | |
| D-1536 | gsasuuuaUfuCfAfGfGfaauccccas | 1627 | asUfsggggAfuuccugAfaUfaaaucs | 2475 |
| | {invAb} | | usu | |
| D-1537 | asusucagGfaAfUfCfCfccauuugas | 1628 | usUfscaaaUfggggauUfcCfugaaus | 2476 |
| | {invAb} | | usu | |
| D-1538 | gsgsaaucCfcCfAfUfUfugaauuugs | 1629 | asCfsaaauUfcaaaugGfgGfauuccs | 2477 |
| | {invAb} | | usu | |
| D-1539 | [GalNAc3]scucccaUfuCfUfUfUfc | 1630 | asGfsccucAfugaaagAfaUfgggags | 2478 |
| | augaggcs{invAb} | | usu | |
| D-1540 | [GalNAc3]saaggaaGfaCfCfGfCfa | 1631 | asAfsuaggAfgugcggUfcUfuccuus | 2479 |
| | cuccuaus{invAb} | | usu | |
| D-1541 | [GalNAc3]sgaaaacCfgAfUfUfUfc | 1632 | asGfsugcaCfugaaauCfgGfuuuucs | 2480 |
| | agugcacs{invAb} | | usu | |
| D-1542 | [GalNAc3]sgggagaGfaCfAfAfGfg | 1633 | asAfsuaagUfcccuugUfcUfcucccs | 2481 |
| | gacuuaus{invAb} | | usu | |
| D-1543 | [GalNAc3]sgauguuAfaUfAfAfCfu | 1634 | asCfscuccAfgaguuaUfuAfacaucs | 2482 |
| | cuggaggs{invAb} | | usu | |
| D-1544 | [GalNAc3]scaccaaAfcUfCfCfCfa | 1635 | usGfsaaagAfaugggaGfuUfuggugs | 2483 |
| | uucuuucs{invAb} | | usu | |
| D-1545 | [GalNAc3]sgcaaccGfgAfGfAfAfc | 1636 | usUfscuaaGfaguucuCfcGfguugcs | 2484 |
| | ucuuagas{invAb} | | usu | |
| D-1546 | [GalNAc3]scacaaaGfaCfGfGfUfa | 1637 | asGfsauuaUfguaccgUfcUfuugugs | 2485 |
| | cauaaucs{invAb} | | usu | |
| D-1547 | [GalNAc3]scggaagUfuUfGfAfAfg | 1638 | asAfsaucuAfucuucaAfaCfuuccgs | 2486 |
| | auagauus{invAb} | | usu | |
| D-1548 | [GalNAc3]sacagagAfgAfUfGfGfu | 1639 | asGfscugcUfuaccauCfuCfucugus | 2487 |
| | aagcagcs{invAb} | | usu | |
| D-1549 | [GalNAc3]sccucggUfuCfUfAfCfg | 1640 | asCfscauaAfgcguagAfaCfcgaggs | 2488 |
| | cuuauggs{invAb} | | usu | |
| D-1550 | [GalNAc3]scuccucGfgUfUfCfUfa | 1641 | asAfsuaagCfguagaaCfcGfaggags | 2489 |
| | cgcuuaus{invAb} | | usu | |
| D-1551 | [GalNAc3]sugcaaaCfgUfGfCfAfu | 1642 | asGfsgcucCfaaugcaCfgUfuugcas | 2490 |
| | uggagccs{invAb} | | usu | |
| D-1552 | [GalNAc3]sgaagacCfgCfAfCfUfc | 1643 | asGfsccauAfggagugCfgGfucuucs | 2491 |
| | cuauggcs{invAb} | | usu | |
| D-1553 | [GalNAc3]sgacaagGfgAfCfUfUfa | 1644 | usUfsuguuGfauaaguCfcCfuugucs | 2492 |
| | ucaacaas{invAb} | | usu | |
| D-1554 | [GalNAc3]sucagaaAfuCfCfAfGfu | 1645 | asGfsuuuaGfuacuggAfuUfucugas | 2493 |
| | acuaaacs{invAb} | | usu | |
| D-1555 | [GalNAc3]saaauacCfaUfCfAfAfa | 1646 | asGfscugcAfuuuugaUfgGfuauuus | 2494 |
| | augcagcs{invAb} | | usu | |
| D-1556 | [GalNAc3]saaugacCfuUfGfCfCfa | 1647 | asCfsggaaUfuuggcaAfgGfucauus | 2495 |
| | aauuccgs{invAb} | | usu | |
| D-1557 | [GalNAc3]scccaauGfgAfUfGfAfu | 1648 | asGfsuauuUfuaucauCfcAfuugggs | 2496 |
| | aaaauacs{invAb} | | usu | |
| D-1558 | [GalNAc3]sagacgaUfaGfCfAfAfu | 1649 | asGfscuucAfcauugcUfaUfcgucus | 2497 |
| | gugaagcs{invAb} | | usu | |
| D-1559 | [GalNAc3]sgaagacGfaUfAfGfCfa | 1650 | asUfsucacAfuugcuaUfcGfucuucs | 2498 |
| | augugaas{invAb} | | usu | |
| D-1560 | [GalNAc3]sucagaaGfaCfGfAfUfa | 1651 | asAfscauuGfcuaucgUfcUfucugas | 2499 |
| | gcaaugus{invAb} | | usu | |
| D-1561 | [GalNAc3]sgggucaGfaAfGfAfCfg | 1652 | asUfsugcuAfucgucuUfcUfgacccs | 2500 |
| | auagcaas{invAb} | | usu | |
| D-1562 | [GalNAc3]saaagacGfgUfAfCfAfu | 1653 | asUfsaggaUfuauguaCfcGfucuuus | 2501 |
| | aauccuas{invAb} | | usu | |
| D-1563 | [GalNAc3]sugucucCfgAfAfGfUfg | 1654 | asAfscuggGfgcacuuCfgGfagacas | 2502 |
| | ccccagus{invAb} | | usu | |
| D-1564 | [GalNAc3]suaguuuGfgAfAfUfUfc | 1655 | asGfsagcaAfagaauuCfcAfaacuas | 2503 |
| | uuugcucs{invAb} | | usu | |
| D-1565 | [GalNAc3]sccaaacUfcCfCfAfUfu | 1656 | asAfsugaaAfgaauggGfaGfuuuggs | 2504 |
| | cuuucaus{invAb} | | usu | |
| D-1566 | [GalNAc3]sguacauAfaUfCfCfUfa | 1657 | usAfsaaccUfguaggaUfuAfuguacs | 2505 |
| | cagguuus{invAb} | | usu | |
| D-1567 | [GalNAc3]scaugcuCfuCfUfCfCfu | 1658 | usAfsgaacCfgaggagAfgAfgcaugs | 2506 |
| | cgguucus{invAb} | | usu | |
| D-1568 | [GalNAc3]saccccaUfgCfUfCfUfc | 1659 | asCfscgagGfagagagCfaUfggggus | 2507 |
| | uccucggs{invAb} | | usu | |
| D-1569 | [GalNAc3]sccgaucAfgCfUfGfUfa | 1660 | usGfsuuguUfcuacagCfuGfaucggs | 2508 |
| | gaacaacs{invAb} | | usu | |
| D-1570 | [GalNAc3]saaccggAfgAfAfCfUfc | 1661 | asUfsuucuAfagaguuCfuCfcgguus | 2509 |
| | uuagaaas{invAb} | | usu | |
| D-1571 | [GalNAc3]sacacagUfaGfUfGfGfa | 1662 | asGfsgaaaGfcuccacUfaCfugugus | 2510 |
| | gcuuuccs{invAb} | | usu | |
| D-1572 | [GalNAc3]saaauacAfuGfUfCfUfa | 1663 | asGfsacagAfcuagacAfuGfuauuus | 2511 |
| | gucugucs{invAb} | | usu | |
| D-1573 | [GalNAc3]sauccggAfaGfUfUfUfg | 1664 | usCfsuaucUfucaaacUfuCfcggaus | 2512 |
| | aagauags{invAb} | | usu | |
| D-1574 | [GalNAc3]sgcaagcCfuAfAfAfCfg | 1665 | asUfsuucuGfacguuuAfgGfcuugcs | 2513 |
| | ucagaaas{invAb} | | usu | |
| D-1575 | [GalNAc3]suugaauAfcUfCfAfGfg | 1666 | usUfscgacUfuccugaGfuAfuucaas | 2514 |
| | aagucgas{invAb} | | usu | |
| D-1576 | [GalNAc3]sguucaaCfcAfCfCfUfu | 1667 | asAfsaucaUfcaagguGfgUfugaacs | 2515 |
| | gaugauus{invAb} | | usu | |
| D-1577 | [GalNAc3]suacuguUfuAfCfAfUfu | 1668 | asAfsaucuGfcaauguAfaAfcaguas | 2516 |
| | gcagauus{invAb} | | usu | |
| D-1578 | [GalNAc3]saccuugCfcAfAfAfUfu | 1669 | asUfscuccGfgaauuuGfgCfaaggus | 2517 |
| | ccggagas{invAb} | | usu | |
| D-1579 | [GalNAc3]scaauggAfuGfAfUfAfa | 1670 | asUfsgguaUfuuuaucAfuCfcauugs | 2518 |
| | aauaccas{invAb} | | usu | |
| D-1580 | [GalNAc3]sggcuccUfgUfUfUfCfc | 1671 | asAfsggcaAfuggaaaCfaGfgagccs | 2519 |
| | auugccus{invAb} | | usu | |
| D-1581 | [GalNAc3]sgaccagAfuUfGfCfUfa | 1672 | usUfsucucAfuuagcaAfuCfuggucs | 2520 |
| | augagaas{invAb} | | usu | |
| D-1582 | [GalNAc3]saggcauUfuUfGfUfAfa | 1673 | asUfsuuccAfauuacaAfaAfugccus | 2521 |
| | uuggaaas{invAb} | | usu | |
| D-1583 | [GalNAc3]suaggcaUfuUfUfGfUfa | 1674 | usUfsuccaAfuuacaaAfaUfgccuas | 2522 |
| | auuggaas{invAb} | | usu | |
| D-1584 | [GalNAc3]sgauuccAfaGfUfCfCfa | 1675 | asCfscucaCfauggacUfuGfgaaucs | 2523 |
| | ugugaggs{invAb} | | usu | |
| D-1585 | [GalNAc3]sccgcacUfcCfUfAfUfg | 1676 | usCfsuucaGfccauagGfaGfugcggs | 2524 |
| | gcugaags{invAb} | | usu | |
| D-1586 | [GalNAc3]scuucggGfaUfUfUfUfg | 1677 | asUfsugucUfucaaaaUfcCfcgaags | 2525 |
| | aagacaas{invAb} | | usu | |
| D-1587 | [GalNAc3]suuugagAfgCfCfCfCfa | 1678 | asUfsucauUfguggggCfuCfucaaas | 2526 |
| | caaugaas{invAb} | | usu | |
| D-1588 | [GalNAc3]saaccgaUfuUfCfAfGfu | 1679 | asAfsucguGfcacugaAfaUfcgguus | 2527 |
| | gcacgaus{invAb} | | usu | |
| D-1589 | [GalNAc3]sacaaggGfaCfUfUfAfu | 1680 | asUfsuuguUfgauaagUfcCfcuugus | 2528 |
| | caacaaas{invAb} | | usu | |
| D-1590 | [GalNAc3]sguaaaaCfuAfUfAfCfu | 1681 | asAfscgggUfcaguauAfgUfuuuacs | 2529 |
| | gacccgus{invAb} | | usu | |
| D-1591 | [GalNAc3]sucuacuGfuUfUfAfCfa | 1682 | asUfscugcAfauguaaAfcAfguagas | 2530 |
| | uugcagas{invAb} | | usu | |
| D-1592 | [GalNAc3]sguuuugGfuUfCfCfCfa | 1683 | usCfsucaaGfuugggaAfcCfaaaacs | 2531 |
| | acuugags{invAb} | | usu | |
| D-1593 | [GalNAc3]suugagcUfaUfGfUfGfu | 1684 | asAfscuucCfaacacaUfaGfcucaas | 2532 |
| | uggaagus{invAb} | | usu | |
| D-1594 | [GalNAc3]suuaaguUfuGfCfUfCfu | 1685 | usAfscgauUfaagagcAfaAfcuuaas | 2533 |
| | uaaucgus{invAb} | | usu | |
| D-1595 | [GalNAc3]sugaccuUfgCfCfAfAfa | 1686 | asUfsccggAfauuuggCfaAfggucas | 2534 |
| | uuccggas{invAb} | | usu | |
| D-1596 | [GalNAc3]suuuaagUfuUfGfCfUfc | 1687 | asCfsgauuAfagagcaAfaCfuuaaas | 2535 |
| | uuaaucgs{invAb} | | usu | |
| D-1597 | [GalNAc3]saccaucCfgAfUfCfAfg | 1688 | usUfscuacAfgcugauCfgGfauggus | 2536 |
| | cuguagas{invAb} | | usu | |
| D-1598 | [GalNAc3]sacaaauGfaCfCfUfUfg | 1689 | asAfsauuuGfgcaaggUfcAfuuugus | 2537 |
| | ccaaauus{invAb} | | usu | |
| D-1599 | [GalNAc3]saaagaaCfcAfUfCfCfg | 1690 | asAfsgcugAfucggauGfgUfucuuus | 2538 |
| | aucagcus{invAb} | | usu | |
| D-1600 | [GalNAc3]scugauuUfuAfUfCfGfu | 1691 | asGfsuguuUfgacgauAfaAfaucags | 2539 |
| | caaacacs{invAb} | | usu | |
| D-1601 | [GalNAc3]sucucucCfuCfGfGfUfu | 1692 | asAfsgcguAfgaaccgAfgGfagagas | 2540 |
| | cuacgcus{invAb} | | usu | |
| D-1602 | [GalNAc3]saguuugAfaGfAfUfAfg | 1693 | asUfsucgaAfucuaucUfuCfaaacus | 2541 |
| | auucgaas{invAb} | | usu | |
| D-1603 | [GalNAc3]suuuacaUfuGfCfAfGfa | 1694 | usAfsuagcAfaucugcAfaUfguaaas | 2542 |
| | uugcuaus{invAb} | | usu | |
| D-1604 | [GalNAc3]sauggucAfcUfCfUfGfa | 1695 | asUfscgguUfuucagaGfuGfaccaus | 2543 |
| | aaaccgas{invAb} | | usu | |
| D-1605 | [GalNAc3]sucucaaUfuCfCfAfCfa | 1696 | usGfsagauCfguguggAfaUfugagas | 2544 |
| | cgaucucs{invAb} | | usu | |
| D-1606 | [GalNAc3]succacaCfgAfUfCfUfc | 1697 | usCfsucucAfugagauCfgUfguggas | 2545 |
| | augagags{invAb} | | usu | |
| D-1607 | [GalNAc3]sgacaauCfaGfGfAfCfg | 1698 | asAfscaagAfccguccUfgAfuugucs | 2546 |
| | gucuugus{invAb} | | usu | |
| D-1608 | [GalNAc3]saagagcCfuAfUfCfCfc | 1699 | asGfsaaagCfagggauAfgGfcucuus | 2547 |
| | ugcuuucs{invAb} | | usu | |
| D-1609 | [GalNAc3]sucaggaCfgGfUfCfUfu | 1700 | asUfsauucAfcaagacCfgUfccugas | 2548 |
| | gugaauas{invAb} | | usu | |
| D-1610 | [GalNAc3]sgucagaAfaUfCfCfAfg | 1701 | asUfsuuagUfacuggaUfuUfcugacs | 2549 |
| | uacuaaas{invAb} | | usu | |
| D-1611 | [GalNAc3]saguuugGfaAfUfUfCfu | 1702 | usAfsgagcAfaagaauUfcCfaaacus | 2550 |
| | uugcucus{invAb} | | usu | |
| D-1612 | [GalNAc3]suuuggaAfuUfCfUfUfu | 1703 | asGfsuagaGfcaaagaAfuUfccaaas | 2551 |
| | gcucuacs{invAb} | | usu | |
| D-1613 | [GalNAc3]scugaaaUfgGfAfCfAfa | 1704 | asAfsggucAfuuugucCfaUfuucags | 2552 |
| | augaccus{invAb} | | usu | |
| D-1614 | [GalNAc3]saagacgGfuAfCfAfUfa | 1705 | usGfsuaggAfuuauguAfcCfgucuus | 2553 |
| | auccuacs{invAb} | | usu | |
| D-1615 | [GalNAc3]sggucuuGfuGfAfAfUfa | 1706 | asCfsuuucCfauauucAfcAfagaccs | 2554 |
| | uggaaags{invAb} | | usu | |
| D-1616 | [GalNAc3]suucaacCfaCfCfUfUfg | 1707 | asCfsaaucAfucaaggUfgGfuugaas | 2555 |
| | augauugs{invAb} | | usu | |
| D-1617 | [GalNAc3]sacuuuaUfuCfUfAfUfa | 1708 | asUfsuugcUfcuauagAfaUfaaagus | 2556 |
| | gagcaaas{invAb} | | usu | |
| D-1618 | [GalNAc3]sagaccgCfaCfUfCfCfu | 1709 | usCfsagccAfuaggagUfgCfggucus | 2557 |
| | auggcugs{invAb} | | usu | |
| D-1619 | [GalNAc3]suauuucCfcCfAfAfUfg | 1710 | usUfsaucaUfccauugGfgGfaaauas | 2558 |
| | gaugauas{invAb} | | usu | |
| D-1620 | [GalNAc3]sgucagaAfgAfCfGfAfu | 1711 | asCfsauugCfuaucguCfuUfcugacs | 2559 |
| | agcaaugs{invAb} | | usu | |
| D-1621 | [GalNAc3]sgcuaauGfaGfAfAfAfg | 1712 | asAfsgagcCfacuuucUfcAfuuagcs | 2560 |
| | uggcucus{invAb} | | usu | |
| D-1622 | [GalNAc3]scauuacCfaGfUfUfAfg | 1713 | usUfsuaaaAfgcuaacUfgGfuaaugs | 2561 |
| | cuuuuaas{invAb} | | usu | |
| D-1623 | [GalNAc3]suugcuaUfaAfUfUfUfc | 1714 | asAfscuccUfugaaauUfaUfagcaas | 2562 |
| | aaggagus{invAb} | | usu | |
| D-1624 | [GalNAc3]sucuacuGfuUfUfAfCfa | 1715 | asUfscugcAfauguaaAfcAfguagas | 2563 |
| | uugcagas{invAb} | | usu | |
| D-1625 | [GalNAc3]saauaaaUfgAfGfAfUfg | 1716 | asUfsuaguAfgcaucuCfaUfuuauus | 2564 |
| | cuacuaas{invAb} | | usu | |
| D-1626 | [GalNAc3]suuauucUfaUfAfGfAfg | 1717 | asAfsaguuUfgcucuaUfaGfaauaas | 2565 |
| | caaacuus{invAb} | | usu | |
| D-1627 | [GalNAc3]saugaugCfaCfUfUfUfa | 1718 | asAfsacauCfcuaaagUfgCfaucaus | 2566 |
| | ggauguus{invAb} | | usu | |
| D-1628 | [GalNAc3]suacauuGfcAfGfAfUfu | 1719 | asUfsuauaGfcaaucuGfcAfauguas | 2567 |
| | gcuauaas{invAb} | | usu | |
| D-1629 | [GalNAc3]suuacauUfgCfAfGfAfu | 1720 | usUfsauagCfaaucugCfaAfuguaas | 2568 |
| | ugcuauas{invAb} | | usu | |
| D-1630 | [GalNAc3]sguuuuaAfaGfGfGfUfc | 1721 | usUfsucucAfcgacccUfuUfaaaacs | 2569 |
| | gugagaas{invAb} | | usu | |
| D-1631 | [GalNAc3]sugagcuAfuGfUfGfUfu | 1722 | asCfsacuuCfcaacacAfuAfgcucas | 2570 |
| | ggaagugs{invAb} | | usu | |
| D-1632 | [GalNAc3]suuuggcAfuCfGfGfCfu | 1723 | asAfsaacaGfgagccgAfuGfccaaas | 2571 |
| | ccuguuus{invAb} | | usu | |
| D-1633 | [GalNAc3]sauccuaCfaGfGfUfUfu | 1724 | asUfsacauUfuaaaccUfgUfaggaus | 2572 |
| | aaauguas{invAb} | | usu | |
| D-1634 | [GalNAc3]suacacaAfaGfAfCfGfg | 1725 | asUfsuaugUfaccgucUfuUfguguas | 2573 |
| | uacauaas{invAb} | | usu | |
| D-1635 | [DCA- | 1726 | usUfscuaaGfaguucuCfcGfguugcs | 2574 |
| | C6]gcaaccGfgAfGfAfAfcucuuaga | | usu | |
| | s{invAb} | | | |
| D-1636 | [DCA- | 1727 | usGfsaaagAfaugggaGfuUfuggugs | 2575 |
| | C6]caccaaAfcUfCfCfCfauucuuuc | | usu | |
| | s{invAb} | | | |
| D-1637 | [DCA- | 1728 | asUfsugucUfucaaaaUfcCfcgaags | 2576 |
| | C6]cuucggGfaUfUfUfUfgaagacaa | | usu | |
| | s{invAb} | | | |
| D-1638 | [DCA- | 1729 | usCfsuaucUfucaaacUfuCfcggaus | 2577 |
| | C6]auccggAfaGfUfUfUfgaagauag | | usu | |
| | s{invAb} | | | |
| D-1639 | [DCA- | 1730 | usUfscuaaGfaguucuCfcGfguusus | 2578 |
| | C6]aaccGfgAfGfAfAfcucuuagaas | | u | |
| | us{invAb} | | | |
| D-1640 | [GalNAc3]saaccGfgAfGfAfAfcuc | 1731 | usUfscuaaGfaguucuCfcGfguusus | 2579 |
| | uuagaasus{invAb} | | u | |
| D-1641 | [DCA- | 1732 | asAfsugaaAfgaauggGfaGfuuuggs | 2580 |
| | C6]ccaaacUfcCfCfAfUfucuuucau | | usu | |
| | s{invAb} | | | |
| D-1642 | [DCA- | 1733 | asGfsuauuUfuaucauCfcAfuugggs | 2581 |
| | C6]cccaauGfgAfUfGfAfuaaaauac | | usu | |
| | s{invAb} | | | |
| D-1643 | [DCA- | 1734 | asCfscauaAfgcguagAfaCfcgaggs | 2582 |
| | C6]ccucggUfuCfUfAfCfgcuuaugg | | usu | |
| | s{invAb} | | | |
| D-1644 | [DCA- | 1735 | usCfsuaucUfucaaacUfuCfcggsus | 2583 |
| | C6]ccggAfaGfUfUfUfgaagauagas | | u | |
| | us{invAb} | | | |
| D-1645 | [GalNAc3]sccggAfaGfUfUfUfgaa | 1736 | usCfsuaucUfucaaacUfuCfcggsus | 2584 |
| | gauagasus{invAb} | | u | |
| D-1646 | [GalNAc3]saaccggAfgAfAfCfUfc | 1737 | usUfscuaaGfaguuCfuCfcGfguusu | 2585 |
| | uuagaaus{invAb} | | su | |
| D-1647 | [GalNAc3]sucggUfuCfUfAfCfgcu | 1738 | asCfscauaAfgcguagAfaCfcgasus | 2586 |
| | uauggusus{invAb} | | u | |
| D-1648 | [GalNAc3]sccaaAfcUfCfCfCfauu | 1739 | usGfsaaagAfaugggaGfuUfuggsus | 2587 |
| | cuuucasus{invAb} | | u | |
| D-1649 | [GalNAc3]sucggGfaUfUfUfUfgaa | 1740 | asUfsugucUfucaaaaUfcCfcgasus | 2588 |
| | gacaausus{invAb} | | u | |
| D-1650 | [GalNAc3]scaauGfgAfUfGfAfuaa | 1741 | asGfsuauuUfuaucauCfcAfuugsus | 2589 |
| | aauacusus{invAb} | | u | |
| D-1651 | [GalNAc3]sucggUfuCfUfAfCfgcu | 1742 | asCfscauaAfgcguAfgAfaCfcgasu | 2590 |
| | uauggusus{invAb} | | su | |
| D-1652 | [GalNAc3]saaccGfgAfGfAfAfcuc | 1743 | usUfscuaaGfaguuCfuCfcGfguusu | 2591 |
| | uuagaasus{invAb} | | su | |
| D-1653 | [GalNAc3]sccaaAfcUfCfCfCfauu | 1744 | usGfsaaagAfauggGfaGfuUfuggsu | 2592 |
| | cuuucasus{invAb} | | su | |
| D-1654 | [GalNAc3]sucggGfaUfUfUfUfgaa | 1745 | asUfsugucUfucaaAfaUfcCfcgasu | 2593 |
| | gacaausus{invAb} | | su | |
| D-1655 | [GalNAc3]scaauGfgAfUfGfAfuaa | 1746 | asGfsuauuUfuaucAfuCfcAfuugsu | 2594 |
| | aauacusus{invAb} | | su | |
| D-1656 | [GalNAc3]sccucggUfuCfUfAfCfg | 1747 | asCfscauAfAfgcguagAfaCfcgagg | 2595 |
| | cuuauggs{invAb} | | susu | |
| D-1657 | [GalNAc3]sgcaaccGfgAfGfAfAfc | 1748 | usUfscuaAfGfaguucuCfcGfguugc | 2596 |
| | ucuuagas{invAb} | | susu | |
| D-1658 | [GalNAc3]scaccaaAfcUfCfCfCfa | 1749 | usGfsaaaGfAfaugggaGfuUfuggug | 2597 |
| | uucuuucs{invAb} | | susu | |
| D-1659 | [GalNAc3]scuucggGfaUfUfUfUfg | 1750 | asUfsuguCfUfucaaaaUfcCfcgaag | 2598 |
| | aagacaas{invAb} | | susu | |
| D-1660 | [GalNAc3]scccaauGfgAfUfGfAfu | 1751 | asGfsuauUfUfuaucauCfcAfuuggg | 2599 |
| | aaaauacs{invAb} | | susu | |
| D-1661 | [GalNAc3]sccucggUfuCfUfAfCfg | 1752 | asCfscauaAfgcguAfgAfaCfcgagg | 2600 |
| | cuuauggs{invAb} | | susu | |
| D-1662 | [GalNAc3]sgcaaccGfgAfGfAfAfc | 1753 | usUfscuaaGfaguuCfuCfcGfguugc | 2601 |
| | ucuuagas{invAb} | | susu | |
| D-1663 | [GalNAc3]scaccaaAfcUfCfCfCfa | 1754 | usGfsaaagAfauggGfaGfuUfuggug | 2602 |
| | uucuuucs{invAb} | | susu | |
| D-1664 | [GalNAc3]scuucggGfaUfUfUfUfg | 1755 | asUfsugucUfucaaAfaUfcCfcgaag | 2603 |
| | aagacaas{invAb} | | susu | |
| D-1665 | [GalNAc3]scccaauGfgAfUfGfAfu | 1756 | asGfsuauuUfuaucAfuCfcAfuuggg | 2604 |
| | aaaauacs{invAb} | | susu | |
| D-1666 | [GalNAc3]sccucggUfuCfUfAfCfg | 1757 | asCfscauaagcguAfgAfaCfcgaggs | 2605 |
| | cuuauggs{invAb} | | usu | |
| D-1667 | [GalNAc3]sgcaaccGfgAfGfAfAfc | 1758 | usUfscuaagaguuCfuCfcGfguugcs | 2606 |
| | ucuuagas{invAb} | | usu | |
| D-1668 | [GalNAc3]scaccaaAfcUfCfCfCfa | 1759 | usGfsaaagaauggGfaGfuUfuggugs | 2607 |
| | uucuuucs{invAb} | | usu | |
| D-1669 | [GalNAc3]scuucggGfaUfUfUfUfg | 1760 | asUfsugucuucaaAfaUfcCfcgaags | 2608 |
| | aagacaas{invAb} | | usu | |
| D-1670 | [DCA- | 1761 | usUfscuaaGfaguuCfuCfcGfguusu | 2609 |
| | C6]aaccggAfgAfAfCfUfcuuagaau | | su | |
| | s{invAb} | | | |
| D-1671 | [DCA- | 1762 | asCfscauaAfgcguagAfaCfcgasus | 2610 |
| | C6]ucggUfuCfUfAfCfgcuuauggus | | u | |
| | us{invAb} | | | |
| D-1672 | [DCA- | 1763 | usGfsaaagAfaugggaGfuUfuggsus | 2611 |
| | C6]ccaaAfcUfCfCfCfauucuuucas | | u | |
| | us{invAb} | | | |
| D-1673 | [DCA- | 1764 | asUfsugucUfucaaaaUfcCfcgasus | 2612 |
| | C6]ucggGfaUfUfUfUfgaagacaaus | | u | |
| | us{invAb} | | | |
| D-1674 | [DCA- | 1765 | asGfsuauuUfuaucauCfcAfuugsus | 2613 |
| | C6]caauGfgAfUfGfAfuaaaauacus | | u | |
| | us{invAb} | | | |
| D-1675 | [DCA- | 1766 | asCfscauaAfgcguAfgAfaCfcgasu | 2614 |
| | C6]ucggUfuCfUfAfCfgcuuauggus | | su | |
| | us{invAb} | | | |
| D-1676 | [DCA- | 1767 | usUfscuaaGfaguuCfuCfcGfguusu | 2615 |
| | C6]aaccGfgAfGfAfAfcucuuagaas | | su | |
| | us{invAb} | | | |
| D-1677 | [DCA- | 1768 | usGfsaaagAfauggGfaGfuUfuggsu | 2616 |
| | C6]ccaaAfcUfCfCfCfauucuuucas | | su | |
| | us{invAb} | | | |
| D-1678 | [DCA- | 1769 | asUfsugucUfucaaAfaUfcCfcgasu | 2617 |
| | C6]ucggGfaUfUfUfUfgaagacaaus | | su | |
| | us{invAb} | | | |
| D-1679 | [DCA- | 1770 | asGfsuauuUfuaucAfuCfcAfuugsu | 2618 |
| | C6]caauGfgAfUfGfAfuaaaauacus | | su | |
| | us{invAb} | | | |
| D-1680 | [DCA- | 1771 | asCfscauAfAfgcguagAfaCfcgagg | 2619 |
| | C6]ccucggUfuCfUfAfCfgcuuaugg | | susu | |
| | s{invAb} | | | |
| D-1681 | [DCA- | 1772 | usUfscuaAfGfaguucuCfcGfguugc | 2620 |
| | C6]gcaaccGfgAfGfAfAfcucuuaga | | susu | |
| | s{invAb} | | | |
| D-1682 | [DCA- | 1773 | usGfsaaaGfAfaugggaGfuUfuggug | 2621 |
| | C6]caccaaAfcUfCfCfCfauucuuuc | | susu | |
| | s{invAb} | | | |
| D-1683 | [DCA- | 1774 | asUfsuguCfUfucaaaaUfcCfcgaag | 2622 |
| | C6]cuucggGfaUfUfUfUfgaagacaa | | susu | |
| | s{invAb} | | | |
| D-1684 | [DCA- | 1775 | asGfsuauUfUfuaucauCfcAfuuggg | 2623 |
| | C6]cccaauGfgAfUfGfAfuaaaauac | | susu | |
| | s{invAb} | | | |
| D-1685 | [DCA- | 1776 | asCfscauaAfgcguAfgAfaCfcgagg | 2624 |
| | C6]ccucggUfuCfUfAfCfgcuuaugg | | susu | |
| | s{invAb} | | | |
| D-1686 | [DCA- | 1777 | usUfscuaaGfaguuCfuCfcGfguugc | 2625 |
| | C6]gcaaccGfgAfGfAfAfcucuuaga | | susu | |
| | s{invAb} | | | |
| D-1687 | [DCA- | 1778 | usGfsaaagAfauggGfaGfuUfuggug | 2626 |
| | C6]caccaaAfcUfCfCfCfauucuuuc | | susu | |
| | s{invAb} | | | |
| D-1688 | [DCA- | 1779 | asUfsugucUfucaaAfaUfcCfcgaag | 2627 |
| | C6]cuucggGfaUfUfUfUfgaagacaa | | susu | |
| | s{invAb} | | | |
| D-1689 | [DCA- | 1780 | asGfsuauuUfuaucAfuCfcAfuuggg | 2628 |
| | C6]cccaauGfgAfUfGfAfuaaaauac | | susu | |
| | s{invAb} | | | |
| D-1690 | [DCA- | 1781 | asCfscauaagcguAfgAfaCfcgaggs | 2629 |
| | C6]ccucggUfuCfUfAfCfgcuuaugg | | usu | |
| | s{invAb} | | | |
| D-1691 | [DCA- | 1782 | usUfscuaagaguuCfuCfcGfguugcs | 2630 |
| | C6]gcaaccGfgAfGfAfAfcucuuaga | | usu | |
| | s{invAb} | | | |
| D-1692 | [DCA- | 1783 | usGfsaaagaauggGfaGfuUfuggugs | 2631 |
| | C6]caccaaAfcUfCfCfCfauucuuuc | | usu | |
| | s{invAb} | | | |
| D-1693 | [DCA- | 1784 | asUfsugucuucaaAfaUfcCfcgaags | 2632 |
| | C6]cuucggGfaUfUfUfUfgaagacaa | | usu | |
| | s{invAb} | | | |
| D-1694 | [DCA- | 1785 | usUfscuacAfgcugauCfgGfauggus | 2633 |
| | C6]accaucCfgAfUfCfAfgcuguaga | | usu | |
| | s{invAb} | | | |
| D-1695 | [DCA- | 1786 | asCfsuuucCfauauucAfcAfagaccs | 2634 |
| | C6]ggucuuGfuGfAfAfUfauggaaag | | usu | |
| | s{invAb} | | | |
| D-1696 | [DCA- | 1787 | asCfsacuuCfcaacacAfuAfgcucas | 2635 |
| | C6]ugagcuAfuGfUfGfUfuggaagug | | usu | |
| | s{invAb} | | | |
| D-1697 | [DCA- | 1788 | usGfsuaggAfuuauguAfcCfgucuus | 2636 |
| | C6]aagacgGfuAfCfAfUfaauccuac | | usu | |
| | s{invAb} | | | |
| D-1698 | [DCA- | 1789 | usAfsgagcAfaagaauUfcCfaaacus | 2637 |
| | C6]aguuugGfaAfUfUfCfuuugcucu | | usu | |
| | s{invAb} | | | |
| D-1699 | [DCA- | 1790 | asGfsuagaGfcaaagaAfuUfccaaas | 2638 |
| | C6]uuuggaAfuUfCfUfUfugcucuac | | usu | |
| | s{invAb} | | | |
| D-1700 | [GalNAc3]scauccgAfuCfAfGfCfu | 1791 | usUfscuacAfgcugAfuCfgGfaugsu | 2639 |
| | guagaasus{invAb} | | su | |
| D-1701 | [GalNAc3]sucuuguGfaAfUfAfUfg | 1792 | asCfsuuucCfauauUfcAfcAfagasu | 2640 |
| | gaaagusus{invAb} | | su | |
| D-1702 | [GalNAc3]sgacgguAfcAfUfAfAfu | 1793 | usGfsuaggAfuuauGfuAfcCfgucsu | 2641 |
| | ccuacasus{invAb} | | su | |
| D-1703 | [GalNAc3]sagcuauGfuGfUfUfGfg | 1794 | asCfsacuuCfcaacAfcAfuAfgcusu | 2642 |
| | aagugusus{invAb} | | su | |
| D-1704 | [GalNAc3]suggaauUfcUfUfUfGfc | 1795 | asGfsuagaGfcaaaGfaAfuUfccasu | 2643 |
| | ucuacusus{invAb} | | su | |
| D-1705 | [GalNAc3]suuuggaAfuUfCfUfUfu | 1796 | usAfsgagcAfaagaAfuUfcCfaaasu | 2644 |
| | gcucuasus{invAb} | | su | |
| D-1706 | [GalNAc3]sgcuauaAfuUfUfCfAfa | 1797 | asAfscuccUfugaaAfuUfaUfagcsu | 2645 |
| | ggaguusus{invAb} | | su | |
| D-1707 | [GalNAc3]scaucCfgAfUfCfAfgcu | 1798 | usUfscuacAfgcugauCfgGfaugsus | 2646 |
| | guagaasus{invAb} | | u | |
| D-1708 | [GalNAc3]sucuuGfuGfAfAfUfaug | 1799 | asCfsuuucCfauauucAfcAfagasus | 2647 |
| | gaaagusus{invAb} | | u | |
| D-1709 | [GalNAc3]sgacgGfuAfCfAfUfaau | 1800 | usGfsuaggAfuuauguAfcCfgucsus | 2648 |
| | ccuacasus{invAb} | | u | |
| D-1710 | [GalNAc3]sagcuAfuGfUfGfUfugg | 1801 | asCfsacuuCfcaacacAfuAfgcusus | 2649 |
| | aagugusus{invAb} | | u | |
| D-1711 | [GalNAc3]suggaAfuUfCfUfUfugc | 1802 | asGfsuagaGfcaaagaAfuUfccasus | 2650 |
| | ucuacusus{invAb} | | u | |
| D-1712 | [GalNAc3]suuugGfaAfUfUfCfuuu | 1803 | usAfsgagcAfaagaauUfcCfaaasus | 2651 |
| | gcucuasus{invAb} | | u | |
| D-1713 | [GalNAc3]sgcuaUfaAfUfUfUfcaa | 1804 | asAfscuccUfugaaauUfaUfagcsus | 2652 |
| | ggaguusus{invAb} | | u | |
| D-1714 | [GalNAc3]scaucCfgAfUfCfAfgcu | 1805 | usUfscuacAfgcugAfuCfgGfaugsu | 2653 |
| | guagaasus{invAb} | | su | |
| D-1715 | [GalNAc3]sucuuGfuGfAfAfUfaug | 1806 | asCfsuuucCfauauUfcAfcAfagasu | 2654 |
| | gaaagusus{invAb} | | su | |
| D-1716 | [GalNAc3]sgacgGfuAfCfAfUfaau | 1807 | usGfsuaggAfuuauGfuAfcCfgucsu | 2655 |
| | ccuacasus{invAb} | | su | |
| D-1717 | [GalNAc3]sagcuAfuGfUfGfUfugg | 1808 | asCfsacuuCfcaacAfcAfuAfgcusu | 2656 |
| | aagugusus{invAb} | | su | |
| D-1718 | [GalNAc3]suggaAfuUfCfUfUfugc | 1809 | asGfsuagaGfcaaaGfaAfuUfccasu | 2657 |
| | ucuacusus{invAb} | | su | |
| D-1719 | [GalNAc3]suuugGfaAfUfUfCfuuu | 1810 | usAfsgagcAfaagaAfuUfcCfaaasu | 2658 |
| | gcucuasus{invAb} | | su | |
| D-1720 | [GalNAc3]sgcuaUfaAfUfUfUfcaa | 1811 | asAfscuccUfugaaAfuUfaUfagcsu | 2659 |
| | ggaguusus{invAb} | | su | |
| D-1721 | [GalNAc3]saccaucCfgAfUfCfAfg | 1812 | usUfscuaCfAfgcugauCfgGfauggu | 2660 |
| | cuguagas{invAb} | | susu | |
| D-1722 | [GalNAc3]sggucuuGfuGfAfAfUfa | 1813 | asCfsuuuCfCfauauucAfcAfagacc | 2661 |
| | uggaaags{invAb} | | susu | |
| D-1723 | [GalNAc3]saagacgGfuAfCfAfUfa | 1814 | usGfsuagGfAfuuauguAfcCfgucuu | 2662 |
| | auccuacs{invAb} | | susu | |
| D-1724 | [GalNAc3]sugagcuAfuGfUfGfUfu | 1815 | asCfsacuUfCfcaacacAfuAfgcuca | 2663 |
| | ggaagugs{invAb} | | susu | |
| D-1725 | [GalNAc3]suuuggaAfuUfCfUfUfu | 1816 | asGfsuagAfGfcaaagaAfuUfccaaa | 2664 |
| | gcucuacs{invAb} | | susu | |
| D-1726 | [GalNAc3]saguuugGfaAfUfUfCfu | 1817 | usAfsgagCfAfaagaauUfcCfaaacu | 2665 |
| | uugcucus{invAb} | | susu | |
| D-1727 | [GalNAc3]suugcuaUfaAfUfUfUfc | 1818 | asAfscucCfUfugaaauUfaUfagcaa | 2666 |
| | aaggagus{invAb} | | susu | |
| D-1728 | [GalNAc3]saccaucCfgAfUfCfAfg | 1819 | usUfscuacAfgcugAfuCfgGfauggu | 2667 |
| | cuguagas{invAb} | | susu | |
| D-1729 | [GalNAc3]sggucuuGfuGfAfAfUfa | 1820 | asCfsuuucCfauauUfcAfcAfagacc | 2668 |
| | uggaaags{invAb} | | susu | |
| D-1730 | [GalNAc3]saagacgGfuAfCfAfUfa | 1821 | usGfsuaggAfuuauGfuAfcCfgucuu | 2669 |
| | auccuacs{invAb} | | susu | |
| D-1731 | [GalNAc3]sugagcuAfuGfUfGfUfu | 1822 | asCfsacuuCfcaacAfcAfuAfgcuca | 2670 |
| | ggaagugs{invAb} | | susu | |
| D-1732 | [GalNAc3]suuuggaAfuUfCfUfUfu | 1823 | asGfsuagaGfcaaaGfaAfuUfccaaa | 2671 |
| | gcucuacs{invAb} | | susu | |
| D-1733 | [GalNAc3]saguuugGfaAfUfUfCfu | 1824 | usAfsgagcAfaagaAfuUfcCfaaacu | 2672 |
| | uugcucus{invAb} | | susu | |
| D-1734 | [GalNAc3]suugcuaUfaAfUfUfUfc | 1825 | asAfscuccUfugaaAfuUfaUfagcaa | 2673 |
| | aaggagus{invAb} | | susu | |
| D-1735 | [GalNAc3]saccaucCfgAfUfCfAfg | 1826 | usUfscuacagcugAfuCfgGfauggus | 2674 |
| | cuguagas{invAb} | | usu | |
| D-1736 | [GalNAc3]sggucuuGfuGfAfAfUfa | 1827 | asCfsuuuccauauUfcAfcAfagaccs | 2675 |
| | uggaaags{invAb} | | usu | |
| D-1737 | [GalNAc3]saagacgGfuAfCfAfUfa | 1828 | usGfsuaggauuauGfuAfcCfgucuus | 2676 |
| | auccuacs{invAb} | | usu | |
| D-1738 | [GalNAc3]sugagcuAfuGfUfGfUfu | 1829 | asCfsacuuccaacAfcAfuAfgcucas | 2677 |
| | ggaagugs{invAb} | | usu | |
| D-1739 | [GalNAc3]suuuggaAfuUfCfUfUfu | 1830 | asGfsuagagcaaaGfaAfuUfccaaas | 2678 |
| | gcucuacs{invAb} | | usu | |
| D-1740 | [GalNAc3]saguuugGfaAfUfUfCfu | 1831 | usAfsgagcaaagaAfuUfcCfaaacus | 2679 |
| | uugcucus{invAb} | | usu | |
| D-1741 | [GalNAc3]suugcuaUfaAfUfUfUfc | 1832 | asAfscuccuugaaAfuUfaUfagcaas | 2680 |
| | aaggagus{invAb} | | usu | |
| D-1742 | [GalNAc3]saccaggAfaAfUfGfUfa | 1833 | usGfsguguCfuuacauUfuCfcuggus | 2681 |
| | agacaccs{invAb} | | usu | |
| D-1743 | [GalNAc3]sccaggaAfaUfGfUfAfa | 1834 | usUfsggugUfcuuacaUfuUfccuggs | 2682 |
| | gacaccas{invAb} | | usu | |
| D-1744 | [GalNAc3]scccucgUfaUfGfUfUfu | 1835 | asAfscuuuCfaaaacaUfaCfgagggs | 2683 |
| | ugaaagus{invAb} | | usu | |
| D-1745 | [GalNAc3]suuucagUfuUfUfAfAfa | 1836 | asAfscgacCfcuuuaaAfaCfugaaas | 2684 |
| | gggucgus{invAb} | | usu | |
| D-1746 | [GalNAc3]sucaguuUfuAfAfAfGfg | 1837 | asUfscacgAfcccuuuAfaAfacugas | 2685 |
| | gucgugas{invAb} | | usu | |
| D-1747 | [GalNAc3]scaguuuUfaAfAfGfGfg | 1838 | usCfsucacGfacccuuUfaAfaacugs | 2686 |
| | ucgugags{invAb} | | usu | |
| D-1748 | [GalNAc3]suuuaaaGfgGfUfCfGfu | 1839 | asGfsuuucUfcacgacCfcUfuuaaas | 2687 |
| | gagaaacs{invAb} | | usu | |
| D-1749 | [GalNAc3]sggaagcUfuGfAfGfCfu | 1840 | asAfsacacAfuagcucAfaGfcuuccs | 2688 |
| | auguguus{invAb} | | usu | |
| D-1750 | [GalNAc3]sgaagcuUfgAfGfCfUfa | 1841 | asCfsaacaCfauagcuCfaAfgcuucs | 2689 |
| | uguguugs{invAb} | | usu | |
| D-1751 | [GalNAc3]sagcuugAfgCfUfAfUfg | 1842 | usUfsccaaCfacauagCfuCfaagcus | 2690 |
| | uguuggas{invAb} | | usu | |
| D-1752 | [GalNAc3]sgcuugaGfcUfAfUfGfu | 1843 | asUfsuccaAfcacauaGfcUfcaagcs | 2691 |
| | guuggaas{invAb} | | usu | |
| D-1753 | [GalNAc3]scuugagCfuAfUfGfUfg | 1844 | asCfsuuccAfacacauAfgCfucaags | 2692 |
| | uuggaags{invAb} | | usu | |
| D-1754 | [GalNAc3]sgagcuaUfgUfGfUfUfg | 1845 | asGfscacuUfccaacaCfaUfagcucs | 2693 |
| | gaagugcs{invAb} | | usu | |
| D-1755 | [GalNAc3]sguguugGfaAfGfUfGfc | 1846 | asAfsaccaGfggcacuUfcCfaacacs | 2694 |
| | ccugguus{invAb} | | usu | |
| D-1756 | [GalNAc3]sggaaguGfcCfCfUfGfg | 1847 | asAfsuuaaAfaccaggGfcAfcuuccs | 2695 |
| | uuuuaaus{invAb} | | usu | |
| D-1757 | [GalNAc3]sacaaagAfcGfGfUfAfc | 1848 | asGfsgauuAfuguaccGfuCfuuugus | 2696 |
| | auaauccs{invAb} | | usu | |
| D-1758 | [GalNAc3]scaaagaCfgGfUfAfCfa | 1849 | usAfsggauUfauguacCfgUfcuuugs | 2697 |
| | uaauccus{invAb} | | usu | |
| D-1759 | [GalNAc3]scauaauCfcUfAfCfAfg | 1850 | asUfsuuaaAfccuguaGfgAfuuaugs | 2698 |
| | guuuaaas{invAb} | | usu | |
| D-1760 | [GalNAc3]suugcucUfaCfUfGfUfu | 1851 | asCfsaaugUfaaacagUfaGfagcaas | 2699 |
| | uacauugs{invAb} | | usu | |
| D-1761 | [GalNAc3]suugcuaUfaAfUfUfUfc | 1852 | asAfscuccUfugaaauUfaUfagcaas | 2700 |
| | aaggagus{invAb} | | usu | |
| D-1762 | [GalNAc3]saaugauGfcAfCfUfUfu | 1853 | asAfscaucCfuaaaguGfcAfucauus | 2701 |
| | aggaugus{invAb} | | usu | |
| D-1763 | [GalNAc3]saugaugCfaCfUfUfUfa | 1854 | asAfsacauCfcuaaagUfgCfaucaus | 2702 |
| | ggauguus{invAb} | | usu | |
| D-1764 | [GalNAc3]sacaugaAfuCfAfUfUfc | 1855 | asGfsucauGfugaaugAfuUfcaugus | 2703 |
| | acaugacs{invAb} | | usu | |
| D-1765 | [GalNAc3]scaugaaUfcAfUfUfCfa | 1856 | usGfsgucaUfgugaauGfaUfucaugs | 2704 |
| | caugaccs{invAb} | | usu | |
| D-1766 | [GalNAc3]saauacaUfgUfCfUfAfg | 1857 | asGfsgacaGfacuagaCfaUfguauus | 2705 |
| | ucuguccs{invAb} | | usu | |
| D-1767 | [GalNAc3]saugucuAfgUfCfUfGfu | 1858 | asUfsuaaaGfgacagaCfuAfgacaus | 2706 |
| | ccuuuaas{invAb} | | usu | |
| D-1768 | [GalNAc3]saaucagAfuCfAfUfUfa | 1859 | asUfsaacuGfguaaugAfuCfugauus | 2707 |
| | ccaguuas{invAb} | | usu | |
| D-1769 | [GalNAc3]sucagauCfaUfUfAfCfc | 1860 | asGfscuaaCfugguaaUfgAfucugas | 2708 |
| | aguuagcs{invAb} | | usu | |
| D-1770 | [GalNAc3]sagaucaUfuAfCfCfAfg | 1861 | asAfsagcuAfacugguAfaUfgaucus | 2709 |
| | uuagcuus{invAb} | | usu | |
| D-1771 | [GalNAc3]sucauuaCfcAfGfUfUfa | 1862 | usUfsaaaaGfcuaacuGfgUfaaugas | 2710 |
| | gcuuuuas{invAb} | | usu | |
| D-1772 | [GalNAc3]saccaguUfaGfCfUfUfu | 1863 | asUfsgcuuUfaaaagcUfaAfcuggus | 2711 |
| | uaaagcas{invAb} | | usu | |
| D-1773 | [GalNAc3]saagcacAfuUfUfGfUfu | 1864 | usAfsgucuUfaaacaaAfuGfugcuus | 2712 |
| | uaagacus{invAb} | | usu | |
| D-1774 | [GalNAc3]sgcacauUfuGfUfUfUfa | 1865 | asAfsuaguCfuuaaacAfaAfugugcs | 2713 |
| | agacuaus{invAb} | | usu | |
| D-1775 | [GalNAc3]sugagauGfcUfAfCfUfa | 1866 | asAfsaacaAfuuaguaGfcAfucucas | 2714 |
| | auuguuus{invAb} | | usu | |
| D-1776 | [GalNAc3]sgauuuaUfuCfAfGfGfa | 1867 | asUfsggggAfuuccugAfaUfaaaucs | 2715 |
| | auccccas{invAb} | | usu | |
| D-1777 | [GalNAc3]sugcuguGfuGfGfCfCfa | 1868 | asUfsuauaAfuuggccAfcAfcagcas | 2716 |
| | auuauaas{invAb} | | usu | |
| D-1778 | [GalNAc3]sauguaaAfaCfUfAfUfa | 1869 | asGfsggucAfguauagUfuUfuacaus | 2717 |
| | cugacccs{invAb} | | usu | |
| D-1779 | [GalNAc3]suaaaacUfaUfAfCfUfg | 1870 | asAfsacggGfucaguaUfaGfuuuuas | 2718 |
| | acccguus{invAb} | | usu | |
| D-1780 | [GalNAc3]sgagaaaCfuGfGfCfUfg | 1871 | asAfsuuggAfccagccAfgUfuucucs | 2719 |
| | guccaaus{invAb} | | usu | |
| D-1781 | [GalNAc3]sccaaugGfgAfUfUfUfa | 1872 | usGfsuugcUfguaaauCfcCfauuggs | 2720 |
| | cagcaacs{invAb} | | usu | |
| D-1782 | [GalNAc3]saguuugCfuCfUfUfAfa | 1873 | asCfsauacGfauuaagAfgCfaaacus | 2721 |
| | ucguaugs{invAb} | | usu | |
| D-1783 | [GalNAc3]sguuugcUfcUfUfAfAfu | 1874 | usCfscauaCfgauuaaGfaGfcaaacs | 2722 |
| | cguauggs{invAb} | | usu | |
| D-1784 | [GalNAc3]suuugcuCfuUfAfAfUfc | 1875 | usUfsccauAfcgauuaAfgAfgcaaas | 2723 |
| | guauggas{invAb} | | usu | |
| D-1785 | [GalNAc3]sugcucuUfaAfUfCfGfu | 1876 | asCfsuuccAfuacgauUfaAfgagcas | 2724 |
| | auggaags{invAb} | | usu | |
| D-1786 | [GalNAc3]scuuaauCfgUfAfUfGfg | 1877 | usCfsaagcUfuccauaCfgAfuuaags | 2725 |
| | aagcuugs{invAb} | | usu | |
| D-1787 | [GalNAc3]suuaaucGfuAfUfGfGfa | 1878 | asUfscaagCfuuccauAfcGfauuaas | 2726 |
| | agcuugas{invAb} | | usu | |
| D-1788 | [GalNAc3]succauaCfaCfAfAfAfg | 1879 | usGfsuaccGfucuuugUfgUfauggas | 2727 |
| | acgguacs{invAb} | | usu | |
| D-1789 | [GalNAc3]scauacaCfaAfAfGfAfc | 1880 | usAfsuguaCfcgucuuUfgUfguaugs | 2728 |
| | gguacaus{invAb} | | usu | |
| D-1790 | [GalNAc3]sugucuaGfuCfUfGfUfc | 1881 | usAfsuuaaAfggacagAfcUfagacas | 2729 |
| | cuuuaaus{invAb} | | usu | |
| D-1791 | [GalNAc3]sucuaguCfuGfUfCfCfu | 1882 | asCfsuauuAfaaggacAfgAfcuagas | 2730 |
| | uuaauags{invAb} | | usu | |
| D-1792 | [GalNAc3]scuagucUfgUfCfCfUfu | 1883 | asGfscuauUfaaaggaCfaGfacuags | 2731 |
| | uaauagcs{invAb} | | usu | |
| D-1793 | [GalNAc3]sgucuguCfcUfUfUfAfa | 1884 | asAfsgagcUfauuaaaGfgAfcagacs | 2732 |
| | uagcucus{invAb} | | usu | |
| D-1794 | [GalNAc3]sgucugcAfaCfCfGfGfa | 1885 | asAfsgaguUfcuccggUfuGfcagacs | 2733 |
| | gaacucus{invAb} | | usu | |
| D-1795 | [GalNAc3]sucugcaAfcCfGfGfAfg | 1886 | usAfsagagUfucuccgGfuUfgcagas | 2734 |
| | aacucuus{invAb} | | usu | |
| D-1796 | [GalNAc3]sugcaacCfgGfAfGfAfa | 1887 | usCfsuaagAfguucucCfgGfuugcas | 2735 |
| | cucuuags{invAb} | | usu | |
| D-1797 | [GalNAc3]saggaugAfaGfUfUfCfg | 1888 | usCfsccauGfucgaacUfuCfauccus | 2736 |
| | acaugggs{invAb} | | usu | |
| D-1798 | [GalNAc3]sgggacuUfaUfCfAfAfc | 1889 | usUfsuucuUfuguugaUfaAfgucccs | 2737 |
| | aaagaaas{invAb} | | usu | |
| D-1799 | [GalNAc3]sauacucCfuUfCfUfGfg | 1890 | asGfsuugaAfcccagaAfgGfaguaus | 2738 |
| | guucaacs{invAb} | | usu | |
| D-1800 | [GalNAc3]scaagccUfaAfAfCfGfu | 1891 | asAfsuuucUfgacguuUfaGfgcuugs | 2739 |
| | cagaaaus{invAb} | | usu | |
| D-1801 | [GalNAc3]saagccuAfaAfCfGfUfc | 1892 | asGfsauuuCfugacguUfuAfggcuus | 2740 |
| | agaaaucs{invAb} | | usu | |
| D-1802 | [GalNAc3]sgguggaCfaCfAfCfUfc | 1893 | asAfsaaugCfugagugUfgUfccaccs | 2741 |
| | agcauuus{invAb} | | usu | |
| D-1803 | [GalNAc3]sgagcuuUfgUfCfUfCfc | 1894 | asGfscacuUfcggagaCfaAfagcucs | 2742 |
| | gaagugcs{invAb} | | usu | |
| D-1804 | [GalNAc3]sugucucCfgAfAfGfUfg | 1895 | asAfscuggGfgcacuuCfgGfagacas | 2743 |
| | ccccagus{invAb} | | usu | |
| D-1805 | [GalNAc3]saugcucUfcUfCfCfUfc | 1896 | asUfsagaaCfcgaggaGfaGfagcaus | 2744 |
| | gguucuas{invAb} | | usu | |
| D-1806 | [GalNAc3]sagcucaCfaCfGfAfAfg | 1897 | usCfsugaaUfccuucgUfgUfgagcus | 2745 |
| | gauucags{invAb} | | usu | |
| D-1807 | [GalNAc3]sagaagaAfgUfAfCfAfg | 1898 | asGfsgaagGfucuguaCfuUfcuucus | 2746 |
| | accuuccs{invAb} | | usu | |
| D-1808 | [GalNAc3]sgaagaaGfuAfCfAfGfa | 1899 | usGfsggaaGfgucuguAfcUfucuucs | 2747 |
| | ccuucccs{invAb} | | usu | |
| D-1809 | [GalNAc3]saagaagUfaCfAfGfAfc | 1900 | asUfsgggaAfggucugUfaCfuucuus | 2748 |
| | cuucccas{invAb} | | usu | |
| D-1810 | [GalNAc3]sucugaaAfuGfGfAfCfa | 1901 | asGfsgucaUfuuguccAfuUfucagas | 2749 |
| | aaugaccs{invAb} | | usu | |
| D-1811 | [GalNAc3]saaaugaCfcUfUfGfCfc | 1902 | asGfsgaauUfuggcaaGfgUfcauuus | 2750 |
| | aaauuccs{invAb} | | usu | |
| D-1812 | [GalNAc3]saccuaaCfuCfCfCfAfg | 1903 | asCfscgcaUfccugggAfgUfuaggus | 2751 |
| | gaugcggs{invAb} | | usu | |
| D-1813 | [GalNAc3]sugcggcAfgCfGfAfAfg | 1904 | asUfsguguUfgcuucgCfuGfccgcas | 2752 |
| | caacacas{invAb} | | usu | |
| D-1814 | [GalNAc3]sacggccGfgUfAfAfCfa | 1905 | usCfsguucUfuuguuaCfcGfgccgus | 2753 |
| | aagaacgs{invAb} | | usu | |
| D-1815 | [GalNAc3]sggccggUfaAfCfAfAfa | 1906 | asUfsucguUfcuuuguUfaCfcggccs | 2754 |
| | gaacgaas{invAb} | | usu | |
| D-1816 | [GalNAc3]sggggucAfgAfAfGfAfc | 1907 | usUfsgcuaUfcgucuuCfuGfaccccs | 2755 |
| | gauagcas{invAb} | | usu | |
| D-1817 | [GalNAc3]sagaaagAfaCfCfAfUfc | 1908 | asCfsugauCfggauggUfuCfuuucus | 2756 |
| | cgaucags{invAb} | | usu | |
| D-1818 | [GalNAc3]sgaaccaUfcCfGfAfUfc | 1909 | asUfsacagCfugaucgGfaUfgguucs | 2757 |
| | agcuguas{invAb} | | usu | |
| D-1819 | [GalNAc3]saucuggAfaCfAfCfUfa | 1910 | asAfsugcuGfauagugUfuCfcagaus | 2758 |
| | ucagcaus{invAb} | | usu | |
| D-1820 | [GalNAc3]saagaaaAfuAfCfUfCfc | 1911 | asCfsccagAfaggaguAfuUfuucuus | 2759 |
| | uucugggs{invAb} | | usu | |
| D-1821 | [GalNAc3]sacucagGfaAfGfUfCfg | 1912 | usAfsccuuUfucgacuUfcCfugagus | 2760 |
| | aaaaggus{invAb} | | usu | |
| D-1822 | [GalNAc3]sagaaagGfaGfCfAfAfg | 1913 | asGfsuuuaGfgcuugcUfcCfuuucus | 2761 |
| | ccuaaacs{invAb} | | usu | |
| D-1823 | [GalNAc3]saggagcAfaGfCfCfUfa | 1914 | asUfsgacgUfuuaggcUfuGfcuccus | 2762 |
| | aacgucas{invAb} | | usu | |
| D-1824 | [GalNAc3]sggagcaAfgCfCfUfAfa | 1915 | usCfsugacGfuuuaggCfuUfgcuccs | 2763 |
| | acgucags{invAb} | | usu | |
| D-1825 | [GalNAc3]sagcaagCfcUfAfAfAfc | 1916 | usUfsucugAfcguuuaGfgCfuugcus | 2764 |
| | gucagaas{invAb} | | usu | |
| D-1826 | [GalNAc3]sgaaaguCfuCfAfAfUfu | 1917 | usCfsguguGfgaauugAfgAfcuuucs | 2765 |
| | ccacacgs{invAb} | | usu | |
| D-1827 | [GalNAc3]sgucucaAfuUfCfCfAfc | 1918 | asAfsgaucGfuguggaAfuUfgagacs | 2766 |
| | acgaucus{invAb} | | usu | |
| D-1828 | [GalNAc3]sucaauuCfcAfCfAfCfg | 1919 | asAfsugagAfucguguGfgAfauugas | 2767 |
| | aucucaus{invAb} | | usu | |
| D-1829 | [GalNAc3]scaauucCfaCfAfCfGfa | 1920 | usCfsaugaGfaucgugUfgGfaauugs | 2768 |
| | ucucaugs{invAb} | | usu | |
| D-1830 | [GalNAc3]sauuccaCfaCfGfAfUfc | 1921 | usCfsucauGfagaucgUfgUfggaaus | 2769 |
| | ucaugags{invAb} | | usu | |
| D-1831 | [GalNAc3]suuccacAfcGfAfUfCfu | 1922 | asUfscucaUfgagaucGfuGfuggaas | 2770 |
| | caugagas{invAb} | | usu | |
| D-1832 | [GalNAc3]sucaugaGfaGfAfAfCfu | 1923 | usCfsagguCfcaguucUfcUfcaugas | 2771 |
| | ggaccugs{invAb} | | usu | |
| D-1833 | [GalNAc3]scaugagAfgAfAfCfUfg | 1924 | asUfscaggUfccaguuCfuCfucaugs | 2772 |
| | gaccugas{invAb} | | usu | |
| D-1834 | [GalNAc3]saucccaGfcCfUfAfUfc | 1925 | usGfsguguCfagauagGfcUfgggaus | 2773 |
| | ugacaccs{invAb} | | usu | |
| D-1835 | [GalNAc3]succcagCfcUfAfUfCfu | 1926 | usUfsggugUfcagauaGfgCfugggas | 2774 |
| | gacaccas{invAb} | | usu | |
| D-1836 | [GalNAc3]scccagcCfuAfUfCfUfg | 1927 | usUfsugguGfucagauAfgGfcugggs | 2775 |
| | acaccaas{invAb} | | usu | |
| D-1837 | [GalNAc3]sagcagaGfaAfAfUfCfa | 1928 | asGfsgcauCfuugauuUfcUfcugcus | 2776 |
| | agaugccs{invAb} | | usu | |
| D-1838 | [GalNAc3]succgaaGfuGfCfCfCfc | 1929 | asUfsccgaCfuggggcAfcUfucggas | 2777 |
| | agucggas{invAb} | | usu | |
| D-1839 | [GalNAc3]sgaagauAfgAfUfUfCfg | 1930 | asUfscuucUfucgaauCfuAfucuucs | 2778 |
| | aagaagas{invAb} | | usu | |
| D-1840 | [GalNAc3]scagcgaAfgCfAfAfCfa | 1931 | asGfsggagUfguguugCfuUfcgcugs | 2779 |
| | cacucccs{invAb} | | usu | |
| D-1841 | [GalNAc3]sgcgaagCfaAfCfAfCfa | 1932 | usUfsggggAfguguguUfgCfuucgcs | 2780 |
| | cuccccas{invAb} | | usu | |
| D-1842 | [GalNAc3]suugaagCfcAfCfAfUfu | 1933 | usAfsgauuCfcaauguGfgCfuucaas | 2781 |
| | ggaaucus{invAb} | | usu | |
| D-1843 | [GalNAc3]scgguaaCfaAfAfGfAfa | 1934 | asCfscguuCfguucuuUfgUfuaccgs | 2782 |
| | cgaacggs{invAb} | | usu | |
| D-1844 | [GalNAc3]saugaagCfcAfCfUfAfu | 1935 | asCfsugucGfuauaguGfgCfuucaus | 2783 |
| | acgacags{invAb} | | usu | |
| D-1845 | [GalNAc3]scacuauAfcGfAfCfAfg | 1936 | asCfscgguAfccugucGfuAfuagugs | 2784 |
| | guaccggs{invAb} | | usu | |
| D-1846 | [DCA- | 1937 | asAfscuccUfugaaauUfaUfagcaas | 2785 |
| | C6]uugcuaUfaAfUfUfUfcaaggagu | | usu | |
| | s{invAb} | | | |
| D-1847 | {DCA- | 2831 | usUfscuaaGfaguuCfuCfcGfguugc | 3085 |
| | sC6}gcaaccGfgAfGfAfAfcucuuag | | susu | |
| | as{invAb} | | | |
| D-1848 | {DCA- | 2832 | asCfscauaAfgcguAfgAfaCfcgagg | 3086 |
| | sC6}ccucggUfuCfUfAfCfgcuuaug | | susu | |
| | gs{invAb} | | | |
| D-1849 | {DCA- | 2833 | usUfscuaagaguuCfuCfcGfguugcs | 3087 |
| | sC6}gcaaccGfgAfGfAfAfcucuuag | | usu | |
| | as{invAb} | | | |
| D-1850 | {DCA- | 2834 | asGfsuauuUfuaucauCfcAfuugsus | 3088 |
| | sC6}caauGfgAfUfGfAfuaaaauacu | | u | |
| | sus{invAb} | | | |
| D-1851 | {DCA- | 2835 | usGfsaaagAfaugggaGfuUfuggsus | 3089 |
| | sC6}ccaaAfcUfCfCfCfauucuuuca | | u | |
| | sus{invAb} | | | |
| D-1852 | {DCA- | 2836 | asCfsuuucCfauauucAfcAfagaccs | 3090 |
| | sC6}ggucuuGfuGfAfAfUfauggaaa | | usu | |
| | gs{invAb} | | | |
| D-1853 | {DCA- | 2837 | usUfscuacAfgcugauCfgGfauggus | 3091 |
| | sC6}accaucCfgAfUfCfAfgcuguag | | usu | |
| | as{invAb} | | | |
| D-1854 | {DCA- | 2838 | asGfsuagaGfcaaagaAfuUfccaaas | 3092 |
| | sC6}uuuggaAfuUfCfUfUfugcucua | | usu | |
| | cs{invAb} | | | |
| D-1855 | {DCA- | 2839 | usAfsgagcAfaagaauUfcCfaaacus | 3093 |
| | sC6}aguuugGfaAfUfUfCfuuugcuc | | usu | |
| | us{invAb} | | | |
| D-1856 | {DCA- | 2840 | usGfsuaggAfuuauguAfcCfgucuus | 3094 |
| | sC6}aagacgGfuAfCfAfUfaauccua | | usu | |
| | cs{invAb} | | | |
| D-1857 | {DCA- | 2841 | asCfsacuuCfcaacacAfuAfgcucas | 3095 |
| | sC6}ugagcuAfuGfUfGfUfuggaagu | | usu | |
| | gs{invAb} | | | |
| D-1858 | {DCA- | 2842 | usGfsaaagAfaugggaGfuUfuggugs | 3096 |
| | sC6}caccaaAfcUfCfCfCfauucuuu | | usu | |
| | cs{invAb} | | | |
| D-1859 | {DCA- | 2843 | usUfscuaaGfaguucuCfcGfguugcs | 3097 |
| | sC6}gcaaccGfgAfGfAfAfcucuuag | | usu | |
| | as{invAb} | | | |
| D-1860 | {DCA- | 2844 | asCfscauaAfgcguagAfaCfcgaggs | 3098 |
| | sC6}ccucggUfuCfUfAfCfgcuuaug | | usu | |
| | gs{invAb} | | | |
| D-1861 | {DCA- | 2845 | asGfsuauuUfuaucauCfcAfuugggs | 3099 |
| | sC6}cccaauGfgAfUfGfAfuaaaaua | | usu | |
| | cs{invAb} | | | |
| D-1862 | {DCA- | 2846 | asAfscuccUfugaaauUfaUfagcaas | 3100 |
| | sC6}uugcuaUfaAfUfUfUfcaaggag | | usu | |
| | us{invAb} | | | |
| D-1863 | {DCA- | 2847 | usGfsuagGfAfuuauguAfcCfgucuu | 3101 |
| | C6}aagacgGfuAfCfAfUfaauccuac | | susu | |
| | s{invAb} | | | |
| D-1864 | {DCA- | 2848 | usAfsgagcAfaagaAfuUfcCfaaacu | 3102 |
| | C6}aguuugGfaAfUfUfCfuuugcucu | | susu | |
| | s{invAb} | | | |
| D-1865 | {DCA- | 2849 | usGfsuaggAfuuauGfuAfcCfgucuu | 3103 |
| | C6}aagacgGfuAfCfAfUfaauccuac | | susu | |
| | s{invAb} | | | |
| D-1866 | {DCA- | 2850 | usGfsuaggauuauGfuAfcCfgucuus | 3104 |
| | C6}aagacgGfuAfCfAfUfaauccuac | | usu | |
| | s{invAb} | | | |
| D-1867 | {DCA- | 2851 | asCfsuuuccauauUfcAfcAfagaccs | 3105 |
| | C6}ggucuuGfuGfAfAfUfauggaaag | | usu | |
| | s{invAb} | | | |
| D-1868 | {DCA- | 2852 | asGfsuagaGfcaaagaAfuUfccasus | 3106 |
| | C6}uggaAfuUfCfUfUfugcucuacus | | u | |
| | us{invAb} | | | |
| D-1869 | {DCA- | 2853 | usGfsuaggAfuuauguAfcCfgucsus | 3107 |
| | C6}gacgGfuAfCfAfUfaauccuacas | | u | |
| | us{invAb} | | | |
| D-1870 | {DCA- | 2854 | usAfsgagcAfaagaAfuUfcCfaaasu | 3108 |
| | C6}uuugGfaAfUfUfCfuuugcucuas | | su | |
| | us{invAb} | | | |
| D-1871 | {DCA- | 2855 | asGfsuagaGfcaaaGfaAfuUfccasu | 3109 |
| | C6}uggaAfuUfCfUfUfugcucuacus | | su | |
| | us{invAb} | | | |
| D-1872 | {DCA- | 2856 | usGfsuaggAfuuauGfuAfcCfgucsu | 3110 |
| | C6}gacgGfuAfCfAfUfaauccuacas | | su | |
| | us{invAb} | | | |
| D-1873 | {DCA- | 2857 | usUfscuacAfgcugAfuCfgGfaugsu | 3111 |
| | C6}caucCfgAfUfCfAfgcuguagaas | | su | |
| | us{invAb} | | | |
| D-1874 | {DCA- | 2858 | asAfscuccUfugaaAfuUfaUfagcsu | 3112 |
| | C6}gcuauaAfuUfUfCfAfaggaguus | | su | |
| | us{invAb} | | | |
| D-1875 | {DCA- | 2859 | usAfsgagcAfaagaAfuUfcCfaaasu | 3113 |
| | C6}uuuggaAfuUfCfUfUfugcucuas | | su | |
| | us{invAb} | | | |
| D-1876 | {DCA- | 2860 | asGfsuagaGfcaaaGfaAfuUfccasu | 3114 |
| | C6}uggaauUfcUfUfUfGfcucuacus | | su | |
| | us{invAb} | | | |
| D-1877 | {DCA- | 2861 | usGfsuaggAfuuauGfuAfcCfgucsu | 3115 |
| | C6}gacgguAfcAfUfAfAfuccuacas | | su | |
| | us{invAb} | | | |
| D-1878 | {DCA- | 2862 | usGfsuaggAfuuauGfuAfcCfgucsu | 3116 |
| | C6}gsacgGfuAfCfAfUfaauccuaca | | su | |
| | sus{invAb} | | | |
| D-1879 | [GalNAc3]sgsacgGfuAfCfAfUfaa | 2863 | usGfsuaggAfuuauGfuAfcCfgucsu | 3117 |
| | uccuacasus{invAb} | | su | |
| D-1880 | {DCA- | 2864 | usGfsuaggAfuuauGfuAfcCfgucsu | 3118 |
| | sC6}gacgGfuAfCfAfUfaauccuaca | | su | |
| | sus{invAb} | | | |
| D-1881 | {DCA- | 2865 | usGfsuaggauuauGfuAfcCfgucuus | 3119 |
| | sC6}aagacgGfuAfCfAfUfaauccua | | usu | |
| | cs{invAb} | | | |
| D-1882 | {DCA- | 2866 | asGfsuagaGfcaaaGfaAfuUfccasu | 3120 |
| | sC6}uggaAfuUfCfUfUfugcucuacu | | su | |
| | sus{invAb} | | | |
| D-1883 | {DCA- | 2867 | usAfsgagcAfaagaAfuUfcCfaaasu | 3121 |
| | sC6}uuugGfaAfUfUfCfuuugcucua | | su | |
| | sus{invAb} | | | |
| D-1884 | {DCA- | 2868 | usGfsuaggAfuuauGfuAfcCfgucsu | 3122 |
| | sC6}gacgguAfcAfUfAfAfuccuaca | | su | |
| | sus{invAb} | | | |
| D-1885 | {DCA- | 2869 | asGfsuagaGfcaaaGfaAfuUfccasu | 3123 |
| | sC6}uggaauUfcUfUfUfGfcucuacu | | su | |
| | sus{invAb} | | | |
| D-1886 | {DCA- | 2870 | usAfsgagcAfaagaAfuUfcCfaaasu | 3124 |
| | sC6}uuuggaAfuUfCfUfUfugcucua | | su | |
| | sus{invAb} | | | |
| D-1887 | {DCA- | 2871 | usGfsuaggAfuuauguAfcCfgucsus | 3125 |
| | sC6}gacgGfuAfCfAfUfaauccuaca | | u | |
| | sus{invAb} | | | |
| D-1888 | {DCA- | 2872 | asGfsuagaGfcaaagaAfuUfccasus | 3126 |
| | sC6}uggaAfuUfCfUfUfugcucuacu | | u | |
| | sus{invAb} | | | |
| D-1889 | | 2873 | | 3127 |
| D-1890 | | 2874 | | 3128 |
| D-1891 | [GalNAc3]saaucagAfuCfAfUfUfa | 2875 | asUfsaacUfGfguaaugAfuCfugauu | 3129 |
| | ccaguuas{invAb} | | susu | |
| D-1892 | [GalNAc3]scuagucUfgUfCfCfUfu | 2876 | asGfscuaUfUfaaaggaCfaGfacuag | 3130 |
| | uaauagcs{invAb} | | susu | |
| D-1893 | [GalNAc3]sgcacauUfuGfUfUfUfa | 2877 | asAfsuagUfCfuuaaacAfaAfugugc | 3131 |
| | agacuaus{invAb} | | susu | |
| D-1894 | [GalNAc3]sccaaugGfgAfUfUfUfa | 2878 | usGfsuugCfUfguaaauCfcCfauugg | 3132 |
| | cagcaacs{invAb} | | susu | |
| D-1895 | [GalNAc3]sguuugcUfcUfUfAfAfu | 2879 | usCfscauAfCfgauuaaGfaGfcaaac | 3133 |
| | cguauggs{invAb} | | susu | |
| D-1896 | [GalNAc3]scccucgUfaUfGfUfUfu | 2880 | asAfscuuUfCfaaaacaUfaCfgaggg | 3134 |
| | ugaaagus{invAb} | | susu | |
| D-1897 | [GalNAc3]saaucagAfuCfAfUfUfa | 2881 | asUfsaacuGfguaaUfgAfuCfugauu | 3135 |
| | ccaguuas{invAb} | | susu | |
| D-1898 | [GalNAc3]scuagucUfgUfCfCfUfu | 2882 | asGfscuauUfaaagGfaCfaGfacuag | 3136 |
| | uaauagcs{invAb} | | susu | |
| D-1899 | [GalNAc3]sgcacauUfuGfUfUfUfa | 2883 | asAfsuaguCfuuaaAfcAfaAfugugc | 3137 |
| | agacuaus{invAb} | | susu | |
| D-1900 | [GalNAc3]sccaaugGfgAfUfUfUfa | 2884 | usGfsuugcUfguaaAfuCfcCfauugg | 3138 |
| | cagcaacs{invAb} | | susu | |
| D-1901 | [GalNAc3]sguuugcUfcUfUfAfAfu | 2885 | usCfscauaCfgauuAfaGfaGfcaaac | 3139 |
| | cguauggs{invAb} | | susu | |
| D-1902 | [GalNAc3]scccucgUfaUfGfUfUfu | 2886 | asAfscuuuCfaaaaCfaUfaCfgaggg | 3140 |
| | ugaaagus{invAb} | | susu | |
| D-1903 | [GalNAc3]saaucagAfuCfAfUfUfa | 2887 | asUfsaacugguaaUfgAfuCfugauus | 3141 |
| | ccaguuas{invAb} | | usu | |
| D-1904 | [GalNAc3]scuagucUfgUfCfCfUfu | 2888 | asGfscuauuaaagGfaCfaGfacuags | 3142 |
| | uaauagcs{invAb} | | usu | |
| D-1905 | [GalNAc3]sgcacauUfuGfUfUfUfa | 2889 | asAfsuagucuuaaAfcAfaAfugugcs | 3143 |
| | agacuaus{invAb} | | usu | |
| D-1906 | [GalNAc3]sccaaugGfgAfUfUfUfa | 2890 | usGfsuugcuguaaAfuCfcCfauuggs | 3144 |
| | cagcaacs{invAb} | | usu | |
| D-1907 | [GalNAc3]sguuugcUfcUfUfAfAfu | 2891 | usCfscauacgauuAfaGfaGfcaaacs | 3145 |
| | cguauggs{invAb} | | usu | |
| D-1908 | [GalNAc3]scccucgUfaUfGfUfUfu | 2892 | asAfscuuucaaaaCfaUfaCfgagggs | 3146 |
| | ugaaagus{invAb} | | usu | |
| D-1909 | [GalNAc3]sucagAfuCfAfUfUfacc | 2893 | asUfsaacuGfguaaugAfuCfugasus | 3147 |
| | aguuausus{invAb} | | u | |
| D-1910 | [GalNAc3]sagucUfgUfCfCfUfuua | 2894 | asGfscuauUfaaaggaCfaGfacusus | 3148 |
| | auagcusus{invAb} | | u | |
| D-1911 | [GalNAc3]sacauUfuGfUfUfUfaag | 2895 | asAfsuaguCfuuaaacAfaAfugusus | 3149 |
| | acuauusus{invAb} | | u | |
| D-1912 | [GalNAc3]saaugGfgAfUfUfUfaca | 2896 | usGfsuugcUfguaaauCfcCfauusus | 3150 |
| | gcaacasus{invAb} | | u | |
| D-1913 | [GalNAc3]suugcUfcUfUfAfAfucg | 2897 | usCfscauaCfgauuaaGfaGfcaasus | 3151 |
| | uauggasus{invAb} | | u | |
| D-1914 | [GalNAc3]scucgUfaUfGfUfUfuug | 2898 | asAfscuuuCfaaaacaUfaCfgagsus | 3152 |
| | aaaguusus{invAb} | | u | |
| D-1915 | [GalNAc3]sucagAfuCfAfUfUfacc | 2899 | asUfsaacuGfguaaUfgAfuCfugasu | 3153 |
| | aguuausus{invAb} | | su | |
| D-1916 | [GalNAc3]sagucUfgUfCfCfUfuua | 2900 | asGfscuauUfaaagGfaCfaGfacusu | 3154 |
| | auagcusus{invAb} | | su | |
| D-1917 | [GalNAc3]sacauUfuGfUfUfUfaag | 2901 | asAfsuaguCfuuaaAfcAfaAfugusu | 3155 |
| | acuauusus{invAb} | | su | |
| D-1918 | [GalNAc3]saaugGfgAfUfUfUfaca | 2902 | usGfsuugcUfguaaAfuCfcCfauusu | 3156 |
| | gcaacasus{invAb} | | su | |
| D-1919 | [GalNAc3]suugcUfcUfUfAfAfucg | 2903 | usCfscauaCfgauuAfaGfaGfcaasu | 3157 |
| | uauggasus{invAb} | | su | |
| D-1920 | [GalNAc3]scucgUfaUfGfUfUfuug | 2904 | asAfscuuuCfaaaaCfaUfaCfgagsu | 3158 |
| | aaaguusus{invAb} | | su | |
| D-1921 | [GalNAc3]sucagauCfaUfUfAfCfc | 2905 | asUfsaacuGfguaaUfgAfuCfugasu | 3159 |
| | aguuausus{invAb} | | su | |
| D-1922 | [GalNAc3]sagucugUfcCfUfUfUfa | 2906 | asGfscuauUfaaagGfaCfaGfacusu | 3160 |
| | auagcusus{invAb} | | su | |
| D-1923 | [GalNAc3]sacauuuGfuUfUfAfAfg | 2907 | asAfsuaguCfuuaaAfcAfaAfugusu | 3161 |
| | acuauusus{invAb} | | su | |
| D-1924 | [GalNAc3]saaugggAfuUfUfAfCfa | 2908 | usGfsuugcUfguaaAfuCfcCfauusu | 3162 |
| | gcaacasus{invAb} | | su | |
| D-1925 | [GalNAc3]suugcucUfuAfAfUfCfg | 2909 | usCfscauaCfgauuAfaGfaGfcaasu | 3163 |
| | uauggasus{invAb} | | su | |
| D-1926 | [GalNAc3]scucguaUfgUfUfUfUfg | 2910 | asAfscuuuCfaaaaCfaUfaCfgagsu | 3164 |
| | aaaguusus{invAb} | | su | |
| D-1927 | [GalNAc3]sasasucagAfuCfAfUfU | 2911 | asUfsaacuGfgUfaaugAfuCfugauu | 3165 |
| | faccaguusas{invAb} | | susu | |
| D-1928 | [GalNAc3]scsusagucUfgUfCfCfU | 2912 | asGfscuauUfaAfaggaCfaGfacuag | 3166 |
| | fuuaauagscs{invAb} | | susu | |
| D-1929 | [GalNAc3]sgscsacauUfuGfUfUfU | 2913 | asAfsuaguCfuUfaaacAfaAfugugc | 3167 |
| | faagacuasus{invAb} | | susu | |
| D-1930 | [GalNAc3]scscsaaugGfgAfUfUfU | 2914 | usGfsuugcUfgUfaaauCfcCfauugg | 3168 |
| | facagcaascs{invAb} | | susu | |
| D-1931 | [GalNAc3]sgsusuugcUfcUfUfAfA | 2915 | usCfscauaCfgAfuuaaGfaGfcaaac | 3169 |
| | fucguaugsgs{invAb} | | susu | |
| D-1932 | [GalNAc3]scscscucgUfaUfGfUfU | 2916 | asAfscuuuCfaAfaacaUfaCfgaggg | 3170 |
| | fuugaaagsus{invAb} | | susu | |
| D-1933 | [GalNAc3]saagaaaAfuAfCfUfCfc | 2917 | asCfsccaGfAfaggaguAfuUfuucuu | 3171 |
| | uucugggs{invAb} | | susu | |
| D-1934 | [GalNAc3]sucagauCfaUfUfAfCfc | 2918 | asGfscuaAfCfugguaaUfgAfucuga | 3172 |
| | aguuagcs{invAb} | | susu | |
| D-1935 | [GalNAc3]saccuaaCfuCfCfCfAfg | 2919 | asCfscgcAfUfccugggAfgUfuaggu | 3173 |
| | gaugcggs{invAb} | | susu | |
| D-1936 | [GalNAc3]saagcacAfuUfUfGfUfu | 2920 | usAfsgucUfUfaaacaaAfuGfugcuu | 3174 |
| | uaagacus{invAb} | | susu | |
| D-1937 | [GalNAc3]sugcuguGfuGfGfCfCfa | 2921 | asUfsuauAfAfuuggccAfcAfcagca | 3175 |
| | auuauaas{invAb} | | susu | |
| D-1938 | [GalNAc3]sucugaaAfuGfGfAfCfa | 2922 | asGfsgucAfUfuuguccAfuUfucaga | 3176 |
| | aaugaccs{invAb} | | susu | |
| D-1939 | [GalNAc3]saagaaaAfuAfCfUfCfc | 2923 | asCfsccagAfaggaGfuAfuUfuucuu | 3177 |
| | uucugggs{invAb} | | susu | |
| D-1940 | [GalNAc3]sucagauCfaUfUfAfCfc | 2924 | asGfscuaaCfugguAfaUfgAfucuga | 3178 |
| | aguuagcs{invAb} | | susu | |
| D-1941 | [GalNAc3]saccuaaCfuCfCfCfAfg | 2925 | asCfscgcaUfccugGfgAfgUfuaggu | 3179 |
| | gaugcggs{invAb} | | susu | |
| D-1942 | [GalNAc3]saagcacAfuUfUfGfUfu | 2926 | usAfsgucuUfaaacAfaAfuGfugcuu | 3180 |
| | uaagacus{invAb} | | susu | |
| D-1943 | [GalNAc3]sugcuguGfuGfGfCfCfa | 2927 | asUfsuauaAfuuggCfcAfcAfcagca | 3181 |
| | auuauaas{invAb} | | susu | |
| D-1944 | [GalNAc3]sucugaaAfuGfGfAfCfa | 2928 | asGfsgucaUfuuguCfcAfuUfucaga | 3182 |
| | aaugaccs{invAb} | | susu | |
| D-1945 | [GalNAc3]saagaaaAfuAfCfUfCfc | 2929 | asCfsccagaaggaGfuAfuUfuucuus | 3183 |
| | uucugggs{invAb} | | usu | |
| D-1946 | [GalNAc3]sucagauCfaUfUfAfCfc | 2930 | asGfscuaacugguAfaUfgAfucugas | 3184 |
| | aguuagcs{invAb} | | usu | |
| D-1947 | [GalNAc3]saccuaaCfuCfCfCfAfg | 2931 | asCfscgcauccugGfgAfgUfuaggus | 3185 |
| | gaugcggs{invAb} | | usu | |
| D-1948 | [GalNAc3]saagcacAfuUfUfGfUfu | 2932 | usAfsgucuuaaacAfaAfuGfugcuus | 3186 |
| | uaagacus{invAb} | | usu | |
| D-1949 | [GalNAc3]sugcuguGfuGfGfCfCfa | 2933 | asUfsuauaauuggCfcAfcAfcagcas | 3187 |
| | auuauaas{invAb} | | usu | |
| D-1950 | [GalNAc3]sucugaaAfuGfGfAfCfa | 2934 | asGfsgucauuuguCfcAfuUfucagas | 3188 |
| | aaugaccs{invAb} | | usu | |
| D-1951 | [GalNAc3]sgaaaAfuAfCfUfCfcuu | 2935 | asCfsccagAfaggaguAfuUfuucsus | 3189 |
| | cugggusus{invAb} | | u | |
| D-1952 | [GalNAc3]sagauCfaUfUfAfCfcag | 2936 | asGfscuaaCfugguaaUfgAfucusus | 3190 |
| | uuagcusus{invAb} | | u | |
| D-1953 | [GalNAc3]scuaaCfuCfCfCfAfgga | 2937 | asCfscgcaUfccugggAfgUfuagsus | 3191 |
| | ugcggusus{invAb} | | u | |
| D-1954 | [GalNAc3]sgcacAfuUfUfGfUfuua | 2938 | usAfsgucuUfaaacaaAfuGfugcsus | 3192 |
| | agacuasus{invAb} | | u | |
| D-1955 | [GalNAc3]scuguGfuGfGfCfCfaau | 2939 | asUfsuauaAfuuggccAfcAfcagsus | 3193 |
| | uauaausus{invAb} | | u | |
| D-1956 | [GalNAc3]sugaaAfuGfGfAfCfaaa | 2940 | asGfsgucaUfuuguccAfuUfucasus | 3194 |
| | ugaccusus{invAb} | | u | |
| D-1957 | [GalNAc3]sgaaaAfuAfCfUfCfcuu | 2941 | asCfsccagAfaggaGfuAfuUfuucsu | 3195 |
| | cugggusus{invAb} | | su | |
| D-1958 | [GalNAc3]sagauCfaUfUfAfCfcag | 2942 | asGfscuaaCfugguAfaUfgAfucusu | 3196 |
| | uuagcusus{invAb} | | su | |
| D-1959 | [GalNAc3]scuaaCfuCfCfCfAfgga | 2943 | asCfscgcaUfccugGfgAfgUfuagsu | 3197 |
| | ugcggusus{invAb} | | su | |
| D-1960 | [GalNAc3]sgcacAfuUfUfGfUfuua | 2944 | usAfsgucuUfaaacAfaAfuGfugcsu | 3198 |
| | agacuasus{invAb} | | su | |
| D-1961 | [GalNAc3]scuguGfuGfGfCfCfaau | 2945 | asUfsuauaAfuuggCfcAfcAfcagsu | 3199 |
| | uauaausus{invAb} | | su | |
| D-1962 | [GalNAc3]sugaaAfuGfGfAfCfaaa | 2946 | asGfsgucaUfuuguCfcAfuUfucasu | 3200 |
| | ugaccusus{invAb} | | su | |
| D-1963 | [GalNAc3]sgaaaauAfcUfCfCfUfu | 2947 | asCfsccagAfaggaGfuAfuUfuucsu | 3201 |
| | cugggusus{invAb} | | su | |
| D-1964 | [GalNAc3]sagaucaUfuAfCfCfAfg | 2948 | asGfscuaaCfugguAfaUfgAfucusu | 3202 |
| | uuagcusus{invAb} | | su | |
| D-1965 | [GalNAc3]scuaacuCfcCfAfGfGfa | 2949 | asCfscgcaUfccugGfgAfgUfuagsu | 3203 |
| | ugcggusus{invAb} | | su | |
| D-1966 | [GalNAc3]sgcacauUfuGfUfUfUfa | 2950 | usAfsgucuUfaaacAfaAfuGfugcsu | 3204 |
| | agacuasus{invAb} | | su | |
| D-1967 | [GalNAc3]scuguguGfgCfCfAfAfu | 2951 | asUfsuauaAfuuggCfcAfcAfcagsu | 3205 |
| | uauaausus{invAb} | | su | |
| D-1968 | [GalNAc3]sugaaauGfgAfCfAfAfa | 2952 | asGfsgucaUfuuguCfcAfuUfucasu | 3206 |
| | ugaccusus{invAb} | | su | |
| D-1969 | [GalNAc3]sasasgaaaAfuAfCfUfC | 2953 | asCfsccagAfaGfgaguAfuUfuucuu | 3207 |
| | fcuucuggsgs{invAb} | | susu | |
| D-1970 | [GalNAc3]suscsagauCfaUfUfAfC | 2954 | asGfscuaaCfuGfguaaUfgAfucuga | 3208 |
| | fcaguuagscs{invAb} | | susu | |
| D-1971 | [GalNAc3]sascscuaaCfuCfCfCfA | 2955 | asCfscgcaUfcCfugggAfgUfuaggu | 3209 |
| | fggaugcgsgs{invAb} | | susu | |
| D-1972 | [GalNAc3]sasasgcacAfuUfUfGfU | 2956 | usAfsgucuUfaAfacaaAfuGfugcuu | 3210 |
| | fuuaagacsus{invAb} | | susu | |
| D-1973 | [GalNAc3]susgscuguGfuGfGfCfC | 2957 | asUfsuauaAfuUfggccAfcAfcagca | 3211 |
| | faauuauasas{invAb} | | susu | |
| D-1974 | [GalNAc3]suscsugaaAfuGfGfAfC | 2958 | asGfsgucaUfuUfguccAfuUfucaga | 3212 |
| | faaaugacscs{invAb} | | susu | |
| D-1975 | {DCA- | 2959 | asAfsuaguCfuuaaacAfaAfugugcs | 3213 |
| | C6}gcacauUfuGfUfUfUfaagacuau | | usu | |
| | s{invAb} | | | |
| D-1976 | {DCA- | 2960 | asAfsuaguCfuuaaacAfaAfugugcs | 3214 |
| | sC6}gcacauUfuGfUfUfUfaagacua | | usu | |
| | us{invAb} | | | |
| D-1977 | [GalNAc3]sgscacauUfuGfUfUfUf | 2961 | asAfsuaguCfuuaaacAfaAfugugcs | 3215 |
| | aagacuaus{invAb} | | usu | |
| D-1978 | [GalNAc3]sasagacgGfuAfCfAfUf | 2962 | usGfsuaggAfuuauguAfcCfgucuus | 3216 |
| | aauccuacs{invAb} | | usu | |
| D-1979 | [GalNAc3]susuuggaAfuUfCfUfUf | 2963 | asGfsuagaGfcaaagaAfuUfccaaas | 3217 |
| | ugcucuacs{invAb} | | usu | |
| D-1980 | [GalNAc3]sasguuugGfaAfUfUfCf | 2964 | usAfsgagcAfaagaauUfcCfaaacus | 3218 |
| | uuugcucus{invAb} | | usu | |
| D-1981 | [GalNAc3]susugcuaUfaAfUfUfUf | 2965 | asAfscuccUfugaaauUfaUfagcaas | 3219 |
| | caaggagus{invAb} | | usu | |
| D-1982 | [GalNAc3]sascauUfuGfUfUfUfaa | 2966 | asAfsuaguCfuuaaAfcAfaAfugusu | 3220 |
| | gacuauusus{invAb} | | su | |
| D-1983 | [GalNAc3]susggaAfuUfCfUfUfug | 2967 | asGfsuagaGfcaaaGfaAfuUfccasu | 3221 |
| | cucuacusus{invAb} | | su | |
| D-1984 | [GalNAc3]susuugGfaAfUfUfCfuu | 2968 | usAfsgagcAfaagaAfuUfcCfaaasu | 3222 |
| | ugcucuasus{invAb} | | su | |
| D-1985 | [GalNAc3]sgscuaUfaAfUfUfUfca | 2969 | asAfscuccUfugaaAfuUfaUfagcsu | 3223 |
| | aggaguusus{invAb} | | su | |
| D-1986 | [GalNAc3]sascauuuGfuUfUfAfAf | 2970 | asAfsuaguCfuuaaAfcAfaAfugusu | 3224 |
| | gacuauusus{invAb} | | su | |
| D-1987 | [GalNAc3]sgsacgguAfcAfUfAfAf | 2971 | usGfsuaggAfuuauGfuAfcCfgucsu | 3225 |
| | uccuacasus{invAb} | | su | |
| D-1988 | [GalNAc3]susggaauUfcUfUfUfGf | 2972 | asGfsuagaGfcaaaGfaAfuUfccasu | 3226 |
| | cucuacusus{invAb} | | su | |
| D-1989 | [GalNAc3]susuuggaAfuUfCfUfUf | 2973 | usAfsgagcAfaagaAfuUfcCfaaasu | 3227 |
| | ugcucuasus{invAb} | | su | |
| D-1990 | [GalNAc3]sgscuauaAfuUfUfCfAf | 2974 | asAfscuccUfugaaAfuUfaUfagcsu | 3228 |
| | aggaguusus{invAb} | | su | |
| D-1991 | [GalNAc3]sascauUfuGfUfUfUfaa | 2975 | asAfsuaguCfuuaaacAfaAfugusus | 3229 |
| | gacuauusus{invAb} | | u | |
| D-1992 | [GalNAc3]sgsacgGfuAfCfAfUfaa | 2976 | usGfsuaggAfuuauguAfcCfgucsus | 3230 |
| | uccuacasus{invAb} | | u | |
| D-1993 | [GalNAc3]susggaAfuUfCfUfUfug | 2977 | asGfsuagaGfcaaagaAfuUfccasus | 3231 |
| | cucuacusus{invAb} | | u | |
| D-1994 | [GalNAc3]susuugGfaAfUfUfCfuu | 2978 | usAfsgagcAfaagaauUfcCfaaasus | 3232 |
| | ugcucuasus{invAb} | | u | |
| D-1995 | [GalNAc3]sgscuaUfaAfUfUfUfca | 2979 | asAfscuccUfugaaauUfaUfagcsus | 3233 |
| | aggaguusus{invAb} | | u | |
| D-1996 | [GalNAc3]sascauuuGfuUfUfAfAf | 2980 | asAfsuaguCfuuAfaAfcAfaaugusu | 3234 |
| | gacuauusus{invAb} | | su | |
| D-1997 | [GalNAc3]sgsacgguAfcAfUfAfAf | 2981 | usGfsuaggAfuuAfuGfuAfccgucsu | 3235 |
| | uccuacasus{invAb} | | su | |
| D-1998 | [GalNAc3]susggaauUfcUfUfUfGf | 2982 | asGfsuagaGfcaAfaGfaAfuuccasu | 3236 |
| | cucuacusus{invAb} | | su | |
| D-1999 | [GalNAc3]susuuggaAfuUfCfUfUf | 2983 | usAfsgagcAfaaGfaAfuUfccaaasu | 3237 |
| | ugcucuasus{invAb} | | su | |
| D-2000 | [GalNAc3]sgscuauaAfuUfUfCfAf | 2984 | asAfscuccUfugAfaAfuUfauagcsu | 3238 |
| | aggaguusus{invAb} | | su | |
| D-2001 | {DCA- | 2985 | asAfsuaguCfuuaaAfcAfaAfugusu | 3239 |
| | sC6}ascauUfuGfUfUfUfaagacuau | | su | |
| | usus{invAb} | | | |
| D-2002 | {DCA- | 2986 | usGfsuaggAfuuauGfuAfcCfgucsu | 3240 |
| | sC6}gsacgGfuAfCfAfUfaauccuac | | su | |
| | asus{invAb} | | | |
| D-2003 | {DCA- | 2987 | asGfsuagaGfcaaaGfaAfuUfccasu | 3241 |
| | sC6}usggaAfuUfCfUfUfugcucuac | | su | |
| | usus{invAb} | | | |
| D-2004 | {DCA- | 2988 | usAfsgagcAfaagaAfuUfcCfaaasu | 3242 |
| | sC6}usuugGfaAfUfUfCfuuugcucu | | su | |
| | asus{invAb} | | | |
| D-2005 | {DCA- | 2989 | asAfscuccUfugaaAfuUfaUfagcsu | 3243 |
| | sC6}gscuaUfaAfUfUfUfcaaggagu | | su | |
| | usus{invAb} | | | |
| D-2006 | [GalNAc3]sascscaucCfgAfUfCfA | 2990 | usUfscuacAfgCfugauCfgGfauggu | 3244 |
| | fgcuguagsas{invAb} | | susu | |
| D-2007 | [GalNAc3]sgsgsucuuGfuGfAfAfU | 2991 | asCfsuuucCfaUfauucAfcAfagacc | 3245 |
| | fauggaaasgs{invAb} | | susu | |
| D-2008 | [GalNAc3]sasasgacgGfuAfCfAfU | 2992 | usGfsuaggAfuUfauguAfcCfgucuu | 3246 |
| | faauccuascs{invAb} | | susu | |
| D-2009 | [GalNAc3]sgscsaaccGfgAfGfAfA | 2993 | usUfscuaaGfaGfuucuCfcGfguugc | 3247 |
| | fcucuuagsas{invAb} | | susu | |
| D-2010 | [GalNAc3]scsasccaaAfcUfCfCfC | 2994 | usGfsaaagAfaUfgggaGfuUfuggug | 3248 |
| | fauucuuuscs{invAb} | | susu | |
| D-2011 | [GalNAc3]susgsagcuAfuGfUfGfU | 2995 | asCfsacuuCfcAfacacAfuAfgcuca | 3249 |
| | fuggaagusgs{invAb} | | susu | |
| D-2012 | [GalNAc3]sususuggaAfuUfCfUfU | 2996 | asGfsuagaGfcAfaagaAfuUfccaaa | 3250 |
| | fugcucuascs{invAb} | | susu | |
| D-2013 | [GalNAc3]sasgsuuugGfaAfUfUfC | 2997 | usAfsgagcAfaAfgaauUfcCfaaacu | 3251 |
| | fuuugcucsus{invAb} | | susu | |
| D-2014 | [GalNAc3]sususgcuaUfaAfUfUfU | 2998 | asAfscuccUfuGfaaauUfaUfagcaa | 3252 |
| | fcaaggagsus{invAb} | | susu | |
| D-2015 | [GalNAc3]sasccauccgAfuCfAfGf | 2999 | usUfscuacAfgcugAfuCfggauggus | 3253 |
| | Cfuguagas{invAb} | | usu | |
| D-2016 | [GalNAc3]sgsgucuuguGfaAfUfAf | 3000 | asCfsuuucCfauauUfcAfcaagaccs | 3254 |
| | Ufggaaags{invAb} | | usu | |
| D-2017 | [GalNAc3]sasagacgguAfcAfUfAf | 3001 | usGfsuaggAfuuauGfuAfccgucuus | 3255 |
| | Afuccuacs{invAb} | | usu | |
| D-2018 | [GalNAc3]sgscaaccggAfgAfAfCf | 3002 | usUfscuaaGfaguuCfuCfcgguugcs | 3256 |
| | Ufcuuagas{invAb} | | usu | |
| D-2019 | [GalNAc3]scsaccaaacUfcCfCfAf | 3003 | usGfsaaagAfauggGfaGfuuuggugs | 3257 |
| | Ufucuuucs{invAb} | | usu | |
| D-2020 | [GalNAc3]susgagcuauGfuGfUfUf | 3004 | asCfsacuuCfcaacAfcAfuagcucas | 3258 |
| | Gfgaagugs{invAb} | | usu | |
| D-2021 | [GalNAc3]susuuggaauUfcUfUfUf | 3005 | asGfsuagaGfcaaaGfaAfuuccaaas | 3259 |
| | Gfcucuacs{invAb} | | usu | |
| D-2022 | [GalNAc3]sasguuuggaAfuUfCfUf | 3006 | usAfsgagcAfaagaAfuUfccaaacus | 3260 |
| | Ufugcucus{invAb} | | usu | |
| D-2023 | [GalNAc3]susugcuauaAfuUfUfCf | 3007 | asAfscuccUfugaaAfuUfauagcaas | 3261 |
| | Afaggagus{invAb} | | usu | |
| D-2024 | [GalNAc3]sauccggAfaGfUfUfUfg | 3008 | usCfsuaucuucaaAfcUfuCfcggaus | 3262 |
| | aagauags{invAb} | | usu | |
| D-2025 | [GalNAc3]sccggAfaGfUfUfUfgaa | 3009 | usCfsuaucUfucaaacUfuCfcggsus | 3263 |
| | gauagasus{invAb} | | u | |
| D-2026 | [GalNAc3]sccggAfaGfUfUfUfgaa | 3010 | usCfsuaucUfucaaAfcUfuCfcggsu | 3264 |
| | gauagasus{invAb} | | su | |
| D-2027 | [GalNAc3]sccggaaGfuUfUfGfAfa | 3011 | usCfsuaucUfucaaAfcUfuCfcggsu | 3265 |
| | gauagasus{invAb} | | su | |
| D-2028 | [GalNAc3]scscggAfaGfUfUfUfga | 3012 | usCfsuaucUfucaaAfcUfuCfcggsu | 3266 |
| | agauagasus{invAb} | | su | |
| D-2029 | [GalNAc3]sasusccggAfaGfUfUfU | 3013 | usCfsuaucUfuCfaaacUfuCfcggau | 3267 |
| | fgaagauasgs{invAb} | | susu | |
| D-2030 | [GalNAc3]sasuccggAfaGfUfUfUf | 3014 | usCfsuaucUfucaaacUfuCfcggaus | 3268 |
| | gaagauags{invAb} | | usu | |
| D-2031 | [GalNAc3]scscggaaGfuUfUfGfAf | 3015 | usCfsuaucUfucaaAfcUfuCfcggsu | 3269 |
| | agauagasus{invAb} | | su | |
| D-2032 | [GalNAc3]scscggAfaGfUfUfUfga | 3016 | usCfsuaucUfucaaacUfuCfcggsus | 3270 |
| | agauagasus{invAb} | | u | |
| D-2033 | [GalNAc3]scscggaaGfuUfUfGfAf | 3017 | usCfsuaucUfucAfaAfcUfuccggsu | 3271 |
| | agauagasus{invAb} | | su | |
| D-2034 | [GalNAc3]sasuccggaaGfuUfUfGf | 3018 | usCfsuaucUfucaaAfcUfuccggaus | 3272 |
| | Afagauags{invAb} | | usu | |
| D-2035 | {DCA- | 3019 | asGfsgucaUfuUfguccAfuUfucaga | 3273 |
| | sC6}uscsugaaAfuGfGfAfCfaaaug | | susu | |
| | acscs{invAb} | | | |
| D-2036 | [GalNAc3]scsacaaagaCfgGfUfAf | 3020 | asGfsauuaUfguacCfgUfcuuugugs | 3274 |
| | Cfauaaucs{invAb} | | usu | |
| D-2037 | [GalNAc3]sascaaagacGfgUfAfCf | 3021 | asGfsgauuAfuguaCfcGfucuuugus | 3275 |
| | Afuaauccs{invAb} | | usu | |
| D-2038 | [GalNAc3]scsaaagacgGfuAfCfAf | 3022 | usAfsggauUfauguAfcCfgucuuugs | 3276 |
| | Ufaauccus{invAb} | | usu | |
| D-2039 | [GalNAc3]sasaagacggUfaCfAfUf | 3023 | asUfsaggaUfuaugUfaCfcgucuuus | 3277 |
| | Afauccuas{invAb} | | usu | |
| D-2040 | [GalNAc3]susaguuuggAfaUfUfCf | 3024 | asGfsagcaAfagaaUfuCfcaaacuas | 3278 |
| | Ufuugcucs{invAb} | | usu | |
| D-2041 | [GalNAc3]scsuguguGfgCfCfAfAf | 3025 | asUfsuauaAfuuGfgccAfcAfcagsu | 3279 |
| | uuauaausus{invAb} | | su | |
| D-2042 | [GalNAc3]sgscacauUfuGfUfUfUf | 3026 | usAfsgucuUfaaAfcaaAfuGfugcsu | 3280 |
| | aagacuasus{invAb} | | su | |
| D-2043 | [GalNAc3]susggaauUfcUfUfUfGf | 3027 | asGfsuagaGfcaAfagaAfuUfccasu | 3281 |
| | cucuacusus{invAb} | | su | |
| D-2044 | [GalNAc3]susuuggaAfuUfCfUfUf | 3028 | usAfsgagcAfaaGfaauUfcCfaaasu | 3282 |
| | ugcucuasus{invAb} | | su | |
| D-2045 | [GalNAc3]scsuguguGfgCfCfAfAf | 3029 | asUfsuauaAfuuggccAfcAfcagsus | 3283 |
| | uuauaausus{invAb} | | u | |
| D-2046 | [GalNAc3]sgscacauUfuGfUfUfUf | 3030 | usAfsgucuUfaaacaaAfuGfugcsus | 3284 |
| | aagacuasus{invAb} | | u | |
| D-2047 | [GalNAc3]susggaauUfcUfUfUfGf | 3031 | asGfsuagaGfcaaagaAfuUfccasus | 3285 |
| | cucuacusus{invAb} | | u | |
| D-2048 | [GalNAc3]susuuggaAfuUfCfUfUf | 3032 | usAfsgagcAfaagaauUfcCfaaasus | 3286 |
| | ugcucuasus{invAb} | | u | |
| D-2049 | [GalNAc3]sgsacgguAfcAfUfAfAf | 3033 | usGfsuaggAfuuauguAfcCfgucsus | 3287 |
| | uccuacasus{invAb} | | u | |
| D-2050 | [GalNAc3]scsuguGfuGfGfCfCfaa | 3034 | asUfsuauaAfuUfggccAfcAfcagsu | 3288 |
| | uuauaausus{invAb} | | su | |
| D-2051 | [GalNAc3]sgscacAfuUfUfGfUfuu | 3035 | usAfsgucuUfaAfacaaAfuGfugcsu | 3289 |
| | aagacuasus{invAb} | | su | |
| D-2052 | [GalNAc3]susggaAfuUfCfUfUfug | 3036 | asGfsuagaGfcAfaagaAfuUfccasu | 3290 |
| | cucuacusus{invAb} | | su | |
| D-2053 | [GalNAc3]susuugGfaAfUfUfCfuu | 3037 | usAfsgagcAfaAfgaauUfcCfaaasu | 3291 |
| | ugcucuasus{invAb} | | su | |
| D-2054 | [GalNAc3]sgsacgGfuAfCfAfUfaa | 3038 | usGfsuaggAfuUfauguAfcCfgucsu | 3292 |
| | uccuacasus{invAb} | | su | |
| D-2055 | [GalNAc3]suscugaaauGfgAfCfAf | 3039 | asGfsgucaUfuuguCfcAfuuucagas | 3293 |
| | Afaugaccs{invAb} | | usu | |
| D-2056 | [GalNAc3]susgaaAfuGfGfAfCfaa | 3040 | asGfsgucaUfuuguCfcAfuUfucasu | 3294 |
| | augaccusus{invAb} | | su | |
| D-2057 | [GalNAc3]susgcuguguGfgCfCfAf | 3041 | asUfsuauaAfuuggCfcAfcacagcas | 3295 |
| | Afuuauaas{invAb} | | usu | |
| D-2058 | [GalNAc3]scsuguGfuGfGfCfCfaa | 3042 | asUfsuauaAfuuggCfcAfcAfcagsu | 3296 |
| | uuauaausus{invAb} | | su | |
| D-2059 | [GalNAc3]susgcuguGfuGfGfCfCf | 3043 | asUfsuauaAfuuggccAfcAfcagcas | 3297 |
| | aauuauaas{invAb} | | usu | |
| D-2060 | [GalNAc3]scsuguguGfgCfCfAfAf | 3044 | asUfsuauaAfuuggCfcAfcAfcagsu | 3298 |
| | uuauaausus{invAb} | | su | |
| D-2061 | [GalNAc3]scsuguguGfgCfCfAfAf | 3045 | asUfsuauaAfuuGfgCfcAfcacagsu | 3299 |
| | uuauaausus{invAb} | | su | |
| D-2062 | [GalNAc3]scsccucguaUfgUfUfUf | 3046 | asAfscuuuCfaaaaCfaUfacgagggs | 3300 |
| | Ufgaaagus{invAb} | | usu | |
| D-2063 | [GalNAc3]scsucgUfaUfGfUfUfuu | 3047 | asAfscuuuCfaaaaCfaUfaCfgagsu | 3301 |
| | gaaaguusus{invAb} | | su | |
| D-2064 | [GalNAc3]scscaaugggAfuUfUfAf | 3048 | usGfsuugcUfguaaAfuCfccauuggs | 3302 |
| | Cfagcaacs{invAb} | | usu | |
| D-2065 | [GalNAc3]sasaugGfgAfUfUfUfac | 3049 | usGfsuugcUfguaaAfuCfcCfauusu | 3303 |
| | agcaacasus{invAb} | | su | |
| D-2066 | [GalNAc3]scscaaugGfgAfUfUfUf | 3050 | usGfsuugcUfguaaauCfcCfauuggs | 3304 |
| | acagcaacs{invAb} | | usu | |
| D-2067 | [GalNAc3]sasaugggAfuUfUfAfCf | 3051 | usGfsuugcUfguaaAfuCfcCfauusu | 3305 |
| | agcaacasus{invAb} | | su | |
| D-2068 | [GalNAc3]sasaugggAfuUfUfAfCf | 3052 | usGfsuugcUfguAfaAfuCfccauusu | 3306 |
| | agcaacasus{invAb} | | su | |
| D-2069 | [GalNAc3]sgsuuugcucUfuAfAfUf | 3053 | usCfscauaCfgauuAfaGfagcaaacs | 3307 |
| | Cfguauggs{invAb} | | usu | |
| D-2070 | [GalNAc3]susugcUfcUfUfAfAfuc | 305 | usCfscauaCfgauuAfaGfaGfcaasu | 3308 |
| | guauggasus{invAb} | | su | |
| D-2071 | [GalNAc3]sgsuuugcUfcUfUfAfAf | 3055 | usCfscauaCfgauuaaGfaGfcaaacs | 3309 |
| | ucguauggs{invAb} | | usu | |
| D-2072 | [GalNAc3]susugcucUfuAfAfUfCf | 3056 | usCfscauaCfgauuAfaGfaGfcaasu | 3310 |
| | guauggasus{invAb} | | su | |
| D-2073 | [GalNAc3]susugcUfcUfUfAfAfuc | 3057 | usCfscauaCfgauuaaGfaGfcaasus | 3311 |
| | guauggasus{invAb} | | u | |
| D-2074 | [GalNAc3]susugcucUfuAfAfUfCf | 3058 | usCfscauaCfgaUfuAfaGfagcaasu | 3312 |
| | guauggasus{invAb} | | su | |
| D-2075 | [GalNAc3]sasaucagauCfaUfUfAf | 3059 | asUfsaacuGfguaaUfgAfucugauus | 3313 |
| | Cfcaguuas{invAb} | | usu | |
| D-2076 | [GalNAc3]suscagaucaUfuAfCfCf | 3060 | asGfscuaaCfugguAfaUfgaucugas | 3314 |
| | Afguuagcs{invAb} | | usu | |
| D-2077 | [GalNAc3]suscagAfuCfAfUfUfac | 3061 | asUfsaacuGfguaaUfgAfuCfugasu | 3315 |
| | caguuausus{invAb} | | su | |
| D-2078 | [GalNAc3]sasgauCfaUfUfAfCfca | 3062 | asGfscuaaCfugguAfaUfgAfucusu | 3316 |
| | guuagcusus{invAb} | | su | |
| D-2079 | [GalNAc3]sgscacAfuUfUfGfUfuu | 3063 | usAfsgucuUfaaacAfaAfuGfugcsu | 3317 |
| | aagacuasus{invAb} | | su | |
| D-2080 | [GalNAc3]sasagcacAfuUfUfGfUf | 3064 | usAfsgucuUfaaacaaAfuGfugcuus | 3318 |
| | uuaagacus{invAb} | | usu | |
| D-2081 | [GalNAc3]sgscacauUfuGfUfUfUf | 3065 | usAfsgucuUfaaacAfaAfuGfugcsu | 3319 |
| | aagacuasus{invAb} | | su | |
| D-2082 | [GalNAc3]sgscacAfuUfUfGfUfuu | 3066 | usAfsgucuUfaaacaaAfuGfugcsus | 3320 |
| | aagacuasus{invAb} | | u | |
| D-2083 | [GalNAc3]sgscacauUfuGfUfUfUf | 3067 | usAfsgucuUfaaAfcAfaAfugugcsu | 3321 |
| | aagacuasus{invAb} | | su | |
| D-2084 | [GalNAc3]sgscacauuuGfuUfUfAf | 3068 | asAfsuaguCfuuaaAfcAfaaugugcs | 3322 |
| | Afgacuaus{invAb} | | usu | |
| D-2085 | {DCA- | 3069 | asUfsuauaAfuuggccAfcAfcagsus | 3323 |
| | sC6}cuguGfuGfGfCfCfaauuauaau | | u | |
| | sus{invAb} | | | |
| D-2086 | [GalNAc3]sgaaucaAfgAfUfGfGfu | 3070 | asUfscuucAfccauCfuUfgauucscs | 3324 |
| | gaagsas{invAb} | | u | |
| D-2087 | {DCA- | 3071 | asUfscuucAfccauCfuUfgauucscs | 3325 |
| | sC6}gaaucaAfgAfUfGfGfugaagsa | | u | |
| | s{invAb} | | | |
| D-2088 | {DCA- | 3072 | usCfscauaCfgauuaaGfaGfcaaacs | 3326 |
| | sC6}guuugcUfcUfUfAfAfucguaug | | usu | |
| | gs{invAb} | | | |
| D-2089 | {DCA- | 3073 | asAfscuuUfCfaaaacaUfaCfgaggg | 3327 |
| | sC6}cccucgUfaUfGfUfUfuugaaag | | susu | |
| | us{invAb} | | | |
| D-2090 | [GalNAc3]sgsacgguAfcAfUfAfAf | 3074 | usGfsuaggAfuuAfuguAfcCfgucsu | 3328 |
| | uccuacasus{invAb} | | su | |
| D-2091 | [GalNAc3]scsuguGfuGfGfCfCfaa | 3075 | asUfsuauaAfuuggccAfcAfcagsus | 3329 |
| | uuauaausus{invAb} | | u | |
| D-2092 | [GalNAc3]sasaugGfgAfUfUfUfac | 3076 | usGfsuugcUfguaaauCfcCfauusus | 3330 |
| | agcaacasus{invAb} | | u | |
| D-2093 | [GalNAc3]sasagcacauUfuGfUfUf | 3077 | usAfsgucuUfaaacAfaAfugugcuus | 3331 |
| | Ufaagacus{invAb} | | usu | |
| D-2094 | csgsaagaCfaGfCfGfAfccccaugcs | 3078 | asGfscaugGfggucgcUfgUfcuucgs | 3332 |
| | {invAb} | | usu | |
| D-2095 | csasuggaCfgGfCfCfGfguaacaaas | 3079 | asUfsuuguUfaccggcCfgUfccaugs | 3333 |
| | {invAb} | | usu | |
| D-2096 | usgscacaUfgCfGfCfAfcgcgcaugs | 3080 | asCfsaugcGfcgugcgCfaUfgugcas | 3334 |
| | {invAb} | | usu | |
| D-2097 | gscsacauGfcGfCfAfCfgcgcaugcs | 3081 | usGfscaugCfgcgugcGfcAfugugcs | 3335 |
| | {invAb} | | usu | |
| D-2098 | csascaugCfgCfAfCfGfcgcaugcas | 3082 | asUfsgcauGfcgcgugCfgCfaugugs | 3336 |
| | {invAb} | | usu | |
| D-2099 | ascsaugcGfcAfCfGfCfgcaugcacs | 3083 | asGfsugcaUfgcgcguGfcGfcaugus | 3337 |
| | {invAb} | | usu | |
| D-2100 | uscsugcaCfuAfAfAfAfuccccaaas | 3084 | asUfsuuggGfgauuuuAfgUfgcagas | 3338 |
| | {invAb} | | usu | |

The methods below were applied to synthesize and purify the RNAi constructs identified in Table 1 and Table 2.

### Synthesis

RNAi constructs were synthesized using solid phase phosphoramidite chemistry. Synthesis was performed on a MerMade synthesizer (Bioautomation). Various chemical modifications, including 2′-fluoro modified nucleotides, 2′-O-methyl modified nucleotides, inverted abasic nucleotides, and phosphorothioate internucleotide linkages, were incorporated into the molecules. The RNAi constructs were generally formatted to be duplexes of 19-21 base pairs when annealed with either no overhangs (double bluntmer) or one or two overhangs of 2 nucleotides at the 3′ end of the antisense strand and/or the sense strand. For in vivo studies, the sense strands of the RNAi constructs were conjugated to either a trivalent N-acetyl-galactosamine (GalNAc) moiety or a hydrophobic moiety (e.g., palmitic acid or docosanoic acid) as described further below.

The materials used in the synthesis of RNAi constructs included:
Acetonitrile (DNA Synthesis Grade, AX0152-2505, EMD)
Capping Reagent A (80:10:10 (v/v/v) tetrahydrofuran/lutidine/acetic anhydride, BIO221/4000, EMD)
Capping Reagent B (16% 1-methylimidazole/tetrahydrofuran, BIO345/4000, EMD)
Activator Solution (0.25 M 5-(ethylthio)-1H-tetrazole (ETT) in acetonitrile, BIO152/0960, EMD)
Detritylation Reagent (3% dichloroacetic acid in dichloromethane, BIO830/4000, EMD)
Oxidation Reagent (0.02 M iodine in 70:20:10 (v/v/v) tetrahydrofuran/pyridine/water, BIO420/4000, EMD)
Diethylamine solution (20% DEA in acetonitrile, NC0017-0505, EMD)
Thiolation Reagent (0.05 M 5-N-[(dimethylamino)methylene]amino-3H-1,2,4-dithiazole-3-thione (BIOSULII/160K) in pyridine)
5′-Aminohexyl linker phosphoramidite and 2′-methoxy and 2′-fluoro phosphoramidites of adenosine, guanosine, and cytosine (Thermo Fisher Scientific), 0.10 M in acetonitrile over Molecular Trap Packs (0.5 g per 30 mL, Bioautomation)
2′-methoxy-uridine phosphoramidite (Thermo Fisher Scientific), 0.10 M in 90:10 (v/v) acetonitrile/DMF over Molecular Trap Packs (0.5 g per 30 mL, Bioautomation)
2′-deoxy-reverse absaic phosphoramidite (ChemGenes), 0.10 M in acetonitrile over Molecular Trap Packs (0.5 g per 30 mL, Bioautomation)
CPG Support (Hi-Load Universal Support, 500A (BH5-3500-G1), 79.6 μmol/g, 0.126 g (10 μmol)) or 1 μmol Universal Synthesis Column, 500A, Pipette Style Body (MM5-3500-1, Bioautomation)
Ammonium hydroxide (concentrated, J. T. Baker)

Reagent solutions, phosphoramidite solutions, and solvents were attached to the MerMade instrument. The columns containing solid support (BioAutomation, Universal Support, 500 Å) were affixed to the instrument and washed with acetonitrile. The synthesis was started using the Poseidon software. The phosphoramidite and reagent solution lines were purged. The synthesis was accomplished by repetition of the deprotection/coupling/capping/oxidation/capping synthesis cycle. To the solid support was added detritylation reagent to remove the 5′-dimethoxytrityl (DMT) protecting group. The solid support was washed with acetonitrile. To the support was added phosphoramidite (4 eq.) and activator solution (20 eq.) to couple the incoming nucleotide to the free 5′-hydroxyl group. The coupling reaction (6 min) was repeated twice. The support was washed with acetonitrile and then added capping reagents A and B to terminate any unreacted oligonucleotide chains. The support was washed with acetonitrile. To the support was added oxidation or thiolation reagent to convert the phosphite triester to the phosphate triester or phosphorothioate. The oxidation reaction was increased from 3 to 5 min. To the support was added capping reagents A and B to dehydrate the support and terminate any unreacted oligonucleotide chains. The solid support was washed with acetonitrile. After the final reaction cycle, the resin was first treated with diethylamine solution to remove the 2-cyanoethyl protecting groups from the phosphate backbone. The support was washed with acetonitrile and the DMT group removed from antisense strands. The 5′ termini of sense strands were left 5′-monomethoxytrityl (MMT) protected.

### Analysis of Crude Synthesized RNAi Constructs

Crude samples were prepared for ion-pairing (IP)-LCMS by making 20-fold dilutions into water (1004 final volume). Samples were analyzed by ion-pairing (IP)-LCMS on an Agilent 1290 analytical HPLC. Samples were eluted from a Waters Xbridge BEH OST C18 column (1.7 um, 2.1×50 mm) using a linear gradient of acetonitrile in 15.7 mM DIEA/50 mM HFIP over 3.5 min with a flowrate of 400 μL/min.

### Conjugation

To facilitate on-resin acylation or conjugation to GalNAc, the MMT group was removed by addition of deprotection solution consisting of trifluoroacetic acid with triisopropylsilane (2% each, v/v) in dichloromethane (DCM). The mixture was gently stirred and let stand for approximately 2-5 min. The mixture was initially gravity filtered until the solution no longer drained then filtered under vacuum. The process repeated 5-10 times until the filtrate was no longer colored. The resin was washed with DCM, neutralized with 5% DIEA in DCM (2×2 min), and washed again with DCM.

When conjugation to docosanoic acid (C22) was desired, docosanoic acid (10 molar equivalents relative to the resin) was dissolved in DCM (70 mM, 34.1 mg, 100 μmol, TCI) and TATU (500 mM DMSO) (32.2 mg, 100 μmol, ChemPep) was added (10 eq) followed by DIEA (500 mM DCM) (25.24 mg, 200 μmol, Aldrich) (20 eq). The solution was mixed and let stand to pre-activate for 5-10 min. The activated ester was added to the oligo-resin and the reaction vessels sealed. The reaction vessels were placed on a vortex mixer at 700 RPM for 14h at room temperature. The solution was drained, and the resin washed with DMF and DCM.

When conjugation to a palmitoyl group was desired, palmitic acid (10 molar equivalents relative to the resin) was dissolved in DCM (300 mM, 25.64 mg, 100 μmol, Aldrich) was transferred to a polypropylene tube (10 molar equivalents relative to the resin) and TATU (500 mM DMSO) (32.2 mg, 100 μmol, ChemPep) was added (10 eq) followed by DIEA (500 mM DCM) (25.24 mg, 200 μmol, Aldrich) (20 eq). The solution was mixed and let stand to pre-activate for 5-10 min. The activated ester was added to the oligo-resin and the reaction vessels sealed. The reaction vessels were placed on a vortex mixer at 700 RPM for 14h at room temperature. The solution was drained, and the resin washed with DMF and DCM.

When conjugation to GalNAc was desired, a solution of GalNAc3-Lys2-Ahx (67 mg, 40 μmol) in DMF (0.5 mL) was prepared in a separate vial. GalNAc3-Lys2-Ahx, which has the structure shown as Formula VII below, was prepared with 1,1,3,3-tetramethyluronium tetrafluoroborate (TATU, 12.83 mg, 40 μmol) and diisopropylethylamine (DIEA, 13.9 μL, 80 μmol). The activated coupling solution was added to the resin, and the column was capped and incubated at room temperature overnight. The resin was washed with DMF, DCM, and dried under vacuum.

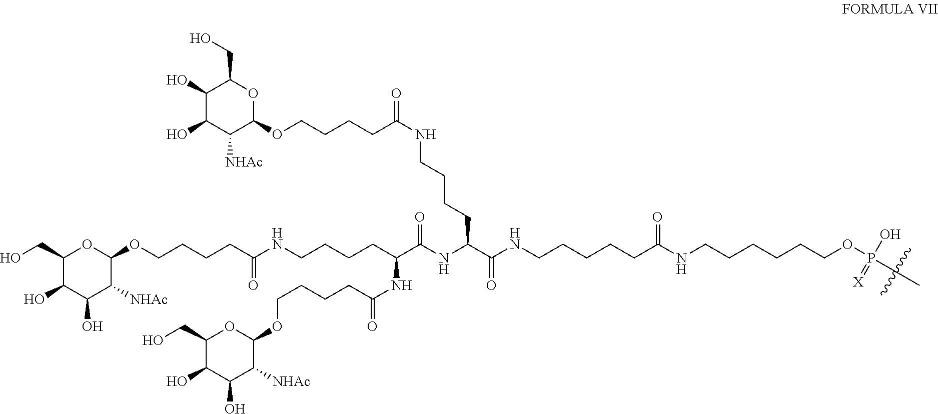

In Formula VII, X═O or S. The squiggly line represents the point of attachment to the 5′ terminal nucleotide of the sense strand of the RNAi construct. The GalNAc moiety was attached to the 5′ carbon of the 5′ terminal nucleotide of the sense strand except where an inverted abasic (invAb) deoxynucleotide was the 5′ terminal nucleotide and linked to the adjacent nucleotide via a 5′-5′ internucleotide linkage, in which case the GalNAc moiety was attached to the 3′ carbon of the inverted abasic deoxynucleotide.

Cleavage from Resin

The columns were placed in a cleavage chuck and to the columns was added 1.2 mL of a solution containing 20% ethanol in concentrated ammonium hydroxide (1:4 v/v). The solvent was allowed to gravity drain through the solid support and filtrates collected into a 24 well plate. The cleavage process was repeated 3 times and the filtrates combined. The plate was sealed in a deprotection chuck and placed in an incubator at 55° C. and let mix at 200 RPM for 20 h. The chuck/plate were let cool to room temperature and samples were taken for LCMS. The plate was placed in a Genevac HT4X and the samples concentrated for 2 hr leaving approximately 2 mL of concentrate

### RP-HPLC Purification of Lipid-Conjugated Oligos

The crude oligo was purified by RP-HPLC using a Phenomenex Oligo-RP C18 column (Sum, 10×250 mm) with a flowrate of 6 mL/min. The mobile phase consisted of 0.02M ammonium bicarbonate with 5% acetonitrile (Buffer-A) & 75% acetonitrile (Buffer-B). The fractions were pooled for desalt as described below.

### Anion Exchange Purification of Oligos

The antisense and GalNAc-conjugated sense strands were purified by anion exchange (AEX) chromatography. Oligos were eluted from a two Tosoh TSK Gel SuperQ-5PW columns in series (21×150 mm, 13 um) with a flowrate of 8 mL/min. using a linear gradient of 1 M sodium bromide in 20 mM sodium phosphate, 15% acetonitrile, pH 8.5. Samples were desalted and UV quantified as described below.

### Desalt

The pooled fractions were desalted by size exclusion chromatography on a GE Akta Pure using a GE Hi-Prep 26/10 column and 19.9% EtOH mobile phase. Desalted samples were analyzed by IP-LCMS, quantified by UV (Nanodrop), and lyophilized in a Genevac S3-HT12.

### Final QC

Samples were analyzed by ion-pairing (IP)-LCMS on an Agilent 1290 analytical HPLC. Samples were eluted from a Waters Xbridge BEH OST C18 column (1.7 um, 2.1×50 mm) using a linear gradient of acetonitrile in 15.7 mM DIEA/50 mM HFIP over 6.5 min. with a flowrate of 400 μL/min.

### Annealing

Single strands were reconstituted in PBS at 2 mM and quantified by UV. Single strands were diluted to 1 mM in PBS and equal volumes combined to anneal the corresponding duplex. The duplex was annealed at 90° C. for 5 min and allowed to cool to room temp. Duplex formation was monitored by analytical AEX and single strands titrated as necessary.

### Example 4: In Vitro Evaluation of FAM13A siRNA Molecules in a Cell-Based Assay

A panel of fully chemically modified siRNAs from Example 3 were prepared and tested for potency and selectivity of FAM13A mRNA knockdown in vitro. Each siRNA duplex consisted of two strands, the sense or ‘passenger’ strand and the antisense or ‘guide’ strand.

RNA FISH (fluorescence in situ hybridization) assay was carried out to measure FAM13A mRNA knockdown by test siRNAs. HUH-7 cells (Sekisui Xenotech JCRB0403) were cultured in Eagle's Minimum Essential Medium (EMEM) (ATCC® 30-2003™) supplemented with 10% fetal bovine serum (FBS, Sigma) and 1% penicillin-streptomycin (P-S, Corning). siRNAs were transfected into cells by reverse transfection using Lipofectamine RNAiMAX transfection reagent (Thermo Fisher Scientific). 1 μL of test siRNAs (in 10 data points for dose with 1:3 dilution starting at 500 nM final concentration) or phosphate-buffered saline (PBS) vehicle and 4 μL of plain EMEM without supplements were added to PDL-coated CellCarrier-384 Ultra assay plates (PerkinElmer) by a Bravo automated liquid handling platform (Agilent). 5 μL of Lipofectamine RNAiMAX (Thermo Fisher Scientific), pre-diluted in plain EMEM without supplements (0.06 μL of RNAiMAX in 5 μL EMEM), was then dispensed into the assay plates by a Multidrop Combi reagent dispenser (Thermo Fisher Scientific). After 20-minute incubation of the siRNA/RNAiMAX mixture at room temperature (RT), 30 μL of HepG2 cells (2000 cells per well) in EMEM supplemented with 10% FBS and 1% P-S were added to the transfection complex using a Multidrop Combi reagent dispenser. The assay plates were incubated at RT for 20 mins prior to being placed in an incubator. Cells were incubated for 72 hrs. at 37° C. and 5% CO₂.

The RNA FISH assay was performed 72 hours after siRNA transfection, using the manufacturer's assay reagents and protocol (QuantiGene® ViewRNA HC Screening Assay from Thermo Fisher Scientific) on an in-house assembled automated FISH assay platform. In brief, cells were fixed in 4% formaldehyde (Thermo Fisher Scientific) for 15 mins at RT, permeabilized with detergent for 3 mins at RT and then treated with protease solution for 10 mins at RT. Target-specific probes (ThermoFisher VA6-3175340-VC) or vehicle (target probe diluent without target probes as negative control) were incubated for 3 hours, whereas preamplifiers, amplifiers, and label probes were incubated for 1 hour each. All hybridization steps were carried out at 40° C. in a Cytomat 2 C-LIN automated incubator (Thermo Fisher Scientific).

After hybridization reactions, cells were stained for 30 mins with Hoechst and CellMask Blue (Thermo Fisher Scientific) and then imaged on an Opera Phenix high-content screening system (PerkinElmer). The images were analyzed using a Columbus image data storage and analysis system (PerkinElmer) to obtain the mean spot count per cell. The mean spot count per cell was normalized using the high (PBS with target probes) and low (PBS without target probes) control wells. The high and low controls have normalized values of 100 and 0, respectively. The normalized values against the test siRNA concentrations were fitted to a 4-parameter sigmoidal model using Genedata Screener data analysis software (Genedata, Basel, Switzerland) to obtain IC50 values and maximum activity.

To verify and compare results, some of the siRNA duplexes were analyzed more than once using the above assay.

The results of the assays are shown in Table 3. FAM13A knockdown provides a percentage of knockdown compared to control samples. Where an siRNA duplex was tested more than once, each test is shown as a separate row in Table 3 as different “runs” of the assay. Negative values indicate a decrease in FAM13A mRNA levels. Undefined means the Genedata Screener software could not fit a curve.

TABLE 3
In vitro inhibition of human FAM13A mRNA in Hep3B cells

| Duplex No. | Run No. | IC50 (nM) | Max FAM13A knockdown (%) |
|---|---|---|---|
| 1001 | | 5.74 | −49.1 |
| 1002 | | 5.95 | −46.1 |
| 1003 | | >500 | −40.0 |
| 1004 | | 3.49 | −38.7 |
| 1005 | Run 1 | Undefined | −33.0 |
| 1005 | Run 2 | 6.51 | −43.9 |
| 1005 | Run 3 | 1.97 | −50.6 |
| 1006 | | >500 | −1.6 |
| 1007 | | 2.4 | −53.3 |
| 1008 | | 1.36 | −52.7 |
| 1009 | | >500 | −15.3 |
| 1010 | | 16.5 | −65.7 |
| 1011 | | 5.85 | −65.5 |
| 1012 | | 13 | −51.9 |
| 1013 | Run 1 | 5.44 | −67.8 |
| 1013 | Run 2 | 4.93 | −63.9 |
| 1014 | | 4.4 | −72.9 |
| 1015 | | 3.99 | −74.7 |
| 1016 | | 2.33 | −67.0 |
| 1017 | | 3.34 | −75.8 |
| 1018 | | 6.84 | −31.9 |
| 1019 | | 2.59 | −78.9 |
| 1020 | | 5.91 | −81.0 |
| 1021 | | 4.43 | −52.3 |
| 1022 | | 5.48 | −70.1 |
| 1023 | | 4.08 | −80.3 |
| 1024 | | 2.47 | −77.4 |
| 1025 | | 6.26 | −81.4 |
| 1026 | | Undefined | −38.7 |
| 1027 | | >500 | −2.1 |
| 1028 | | 3.77 | −81.2 |
| 1029 | | 3.51 | −70.5 |
| 1030 | | 16 | −50.6 |
| 1031 | | 2.67 | −43.7 |
| 1032 | | >500 | −25.7 |
| 1033 | | Undefined | −37.0 |
| 1034 | | 3.92 | −74.3 |
| 1035 | Run 1 | 1.95 | −41.4 |
| 1035 | Run 2 | 9.61 | −31.8 |
| 1036 | | Undefined | −39.3 |
| 1037 | | 1.23 | −77.7 |
| 1038 | Run 1 | 4.72 | −80.8 |
| 1038 | Run 2 | 6.55 | −76.5 |
| 1038 | Run 3 | 4.56 | −71.9 |
| 1039 | | 14.1 | −69.2 |
| 1040 | | 2.55 | −82.9 |
| 1041 | | 3.91 | −71.3 |
| 1042 | | 8.71 | −72.6 |
| 1043 | | 2.46 | −78.6 |
| 1044 | | 4.49 | −77.6 |
| 1045 | | 1.47 | −79.6 |
| 1046 | | 5.89 | −65.6 |
| 1047 | | 6.16 | −73.2 |
| 1048 | | 4.7 | −69.8 |
| 1049 | | 6.59 | −76.0 |
| 1050 | | 5.19 | −86.4 |
| 1051 | | 6.74 | −68.7 |
| 1052 | | 3.77 | −69.5 |
| 1053 | | 18.9 | −62.2 |
| 1054 | | 5.86 | −72.6 |
| 1055 | | Undefined | −60.1 |
| 1056 | | 5.25 | −55.4 |
| 1057 | | 4.01 | −76.3 |
| 1058 | | 4.31 | −74.9 |
| 1059 | | 10.5 | −71.2 |
| 1060 | | 4.44 | −65.1 |
| 1061 | | 11.2 | −82.2 |
| 1062 | | 5.75 | −95.0 |
| 1063 | | 16.5 | −68.1 |
| 1064 | | 11.3 | −56.1 |
| 1065 | | 7.73 | −55.6 |
| 1066 | | >500 | −18.5 |
| 1067 | | 4.9 | −68.4 |
| 1068 | | Undefined | −36.8 |
| 1069 | | 20.2 | −72.1 |
| 1070 | | 3.7 | −84.3 |
| 1071 | | 2.78 | −52.0 |
| 1072 | | 2.72 | −75.8 |
| 1073 | | >500 | −9.7 |
| 1074 | | 4.12 | −76.3 |
| 1075 | | 1.62 | −81.9 |
| 1076 | | 5.71 | −78.7 |
| 1077 | | 2.92 | −60.1 |
| 1078 | | 3.14 | −71.9 |
| 1079 | | 4.53 | −49.4 |
| 1080 | | 8.67 | −79.1 |
| 1081 | | 3.97 | −82.1 |
| 1082 | | 2.73 | −75.3 |
| 1083 | | 2.48 | −72.7 |
| 1084 | | 2.45 | −60.5 |
| 1085 | | 2.93 | −63.7 |
| 1086 | | 11.8 | −81.7 |
| 1087 | | 8.19 | −77.0 |
| 1088 | | Undefined | −28.5 |
| 1089 | | >500 | 4.8 |
| 1090 | | 3.22 | −75.1 |
| 1091 | | 3.5 | −82.7 |
| 1092 | | 2.81 | −70.1 |
| 1093 | | 7.01 | −66.3 |
| 1094 | Run 1 | 27.7 | −70.5 |
| 1094 | Run 2 | 28 | −80.9 |
| 1094 | Run 3 | 6.51 | −72.5 |
| 1095 | | 14.2 | −76.8 |
| 1096 | | 2.98 | −81.7 |
| 1097 | | 3.39 | −79.6 |
| 1098 | | 6.78 | −56.7 |
| 1099 | | >500 | −10.8 |
| 1100 | | 2.42 | −74.3 |
| 1101 | | 3.49 | −64.2 |
| 1102 | Run 1 | 19.9 | −54.2 |
| 1102 | Run 2 | 18.8 | −73.3 |
| 1102 | Run 3 | 26.2 | −70.0 |
| 1103 | | 8.52 | −59.5 |
| 1104 | | 5.59 | −79.1 |
| 1105 | | 6.73 | −38.8 |
| 1106 | | 3.44 | −44.4 |
| 1107 | | 6.25 | −88.4 |
| 1108 | | 17.6 | −42.5 |
| 1109 | | 2.7 | −44.7 |
| 1110 | | >500 | −23.1 |
| 1111 | | >500 | 28.7 |
| 1112 | | 4.27 | −49.4 |
| 1113 | | 4.03 | −54.1 |
| 1114 | | 5.59 | −79.4 |
| 1115 | | 15.8 | −76.0 |
| 1116 | | 6.72 | −70.9 |
| 1117 | | 52.3 | −48.3 |
| 1118 | Run 1 | 7.89 | −58.9 |
| 1118 | Run 2 | 1.42 | −46.1 |
| 1119 | | 5.51 | −78.9 |
| 1120 | | 4.28 | −39.5 |
| 1121 | | >500 | 0.0 |
| 1122 | | 3.2 | −74.3 |
| 1123 | | >18.5 | −24.6 |
| 1124 | | 2.95 | −80.7 |
| 1125 | | 8.53 | −37.6 |
| 1126 | | 1.86 | −85.5 |
| 1127 | | 3.94 | −82.0 |
| 1128 | | 2.92 | −83.9 |
| 1129 | | >500 | −20.2 |
| 1130 | | >500 | 7.8 |
| 1131 | | >500 | −22.7 |
| 1132 | | >500 | −0.8 |
| 1133 | | 1.36 | −38.9 |
| 1134 | | 28.9 | −55.7 |
| 1135 | | Undefined | −57.6 |
| 1136 | | >500 | −18.4 |
| 1137 | | >167 | −19.0 |
| 1138 | | 9.39 | −51.8 |
| 1139 | | >500 | −21.7 |
| 1140 | | 2.61 | −70.0 |
| 1141 | | 1.48 | −75.0 |
| 1142 | | 3.9 | −93.1 |
| 1143 | | 3.55 | −85.1 |
| 1144 | | 6.44 | −54.5 |
| 1145 | | 3.35 | −89.3 |
| 1146 | | 27.9 | −65.8 |
| 1147 | | 1.83 | −64.6 |
| 1148 | | 4.16 | −63.8 |
| 1149 | | 3.07 | −61.7 |
| 1150 | | 3.65 | −79.0 |
| 1151 | | 12.3 | −80.2 |
| 1152 | | 7.56 | −69.6 |
| 1153 | | 6.31 | −87.0 |
| 1154 | | 4.23 | −80.4 |
| 1155 | | 24.9 | −50.3 |
| 1156 | | 6.43 | −72.3 |
| 1157 | | >500 | 3.9 |
| 1158 | | 4.07 | −76.2 |
| 1159 | | 2.42 | −78.5 |
| 1160 | | 3.8 | −31.4 |
| 1161 | | 46.6 | −43.0 |
| 1162 | | Undefined | −39.5 |
| 1163 | | 6.17 | −70.2 |
| 1164 | | 20.5 | −65.9 |
| 1165 | | 19 | −56.7 |
| 1166 | Run 1 | 3.29 | −72.1 |
| 1166 | Run 2 | 3.27 | −75.1 |
| 1167 | | 3.88 | −79.2 |
| 1168 | | 18.2 | −58.5 |
| 1169 | | >500 | −15.2 |
| 1170 | | 8.32 | −58.4 |
| 1171 | | 1.71 | −84.5 |
| 1172 | | 5.3 | −90.7 |
| 1173 | | Undefined | −31.5 |
| 1174 | | 3.51 | −73.5 |
| 1175 | | 12.8 | −85.8 |
| 1176 | | >500 | 8.5 |
| 1177 | | >500 | 6.5 |
| 1178 | | >500 | 11.2 |
| 1179 | Run 1 | >500 | 11.6 |
| 1179 | Run 2 | Undefined | −34.1 |
| 1180 | | Undefined | −52.7 |
| 1181 | | 10.2 | −46.4 |
| 1182 | | 26.2 | −42.9 |
| 1183 | | 5.71 | −50.8 |
| 1184 | | >500 | 21.2 |
| 1185 | | 5.34 | −65.0 |
| 1186 | | 6.12 | −47.3 |
| 1187 | | 12.1 | −64.8 |
| 1188 | | 4.44 | −45.4 |
| 1189 | | 6.12 | −42.4 |
| 1190 | | 3.52 | −74.4 |
| 1191 | | 3.77 | −67.5 |
| 1192 | | >500 | 7.5 |
| 1193 | | 16.6 | −73.9 |
| 1194 | | 33.4 | −40.3 |
| 1195 | | 9.77 | −42.8 |
| 1196 | | >500 | −3.6 |
| 1197 | | 9.38 | −39.0 |
| 1198 | | 9.41 | −55.6 |
| 1199 | | 6.51 | −64.1 |
| 1200 | | >500 | −37.7 |
| 1201 | | >500 | −19.0 |
| 1202 | | >167 | −24.0 |
| 1203 | | 11.6 | −58.5 |
| 1204 | | 0.593 | −44.6 |
| 1205 | | 6.19 | −41.3 |
| 1206 | | 3.99 | −65.8 |
| 1207 | | 7.26 | −49.6 |
| 1208 | | 3.91 | −50.1 |
| 1209 | | 2.55 | −65.0 |
| 1210 | | 3.24 | −84.2 |
| 1211 | | 7.45 | −77.8 |
| 1212 | | 2.09 | −82.1 |
| 1213 | | 5.31 | −83.7 |
| 1214 | | 3.75 | −84.4 |
| 1215 | | 7.38 | −84.0 |
| 1216 | | 2.48 | −74.3 |
| 1217 | | 3.61 | −56.6 |
| 1218 | | 1.22 | −79.7 |
| 1219 | Run 1 | 5.07 | −93.2 |
| 1219 | Run 2 | 1.84 | −88.2 |
| 1219 | Run 3 | 11.1 | −87.5 |
| 1220 | | 3.89 | −88.0 |
| 1221 | | 1.97 | −89.0 |
| 1222 | | 12.9 | −80.1 |
| 1223 | | 57.4 | −31.0 |
| 1224 | | 2.42 | −94.2 |
| 1225 | | 3.62 | −85.0 |
| 1226 | | 3.12 | −87.7 |
| 1227 | | 1.62 | −71.5 |
| 1228 | | >500 | −29.3 |
| 1229 | | >500 | −10.4 |
| 1230 | | 5.23 | −37.0 |
| 1231 | | >500 | 8.4 |
| 1232 | | 1.35 | −78.4 |
| 1233 | | Undefined | −29.0 |
| 1234 | | 4.38 | −42.8 |
| 1235 | | 3.42 | −80.9 |
| 1236 | | 7.33 | −59.6 |
| 1237 | | 8.96 | −51.5 |
| 1238 | | 2.27 | −82.2 |
| 1239 | | 2.74 | −83.0 |
| 1240 | | 2.33 | −77.5 |
| 1241 | | 2.77 | −76.6 |
| 1242 | | 6.71 | −63.5 |
| 1243 | | 2.29 | −86.4 |
| 1244 | | 46.8 | −72.2 |
| 1245 | | 18 | −57.2 |
| 1246 | | >500 | 20.7 |
| 1247 | | >500 | −0.9 |
| 1248 | | >500 | −20.0 |
| 1249 | | 2.99 | −57.3 |
| 1250 | | 4.38 | −72.3 |
| 1251 | | 4.87 | −53.2 |
| 1252 | | 2.3 | −44.6 |
| 1253 | | 3.27 | −66.6 |
| 1254 | | Undefined | −53.4 |
| 1255 | | 3.68 | −51.2 |
| 1256 | | 3.2 | −57.2 |
| 1257 | | 21.6 | −57.7 |
| 1258 | | >500 | −15.1 |
| 1259 | | 9.5 | −76.0 |
| 1260 | | 2.47 | −67.7 |
| 1261 | | 2.41 | −63.4 |
| 1262 | | 12.3 | −78.3 |
| 1263 | | 11.7 | −83.0 |
| 1264 | | 2.02 | −77.8 |
| 1265 | | 6.7 | −62.0 |
| 1266 | | 3.99 | −70.0 |
| 1267 | | 5.48 | −69.7 |
| 1268 | | 4.6 | −55.0 |
| 1269 | | Undefined | −31.8 |
| 1270 | | 5.58 | −64.8 |
| 1271 | | 6.02 | −59.9 |
| 1272 | | 2.32 | −57.8 |
| 1273 | Run 1 | 5.34 | −78.2 |
| 1273 | Run 2 | 4.11 | −72.8 |
| 1274 | | 1.88 | −62.2 |
| 1275 | | 2.35 | −72.0 |
| 1276 | | 1.57 | −72.1 |
| 1277 | | 3.79 | −79.0 |
| 1278 | | 1.81 | −73.0 |
| 1279 | | 1.81 | −71.1 |
| 1280 | | 1.44 | −64.8 |
| 1281 | | 1.95 | −67.7 |
| 1282 | | 4.44 | −71.4 |
| 1283 | | 2.71 | −77.3 |
| 1284 | Run 1 | 3.55 | −67.3 |
| 1284 | Run 2 | 3.28 | −72.0 |
| 1285 | | 8.75 | −66.3 |
| 1286 | | 4.65 | −78.9 |
| 1287 | | 10.3 | −76.5 |
| 1288 | | 4.76 | −74.0 |
| 1289 | | 5.13 | −68.5 |
| 1290 | | >500 | −22.5 |
| 1291 | | 15.6 | −52.2 |
| 1292 | | 2.3 | −37.6 |
| 1293 | | 5.38 | −63.6 |
| 1294 | | >500 | −25.6 |
| 1295 | | 6.02 | −60.4 |
| 1296 | | 5.31 | −83.4 |
| 1297 | | 1.62 | −73.4 |
| 1298 | | Undefined | −30.6 |
| 1299 | | 2.32 | −63.6 |
| 1300 | | 1.19 | −54.6 |
| 1301 | | 1.78 | −64.7 |
| 1302 | | 4.79 | −55.9 |
| 1303 | | 1.83 | −57.3 |
| 1304 | | 9.89 | −75.5 |
| 1305 | | 4.19 | −69.5 |
| 1306 | | 3.65 | −74.3 |
| 1307 | | 12.6 | −55.7 |
| 1308 | | 12.6 | −61.5 |
| 1309 | | 2.83 | −50.6 |
| 1310 | | 4.94 | −55.4 |
| 1311 | | 1.74 | −50.9 |
| 1312 | | 4.25 | −58.2 |
| 1313 | | 5.44 | −71.6 |
| 1314 | | 2.28 | −78.4 |
| 1315 | | 3.42 | −73.2 |
| 1316 | | 8.48 | −43.1 |
| 1317 | | Undefined | −39.4 |
| 1318 | | 9.04 | −51.5 |
| 1319 | | 4.55 | −58.6 |
| 1320 | | 5.69 | −63.2 |
| 1321 | | Undefined | −37.6 |
| 1322 | | 4.82 | −56.4 |
| 1323 | Run 1 | Undefined | −49.9 |
| 1323 | Run 2 | >500 | −8.8 |
| 1324 | | 9.43 | −50.7 |
| 1325 | | 16.3 | −51.9 |
| 1326 | | 15.8 | −42.6 |
| 1327 | | 7.09 | −54.5 |
| 1328 | Run 1 | 22.5 | −63.9 |
| 1328 | Run 2 | 34.1 | −47.5 |
| 1329 | | >500 | 27.5 |
| 1330 | | 4.47 | −39.0 |
| 1331 | | 11.6 | −83.7 |
| 1332 | | 5.14 | −56.8 |
| 1333 | | 3.43 | −44.6 |
| 1334 | | 2.79 | −65.6 |
| 1335 | Run 1 | 5.17 | −55.2 |
| 1335 | Run 2 | 5.78 | −58.2 |
| 1336 | Run 1 | 5.63 | −54.8 |
| 1336 | Run 2 | Undefined | −50.7 |
| 1337 | | 13.9 | −68.8 |
| 1338 | | 13 | −61.6 |
| 1339 | | 12.7 | −66.6 |
| 1340 | | 2.85 | −68.3 |
| 1341 | Run 1 | 3.41 | −68.1 |
| 1341 | Run 2 | 1.97 | −64.3 |
| 1342 | Run 1 | 0.113 | −76.1 |
| 1342 | Run 2 | 3.75 | −81.6 |
| 1343 | | 3.08 | −78.4 |
| 1344 | | 14.1 | −87.2 |
| 1345 | | 2.92 | −84.4 |
| 1346 | | 9.04 | −84.3 |
| 1347 | | 6.71 | −75.3 |
| 1348 | | 11.4 | −75.0 |
| 1349 | | 8.35 | −57.0 |
| 1350 | Run 1 | 9.86 | −79.2 |
| 1350 | Run 2 | 6.72 | −74.6 |
| 1351 | | 6.46 | −72.8 |
| 1352 | Run 1 | Undefined | −31.0 |
| 1352 | Run 2 | 23.5 | −36.5 |
| 1353 | | 8.35 | −57.1 |
| 1354 | | Undefined | −32.8 |
| 1355 | | >500 | −1.4 |
| 1356 | | >500 | −19.1 |
| 1357 | | Undefined | −53.8 |
| 1358 | | 10.9 | −63.6 |
| 1359 | | 7.91 | −57.3 |
| 1360 | | 10.5 | −63.8 |
| 1361 | | 3.53 | −37.2 |
| 1362 | | >500 | −3.7 |
| 1363 | | >500 | −19.6 |
| 1364 | Run 1 | Undefined | −36.2 |
| 1364 | Run 2 | 40.9 | −40.0 |
| 1365 | | 26.2 | −50.3 |
| 1366 | Run 1 | 4.25 | −50.8 |
| 1366 | Run 2 | 1.76 | −52.4 |
| 1367 | | >500 | 23.8 |
| 1368 | | Undefined | −39.2 |
| 1369 | | >500 | −32.2 |
| 1370 | | 16.3 | −49.5 |
| 1371 | | Undefined | −51.8 |
| 1372 | | 43.5 | −73.6 |
| 1373 | | 4.18 | −60.3 |
| 1374 | | 11.9 | −62.0 |
| 1375 | | 9.86 | −61.2 |
| 1376 | | 7.68 | −61.3 |
| 1377 | | 4.8 | −50.1 |
| 1378 | Run 1 | 2.32 | −61.0 |
| 1378 | Run 2 | 1.91 | −65.2 |
| 1379 | | 7.06 | −59.1 |
| 1380 | | 14.4 | −64.5 |
| 1381 | Run 1 | Undefined | −31.4 |
| 1381 | Run 2 | >500 | −3.3 |
| 1382 | | 6.69 | −36.3 |
| 1383 | | >500 | −14.2 |
| 1384 | | 24.7 | −72.1 |
| 1385 | | 7.57 | −65.4 |
| 1386 | | 5.78 | −80.7 |
| 1387 | | 13 | −60.6 |
| 1388 | | 18.1 | −71.0 |
| 1389 | | 6.48 | −72.6 |
| 1390 | | 1.79 | −68.7 |
| 1391 | | 3.7 | −77.4 |
| 1392 | | 7.54 | −74.5 |
| 1393 | | 2.25 | −66.4 |
| 1394 | | 4.24 | −69.7 |
| 1395 | | 2.06 | −64.5 |
| 1396 | Run 1 | 1.01 | −69.3 |
| 1396 | Run 2 | 0.597 | −71.2 |
| 1397 | | 2.63 | −70.2 |
| 1398 | | 5.5 | −74.8 |
| 1399 | | 1.93 | −70.0 |
| 1400 | | 3.14 | −74.4 |
| 1401 | | 9.12 | −70.5 |
| 1402 | | Undefined | −43.8 |
| 1403 | | 8.46 | −70.3 |
| 1404 | | 9.15 | −75.0 |
| 1405 | | 14.5 | −63.9 |
| 1406 | | 4.89 | −60.6 |
| 1407 | | 5.41 | −52.0 |
| 1408 | Run 1 | 2.46 | −42.3 |
| 1408 | Run 2 | 1.75 | −67.0 |
| 1409 | | 19.5 | −64.5 |
| 1410 | | >500 | −28.7 |
| 1411 | | >500 | −14.2 |
| 1412 | | >500 | −3.4 |
| 1413 | | 4.59 | −58.6 |
| 1414 | | Undefined | −43.9 |
| 1415 | | Undefined | −54.5 |
| 1416 | Run 1 | >500 | 22.8 |
| 1416 | Run 2 | >500 | 13.1 |
| 1417 | | >500 | 0.5 |
| 1418 | | >500 | 43.6 |
| 1419 | | 77.3 | −64.7 |
| 1420 | Run 1 | >500 | 9.3 |
| 1420 | Run 2 | >500 | −4.3 |
| 1421 | Run 1 | 7.54 | −47.1 |
| 1421 | Run 2 | 26.3 | −43.0 |
| 1422 | | 4.9 | −53.3 |
| 1423 | | 2.06 | −54.0 |
| 1424 | | Undefined | −49.9 |
| 1425 | | 3.79 | −55.6 |
| 1426 | | Undefined | −45.5 |
| 1427 | | 0.569 | −46.2 |
| 1428 | | 7.11 | −72.7 |
| 1429 | | 0.653 | −63.3 |
| 1430 | | 1.07 | −66.8 |
| 1431 | | 4.47 | −70.9 |
| 1432 | | 7.62 | −63.1 |
| 1433 | | 15.8 | −66.5 |
| 1434 | | 4.22 | −64.0 |
| 1435 | | 5.61 | −57.6 |
| 1436 | | Undefined | −45.5 |
| 1437 | | Undefined | −39.1 |
| 1438 | | 32.2 | −69.4 |
| 1439 | | 4.03 | −48.3 |
| 1440 | | Undefined | −40.6 |
| 1441 | Run 1 | 13.7 | −45.1 |
| 1441 | Run 2 | Undefined | −61.3 |
| 1442 | | 1.48 | −57.2 |
| 1443 | | 4.08 | −70.3 |
| 1444 | | 6.59 | −68.0 |
| 1445 | | 5.2 | −44.5 |
| 1446 | Run 1 | 16.2 | −77.6 |
| 1446 | Run 2 | 18 | −82.3 |
| 1447 | | 3.61 | −64.6 |
| 1448 | | 8.4 | −44.0 |
| 1449 | | >500 | −21.8 |
| 1450 | | 28.4 | −67.6 |
| 1451 | | 8.59 | −57.0 |
| 1452 | | 7.12 | −62.2 |
| 1453 | | 3.53 | −62.4 |
| 1454 | Run 1 | 16.5 | −76.5 |
| 1454 | Run 2 | 6.29 | −51.7 |
| 1455 | | 3.26 | −61.8 |
| 1456 | | 2.62 | −58.2 |
| 1457 | | 9.92 | −55.2 |
| 1458 | | 6.52 | −80.4 |
| 1459 | Run 1 | 1.57 | −64.4 |
| 1459 | Run 2 | 4.65 | −63.4 |
| 1460 | | 3.82 | −61.8 |
| 1461 | | 4.83 | −80.9 |
| 1462 | | 3.11 | −82.0 |
| 1463 | | 3.2 | −78.5 |
| 1464 | | 3.85 | −72.9 |
| 1465 | | 2.94 | −79.1 |
| 1466 | | 2.73 | −70.7 |
| 1467 | | 2.78 | −67.1 |
| 1468 | | 3.24 | −64.2 |
| 1469 | | 8.53 | −71.2 |
| 1470 | | 7.92 | −73.5 |
| 1471 | | 4.63 | −67.0 |
| 1472 | | 7.7 | −66.0 |
| 1473 | | 5.15 | −67.9 |
| 1474 | | 7.04 | −76.4 |
| 1475 | | 3.17 | −74.5 |
| 1476 | | 1.86 | −72.8 |
| 1477 | | 6.87 | −62.9 |
| 1478 | | 19.1 | −61.4 |
| 1479 | | 3.31 | −79.0 |
| 1480 | | 5.12 | −74.9 |
| 1481 | | 2.39 | −74.5 |
| 1482 | | 7.5 | −69.0 |
| 1483 | | 3.6 | −70.0 |
| 1484 | | 1.97 | −72.2 |
| 1485 | | 5.47 | −79.8 |
| 1486 | | 6.5 | −73.7 |
| 1487 | | 5.19 | −59.7 |
| 1488 | | 1.78 | −81.0 |
| 1489 | | 1.99 | −61.5 |
| 1490 | | 0.976 | −60.4 |
| 1491 | | 2.58 | −73.5 |
| 1492 | | 0.878 | −81.6 |
| 1493 | | 3.86 | −81.7 |
| 1494 | | 2.53 | −64.3 |
| 1495 | | 3.27 | −75.3 |
| 1496 | | 1.24 | −90.2 |
| 1497 | | 1.26 | −81.8 |
| 1498 | | 1.44 | −86.2 |
| 1499 | | 1.14 | −69.0 |
| 1500 | Run 1 | 1.85 | −75.9 |
| 1500 | Run 2 | 1.07 | −71.1 |
| 1500 | Run 3 | 1.73 | −80.0 |
| 1501 | | 1.31 | −81.6 |
| 1502 | | 1.91 | −71.2 |
| 1503 | | 4.05 | −77.5 |
| 1504 | | 3.64 | −75.5 |
| 1505 | | 1.22 | −76.0 |
| 1506 | | 0.925 | −57.1 |
| 1507 | Run 1 | 2.22 | −74.5 |
| 1507 | Run 2 | 2.84 | −72.6 |
| 1507 | Run 3 | 3.58 | −64.9 |
| 1508 | | 3.57 | −73.6 |
| 1509 | Run 1 | 2.55 | −76.4 |
| 1509 | Run 2 | 3.9 | −72.5 |
| 1510 | Run 1 | 0.449 | −73.0 |
| 1510 | Run 2 | 0.715 | −65.6 |
| 1511 | | 3.23 | −74.2 |
| 1512 | Run 1 | 2.08 | −77.9 |
| 1512 | Run 2 | 2.54 | −62.8 |
| 1513 | | 4.14 | −68.2 |
| 1514 | Run 1 | 5.72 | −67.8 |
| 1514 | Run 2 | 5.64 | −77.0 |
| 1515 | | 2.7 | −75.4 |
| 1516 | | 2.5 | −66.4 |
| 1517 | | 2.83 | −66.8 |
| 1518 | Run 1 | 1.91 | −66.6 |
| 1518 | Run 2 | 5.63 | −70.0 |
| 1518 | Run 3 | 3.13 | −76.4 |
| 1519 | | 3.52 | −63.2 |
| 1520 | | 1.05 | −70.7 |
| 1521 | | 1.41 | −56.4 |
| 1522 | | 2.37 | −68.2 |
| 1523 | | 0.935 | −68.4 |
| 1524 | Run 1 | 4.41 | −75.7 |
| 1524 | Run 2 | 0.914 | −70.3 |
| 1525 | | 3.18 | −62.3 |
| 1526 | | 4.5 | −66.1 |
| 1527 | | 1.93 | −73.6 |
| 1528 | | 1.35 | −79.4 |
| 1529 | | 1.8 | −72.2 |
| 1530 | Run 1 | 0.598 | −58.8 |
| 1530 | Run 2 | 1.12 | −69.6 |
| 1531 | | 1.59 | −76.4 |
| 1532 | | 2.1 | −67.7 |
| 1533 | | 2.56 | −41.1 |
| 1534 | | 5.96 | −57.4 |
| 1535 | Run 1 | 1.75 | −61.3 |
| 1535 | Run 2 | 0.449 | −58.7 |
| 1536 | | 14.7 | −66.5 |
| 1537 | | 0.47 | −44.4 |
| 1538 | | 2.27 | −45.6 |

### Example 5: In Vivo Efficacy of siRNA Molecules in an AAV Human FAM13A Mouse Model

To assess the efficacy of the FAM13A siRNA molecules, the top performing FAM13A siRNA molecules from the in vitro activity assays described in Example 4 were evaluated for in vivo efficacy and durability in a C57BL/6 mouse model. Broadly, the FAM13A siRNA molecules were administered to mice expressing a portion of the human FAM13A gene. For these experiments, the sense strand in each tested siRNA molecule was conjugated to the trivalent GalNAc moiety shown in Formula VII or to docosanoic acid (C22), using the methods described in Example 3. In some experiments, FAM13A siRNA molecules were evaluated for in vivo efficacy and durability with altered chemical modification patterns.

The mouse model used was an AAV human FAM13A mouse model. In advance of siRNA injection, 10-12-week-old C57BL/6 mice (The Jackson Laboratory) were fed standard chow (Harlan, 2020× Teklad global soy protein-free extruded rodent diet). Female C57Bl6 mice 10-14 weeks old were intravenously (i.v.) injected with an adeno-associated virus (AAV) engineered to coexpress both eGFP and a portion of the human FAM13A gene transcript. The construct used was: AAV-hFAM13A-1 (encoding nucleotides 1200-2900 of SEQ ID NO: 1; “AAV1”), AAV-hFAM13A-2 (encoding nucleotides 2800-4500 of SEQ ID NO: 1; “AAV2”), AAV-hFAM13A-3 (encoding nucleotides 4400-6100 of SEQ ID NO: 1; “AAV3”), AAV-hFAM13A-9span (encoding selected portions of SEQ ID NO: 1 that contain SEQ ID NOs: 15, 24, 125, 127, 222, 233, 481, 498, 503, 504, and 513, connected by linkers; “AAV-9span”), or AAV-FAM13A-22span (encoding selected portions of SEQ ID NO: 1 that contain SEQ ID NOs: 15, 24, 41, 125, 127, 150, 164, 222, 233, 406, 448, 466, 470, 481, 498, 503, 504, 513, 523, 526, 527, 533, and 534, connected by linkers; “AAV-22span”).

Each mouse was injected with a single AAV at a dose of 1×10¹² genome copies (GC) per animal. Two weeks following AAV injection, mice received a single subcutaneous (s.c.) injection of buffer (PBS) or the FAM13A siRNA molecule at a dose of 0.5 mg/kg, 1 mg/kg, 3 mg/kg, 5 mg/kg, 15 mg/kg, or 20 mg/kg body weight in PBS (n=3 or 4 mice per group, as indicated below).

Liver and subcutaneous white adipose tissue (ScWAT) were collected 2 or 4 weeks following siRNA administration and analyzed. RNA from harvested animal tissues was processed for qPCR analysis. RNA was isolated from 50-100 mg tissue using RNeasy 96 universal tissue kit RNA isolation protocol following manufacturer's instructions (Qiagen) or using a KingFisher Apex system and the MagMAX mirVana Total RNA Isolation Kit according to the manufacturer's instructions (ThermoFisher). Real-time PCR was performed using TaqMan® RNA-to-Ct™ 1-Step Kit following manufacturer's instructions (ThermoFisher) with 50 ng RNA per reaction and the following primer probe sets: (1) eGFP1 Forward primer: CTATGTGCAGGAGAGAACCATC (Sense; SEQ ID NO: 2798); Reverse primer: GCCCTTCAGCTCGATTCTATT (Antisense; SEQ ID NO: 2799); Probe: 5′-6FAM-TACAAGACCCGCGCTGAAGTCAAG TAMRA-3′ (Sense; SEQ ID NO: 2800); (2) eGFP2 Forward primer: TCATCTGCACCACTGGAAAG (Sense; SEQ ID NO: 2801); Reverse primer: CTGCTTCATATGGTCTGGGTATC (Antisense; SEQ ID NO: 2802); Probe: 5′-6FAM CCAACACTGGTCACTACCCTCACC TAMRA-3′ (Sense; SEQ ID NO: 2803); (3) BGH Forward primer: 5′-GCCAGCCATCTGTTGT-3′ (SEQ ID NO: 2804); Reverse primer: 5′-GGAGTGGCACCTTCCA-3′ (SEQ ID NO: 2805); Probe: 5′-6FAM-TCCCCCGTGCCTTCCTTGACC TAMRA-3′ (Sense; SEQ ID NO: 2806); and (4) mPpib TaqMan® gene expression assay (Mm00478295 Thermo Fisher). Knockdown of mRNA levels were quantified using primer sets targeting either the eGFP sequence in the 5′ end of the construct (i.e., eGFP primer set #1 or eGFP primer set #2) or the bovine growth hormone polyadenylation signal present in the viral mRNA (BGHpA primer set) at the 3′ end of construct. The knockdown efficiency of the siRNA triggers was determined using semi-quantitative real-time polymerase chain reactions on a QuantStudio 7 Flex real time thermocycler. Gene expression was calculated using the ΔΔCt approach while utilizing cyclophilin (PPIB) as the reference gene. A percentage change in human FAM13A mRNA in liver or ScWAT for each animal was calculated relative to the level of human FAM13A mRNA in the liver or ScWAT of control animals. The control animals used to calculate the percentage change expressed the same human FAM13A mRNA but received the buffer only injection in place of an siRNA injection.

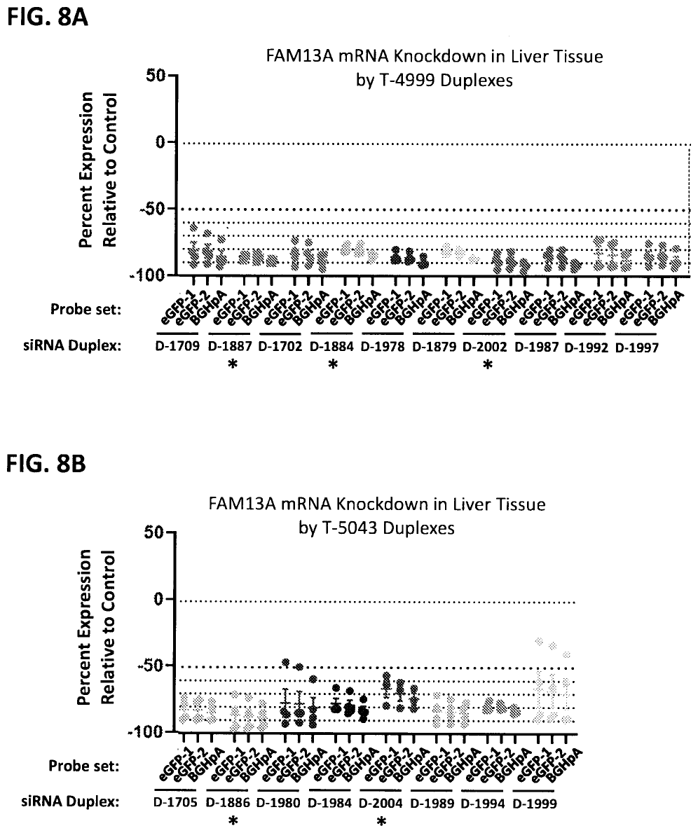

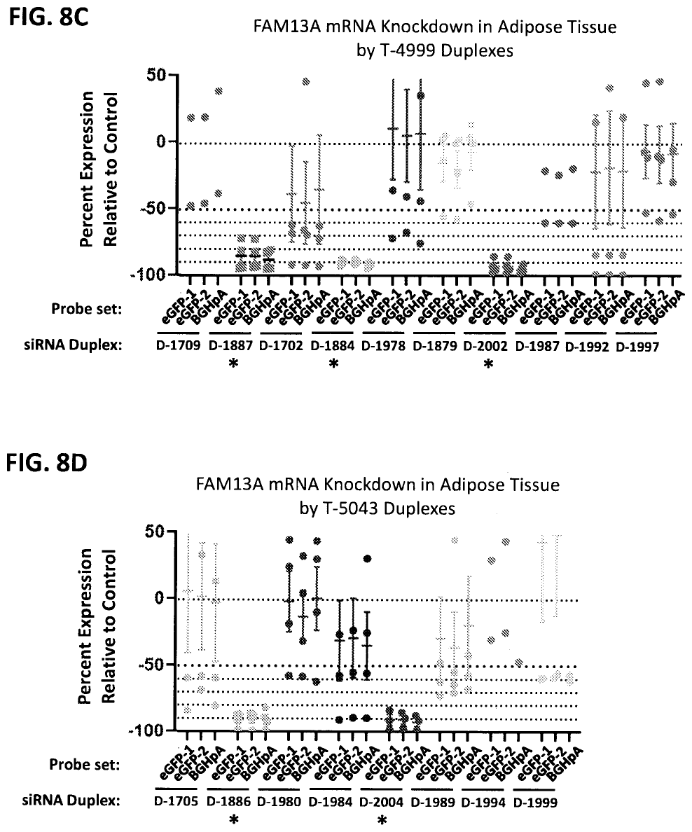

Results of the studies in the AAV-FAM13A mouse model with different FAM13A siRNA molecules are shown in Tables 4-17 below. Data are expressed as average percent change from control at week 4 or 6 of each study (i.e., 2 or 4 weeks after siRNA injection as indicated) for each treatment group (n=3 or 4 animals/group as indicated). The trigger family refers to the first nucleotide in the range of nucleotides of SEQ ID NO: 1 that is targeted by a given siRNA molecule. If a FAM13A siRNA molecule has the same trigger family designation as another FAM13A siRNA molecule but differs in duplex number, then the two molecules have the same core sequence (i.e., the siRNA molecules target the same region of the FAM13A transcript) but differ in chemical modification pattern as detailed in Table 2. A chart of a subset of this data is also shown in FIGS. 8A-8D.

TABLE 4
siRNA Efficacy in Liver 4 Weeks Following siRNA Injection

| SIRNA | Trigger | Dose | | AAV | | Avg. % Change in mRNA ± STD | | |
|---|---|---|---|---|---|---|---|---|
| Duplex | Family | (mg/kg) | N | No. | Carrier | eGFP-1 | eGFP-2 | BGHpA |
| D-1545 | 1309 | 1 | 3 | AAV1 | GalNAc | −42.71 ± 5.394 | −42.27 ± 6.616 | −43.14 ± 7.444 |
| D-1570 | 1311 | 1 | 4 | AAV1 | GalNAc | −19.4 ± 22.73 | −20.06 ± 23.37 | −11.63 ± 22.15 |
| D-1569 | 1338 | 1 | 4 | AAV1 | GalNAc | −11.79 ± 13.66 | −9.43 ± 15.27 | −2.923 ± 19.6 |
| D-1543 | 1366 | 1 | 4 | AAV1 | GalNAc | 6.133 ± 27.89 | −6.135 ± 26.59 | −0.283 ± 29.62 |
| D-1542 | 1489 | 1 | 4 | AAV1 | GalNAc | 35.76 ± 43.79 | −9.96 ± 10.4 | −3.553 ± 24.72 |
| D-1553 | 1495 | 1 | 4 | AAV1 | GalNAc | 23.78 ± 27.21 | −19.31 ± 9.899 | −22.28 ± 11.59 |
| D-1576 | 1533 | 1 | 4 | AAV1 | GalNAc | 24.96 ± 21.55 | 23.82 ± 31.71 | −10.03 ± 7.092 |
| D-1575 | 1558 | 1 | 4 | AAV1 | GalNAc | 33.74 ± 49.25 | 13.87 ± 59.23 | −6.078 ± 39.15 |
| D-1574 | 1619 | 1 | 4 | AAV1 | GalNAc | 40.25 ± 54.93 | −34.08 ± 31.01 | −36.47 ± 25.89 |
| D-1554 | 1632 | 1 | 4 | AAV1 | GalNAc | −6.793 ± 44.9 | −31.49 ± 35.05 | −35.24 ± 28.19 |
| D-1563 | 1896 | 1 | 4 | AAV1 | GalNAc | 29.29 ± 45.3 | −8.05 ± 32.28 | −13.19 ± 25.73 |
| D-1568 | 2066 | 1 | 4 | AAV1 | GalNAc | 21.07 ± 48.89 | −13.38 ± 36.29 | −18.64 ± 22.78 |
| D-1567 | 2070 | 1 | 4 | AAV1 | GalNAc | −23.67 ± 22.87 | −21.89 ± 19.75 | −29.35 ± 12.26 |
| D-1550 | 2078 | 1 | 4 | AAV1 | GalNAc | −25.78 ± 25.17 | −15.87 ± 29.37 | −19.85 ± 24.99 |
| D-1549 | 2080 | 1 | 4 | AAV1 | GalNAc | −44.66 ± 24.88 | −37.31 ± 28.36 | −33.42 ± 23.16 |
| D-1544 | 2144 | 1 | 4 | AAV1 | GalNAc | −28.04 ± 10.28 | −23.07 ± 13.65 | −36.02 ± 7.789 |
| D-1565 | 2146 | 1 | 4 | AAV1 | GalNAc | −1.398 ± 16.51 | 3.045 ± 16.82 | −17.82 ± 16.44 |
| D-1539 | 2151 | 1 | 3 | AAV1 | GalNAc | −32.85 ± 3.319 | −27.82 ± 3.752 | −35.23 ± 5.315 |
| D-1573 | 2263 | 1 | 4 | AAV1 | GalNAc | −39.7 ± 15.67 | −37.2 ± 17.08 | −60.35 ± 10.09 |
| D-1547 | 2266 | 1 | 4 | AAV1 | GalNAc | 26.08 ± 32.18 | 5.21 ± 30.99 | −37.34 ± 10.9 |
| D-1556 | 2356 | 1 | 4 | AAV1 | GalNAc | 62.47 ± 17.62 | 38.02 ± 19.36 | −6.388 ± 11.61 |
| D-1578 | 2360 | 1 | 4 | AAV1 | GalNAc | 13.18 ± 30.52 | −6.565 ± 26.66 | −36.01 ± 14.96 |
| D-1581 | 2623 | 1 | 4 | AAV1 | GalNAc | −1.75 ± 5.865 | −22.66 ± 7.519 | −57.66 ± 5.097 |
| D-1561 | 2887 | 1 | 4 | AAV1 | GalNAc | 16.06 ± 19.7 | −2.823 ± 18.4 | −40.04 ± 5.249 |
| D-1561 | 2887 | 1 | 3 | AAV2 | GalNAc | −23.87 ± 27.24 | −20.19 ± 29.58 | −36.03 ± 9.772 |
| D-1620 | 2889 | 1 | 3 | AAV1 | GalNAc | 40.81 ± 24.72 | 15.21 ± 24.44 | −39.23 ± 10.53 |
| D-1620 | 2889 | 1 | 3 | AAV2 | GalNAc | 52.21 ± 70.23 | 47.44 ± 78.78 | −39.71 ± 28.42 |
| D-1560 | 2890 | 1 | 3 | AAV2 | GalNAc | −17.63 ± 29.61 | −12.89 ± 40.28 | −19.9 ± 24.63 |
| D-1559 | 2893 | 1 | 3 | AAV2 | GalNAc | −27.2 ± 15.47 | −25.82 ± 14.26 | −38.13 ± 14.9 |
| D-1558 | 2895 | 1 | 4 | AAV2 | GalNAc | −4.058 ± 33.95 | −1.795 ± 34.36 | −18.15 ± 26.74 |
| D-1604 | 2923 | 1 | 4 | AAV2 | GalNAc | −18.23 ± 34.59 | −14.74 ± 36.79 | −27.4 ± 32.69 |
| D-1541 | 2934 | 1 | 4 | AAV2 | GalNAc | −24.22 ± 24.87 | −17.93 ± 26.09 | −32.21 ± 24.58 |
| D-1588 | 2937 | 1 | 4 | AAV2 | GalNAc | −6.588 ± 17.49 | −2.385 ± 20.55 | −20.34 ± 14.32 |
| D-1619 | 2994 | 1 | 4 | AAV2 | GalNAc | −4.965 ± 29.63 | −0.955 ± 31.88 | −16.1 ± 23.98 |
| D-1557 | 3000 | 1 | 4 | AAV2 | GalNAc | −38.01 ± 36.01 | −38.72 ± 32.87 | −45.02 ± 26.58 |
| D-1579 | 3002 | 1 | 4 | AAV2 | GalNAc | −29.78 ± 13.38 | −23.06 ± 12.39 | −29.02 ± 11.54 |
| D-1555 | 3014 | 1 | 4 | AAV2 | GalNAc | −33.3 ± 38.16 | −33.52 ± 35.11 | −27.61 ± 45.38 |
| D-1586 | 3133 | 1 | 3 | AAV2 | GalNAc | −14.15 ± 24.03 | −14.48 ± 32.6 | −32.73 ± 22.7 |
| D-1540 | 3184 | 1 | 4 | AAV2 | GalNAc | 49.01 ± 85.72 | 47.35 ± 84.39 | −15.39 ± 48 |
| D-1552 | 3187 | 1 | 4 | AAV2 | GalNAc | −23.01 ± 40.18 | −23.16 ± 41.09 | −57.91 ± 23.56 |
| D-1618 | 3189 | 1 | 4 | AAV2 | GalNAc | −15.8 ± 32.14 | −11.32 ± 38.42 | −55.8 ± 15.9 |
| D-1585 | 3192 | 1 | 4 | AAV2 | GalNAc | −23.44 ± 22.34 | −25.44 ± 24.5 | −32.71 ± 15.77 |
| D-1584 | 3283 | 1 | 4 | AAV2 | GalNAc | 51.7 ± 81.84 | 48.43 ± 75.58 | −28.35 ± 32.39 |
| D-1580 | 3438 | 1 | 3 | AAV2 | GalNAc | 37.73 ± 23.51 | 37.64 ± 14.87 | −41.3 ± 12.94 |
| D-1583 | 3498 | 1 | 4 | AAV2 | GalNAc | 61.43 ± 51.09 | 70.94 ± 52.52 | −40.57 ± 15.94 |
| D-1582 | 3499 | 1 | 4 | AAV2 | GalNAc | 3.543 ± 40.05 | 5.378 ± 33.08 | −50.74 ± 20.44 |
| D-1571 | 3569 | 1 | 4 | AAV2 | GalNAc | 87.02 ± 48.83 | 105.1 ± 53.36 | −23.29 ± 23.41 |
| D-1551 | 3777 | 1 | 4 | AAV2 | GalNAc | 32.18 ± 41.17 | 38.02 ± 52.05 | −37.14 ± 25.23 |
| D-1548 | 4008 | 1 | 4 | AAV2 | GalNAc | 55.32 ± 23.06 | 52.73 ± 30.99 | −42 ± 8.405 |
| D-1600 | 4109 | 1 | 4 | AAV2 | GalNAc | −0.87 ± 54.04 | 4.31 ± 64.12 | −53.28 ± 23.72 |

TABLE 5
siRNA Dose-Response in Liver 2 Weeks Following siRNA Injection

| SIRNA | Trigger | Dose | | AAV | | Avg. % Change in mRNA ± STD | | |
|---|---|---|---|---|---|---|---|---|
| Duplex | Family | (mg/kg) | N | No. | Carrier | eGFP-1 | eGFP-2 | BGHpA |
| D-1545 | 1309 | 0.5 | 4 | AAV1 | GalNAc | 32.32 ± 41.77 | 27.47 ± 45.03 | 60.59 ± 56.66 |
| D-1545 | 1309 | 1 | 3 | AAV1 | GalNAc | −43.11 ± 49.6 | −43.36 ± 48.77 | −21.68 ± 72.64 |
| D-1545 | 1309 | 3 | 3 | AAV1 | GalNAc | −38.58 ± 8.37 | −38.32 ± 8.48 | −4.04 ± 17.42 |
| D-1635 | 1309 | 5 | 4 | AAV1 | C22 | −33.1 ± 36.41 | −38.7 ± 34.27 | −36.57 ± 31.56 |
| D-1635 | 1309 | 15 | 4 | AAV1 | C22 | −9.79 ± 18.62 | −11.38 ± 16.78 | −6.75 ± 28.58 |
| D-1639 | 1309 | 5 | 4 | AAV1 | C22 | 40.4 ± 22.62 | 31.4 ± 25.97 | 19.65 ± 19.04 |
| D-1639 | 1309 | 15 | 4 | AAV1 | C22 | −3.26 ± 22.12 | −8.76 ± 21.44 | −8.45 ± 16.66 |
| D-1640 | 1309 | 0.5 | 4 | AAV1 | GalNAc | −28.7 ± 46.06 | −32.11 ± 44.06 | −2.33 ± 58.61 |
| D-1640 | 1309 | 1 | 4 | AAV1 | GalNAc | −46.12 ± 29.63 | −47.19 ± 29.99 | −19.78 ± 50.46 |
| D-1640 | 1309 | 3 | 3 | AAV1 | GalNAc | −45.55 ± 4.81 | −46.32 ± 6.37 | −19.31 ± 10.39 |
| D-1549 | 2080 | 0.5 | 3 | AAV1 | GalNAc | −8.73 ± 17.94 | −11.9 ± 18.81 | −10.33 ± 22.34 |
| D-1549 | 2080 | 1 | 3 | AAV1 | GalNAc | −60.51 ± 24.19 | −53.49 ± 11.13 | −52.03 ± 14.74 |
| D-1549 | 2080 | 3 | 3 | AAV1 | GalNAc | −47.02 ± 17.61 | −47.06 ± 20.21 | −47.44 ± 14.68 |
| D-1643 | 2080 | 5 | 3 | AAV1 | C22 | 9.49 ± 25.88 | −0.76 ± 26.26 | −11.85 ± 13.08 |
| D-1643 | 2080 | 15 | 3 | AAV1 | C22 | 6.1 ± 41.35 | 2.91 ± 39.69 | −6.65 ± 37.28 |
| D-1544 | 2144 | 0.5 | 4 | AAV1 | GalNAc | −34.12 ± 38.75 | −39.14 ± 36.58 | −10.02 ± 46.03 |
| D-1544 | 2144 | 1 | 4 | AAV1 | GalNAc | −5.52 ± 24.49 | −3.87 ± 28.72 | 23.6 ± 37.54 |
| D-1544 | 2144 | 3 | 3 | AAV1 | GalNAc | −52.79 ± 51.1 | −54.53 ± 49.23 | −28.81 ± 78.9 |
| D-1636 | 2144 | 5 | 4 | AAV1 | C22 | −15.47 ± 42.39 | −23.31 ± 36.14 | −16.14 ± 34.22 |
| D-1636 | 2144 | 15 | 4 | AAV1 | C22 | −43.75 ± 19.47 | −44.85 ± 17.58 | −39.17 ± 18.75 |
| D-1539 | 2151 | 1 | 4 | AAV1 | GalNAc | 13.36 ± 33.44 | 4.59 ± 30.4 | −0.61 ± 23.31 |
| D-1539 | 2151 | 3 | 4 | AAV1 | GalNAc | −15.59 ± 26.47 | −16.46 ± 26.89 | −15.8 ± 20.8 |
| D-1573 | 2263 | 0.5 | 3 | AAV1 | GalNAc | −2.52 ± 64.76 | −5.55 ± 66.02 | 3.78 ± 43.72 |
| D-1573 | 2263 | 1 | 3 | AAV1 | GalNAc | −36.45 ± 14.44 | −36.88 ± 13.96 | −35.55 ± 7.81 |
| D-1573 | 2263 | 3 | 4 | AAV1 | GalNAc | −16.91 ± 14.29 | −16.85 ± 11.81 | −19.48 ± 14.82 |
| D-1638 | 2263 | 5 | 3 | AAV1 | C22 | 6.18 ± 39.91 | 1.64 ± 38.82 | 0.64 ± 24.22 |
| D-1638 | 2263 | 15 | 4 | AAV1 | C22 | −14.48 ± 14.06 | −15.34 ± 16.81 | −32.49 ± 23.85 |
| D-1644 | 2263 | 5 | 3 | AAV1 | C22 | −4.49 ± 28.46 | −8.13 ± 28.85 | −9.74 ± 20.2 |
| D-1644 | 2263 | 15 | 3 | AAV1 | C22 | −40.62 ± 32.29 | −41.72 ± 34.15 | −45.04 ± 28.65 |
| D-1645 | 2263 | 0.5 | 3 | AAV1 | GalNAc | 17.05 ± 15.72 | 13.25 ± 12.64 | 5.3 ± 8.75 |
| D-1645 | 2263 | 1 | 3 | AAV1 | GalNAc | 11.58 ± 11.59 | 12 ± 9.78 | 19.45 ± 15.6 |
| D-1645 | 2263 | 3 | 4 | AAV1 | GalNAc | −30.31 ± 8.48 | −31.96 ± 5.84 | −34.08 ± 16.03 |
| D-1557 | 3000 | 1 | 3 | AAV2 | GalNAc | 3.82 ± 37.62 | −4.13 ± 31.55 | 5.9 ± 34.59 |
| D-1557 | 3000 | 3 | 3 | AAV2 | GalNAc | 9.8 ± 49.78 | 6.24 ± 48.34 | 27.57 ± 48.71 |
| D-1642 | 3000 | 15 | 3 | AAV2 | C22 | −60.07 ± 23.8 | −58.2 ± 24.84 | −52.11 ± 26.5 |
| D-1586 | 3133 | 1 | 4 | AAV2 | GalNAc | −9.95 ± 20.45 | −7.39 ± 16.97 | −6.25 ± 21.94 |
| D-1586 | 3133 | 3 | 3 | AAV2 | GalNAc | −46.14 ± 14.71 | −42.98 ± 17.58 | −39.14 ± 15.32 |
| D-1637 | 3133 | 15 | 3 | AAV2 | C22 | −17.3 ± 10.47 | −17.27 ± 10.65 | −16.31 ± 9.96 |

TABLE 6
siRNA Efficacy in Liver 4 Weeks Following siRNA Injection

| SIRNA | Trigger | Dose | | AAV | | Avg. % Change in mRNA ± STD | | |
|---|---|---|---|---|---|---|---|---|
| Duplex | Family | (mg/kg) | N | No. | Carrier | eGFP-1 | eGFP-2 | BGHpA |
| D-1599 | 1328 | 1 | 4 | AAV1 | GalNAc | −45.95 ± 34.12 | −51.22 ± 29.38 | −43.47 ± 38.85 |
| D-1597 | 1333 | 1 | 4 | AAV1 | GalNAc | −62.57 ± 20.24 | −64.15 ± 18.92 | −62.8 ± 14.32 |
| D-1589 | 1496 | 1 | 4 | AAV1 | GalNAc | 14.3 ± 63.6 | 10.78 ± 62.99 | −4.3 ± 34.03 |
| D-1616 | 1534 | 1 | 4 | AAV1 | GalNAc | −11.65 ± 14.21 | −16.95 ± 16.59 | −11.05 ± 10.43 |
| D-1610 | 1631 | 1 | 4 | AAV1 | GalNAc | −48.26 ± 55.95 | −52.2 ± 51.51 | −53.22 ± 49.44 |
| D-1607 | 1666 | 1 | 4 | AAV1 | GalNAc | −49.8 ± 32.9 | −51.01 ± 30.93 | −54.32 ± 27.54 |
| D-1609 | 1671 | 1 | 4 | AAV1 | GalNAc | −4.39 ± 32.6 | −17.13 ± 26.03 | −19.54 ± 26.39 |
| D-1615 | 1678 | 1 | 4 | AAV1 | GalNAc | −41.13 ± 53.81 | −49.28 ± 45.76 | −45.21 ± 48.78 |
| D-1605 | 1698 | 1 | 4 | AAV1 | GalNAc | −33.95 ± 19.59 | −36.11 ± 16.39 | −36.42 ± 16.43 |
| D-1606 | 1705 | 1 | 4 | AAV1 | GalNAc | −17.39 ± 28.54 | −19.93 ± 31.05 | −25.46 ± 25.93 |
| D-1587 | 1801 | 1 | 4 | AAV1 | GalNAc | 45.99 ± 35.41 | 32.63 ± 29.48 | 14.33 ± 25.53 |
| D-1608 | 1952 | 1 | 4 | AAV1 | GalNAc | 21.23 ± 45.64 | 18.69 ± 46.27 | 12.11 ± 38.75 |
| D-1601 | 2075 | 1 | 4 | AAV1 | GalNAc | 3.48 ± 52.45 | 11.81 ± 55.2 | −28.69 ± 27.8 |
| D-1602 | 2270 | 1 | 4 | AAV1 | GalNAc | −31.34 ± 30.1 | −29.49 ± 32.27 | −29.03 ± 29.76 |
| D-1613 | 2344 | 1 | 4 | AAV1 | GalNAc | −16.52 ± 26.48 | −5.98 ± 37.64 | −16.72 ± 16.64 |
| D-1598 | 2353 | 1 | 3 | AAV1 | GalNAc | −6.46 ± 79.75 | 2.57 ± 87.09 | −11.44 ± 74.95 |
| D-1595 | 2358 | 1 | 3 | AAV1 | GalNAc | −17.2 ± 28.13 | −8.88 ± 30.22 | −8.88 ± 30.22 |
| D-1592 | 2462 | 1 | 4 | AAV1 | GalNAc | −19.82 ± 24.34 | −16.13 ± 26.34 | 98.03 ± 8.2 |
| D-1621 | 2632 | 1 | 4 | AAV1 | GalNAc | −13.73 ± 45.73 | −9.86 ± 49.02 | 64.93 ± 59.16 |
| D-1620 | 2889 | 1 | 4 | AAV2 | GalNAc | −37.46 ± 43.63 | −40.78 ± 35.94 | −45.42 ± 32.5 |
| D-1604 | 2923 | 1 | 4 | AAV2 | GalNAc | 39.14 ± 72.97 | 25.32 ± 61.13 | 15.5 ± 48.93 |
| D-1588 | 2937 | 1 | 3 | AAV2 | GalNAc | 6.07 ± 22.94 | 5.99 ± 30.89 | −5.12 ± 13.34 |
| D-1619 | 2994 | 1 | 4 | AAV2 | GalNAc | 39.67 ± 48.64 | 44.18 ± 65.89 | 18.49 ± 38.81 |
| D-1618 | 3189 | 1 | 4 | AAV2 | GalNAc | 10.19 ± 27.42 | 3.71 ± 24.64 | 0.48 ± 24.64 |
| D-1632 | 3429 | 1 | 4 | AAV2 | GalNAc | 45.62 ± 34.77 | 41.55 ± 34.47 | 23.62 ± 31.38 |
| D-1617 | 3717 | 1 | 4 | AAV2 | GalNAc | 38.84 ± 47.24 | 37.53 ± 45.21 | 19 ± 42.75 |
| D-1626 | 3720 | 1 | 4 | AAV2 | GalNAc | −6.37 ± 40.83 | −11.26 ± 36.19 | −17.84 ± 31.67 |
| D-1600 | 4109 | 1 | 4 | AAV2 | GalNAc | 27.89 ± 36.42 | 22.01 ± 32.66 | 12.46 ± 28.14 |
| D-1590 | 4779 | 1 | 3 | AAV3 | GalNAc | −33.73 ± 13 | −38.93 ± 12.77 | 6 ± 19.5 |
| D-1630 | 4804 | 1 | 4 | AAV3 | GalNAc | −14.77 ± 6.49 | −12.14 ± 2.53 | −30.29 ± 35.81 |
| D-1596 | 4927 | 1 | 4 | AAV3 | GalNAc | −42.19 ± 18.45 | −41.27 ± 17.05 | −42.41 ± 18.48 |
| D-1594 | 4928 | 1 | 4 | AAV3 | GalNAc | −2.76 ± 27.16 | −1.57 ± 31.74 | −39.67 ± 30.85 |
| D-1593 | 4956 | 1 | 4 | AAV3 | GalNAc | 8.35 ± 10.98 | 14.89 ± 12.4 | 36.47 ± 110.18 |
| D-1631 | 4957 | 1 | 4 | AAV3 | GalNAc | −57.28 ± 10.53 | −54.33 ± 14.16 | −61.59 ± 11.91 |
| D-1634 | 4993 | 1 | 4 | AAV3 | GalNAc | −34.13 ± 23.86 | −29.69 ± 22.95 | −42.05 ± 16.41 |
| D-1614 | 4999 | 1 | 4 | AAV3 | GalNAc | −72.8 ± 18.55 | −70.72 ± 19.27 | −75.89 ± 15.31 |
| D-1633 | 5012 | 1 | 4 | AAV3 | GalNAc | −44.29 ± 14.87 | −41.01 ± 14.68 | −47.43 ± 12.41 |
| D-1611 | 5043 | 1 | 4 | AAV3 | GalNAc | −67.5 ± 14.12 | −64.75 ± 15.77 | −70.05 ± 12.45 |
| D-1612 | 5045 | 1 | 4 | AAV3 | GalNAc | −57.91 ± 11.94 | −56.76 ± 12.24 | −72.15 ± 19.53 |
| D-1591 | 5060 | 1 | 4 | AAV3 | GalNAc | −37.88 ± 21.07 | −36.57 ± 20.16 | −43.71 ± 19.3 |
| D-1603 | 5067 | 1 | 4 | AAV3 | GalNAc | −27.08 ± 12.11 | −21.28 ± 12.91 | −29.76 ± 9.66 |
| D-1629 | 5068 | 1 | 4 | AAV3 | GalNAc | −25.94 ± 28.07 | −22.16 ± 29.33 | −27.4 ± 24.84 |
| D-1628 | 5069 | 1 | 4 | AAV3 | GalNAc | −36.64 ± 25.96 | −33.81 ± 25.97 | −37.94 ± 22.7 |
| D-1623 | 5080 | 1 | 4 | AAV3 | GalNAc | −34.14 ± 2.45 | −29.15 ± 4.19 | −40.61 ± 6.41 |
| D-1627 | 5115 | 1 | 4 | AAV3 | GalNAc | −9.14 ± 24.05 | −4.67 ± 24.43 | −12.81 ± 27.17 |
| D-1622 | 5255 | 1 | 4 | AAV3 | GalNAc | −36.04 ± 17.88 | −33.61 ± 19.59 | −34.26 ± 15.69 |
| D-1625 | 5338 | 1 | 4 | AAV3 | GalNAc | −26.25 ± 35.36 | −24.19 ± 36.13 | −29.8 ± 29.64 |

TABLE 7
siRNA Efficacy in Liver and Adipose Tissue 4 Weeks Following siRNA Injection*

| siRNA | Trigger | Dose | | | | Avg. % Change in mRNA ± STD | | |
|---|---|---|---|---|---|---|---|---|
| Duplex | Family | (mg/kg) | N | Carrier | Tissue | eGFP-1 | eGFP-2 | BGHpA |
| D-1597 | 1333 | 3 | 4 | GalNAc | Liver | −56.3 ± 21.55 | −55.72 ± 21.86 | −51.8 ± 24.69 |
| D-1545 | 1309 | 3 | 3 | GalNAc | Liver | −38.49 ± 7.6 | −38.32 ± 10.46 | −35.9 ± 12.63 |
| D-1640 | 1309 | 3 | 4 | GalNAc | Liver | −27.46 ± 16.52 | −30.18 ± 15.06 | −26.35 ± 9.34 |
| D-1646 | 1309 | 3 | 4 | GalNAc | Liver | −33.27 ± 15.52 | −35.26 ± 16.91 | −36.1 ± 11.18 |
| D-1652 | 1309 | 3 | 4 | GalNAc | Liver | −37.03 ± 10.19 | −37.27 ± 11.97 | −40.53 ± 10.69 |
| D-1657 | 1309 | 3 | 4 | GalNAc | Liver | −42.36 ± 27.93 | −44.67 ± 25.92 | −47.49 ± 20.08 |
| D-1662 | 1309 | 3 | 4 | GalNAc | Liver | −49.31 ± 3.2 | −49.12 ± 3.99 | −36.17 ± 6.86 |
| D-1667 | 1309 | 3 | 4 | GalNAc | Liver | 64.41 ± 14.9 | −64.39 ± 13.81 | −53.08 ± 18.99 |
| D-1549 | 2080 | 3 | 3 | GalNAc | Liver | −42.27 ± 1.69 | −37.74 ± 6.96 | −36.56 ± 12.45 |
| D-1647 | 2080 | 3 | 4 | GalNAc | Liver | −9.81 ± 14.47 | −14.38 ± 34.58 | −15 ± 35.29 |
| D-1651 | 2080 | 3 | 4 | GalNAc | Liver | −14.14 ± 38.82 | −15.22 ± 33.33 | −7.23 ± 42.67 |
| D-1656 | 2080 | 3 | 4 | GalNAc | Liver | −26.48 ± 28.43 | 45.17 ± 17.46 | −46.61 ± 18.99 |
| D-1661 | 2080 | 3 | 4 | GalNAc | Liver | −57.19 ± 35.09 | −57.87 ± 34.18 | −46.61 ± 38.09 |
| D-1666 | 2080 | 3 | 4 | GalNAc | Liver | −10.47 ± 28.04 | −23.11 ± 34.64 | −21.77 ± 37.38 |
| D-1544 | 2144 | 3 | 4 | GalNAc | Liver | −43.5 ± 14.87 | −40.65 ± 14.86 | −27.59 ± 20.88 |
| D-1648 | 2144 | 3 | 4 | GalNAc | Liver | −53.99 ± 29.24 | −53.2 ± 28.75 | −46.51 ± 30.91 |
| D-1653 | 2144 | 3 | 4 | GalNAc | Liver | −36.13 ± 14.87 | −34.76 ± 16.33 | −25.79 ± 18.55 |
| D-1658 | 2144 | 3 | 4 | GalNAc | Liver | −39.78 ± 25.74 | −34.7 ± 27.83 | −32.37 ± 17.93 |
| D-1663 | 2144 | 3 | 4 | GalNAc | Liver | −27.73 ± 16.32 | −24.24 ± 16.47 | −23.95 ± 12.37 |
| D-1668 | 2144 | 3 | 4 | GalNAc | Liver | −29.31 ± 14.33 | −30.61 ± 11.87 | −19.7 ± 18.95 |
| D-1635 | 1309 | 20 | 4 | C22 | Liver | −31.24 ± 27.79 | −31.05 ± 29.03 | −23.73 ± 19.73 |
| D-1639 | 1309 | 20 | 4 | C22 | Liver | −43.19 ± 40.95 | −36.31 ± 47.36 | −40.72 ± 42.52 |
| D-1670 | 1309 | 20 | 4 | C22 | Liver | −18.18 ± 16.92 | −8.04 ± 17.92 | −16.58 ± 9.32 |
| D-1676 | 1309 | 20 | 4 | C22 | Liver | −22.36 ± 34.59 | −17.1 ± 39.02 | −11.76 ± 38.63 |
| D-1681 | 1309 | 20 | 3 | C22 | Liver | 47.73 ± 7.7 | −52.39 ± 10.5 | −55.07 ± 10.11 |
| D-1686 | 1309 | 20 | 3 | C22 | Liver | −46.26 ± 7.27 | −46.39 ± 8.07 | −44.22 ± 16.43 |
| D-1691 | 1309 | 20 | 4 | C22 | Liver | −32.27 ± 13.41 | −24.45 ± 15.05 | −28.15 ± 14.13 |
| D-1635 | 1309 | 20 | 4 | C22 | Adipose | 7.94 ± 65.35 | 9.18 ± 74.04 | −9.4 ± 78.81 |
| D-1639 | 1309 | 20 | 4 | C22 | Adipose | −61.2 ± 32.62 | −61.1 ± 33.16 | −61.15 ± 36.91 |
| D-1670 | 1309 | 20 | 4 | C22 | Adipose | −56.88 ± 23.59 | −51.24 ± 33.29 | −59.98 ± 24.89 |
| D-1676 | 1309 | 20 | 4 | C22 | Adipose | 63.2 ± 100.25 | 42.97 ± 75.56 | 24.61 ± 57.61 |
| D-1681 | 1309 | 20 | 3 | C22 | Adipose | 29.33 ± 103.09 | 19.17 ± 93.9 | −9.62 ± 76.91 |
| D-1686 | 1309 | 20 | 3 | C22 | Adipose | −79.73 ± 13.34 | −80.1 ± 13.72 | −80.3 ± 11.86 |
| D-1691 | 1309 | 20 | 4 | C22 | Adipose | −18.98 ± 17.2 | −20.98 ± 11.52 | −26.98 ± 11.76 |

*Table 7 contains data only from mice infected with the AAV1 viral construct

TABLE 8
siRNA Efficacy in Liver and Adipose Tissue 4 Weeks Following siRNA Injection

| SIRNA | Trigger | Dose | | AAV | | | Avg. % Change in mRNA ± STD | | |
|---|---|---|---|---|---|---|---|---|---|
| Duplex | Family | (mg/kg) | N | No. | Carrier | Tissue | eGFP-1 | eGFP-2 | BGHpA |
| D-1597 | 1333 | 1 | 4 | AAV1 | GalNAc | Liver | −37.5 ± 14.1 | −30.9 ± 13.3 | −47.3 ± 6.1 |
| D-1615 | 1678 | 1 | 4 | AAV1 | GalNAc | Liver | −37.9 ± 18 | −34.7 ± 19.1 | −38.8 ± 17.8 |
| D-1631 | 4957 | 1 | 4 | AAV3 | GalNAc | Liver | −32.1 ± 10.7 | −26.1 ± 17.3 | −38.3 ± 15.5 |
| D-1614 | 4999 | 1 | 4 | AAV3 | GalNAc | Liver | −53.2 ± 12.8 | −49.7 ± 17 | −56.2 ± 9 |
| D-1611 | 5043 | 1 | 4 | AAV3 | GalNAc | Liver | −34.3 ± 33.8 | −33.3 ± 32 | −44.5 ± 21.2 |
| D-1612 | 5045 | 1 | 4 | AAV3 | GalNAc | Liver | −31.1 ± 27.1 | −30.1 ± 27.3 | −34.5 ± 18.5 |
| D-1597 | 1333 | 3 | 4 | AAV1 | GalNAc | Liver | −72.3 ± 10.6 | −69.5 ± 12.8 | −72.2 ± 7.4 |
| D-1615 | 1678 | 3 | 4 | AAV1 | GalNAc | Liver | −65.5 ± 6.1 | −63.2 ± 7 | −63.9 ± 4.7 |
| D-1631 | 4957 | 3 | 4 | AAV3 | GalNAc | Liver | −68 ± 6.9 | −65.3 ± 7.7 | −65.5 ± 7.2 |
| D-1614 | 4999 | 3 | 4 | AAV3 | GalNAc | Liver | −67.3 ± 2.4 | −64.9 ± 2.6 | −65.2 ± 0.6 |
| D-1611 | 5043 | 3 | 4 | AAV3 | GalNAc | Liver | −66.8 ± 17.1 | −65.2 ± 17.7 | −66.4 ± 14.9 |
| D-1612 | 5045 | 3 | 4 | AAV3 | GalNAc | Liver | −83 ± 13 | −82.2 ± 14 | −82.6 ± 14.2 |
| D-1694 | 1333 | 20 | 4 | AAV1 | C22 | Liver | −73.3 ± 9.4 | −71.3 ± 10.4 | −72.2 ± 3.7 |
| D-1695 | 1678 | 20 | 4 | AAV1 | C22 | Liver | −51.9 ± 33.7 | −51 ± 33.5 | −56.8 ± 29.8 |
| D-1696 | 4957 | 20 | 4 | AAV3 | C22 | Liver | −34.7 ± 29.7 | −34.3 ± 31.1 | −43.6 ± 21.4 |
| D-1697 | 4999 | 20 | 4 | AAV3 | C22 | Liver | −3.3 ± 53.7 | 1.1 ± 55.4 | −18.9 ± 43.9 |
| D-1698 | 5043 | 20 | 4 | AAV3 | C22 | Liver | −32.6 ± 15.1 | −31 ± 15.9 | −43.7 ± 10.3 |
| D-1699 | 5045 | 20 | 4 | AAV3 | C22 | Liver | −18.6 ± 16.2 | −18.7 ± 13.7 | −28.2 ± 11.7 |
| D-1694 | 1333 | 20 | 4 | AAV1 | C22 | Adipose | −66.4 ± 8 | −67.6 ± 9.6 | −75.2 ± 7 |
| D-1695 | 1678 | 20 | 4 | AAV1 | C22 | Adipose | −19 ± 55.4 | −17.5 ± 59.3 | −43.7 ± 43 |
| D-1696 | 4957 | 20 | 4 | AAV3 | C22 | Adipose | −75.6 ± 34.8 | −75.3 ± 34.7 | −78.1 ± 27.5 |
| D-1697 | 4999 | 20 | 4 | AAV3 | C22 | Adipose | −79.7 ± 5.8 | −78.5 ± 6.4 | −73.2 ± 9.1 |
| D-1698 | 5043 | 20 | 4 | AAV3 | C22 | Adipose | −69 ± 21.9 | −67.5 ± 25.3 | −68.4 ± 19.7 |
| D-1699 | 5045 | 20 | 4 | AAV3 | C22 | Adipose | −72.2 ± 15.9 | −69.1 ± 19.5 | −54.4 ± 17.1 |

TABLE 9
siRNA Efficacy in Liver and Adipose Tissue 4 Weeks Following siRNA Injection

| SIRNA | Trigger | Dose | | AAV | | | Avg. % Change in mRNA ± STD | | |
|---|---|---|---|---|---|---|---|---|---|
| Duplex | Family | (mg/kg) | N | No. | Carrier | Tissue | eGFP-1 | eGFP-2 | BGHpA |
| D-1557 | 3000 | 3 | 4 | AAV2 | GalNAc | Liver | −52.1 ± 15.3 | −51.3 ± 15.8 | −46.9 ± 19.4 |
| D-1650 | 3000 | 3 | 4 | AAV2 | GalNAc | Liver | −54.1 ± 10 | −53.1 ± 10.5 | −52.4 ± 15.2 |
| D-1655 | 3000 | 3 | 4 | AAV2 | GalNAc | Liver | −44.6 ± 9.6 | −42.8 ± 10 | −42.3 ± 7.1 |
| D-1660 | 3000 | 3 | 4 | AAV2 | GalNAc | Liver | −39.3 ± 8.9 | −42.9 ± 3.7 | −43.4 ± 8.3 |
| D-1665 | 3000 | 3 | 4 | AAV2 | GalNAc | Liver | −20.5 ± 22 | −19.6 ± 21.1 | −22.1 ± 17.8 |
| D-1586 | 3133 | 3 | 4 | AAV2 | GalNAc | Liver | −38.6−9.4 | −36.8 ± 8.5 | −40 ± 2.3 |
| D-1649 | 3133 | 3 | 4 | AAV2 | GalNAc | Liver | −43.4 ± 5.6 | −37.6 ± 6.5 | −42.5 ± 12.4 |
| D-1654 | 3133 | 3 | 4 | AAV2 | GalNAc | Liver | −27.4 ± 46.7 | −22 ± 49.3 | −20.8 ± 46.1 |
| D-1659 | 3133 | 3 | 4 | AAV2 | GalNAc | Liver | −41.5 ± 2.9 | −36.8 ± 5.7 | −42.1 ± 6.3 |
| D-1664 | 3133 | 3 | 4 | AAV2 | GalNAc | Liver | −43.7 ± 14.8 | −41.6 ± 15.9 | −45.5 ± 12.1 |
| D-1669 | 3133 | 3 | 4 | AAV2 | GalNAc | Liver | −45.7 ± 27.1 | −40.7 ± 29.6 | −46.4 ± 28.6 |
| D-1623 | 5080 | 3 | 4 | AAV3 | GalNAc | Liver | −27.1 ± 25.9 | −22.4 ± 30.4 | −27.1 ± 24.9 |
| D-1643 | 2080 | 20 | 4 | AAV1 | C22 | Liver | −14.9 ± 19.5 | −14.6 ± 21.1 | −11.6 ± 17.1 |
| D-1671 | 2080 | 20 | 4 | AAV1 | C22 | Liver | −20.1 ± 23.6 | −20.5 ± 23.4 | −17 ± 25.9 |
| D-1675 | 2080 | 20 | 4 | AAV1 | C22 | Liver | −32.6 ± 11.2 | −31.9 ± 14 | −19.5 ± 15.8 |
| D-1680 | 2080 | 20 | 4 | AAVI | C22 | Liver | −52.1 ± 13.6 | −50 ± 12.9 | −42.1 ± 11.9 |
| D-1685 | 2080 | 20 | 4 | AAV1 | C22 | Liver | −54 ± 25.8 | −53.5 ± 26.2 | −46.7 ± 29 |
| D-1690 | 2080 | 20 | 4 | AAVI | C22 | Liver | −40 ± 10.9 | −36.2 ± 8.7 | −33.9 ± 13.3 |
| D-1636 | 2144 | 20 | 4 | AAV1 | C22 | Liver | −48.8 ± 30 | −44.2 ± 33.6 | −40.9 ± 33.7 |
| D-1672 | 2144 | 20 | 4 | AAV1 | C22 | Liver | −11 ± 16.5 | −7.5 ± 16.9 | −20 ± 6.3 |
| D-1677 | 2144 | 20 | 4 | AAV1 | C22 | Liver | −23.6 ± 9.1 | −20.2 ± 7.2 | −17.5 ± 9.3 |
| D-1682 | 2144 | 20 | 4 | AAV1 | C22 | Liver | −14.4 ± 9.8 | −13 ± 9.2 | −29.9 ± 5.7 |
| D-1687 | 2144 | 20 | 4 | AAV1 | C22 | Liver | −59.5 ± 33.4 | −61 ± 32.4 | −74.3 ± 18.5 |
| D-1692 | 2144 | 20 | 4 | AAVI | C22 | Liver | −60.5 ± 21.6 | −60.1 ± 23.3 | −73 ± 6.7 |
| D-1846 | 5080 | 20 | 4 | AAV3 | C22 | Liver | −76.6 ± 36 | −79.6 ± 29.9 | −83.5 ± 27.2 |
| D-1643 | 2080 | 20 | 4 | AAVI | C22 | Adipose | −53.4 ± 23.5 | −55.1 ± 23.9 | −65.8 ± 16.6 |
| D-1671 | 2080 | 20 | 4 | AAV1 | C22 | Adipose | −74.9 ± 7.5 | −79.2 ± 5 | −76.1 ± 10.3 |
| D-1675 | 2080 | 20 | 4 | AAV1 | C22 | Adipose | −64.9 ± 14.4 | −67.8 ± 14.9 | −71 ± 13.7 |
| D-1680 | 2080 | 20 | 4 | AAVI | C22 | Adipose | −69.6 ± 26.1 | −66.3 ± 29 | −83.1 ± 11.1 |
| D-1685 | 2080 | 20 | 4 | AAV1 | C22 | Adipose | −70.2 ± 32 | −67.3 ± 35.7 | −80.4 ± 20 |
| D-1690 | 2080 | 20 | 4 | AAV1 | C22 | Adipose | −81.5 ± 11.6 | −82.6 ± 10.5 | −82.1 ± 16.7 |
| D-1636 | 2144 | 20 | 4 | AAV1 | C22 | Adipose | −65.7 ± 40.7 | −60 ± 47.4 | −78.5 ± 26.6 |
| D-1672 | 2144 | 20 | 4 | AAV1 | C22 | Adipose | −63.3 ± 21.1 | −65.6 ± 19.9 | −59.1 ± 27.6 |
| D-1677 | 2144 | 20 | 4 | AAV1 | C22 | Adipose | −44.2 ± 19.2 | −35.8 ± 26 | −56.6 ± 18.2 |
| D-1682 | 2144 | 20 | 4 | AAV1 | C22 | Adipose | −85.5 ± 7.7 | −81.1 ± 8.9 | −78.3 ± 14 |
| D-1687 | 2144 | 20 | 4 | AAV1 | C22 | Adipose | −52.1 ± 15.3 | −51.3 ± 15.8 | −46.9 ± 19.4 |
| D-1692 | 2144 | 20 | 4 | AAV1 | C22 | Adipose | −54.1 ± 10 | −53.1 ± 10.5 | −52.4 ± 15.2 |
| D-1846 | 5080 | 20 | 4 | AAV3 | C22 | Adipose | −44.6 ± 9.6 | −42.8 ± 10 | −42.3 ± 7.1 |

TABLE 10
siRNA Efficacy in Liver 4 Weeks Following siRNA Injection

| SIRNA | Trigger | Dose | | AAV | | Avg. % Change in mRNA ± STD | | |
|---|---|---|---|---|---|---|---|---|
| Duplex | Family | (mg/kg) | N | No. | Carrier | eGFP-1 | eGFP-2 | BGHpA |
| D-1597 | 1333 | 3 | 4 | 1 | GalNAc | −57.5 ± 17.2 | −54.9 ± 17.5 | −55.1 ± 15.5 |
| D-1721 | 1333 | 3 | 4 | 1 | GalNAc | −35.5 ± 4 | −31.5 ± 8.3 | −34 ± 3.1 |
| D-1728 | 1333 | 3 | 4 | 1 | GalNAc | −32.5 ± 18.5 | −28.1 ± 18.5 | −36.8 ± 13.8 |
| D-1735 | 1333 | 3 | 4 | 1 | GalNAc | −36.3 ± 11.7 | −35 ± 7.2 | −36.3 ± 8.6 |
| D-1707 | 1333 | 3 | 4 | 1 | GalNAc | −37.8 ± 10.4 | −39.3 ± 9.7 | −34.5 ± 4.1 |
| D-1714 | 1333 | 3 | 4 | 1 | GalNAc | −50.6 ± 18.8 | −49.1 ± 19.4 | −45.8 ± 18 |
| D-1700 | 1333 | 3 | 4 | 1 | GalNAc | −44.5 ± 15.2 | −40.9 ± 17.9 | −45.8 ± 18.5 |
| D-1615 | 1678 | 3 | 4 | 1 | GalNAc | −53.7 ± 19.8 | −52 ± 20.9 | −47.8 ± 14.9 |
| D-1722 | 1678 | 3 | 4 | 1 | GalNAc | −45.8 ± 22 | −40.4 ± 24.7 | −46.9 ± 14.7 |
| D-1729 | 1678 | 3 | 4 | 1 | GalNAc | −31.8 ± 15.6 | −27.5 ± 16.4 | −43.2 ± 4.9 |
| D-1736 | 1678 | 3 | 4 | 1 | GalNAc | −51.7 ± 8.2 | −50.6 ± 7.3 | −43.2 ± 5.4 |
| D-1708 | 1678 | 3 | 4 | 1 | GalNAc | −36.1 ± 9.7 | −32.3 ± 11.1 | −35.2 ± 9.7 |
| D-1715 | 1678 | 3 | 4 | 1 | GalNAc | −28.1 ± 28.8 | −22.6 ± 30.8 | −32.9 ± 21 |
| D-1701 | 1678 | 3 | 4 | 1 | GalNAc | −27.8 ± 20.9 | −25.6 ± 18.1 | −17 ± 18.8 |
| D-1631 | 4957 | 3 | 4 | 3 | GalNAc | −55.3 ± 18.1 | −56.1 ± 19.4 | −52.7 ± 18.3 |
| D-1724 | 4957 | 3 | 4 | 3 | GalNAc | −62.3 ± 8.1 | −62.5 ± 7 | −59.1 ± 7.3 |
| D-1731 | 4957 | 3 | 4 | 3 | GalNAc | −64 ± 11.9 | −59.9 ± 14.4 | −58.9 ± 15.6 |
| D-1738 | 4957 | 3 | 4 | 3 | GalNAc | −42.1 ± 9.4 | −40.2 ± 11.3 | −46.7 ± 9 |
| D-1710 | 4957 | 3 | 4 | 3 | GalNAc | −57.9 ± 6.3 | −57.3 ± 6.3 | −49.7 ± 4.3 |
| D-1717 | 4957 | 3 | 4 | 3 | GalNAc | −61.4 ± 24.8 | −58.5 ± 28.6 | −59.3 ± 23.8 |
| D-1703 | 4957 | 3 | 4 | 3 | GalNAc | −61.2 ± 10 | −62.1 ± 9 | −54.3 ± 12.5 |
| D-1614 | 4999 | 3 | 4 | 3 | GalNAc | −71 ± 21.5 | −70.7 ± 21.7 | −73.5 ± 16.8 |
| D-1723 | 4999 | 3 | 4 | 3 | GalNAc | −70.8 ± 3.4 | −70 ± 2.2 | −60.8 ± 1.5 |
| D-1730 | 4999 | 3 | 4 | 3 | GalNAc | −72.4 ± 15 | −72.5 ± 16.2 | −67.2 ± 11.7 |
| D-1737 | 4999 | 3 | 4 | 3 | GalNAc | −71.5 ± 3.9 | −74.5 ± 5 | −65.1 ± 1.5 |
| D-1709 | 4999 | 3 | 4 | 3 | GalNAc | −73 ± 5 | −71.6 ± 5.3 | −67.5 ± 6.2 |
| D-1716 | 4999 | 3 | 4 | 3 | GalNAc | −76 ± 4 | −75.5 ± 4.2 | −69.4 ± 4.1 |
| D-1702 | 4999 | 3 | 4 | 3 | GalNAc | −72.7 ± 4.9 | −72.1 ± 4.7 | −67.9 ± 2.1 |
| D-1611 | 5043 | 3 | 4 | 3 | GalNAc | −71.6 ± 11.4 | −69.4 ± 12 | −65.2 ± 10.6 |
| D-1726 | 5043 | 3 | 4 | 3 | GalNAc | −59.8 ± 17.1 | −57.3 ± 18.3 | −58.8 ± 13.7 |
| D-1733 | 5043 | 3 | 4 | 3 | GalNAc | −70.8 ± 8.1 | −68.3 ± 9.2 | −64.3 ± 8.7 |
| D-1740 | 5043 | 3 | 4 | 3 | GalNAc | −61.6 ± 10.8 | −57.9 ± 12.3 | −56.6 ± 6.9 |
| D-1712 | 5043 | 3 | 4 | 3 | GalNAc | −67.8 ± 9 | −64.7 ± 10.4 | −69.6 ± 6.3 |
| D-1719 | 5043 | 3 | 4 | 3 | GalNAc | −69.4 ± 6.2 | −68.9 ± 5.6 | −68.1 ± 3.3 |
| D-1705 | 5043 | 3 | 4 | 3 | GalNAc | −81.4 ± 4.9 | −77.9 ± 8.8 | −79.7 ± 8.6 |
| D-1612 | 5045 | 3 | 3 | 3 | GalNAc | −67.5 ± 15.5 | −69.6 ± 14.1 | −75.3 ± 7.4 |
| D-1725 | 5045 | 3 | 4 | 3 | GalNAc | −64.8 ± 5.2 | −66.7 ± 5.4 | −67.1 ± 3.8 |
| D-1732 | 5045 | 3 | 4 | 3 | GalNAc | −66 ± 9.9 | −64.2 ± 10.6 | −69.5 ± 5.7 |
| D-1739 | 5045 | 3 | 4 | 3 | GalNAc | −66.8 ± 12.9 | −65.5 ± 14.7 | −67.6 ± 11 |
| D-1711 | 5045 | 3 | 4 | 3 | GalNAc | −75.7 ± 6.7 | −75.8 ± 5.1 | −71.5 ± 5.1 |
| D-1718 | 5045 | 3 | 4 | 3 | GalNAc | −76.6 ± 4.1 | −75.7 ± 4.4 | −75.6 ± 2.8 |
| D-1704 | 5045 | 3 | 4 | 3 | GalNAc | −80.1 ± 12.2 | −80.1 ± 12.1 | −79.9 ± 12.4 |
| D-1623 | 5080 | 3 | 4 | 3 | GalNAc | −72.9 ± 7.6 | −72.6 ± 7.1 | −75.9 ± 7.1 |
| D-1727 | 5080 | 3 | 4 | 3 | GalNAc | −59 ± 3.6 | −53.4 ± 12.7 | −49.3 ± 20.9 |
| D-1734 | 5080 | 3 | 4 | 3 | GalNAc | −59.6 ± 7.4 | −60.3 ± 7 | −61.2 ± 6.2 |
| D-1741 | 5080 | 3 | 4 | 3 | GalNAc | −59.9 ± 4.8 | −59.4 ± 4.1 | −61 ± 5.8 |
| D-1713 | 5080 | 3 | 4 | 3 | GalNAc | −67.9 ± 11.1 | −67.5 ± 8.7 | −69 ± 4.9 |
| D-1720 | 5080 | 3 | 4 | 3 | GalNAc | −55.3 ± 6.4 | −57 ± 6.5 | −60.7 ± 5.6 |
| D-1706 | 5080 | 3 | 4 | 3 | GalNAc | −66.2 ± 9 | −67.7 ± 7.8 | −69.1 ± 6.7 |

TABLE 11
siRNA Efficacy in Liver and Adipose Tissue 4 Weeks Following siRNA Injection

| SIRNA | Trigger | Dose | | AAV | | | Avg. % Change in mRNA ± STD | | |
|---|---|---|---|---|---|---|---|---|---|
| Duplex | Family | (mg/kg) | N | No. | Carrier | Tissue | eGFP-1 | eGFP-2 | BGHpA |
| D-1714 | 1333 | 3 | 4 | 1 | GalNAc | Liver | −50.6 ± 13.9 | −49.2 ± 14.5 | −46.6 ± 17.4 |
| D-1615 | 1678 | 3 | 4 | 1 | GalNAc | Liver | −52.5 ± 11.4 | −53.1 ± 10.4 | −57.9 ± 6.1 |
| D-1736 | 1678 | 3 | 4 | 1 | GalNAc | Liver | −44.9 ± 8.9 | −43.1 ± 9.7 | −50 ± 7.7 |
| D-1648 | 2144 | 3 | 4 | 1 | GalNAc | Liver | −39.4 ± 13 | −37.3 ± 13.2 | −38.5 ± 10.9 |
| D-1557 | 3000 | 3 | 4 | 2 | GalNAc | Liver | −45.8 ± 11 | −44.6 ± 12.7 | −46.8 ± 13.2 |
| D-1650 | 3000 | 3 | 4 | 2 | GalNAc | Liver | −32.2 ± 30.3 | −27.5 ± 32.7 | −35.5 ± 20.2 |
| D-1664 | 3133 | 3 | 4 | 2 | GalNAc | Liver | −38.4 ± 23.9 | −31.8 ± 26.2 | −46.1 ± 11.7 |
| D-1614 | 4999 | 3 | 4 | 3 | GalNAc | Liver | −66.3 ± 13 | −59.8 ± 14.3 | −58.7 ± 13.9 |
| D-1723 | 4999 | 3 | 4 | 3 | GalNAc | Liver | −65.3 ± 16.8 | −58.7 ± 19 | −58.5 ± 14.9 |
| D-1737 | 4999 | 3 | 4 | 3 | GalNAc | Liver | −57.2 ± 22.1 | −51.3 ± 21 | −47.9 ± 15.1 |
| D-1730 | 4999 | 3 | 4 | 3 | GalNAc | Liver | −61 ± 24.9 | −56.6 ± 25.7 | −48.8 ± 24 |
| D-1709 | 4999 | 3 | 4 | 3 | GalNAc | Liver | −73 ± 6.3 | −67.4 ± 8 | −68.1 ± 16.8 |
| D-1716 | 4999 | 3 | 4 | 3 | GalNAc | Liver | −64.8 ± 14.9 | −57.1 ± 19.5 | −56.6 ± 10.9 |
| D-1702 | 4999 | 3 | 3 | 3 | GalNAc | Liver | −55.6 ± 13.3 | −45.7 ± 15.6 | −50.4 ± 7.6 |
| D-1879 | 4999 | 3 | 4 | 3 | GalNAc | Liver | −70.1 ± 10.9 | −62.6 ± 13.7 | −55.6 ± 15.7 |
| D-1611 | 5043 | 3 | 4 | 3 | GalNAc | Liver | −77.2 ± 9.6 | −78.2 ± 9.1 | −69.5 ± 13.8 |
| D-1733 | 5043 | 3 | 4 | 3 | GalNAc | Liver | −75.3 ± 9.1 | −75.7 ± 9.3 | −69.2 ± 10.8 |
| D-1719 | 5043 | 3 | 4 | 3 | GalNAc | Liver | −70.9 ± 14 | −71.2 ± 12.7 | −70.9 ± 9.4 |
| D-1705 | 5043 | 3 | 4 | 3 | GalNAc | Liver | −75.7 ± 10.3 | −76.7 ± 9.8 | −74.4 ± 7.9 |
| D-1612 | 5045 | 3 | 3 | 3 | GalNAc | Liver | −72.1 ± 6 | −71.2 ± 5.3 | −65.2 ± 7.2 |
| D-1711 | 5045 | 3 | 4 | 3 | GalNAc | Liver | −73 ± 3.8 | −72.4 ± 3.4 | −70.4 ± 4.4 |
| D-1718 | 5045 | 3 | 4 | 3 | GalNAc | Liver | −75.4 ± 14.3 | −75.4 ± 14.5 | −70.1 ± 16.3 |
| D-1704 | 5045 | 3 | 4 | 3 | GalNAc | Liver | −76.4 ± 5.3 | −73.9 ± 6.2 | −71.6 ± 5.6 |
| D-1876 | 5045 | 20 | 4 | 3 | C22 | Liver | −31 ± 19.2 | −34 ± 17.4 | −38 ± 14.6 |
| D-1623 | 5080 | 3 | 3 | 3 | GalNAc | Liver | −71.4 ± 3.3 | −65.6 ± 4.9 | −65.9 ± 2.3 |
| D-1706 | 5080 | 3 | 4 | 3 | GalNAc | Liver | 64.9 ± 12.7 | −63.6 ± 16.6 | 64.9 ± 8.5 |
| D-1873 | 1333 | 20 | 4 | 1 | C22 | Liver | −37.5 ± 21.5 | −33 ± 24.6 | −53.8 ± 12.3 |
| D-1695 | 1678 | 20 | 4 | 1 | C22 | Liver | −22 ± 15.4 | −22.4 ± 13.1 | −31.6 ± 12.9 |
| D-1867 | 1678 | 20 | 3 | 1 | C22 | Liver | −48.2 ± 10.4 | −49.7 ± 6.3 | −51.4 ± 5.3 |
| D-1672 | 2144 | 20 | 4 | 1 | C22 | Liver | −48 ± 17.3 | −47.1 ± 17.8 | −49.6 ± 15.8 |
| D-1642 | 3000 | 20 | 4 | 2 | C22 | Liver | −38 ± 15.5 | −35.7 ± 16.1 | −36.2 ± 9.4 |
| D-1674 | 3000 | 20 | 3 | 2 | C22 | Liver | −44.8 ± 11.5 | −45.9 ± 9 | 45.3 ± 15.1 |
| D-1688 | 3133 | 20 | 4 | 2 | C22 | Liver | −41 ± 28.5 | −32.2 ± 32.9 | −47.4 ± 21.9 |
| D-1697 | 4999 | 20 | 4 | 3 | C22 | Liver | −22.4 ± 23 | −14 ± 27.2 | −15.4 ± 25.6 |
| D-1865 | 4999 | 20 | 4 | 3 | C22 | Liver | −16.8 ± 47.2 | −4.9 ± 53.2 | −14.1 ± 33.6 |
| D-1863 | 4999 | 20 | 4 | 3 | C22 | Liver | −0.6 ± 33.2 | 16.7 ± 46.2 | 1.2 ± 32.6 |
| D-1866 | 4999 | 20 | 4 | 3 | C22 | Liver | −11.7 ± 61.1 | −2.2 ± 65.8 | −7.8 ± 45.2 |
| D-1869 | 4999 | 20 | 4 | 3 | C22 | Liver | −78 ± 7.7 | −73.1 ± 9.9 | −66.4 ± 9.2 |
| D-1872 | 4999 | 20 | 4 | 3 | C22 | Liver | 65.6 ± 11.2 | −56.5 ± 14.2 | −54.4 ± 12.2 |
| D-1877 | 4999 | 20 | 4 | 3 | C22 | Liver | −68.4 ± 7.9 | −60.1 ± 12.1 | −58 ± 11.8 |
| D-1878 | 4999 | 20 | 3 | 3 | C22 | Liver | −58.2 ± 9.1 | −49.9 ± 8.6 | −52.2 ± 7.5 |
| D-1698 | 5043 | 20 | 4 | 3 | C22 | Liver | −72.2 ± 15 | −72.8 ± 15.1 | −70.5 ± 18.3 |
| D-1864 | 5043 | 20 | 4 | 3 | C22 | Liver | −35.4 ± 11.3 | −39.2 ± 8.5 | −41.2 ± 12.7 |
| D-1870 | 5043 | 20 | 4 | 3 | C22 | Liver | −30.4 ± 11.4 | −25.1 ± 9.3 | −32.1 ± 8.7 |
| D-1875 | 5043 | 20 | 4 | 3 | C22 | Liver | −61.5 ± 19.5 | −61.1 ± 20.5 | −60.9 ± 18.2 |
| D-1699 | 5045 | 20 | 4 | 3 | C22 | Liver | −40.1 ± 10.8 | −42.4 ± 10.7 | −38.2 ± 9.6 |
| D-1868 | 5045 | 20 | 4 | 3 | C22 | Liver | −52.7 ± 11.7 | −45.4 ± 11.3 | −48.7 ± 8.4 |
| D-1871 | 5045 | 20 | 4 | 3 | C22 | Liver | −65.4 ± 5.2 | −65.3 ± 4 | −66.8 ± 5.2 |
| D-1846 | 5080 | 20 | 4 | 3 | C22 | Liver | −25.8 ± 17.6 | −12.7 ± 20.8 | −22.9 ± 17.3 |
| D-1874 | 5080 | 20 | 4 | 3 | C22 | Liver | 41.6 ± 10.4 | −36.9 ± 11.9 | −40.6 ± 7.5 |
| D-1873 | 1333 | 20 | 4 | 1 | C22 | Adipose | 37.4 ± 68 | 31.4 ± 63.8 | 6.3 ± 35.5 |
| D-1695 | 1678 | 20 | 4 | 1 | C22 | Adipose | −19.2 ± 12.7 | −28 ± 12.9 | −25.3 ± 7.2 |
| D-1867 | 1678 | 20 | 3 | 1 | C22 | Adipose | −67.3 ± 35.7 | −69.4 ± 35.3 | −71.5 ± 24.1 |
| D-1672 | 2144 | 20 | 4 | 1 | C22 | Adipose | −43 ± 26.1 | −43.1 ± 31.7 | −32.6 ± 37.7 |
| D-1642 | 3000 | 20 | 4 | 2 | C22 | Adipose | −37.2 ± 40.5 | −30.3 ± 45.9 | −15.8 ± 61.1 |
| D-1674 | 3000 | 20 | 3 | 2 | C22 | Adipose | −70.7 ± 19.9 | −68.6 ± 21.7 | −71.4 ± 19.9 |
| D-1688 | 3133 | 20 | 4 | 2 | C22 | Adipose | −39.6 ± 18.4 | −39.7 ± 21.7 | −28.9 ± 44.3 |
| D-1697 | 4999 | 20 | 4 | 3 | C22 | Adipose | −85.9 ± 7.3 | −89.5 ± 5.6 | −81.5 ± 8.1 |
| D-1865 | 4999 | 20 | 4 | 3 | C22 | Adipose | −78.6 ± 25.5 | −78.2 ± 29.5 | −81.7 ± 21 |
| D-1863 | 4999 | 20 | 4 | 3 | C22 | Adipose | −84.6 ± 8.1 | −87.1 ± 6.9 | −72.9 ± 15.4 |
| D-1866 | 4999 | 20 | 4 | 3 | C22 | Adipose | −88.5 ± 10.9 | −91.6 ± 6.7 | −86.6 ± 13.3 |
| D-1869 | 4999 | 20 | 4 | 3 | C22 | Adipose | −90 ± 8.2 | −92.4 ± 6.7 | −85.3 ± 10.6 |
| D-1872 | 4999 | 20 | 4 | 3 | C22 | Adipose | −66.2 ± 15.1 | −75.9 ± 9.4 | −72.9 ± 11.9 |
| D-1877 | 4999 | 20 | 4 | 3 | C22 | Adipose | −73.7 ± 6.7 | −79.6 ± 5 | −62.8 ± 14.3 |
| D-1878 | 4999 | 20 | 3 | 3 | C22 | Adipose | −85.2 ± 12 | −88.8 ± 9.3 | −84.7 ± 10.5 |
| D-1698 | 5043 | 20 | 4 | 3 | C22 | Adipose | −93 ± 9 | −91.5 ± 11.3 | −91.4 ± 10.7 |
| D-1864 | 5043 | 20 | 4 | 3 | C22 | Adipose | −87.6 ± 7.4 | −86.7 ± 7.2 | −84.6 ± 9.5 |
| D-1870 | 5043 | 20 | 4 | 3 | C22 | Adipose | −81.6 ± 15.5 | −81 ± 16.7 | −80.5 ± 14 |
| D-1875 | 5043 | 20 | 4 | 3 | C22 | Adipose | −85.6 ± 9.1 | −86.9 ± 8.2 | −81.3 ± 12.9 |
| D-1699 | 5045 | 20 | 4 | 3 | C22 | Adipose | −78.8 ± 9.5 | −78.7 ± 10.6 | −79.8 ± 7.7 |
| D-1868 | 5045 | 20 | 4 | 3 | C22 | Adipose | −82 ± 9 | −79.4 ± 11.7 | −79.6 ± 9.2 |
| D-1871 | 5045 | 20 | 4 | 3 | C22 | Adipose | −90 ± 15.1 | −91.9 ± 12 | −90.9 ± 12.9 |
| D-1876 | 5045 | 20 | 4 | 3 | C22 | Adipose | −76 ± 24.4 | −76.9 ± 22.6 | −75.2 ± 25.4 |
| D-1846 | 5080 | 20 | 4 | 3 | C22 | Adipose | −82.9 ± 8.8 | −87.2 ± 7.7 | −75.9 ± 14.9 |
| D-1874 | 5080 | 20 | 4 | 3 | C22 | Adipose | −71.2 ± 9.7 | −72.8 ± 8.8 | −74.2 ± 5.7 |

TABLE 12
siRNA Efficacy in Liver and Adipose Tissue 4 Weeks Following siRNA Injection

| SIRNA | Trigger | Dose | | AAV | | | Avg. % Change in mRNA ± STD | | |
|---|---|---|---|---|---|---|---|---|---|
| Duplex | Family | (mg/kg) | N | No. | Carrier | Tissue | eGFP-1 | eGFP-2 | BGHpA |
| D-1686 | 1309 | 20 | 4 | 1 | C22 | Liver | −39.9 ± 38.8 | −39.9 ± 40.6 | −51 ± 29.2 |
| D-1691 | 1309 | 20 | 4 | 1 | C22 | Liver | −40.1 ± 18.4 | −40 ± 16.9 | −49.8 ± 10.6 |
| D-1635 | 1309 | 20 | 4 | 1 | C22 | Liver | −38.4 ± 30.6 | −38.8 ± 29.3 | −45.9 ± 19.5 |
| D-1694 | 1333 | 20 | 4 | 1 | C22 | Liver | −25 ± 20.4 | −26 ± 21.8 | −43.4 ± 13.7 |
| D-1695 | 1678 | 20 | 4 | 1 | C22 | Liver | −11.9 ± 10.8 | −14 ± 11.7 | −19.5 ± 17.5 |
| D-1643 | 2080 | 20 | 4 | 1 | C22 | Liver | −44.2 ± 17.3 | −44.1 ± 17.4 | −46.2 ± 11.4 |
| D-1672 | 2144 | 20 | 4 | 1 | C22 | Liver | −58.5 ± 9.6 | −55.6 ± 9.9 | −52 ± 10.2 |
| D-1636 | 2144 | 20 | 4 | 1 | C22 | Liver | −65.3 ± 31.3 | −66.3 ± 30.2 | −63.4 ± 32.9 |
| D-1847 | 1309 | 20 | 4 | 1 | C22 | Liver | −43 ± 14.5 | −39.9 ± 17.1 | −49.9 ± 12.1 |
| D-1849 | 1309 | 20 | 4 | 1 | C22 | Liver | −23.5 ± 13.9 | −19.6 ± 14.1 | −14.8 ± 11.6 |
| D-1859 | 1309 | 20 | 4 | 1 | C22 | Liver | −33.1 ± 15.6 | −33.3 ± 13.8 | −36.9 ± 15.7 |
| D-1853 | 1333 | 20 | 4 | 1 | C22 | Liver | −42.2 ± 22.9 | −41 ± 19.8 | −44.1 ± 20.7 |
| D-1852 | 1678 | 20 | 4 | 1 | C22 | Liver | −51.2 ± 21.2 | −44.9 ± 23.4 | −38 ± 21.7 |
| D-1860 | 2080 | 20 | 4 | 1 | C22 | Liver | −14.1 ± 23.1 | −11.5 ± 21.8 | −5.5 ± 34.2 |
| D-1851 | 2144 | 20 | 4 | 1 | C22 | Liver | −61.6 ± 15.8 | −55.8 ± 17.8 | −52.5 ± 16.3 |
| D-1858 | 2144 | 20 | 4 | 1 | C22 | Liver | −47.6 ± 10.8 | −44.7 ± 10.2 | −46.2 ± 1.9 |
| D-1666 | 4999 | 20 | 4 | 3 | C22 | Liver | −35 ± 11.7 | −35.7 ± 11.2 | −38.6 ± 11.7 |
| D-1872 | 4999 | 20 | 4 | 3 | C22 | Liver | −78.4 ± 5.1 | −76.2 ± 6.1 | −71.3 ± 4.3 |
| D-1877 | 4999 | 20 | 4 | 3 | C22 | Liver | −84.6 ± 1.7 | −82.1 ± 1.9 | −79.9 ± 2.4 |
| D-1697 | 4999 | 20 | 4 | 3 | C22 | Liver | −40.9 ± 9.3 | −37.5 ± 10.5 | −38.9 ± 8.3 |
| D-1846 | 5080 | 20 | 4 | 3 | C22 | Liver | −43.3 ± 6.8 | 43.8 ± 6.3 | −40 ± 4.5 |
| D-1881 | 4999 | 20 | 4 | 3 | C22 | Liver | −80.2 ± 2.7 | −77.3 ± 3.3 | −71 ± 2.8 |
| D-1887 | 4999 | 20 | 4 | 3 | C22 | Liver | −83.2 ± 3.3 | −80.2 ± 3.8 | −75.6 ± 4.5 |
| D-1880 | 4999 | 20 | 4 | 3 | C22 | Liver | −83.6 ± 2.7 | −82.1 ± 2.8 | −81.5 ± 2.9 |
| D-1884 | 4999 | 20 | 4 | 3 | C22 | Liver | −88 ± 2.9 | −85.5 ± 3.2 | −86 ± 2.3 |
| D-1856 | 4999 | 20 | 4 | 3 | C22 | Liver | −82.3 ± 4.5 | −79.7 ± 5 | −74.3 ± 5.8 |
| D-1862 | 5080 | 20 | 4 | 3 | C22 | Liver | −72.3 ± 3.5 | −69.3 ± 4.1 | −65 ± 3.8 |
| D-1869 | 4999 | 20 | 4 | 3 | C22 | Liver | −78.8 ± 2.7 | −73.6 ± 2.9 | −68.7 ± 1 |
| D-1869 | 4999 | 10 | 4 | 3 | C22 | Liver | −72 ± 3.1 | −69.4 ± 2.9 | −67.1 ± 3.3 |
| D-1869 | 4999 | 5 | 4 | 3 | C22 | Liver | −63.3 ± 6.4 | −60.9 ± 6.8 | −57.3 ± 7 |
| D-1709 | 4999 | 3 | 4 | 3 | GalNAc | Liver | −81.1 ± 4 | −77.1 ± 4.7 | −75.4 ± 5.2 |
| D-1709 | 4999 | 1 | 4 | 3 | GalNAc | Liver | −42.9 ± 7 | −39 ± 7.6 | −43.8 ± 7.5 |
| D-1709 | 4999 | 0.5 | 4 | 3 | GalNAc | Liver | −60.9 ± 6 | −58.1 ± 5.9 | −57.8 ± 5.8 |
| D-1774 | 5276 | 3 | 4 | 3 | GalNAc | Liver | −66.1 ± 4.5 | −59.6 ± 5.6 | −57.6 ± 4.7 |
| D-1975 | 5276 | 20 | 4 | 3 | C22 | Liver | −73.5 ± 3.6 | −70.7 ± 3.5 | −65.4 ± 3.3 |
| D-1976 | 5276 | 20 | 3 | 3 | C22 | Liver | −80.2 ± 0.4 | −76.7 ± 0.3 | −69.3 ± 0.7 |
| D-1870 | 5043 | 20 | 4 | 3 | C22 | Liver | −49 ± 11.5 | −47.6 ± 11.6 | −49.4 ± 11 |
| D-1698 | 5043 | 20 | 4 | 3 | C22 | Liver | −10.2 ± 15.9 | −13.2 ± 16 | −27.4 ± 9.6 |
| D-1875 | 5043 | 20 | 4 | 3 | C22 | Liver | −41.9 ± 9 | −40.1 ± 10.4 | −44.8 ± 6.9 |
| D-1868 | 5045 | 20 | 4 | 3 | C22 | Liver | −33 ± 10.8 | −32.2 ± 11.1 | −35.7 ± 9.5 |
| D-1871 | 5045 | 20 | 4 | 3 | C22 | Liver | −26 ± 7.2 | −27.4 ± 7.4 | −27.7 ± 6.2 |
| D-1876 | 5045 | 20 | 4 | 3 | C22 | Liver | −32 ± 3.2 | −25.4 ± 2.7 | −29.9 ± 3.8 |
| D-1699 | 5045 | 20 | 4 | 3 | C22 | Liver | −22.6 ± 16.4 | −20.2 ± 16.8 | −21.4 ± 15.4 |
| D-1883 | 5043 | 20 | 4 | 3 | C22 | Liver | −64.8 ± 6.2 | −63.2 ± 6.4 | −64.8 ± 6 |
| D-1886 | 5043 | 20 | 4 | 3 | C22 | Liver | −73.5 ± 5.6 | −70.4 ± 6.6 | −70.9 ± 6.2 |
| D-1855 | 5045 | 20 | 4 | 3 | C22 | Liver | −61 ± 5.4 | −57.8 ± 6.1 | −53.2 ± 2.9 |
| D-1888 | 5045 | 20 | 4 | 3 | C22 | Liver | −68.3 ± 3.5 | −65.1 ± 2.8 | −67.2 ± 2.4 |
| D-1882 | 5045 | 20 | 4 | 3 | C22 | Liver | −69.1 ± 3.3 | −64.7 ± 5 | −63.7 ± 4.1 |
| D-1885 | 5045 | 20 | 4 | 3 | C22 | Liver | −66 ± 1.9 | −62.4 ± 2.1 | −59 ± 2 |
| D-1611 | 5043 | 3 | 4 | 3 | GalNAc | Liver | −70.5 ± 6.1 | −71.5 ± 6.1 | −70.3 ± 5.7 |
| D-1611 | 5043 | 1 | 4 | 3 | GalNAc | Liver | −17.9 ± 13.6 | −21.3 ± 15.3 | −28.7 ± 8.1 |
| D-1611 | 5043 | 0.5 | 4 | 3 | GalNAc | Liver | 5.7 ± 5.4 | −3.3 ± 4.3 | −5.7 ± 6.5 |
| D-1718 | 5045 | 3 | 4 | 3 | GalNAc | Liver | −63.1 ± 1.5 | −56.3 ± 1.9 | −62.1 ± 1.1 |
| D-1718 | 5045 | 1 | 4 | 3 | GalNAc | Liver | −37.8 ± 21.9 | −30.9 ± 26.4 | −33.7 ± 19.9 |
| D-1718 | 5045 | 0.5 | 4 | 3 | GalNAc | Liver | −21 ± 13.3 | −16.5 ± 16.5 | −21.2 ± 16.9 |
| D-1866 | 4999 | 20 | 4 | 3 | C22 | Adipose | −94.3 ± 1.3 | −93.9 ± 1.3 | −90.9 ± 1.4 |
| D-1873 | 4999 | 20 | 4 | 3 | C22 | Adipose | −94.3 ± 1.7 | −93.7 ± 1.8 | −89.8 ± 3.3 |
| D-1877 | 4999 | 20 | 4 | 3 | C22 | Adipose | −95 ± 1.7 | −94.7 ± 1.8 | −94.9 ± 1.7 |
| D-1697 | 4999 | 20 | 4 | 3 | C22 | Adipose | −96.9 ± 1.2 | −96.6 ± 1.4 | −96.1 ± 1.5 |
| D-1846 | 5080 | 20 | 4 | 3 | C22 | Adipose | −92.2 ± 2.4 | −90.8 ± 3 | −90.9 ± 2.6 |
| D-1881 | 4999 | 20 | 4 | 3 | C22 | Adipose | −97.3 ± 0.7 | −97.2 ± 0.7 | −96.6 ± 0.9 |
| D-1887 | 4999 | 20 | 4 | 3 | C22 | Adipose | −93.9 ± 4 | −93.2 ± 4.7 | −95.8 ± 2.3 |
| D-1880 | 4999 | 20 | 4 | 3 | C22 | Adipose | −96.1 ± 1.8 | −95.6 ± 2 | −96.3 ± 1.4 |
| D-1884 | 4999 | 20 | 4 | 3 | C22 | Adipose | −98.6 ± 0.5 | −98.5 ± 0.6 | −98.4 ± 0.5 |
| D-1856 | 4999 | 20 | 4 | 3 | C22 | Adipose | −98.3 ± 0.6 | −98.1 ± 0.7 | −97.5 ± 0.9 |
| D-1862 | 5080 | 20 | 4 | 3 | C22 | Adipose | −96.1 ± 1.2 | −95.7 ± 1.5 | −95.4 ± 1.2 |
| D-1869 | 4999 | 20 | 4 | 3 | C22 | Adipose | −93.9 ± 1.6 | −92.5 ± 2.2 | −92.8 ± 0.5 |
| D-1869 | 4999 | 10 | 4 | 3 | C22 | Adipose | −95.5 ± 1.3 | −94.8 ± 1.6 | −95.6 ± 1.3 |
| D-1869 | 4999 | 5 | 4 | 3 | C22 | Adipose | −87.7 ± 4.9 | −86.2 ± 5.1 | −88.6 ± 4.1 |
| D-1975 | 5276 | 20 | 4 | 3 | C22 | Adipose | −95.9 ± 1.4 | −95.2 ± 1.4 | −95.9 ± 1.3 |
| D-1976 | 5276 | 20 | 3 | 3 | C22 | Adipose | −94.1 ± 2.4 | −93.1 ± 2.6 | −92.2 ± 2.7 |
| D-1870 | 5043 | 20 | 4 | 3 | C22 | Adipose | −84.4 ± 6.6 | −85.6 ± 6.3 | −83.9 ± 6.8 |
| D-1698 | 5043 | 20 | 4 | 3 | C22 | Adipose | −70.8 ± 5.6 | −71.8 ± 5.3 | −63.4 ± 7.9 |
| D-1875 | 5043 | 20 | 4 | 3 | C22 | Adipose | −87.5 ± 4 | −88.9 ± 3.5 | −83.6 ± 6.1 |
| D-1868 | 5045 | 20 | 4 | 3 | C22 | Adipose | −72.1 ± 13.2 | −73.5 ± 13.3 | −68.2 ± 10.6 |
| D-1871 | 5045 | 20 | 4 | 3 | C22 | Adipose | −61.6 ± 12.1 | −64 ± 11.3 | −62.7 ± 6.2 |
| D-1876 | 5045 | 20 | 4 | 3 | C22 | Adipose | −82.3 ± 5.1 | −82.5 ± 5.1 | −71.8 ± 6.4 |
| D-1699 | 5045 | 20 | 4 | 3 | C22 | Adipose | −81.5 ± 4.5 | −82.6 ± 4.6 | −65.5 ± 10.1 |
| D-1883 | 5043 | 20 | 4 | 3 | C22 | Adipose | −90 ± 5.9 | −90.7 ± 5.7 | −85.9 ± 8.2 |
| D-1886 | 5043 | 20 | 4 | 3 | C22 | Adipose | −84.9 ± 7.6 | −84 ± 7.9 | −81.7 ± 9.8 |
| D-1855 | 5045 | 20 | 4 | 3 | C22 | Adipose | −76.9 ± 8.7 | −77.7 ± 8.4 | −68.8 ± 5.7 |
| D-1888 | 5045 | 20 | 4 | 3 | C22 | Adipose | −82.1 ± 3 | −83.1 ± 3 | −70.2 ± 5 |
| D-1882 | 5045 | 20 | 4 | 3 | C22 | Adipose | −74.7 ± 6.7 | −74.7 ± 6.7 | −59.7 ± 9.4 |
| D-1885 | 5045 | 20 | 4 | 3 | C22 | Adipose | −86.1 ± 1.6 | −86.8 ± 1.6 | −78.6 ± 3 |

TABLE 13
siRNA Efficacy in Liver 4 Weeks Following siRNA Injection

| SIRNA | Trigger | Dose | | | | Avg. % Change in mRNA ± STD | | |
|---|---|---|---|---|---|---|---|---|
| Duplex | Family | (mg/kg) | N | AAV No. | Carrier | eGFP-1 | eGFP-2 | BGHpA |
| D-1933 | 1514 | 3 | 4 | 1 | GalNAc | 3.7 ± 14.8 | 12.5 ± 17 | 2.9 ± 18.1 |
| D-1939 | 1514 | 3 | 4 | 1 | GalNAc | 18.7 ± 10.1 | 23.6 ± 11.5 | 21.7 ± 15.2 |
| D-1945 | 1514 | 3 | 4 | 1 | GalNAc | −33.5 ± 16.6 | −26.5 ± 18.2 | −25.2 ± 9.8 |
| D-1951 | 1514 | 3 | 4 | 1 | GalNAc | 7.5 ± 65.3 | 15.5 ± 67.9 | 4.3 ± 46.6 |
| D-1957 | 1514 | 3 | 4 | 1 | GalNAc | 12.1 ± 38.9 | 17.4 ± 37.8 | 5.6 ± 35.6 |
| D-1963 | 1514 | 3 | 4 | 1 | GalNAc | −7.4 ± 45.8 | 4.7 ± 55.1 | −11.4 ± 41.8 |
| D-1969 | 1514 | 3 | 4 | 1 | GalNAc | −12.6 ± 14.6 | −6.2 ± 11.6 | −7.7 ± 12 |
| D-1820 | 1514 | 3 | 4 | 1 | GalNAc | −24.8 ± 19 | −14.5 ± 27.1 | −17.8 ± 21.1 |
| D-1938 | 2343 | 3 | 4 | 1 | GalNAc | −37.7 ± 14.9 | −32.9 ± 17.1 | −13.3 ± 22.1 |
| D-1944 | 2343 | 3 | 4 | 1 | GalNAc | −35 ± 3.2 | −26 ± 7.1 | −21 ± 16.2 |
| D-1950 | 2343 | 3 | 4 | 1 | GalNAc | −18.4 ± 11 | −9.8 ± 10.8 | −9.8 ± 7.6 |
| D-1956 | 2343 | 3 | 4 | 1 | GalNAc | −48.5 ± 43 | −42.3 ± 51.4 | −40.4 ± 49.1 |
| D-1962 | 2343 | 3 | 4 | 1 | GalNAc | −10.4 ± 19.6 | 2.8 ± 27.4 | −10.1 ± 19.3 |
| D-1968 | 2343 | 3 | 4 | 1 | GalNAc | −44.2 ± 16.5 | −36.9 ± 20 | −27 ± 21.7 |
| D-1974 | 2343 | 3 | 4 | 1 | GalNAc | −52.5 ± 5.8 | −46.5 ± 4.2 | −33.5 ± 6.4 |
| D-1810 | 2343 | 3 | 4 | 1 | GalNAc | −42.8 ± 41.3 | −38.5 ± 44.2 | −30 ± 47.6 |
| D-1935 | 2417 | 3 | 4 | 1 | GalNAc | −20.7 ± 16.2 | −13.1 ± 23.6 | −34.4 ± 16.2 |
| D-1941 | 2417 | 3 | 4 | 1 | GalNAc | −27.7 ± 27.6 | −24.9 ± 29.4 | −28.4 ± 29.4 |
| D-1947 | 2417 | 3 | 4 | 1 | GalNAc | −24 ± 41.1 | −18.9 ± 46.2 | −28.4 ± 32.2 |
| D-1953 | 2417 | 3 | 4 | 1 | GalNAc | −48.3 ± 21.8 | −40.7 ± 25.3 | −54.8 ± 16.5 |
| D-1959 | 2417 | 3 | 4 | 1 | GalNAc | −31.3 ± 10.2 | −27.3 ± 8.9 | −42.4 ± 9.6 |
| D-1965 | 2417 | 3 | 4 | 1 | GalNAc | −30.7 ± 13.8 | −25.7 ± 13.8 | −33.8 ± 12 |
| D-1971 | 2417 | 3 | 4 | 1 | GalNAc | −25 ± 22 | −10.9 ± 28.5 | −25.4 ± 33 |
| D-1812 | 2417 | 3 | 4 | 1 | GalNAc | −29.2 ± 17.7 | −27 ± 17.8 | −39 ± 14 |
| D-1937 | 4412 | 3 | 3 | 3 | GalNAc | −74 ± 6.9 | −70.8 ± 6.4 | −66.4 ± 6.8 |
| D-1943 | 4412 | 3 | 4 | 3 | GalNAc | −68 ± 12.7 | −64.2 ± 15 | −66.3 ± 11 |
| D-1949 | 4412 | 3 | 4 | 3 | GalNAc | −54.8 ± 7.8 | −52.2 ± 6.7 | −55.5 ± 5.7 |
| D-1955 | 4412 | 3 | 4 | 3 | GalNAc | −84.5 ± 1.6 | −82.6 ± 2.2 | −76.7 ± 3.5 |
| D-1961 | 4412 | 3 | 4 | 3 | GalNAc | −78 ± 12 | −75.7 ± 13.3 | −73 ± 13.1 |
| D-1967 | 4412 | 3 | 4 | 3 | GalNAc | −72.2 ± 24 | −70 ± 25.1 | −64.3 ± 22.4 |
| D-1973 | 4412 | 3 | 4 | 3 | GalNAc | −62.5 ± 7.3 | −56.8 ± 7.3 | −58.2 ± 3 |
| D-1777 | 4412 | 3 | 4 | 3 | GalNAc | −74 ± 17.9 | −72.8 ± 20 | −72 ± 20.7 |
| D-1934 | 5249 | 3 | 4 | 3 | GalNAc | −10.6 ± 25.9 | −4.4 ± 25.7 | −21.6 ± 24.3 |
| D-1940 | 5249 | 3 | 4 | 3 | GalNAc | −41.4 ± 11.5 | −39.9 ± 9.3 | −46.1 ± 10.9 |
| D-1946 | 5249 | 3 | 4 | 3 | GalNAc | −45.9 ± 12.7 | −43.7 ± 13.7 | −50 ± 7.2 |
| D-1952 | 5249 | 3 | 4 | 3 | GalNAc | −72.6 ± 10.9 | −72.3 ± 11.8 | −74.3 ± 12.6 |
| D-1958 | 5249 | 3 | 4 | 3 | GalNAc | −45.9 ± 26.4 | −44.7 ± 27.7 | −56.1 ± 17.1 |
| D-1964 | 5249 | 3 | 4 | 3 | GalNAc | −56.3 ± 19.1 | −56.1 ± 17.1 | −60.4 ± 12.1 |
| D-1970 | 5249 | 3 | 4 | 3 | GalNAc | −79.4 ± 15 | −79.3 ± 14.9 | −80.1 ± 14 |
| D-1769 | 5249 | 3 | 4 | 3 | GalNAc | −67.2 ± 3.2 | −64.9 ± 4.7 | −72.2 ± 3.8 |
| D-1936 | 5274 | 3 | 4 | 3 | GalNAc | −61.7 ± 13 | −60.9 ± 13.1 | −67.3 ± 10.4 |
| D-1942 | 5274 | 3 | 4 | 3 | GalNAc | −51.7 ± 36.3 | −50.8 ± 37.5 | −63.6 ± 19.5 |
| D-1948 | 5274 | 3 | 4 | 3 | GalNAc | −70.5 ± 8.4 | −67.8 ± 11 | −71.7 ± 6.5 |
| D-1954 | 5274 | 3 | 4 | 3 | GalNAc | −71.9 ± 8.4 | −70.6 ± 9.6 | −74 ± 6.1 |
| D-1960 | 5274 | 3 | 4 | 3 | GalNAc | −69.7 ± 15.5 | −67.5 ± 16.3 | −77.9 ± 5.6 |
| D-1966 | 5274 | 3 | 4 | 3 | GalNAc | −76.8 ± 12.6 | −74.1 ± 14 | −76.2 ± 11.3 |
| D-1972 | 5274 | 3 | 4 | 3 | GalNAc | −79.7 ± 8.6 | −77.4 ± 10 | −77 ± 10.5 |
| D-1773 | 5274 | 3 | 4 | 3 | GalNAc | −69.5 ± 13.7 | −66.8 ± 14.1 | −73.9 ± 11.7 |
| D-1709 | 4999 | 3 | 4 | 3 | GalNAc | −86.7 ± 8.9 | −85.2 ± 9.8 | −84 ± 10.7 |
| D-1705 | 5043 | 3 | 4 | 3 | GalNAc | −78.5 ± 2.3 | −77.1 ± 2.2 | −75.3 ± 2.7 |
| D-1597 | 1333 | 3 | 4 | 9-span | GalNAc | −81.3 ± 6.9 | −81.6 ± 7.7 | −81.6 ± 6.7 |
| D-1709 | 4999 | 3 | 4 | 9-span | GalNAc | −93.4 ± 2.5 | −93.7 ± 2 | −94.5 ± 1.9 |
| D-1705 | 5043 | 3 | 4 | 9-span | GalNAc | −94.4 ± 0.4 | −94.2 ± 0.5 | −94.7 ± 0.4 |

TABLE 14
siRNA Efficacy in Liver and Adipose Tissue 4 Weeks Following siRNA Injection

| SIRNA | Trigger | Dose | | AAV | | | Avg. % Change in mRNA ± STD | | |
|---|---|---|---|---|---|---|---|---|---|
| Duplex | Family | (mg/kg) | N | No. | Tissue | Carrier | eGFP-1 | eGFP-2 | BGHpA |
| D-1709 | 4999 | 3 | 4 | 22-span | Liver | GalNAc | −80.8 ± 12 | −81.6 ± 9.7 | −85.7 ± 8.9 |
| D-1887 | 4999 | 20 | 4 | 22-span | Liver | C22 | −85.6 ± 2.1 | −85.9 ± 2.7 | −88.9 ± 1.7 |
| D-1702 | 4999 | 3 | 4 | 22-span | Liver | GalNAc | −83.6 ± 8.3 | −84.3 ± 7.4 | −88.3 ± 5.2 |
| D-1884 | 4999 | 20 | 4 | 22-span | Liver | C22 | −79.5 ± 2.9 | −80.2 ± 3.4 | −85.1 ± 2.2 |
| D-1978 | 4999 | 3 | 4 | 22-span | Liver | GalNAc | −85.9 ± 3.8 | −86.4 ± 3.4 | −89.8 ± 3 |
| D-1879 | 4999 | 3 | 4 | 22-span | Liver | GalNAc | −81.3 ± 3 | −82.6 ± 2.1 | −87.1 ± 0.7 |
| D-2002 | 4999 | 20 | 4 | 22-span | Liver | C22 | −88.5 ± 5.5 | −88.5 ± 5.4 | −92.1 ± 3.2 |
| D-1987 | 4999 | 3 | 4 | 22-span | Liver | GalNAc | −85.9 ± 5.6 | −86.4 ± 5.2 | −91.6 ± 2.2 |
| D-1992 | 4999 | 3 | 4 | 22-span | Liver | GalNAc | −82.7 ± 11.3 | −84 ± 9.9 | −88.1 ± 5.8 |
| D-1997 | 4999 | 3 | 4 | 22-span | Liver | GalNAc | −84.7 ± 7.1 | −84.8 ± 6.2 | −88.1 ± 6.2 |
| D-1705 | 5043 | 3 | 4 | 22-span | Liver | GalNAc | −82.3 ± 8.1 | −82.4 ± 8.1 | −84.2 ± 7 |
| D-1886 | 5043 | 20 | 4 | 22-span | Liver | C22 | −86.2 ± 11.9 | −87.3 ± 10.7 | −87.9 ± 8.7 |
| D-1980 | 5043 | 3 | 4 | 22-span | Liver | GalNAc | −76.9 ± 20.9 | −77.6 ± 19 | −80.4 ± 15.2 |
| D-1984 | 5043 | 3 | 4 | 22-span | Liver | GalNAc | −77.3 ± 8 | −78.5 ± 7.6 | −82.3 ± 6.2 |
| D-2004 | 5043 | 20 | 3 | 22-span | Liver | C22 | −65.8 ± 11.7 | −69.4 ± 10 | −73.7 ± 7.7 |
| D-1989 | 5043 | 3 | 4 | 22-span | Liver | GalNAc | −82.8 ± 9.6 | −83.4 ± 8 | −84.3 ± 7.3 |
| D-1994 | 5043 | 3 | 4 | 22-span | Liver | GalNAc | −79.1 ± 3.3 | −79.5 ± 2.2 | −83 ± 2.2 |
| D-1999 | 5043 | 3 | 4 | 22-span | Liver | GalNAc | −66.2 ± 27.4 | −67.7 ± 25.4 | −69.6 ± 23.7 |
| D-1704 | 5045 | 3 | 4 | 22-span | Liver | GalNAc | −83 ± 10.2 | −83.9 ± 10.1 | −88.6 ± 5.4 |
| D-1885 | 5045 | 20 | 4 | 22-span | Liver | C22 | −89.1 ± 5.5 | −90.3 ± 4.9 | −90.1 ± 7 |
| D-1979 | 5045 | 3 | 4 | 22-span | Liver | GalNAc | −92.3 ± 3.3 | −93 ± 3 | −93.7 ± 2.4 |
| D-1983 | 5045 | 3 | 4 | 22-span | Liver | GalNAc | −71.8 ± 8.3 | −74.6 ± 7.4 | −83.6 ± 4.8 |
| D-2003 | 5045 | 20 | 4 | 22-span | Liver | C22 | −86.7 ± 6.2 | −88.5 ± 5.2 | −90.2 ± 4.8 |
| D-1988 | 5045 | 3 | 4 | 22-span | Liver | GalNAc | −84.4 ± 16.7 | −85 ± 15.7 | −89.6 ± 9.9 |
| D-1993 | 5045 | 3 | 4 | 22-span | Liver | GalNAc | −85 ± 7.4 | −86.1 ± 6.7 | −89.9 ± 4 |
| D-1998 | 5045 | 3 | 4 | 22-span | Liver | GalNAc | −95.3 ± 1.8 | −95.6 ± 1.4 | −96 ± 1.5 |
| D-1623 | 5080 | 3 | 4 | 22-span | Liver | GalNAc | −77.5 ± 13.5 | −78.8 ± 14.7 | −82.5 ± 10.1 |
| D-1862 | 5080 | 20 | 4 | 22-span | Liver | C22 | −79 ± 16 | −81.1 ± 14.7 | −83 ± 11.3 |
| D-1981 | 5080 | 3 | 4 | 22-span | Liver | GalNAc | −82.2 ± 14.2 | −83.2 ± 13.7 | −86.4 ± 10.4 |
| D-1985 | 5080 | 3 | 4 | 22-span | Liver | GalNAc | −90.3 ± 5.4 | −91.3 ± 4.7 | −91.5 ± 4.2 |
| D-2005 | 5080 | 20 | 4 | 22-span | Liver | C22 | −88.9 ± 8 | −89.4 ± 7.3 | −90.3 ± 5.3 |
| D-1990 | 5080 | 3 | 4 | 22-span | Liver | GalNAc | −81.7 ± 8.7 | −84.1 ± 7.3 | −86.1 ± 5.4 |
| D-1995 | 5080 | 3 | 4 | 22-span | Liver | GalNAc | −80.9 ± 8.8 | −84 ± 7.1 | −82.8 ± 7.6 |
| D-2000 | 5080 | 3 | 4 | 22-span | Liver | GalNAc | −78.3 ± 6.4 | −81.5 ± 6.2 | −81.9 ± 4.5 |
| D-1955 | 4412 | 3 | 4 | 22-span | Liver | GalNAc | −74.8 ± 8.4 | −76.7 ± 7.7 | −78.3 ± 9.3 |
| D-1970 | 5249 | 3 | 4 | 22-span | Liver | GalNAc | −66.9 ± 24 | −71.3 ± 21.6 | −74.5 ± 14.7 |
| D-1972 | 5274 | 3 | 4 | 22-span | Liver | GalNAc | −77 ± 10.9 | −78.1 ± 9.6 | −80.5 ± 9.2 |
| D-1774 | 5276 | 3 | 3 | 22-span | Liver | GalNAc | −73.8 ± 10 | −74.7 ± 9.7 | −77.9 ± 10.6 |
| D-1976 | 5276 | 20 | 4 | 22-span | Liver | C22 | −83.6 ± 6.4 | −83.9 ± 6.9 | −87.9 ± 5.6 |
| D-1977 | 5276 | 3 | 4 | 22-span | Liver | GalNAc | −89.5 ± 1.5 | −89.2 ± 1.4 | −90.7 ± 1.1 |
| D-1982 | 5276 | 3 | 4 | 22-span | Liver | GalNAc | −75.7 ± 6.9 | −74.6 ± 7.8 | −80.5 ± 5.3 |
| D-2001 | 5276 | 20 | 4 | 22-span | Liver | C22 | −83.7 ± 4.4 | −83.5 ± 4.9 | −86.7 ± 3 |
| D-1986 | 5276 | 3 | 4 | 22-span | Liver | GalNAc | −69.6 ± 9.2 | −71.4 ± 7.8 | −77.6 ± 7.3 |
| D-1991 | 5276 | 3 | 4 | 22-span | Liver | GalNAc | −83.7 ± 5.2 | −83 ± 5.4 | −86.6 ± 4.1 |
| D-1996 | 5276 | 3 | 4 | 22-span | Liver | GalNAc | −83.3 ± 11.2 | −84.3 ± 11.2 | −85.9 ± 8.9 |
| D-2017 | 1333 | 3 | 4 | 22-span | Liver | GalNAc | −93.1 ± 2 | −92.7 ± 2 | −94.7 ± 1.8 |
| D-1597 | 1333 | 3 | 4 | 22-span | Liver | GalNAc | −87 ± 4.2 | −87.6 ± 3.7 | −89.1 ± 3.5 |
| D-1853 | 1333 | 20 | 4 | 22-span | Liver | C22 | −87.2 ± 3.1 | −86.5 ± 3.1 | −89.4 ± 2.6 |
| D-1667 | 1309 | 3 | 4 | 22-span | Liver | GalNAc | −84.2 ± 10.2 | −84.3 ± 9.6 | −86.5 ± 10.6 |
| D-1849 | 1309 | 20 | 4 | 22-span | Liver | C22 | −91.1 ± 8.5 | −91 ± 7.9 | −92.3 ± 6.2 |
| D-1636 | 2144 | 3 | 4 | 22-span | Liver | GalNAc | −78.2 ± 5.2 | −77.8 ± 5.3 | −77.7 ± 4.4 |
| D-1858 | 2144 | 20 | 4 | 22-span | Liver | C22 | −85.5 ± 6.7 | −85.4 ± 6.1 | −90.7 ± 2.5 |
| D-1650 | 3000 | 3 | 4 | 22-span | Liver | GalNAc | −87.5 ± 6 | −87 ± 5.9 | −89.6 ± 4.4 |
| D-2035 | 3000 | 20 | 4 | 22-span | Liver | C22 | −50.4 ± 29.9 | −48.7 ± 30.8 | −46.6 ± 30.8 |
| D-1557 | 3000 | 3 | 4 | 22-span | Liver | GalNAc | −85.3 ± 14.9 | −85.2 ± 14.5 | −88.3 ± 10.7 |
| D-1861 | 3000 | 20 | 4 | 22-span | Liver | C22 | −92.8 ± 3.2 | −92.2 ± 3.4 | −94.3 ± 2.2 |
| D-1709 | 4999 | 3 | 4 | 22-span | Adipose | GalNAc | 54.7 ± 86.1 | 53.3 ± 88.7 | 59.4 ± 77 |
| D-1887 | 4999 | 20 | 4 | 22-span | Adipose | C22 | −85.4 ± 10.7 | −85.3 ± 10 | −88.2 ± 7.5 |
| D-1702 | 4999 | 3 | 4 | 22-span | Adipose | GalNAc | −38.1 ± 72.9 | −45 ± 62.5 | −34.8 ± 83 |
| D-1884 | 4999 | 20 | 4 | 22-span | Adipose | C22 | −90.2 ± 1.9 | −90.2 ± 1.6 | −92.3 ± 1.9 |
| D-1978 | 4999 | 3 | 4 | 22-span | Adipose | GalNAc | 11.8 ± 77.3 | 6.2 ± 70.4 | 7.8 ± 84.9 |
| D-1879 | 4999 | 3 | 4 | 22-span | Adipose | GalNAc | −14.4 ± 28.1 | −19 ± 28 | −6.3 ± 26.8 |
| D-2002 | 4999 | 20 | 4 | 22-span | Adipose | C22 | −92.9 ± 5.2 | −93.1 ± 5.4 | −95.3 ± 3 |
| D-1987 | 4999 | 3 | 4 | 22-span | Adipose | GalNAc | 73.5 ± 135.8 | 79 ± 140.5 | 68.6 ± 125.8 |
| D-1992 | 4999 | 3 | 4 | 22-span | Adipose | GalNAc | −21.1 ± 86.3 | −17.9 ± 86.4 | −20.7 ± 86 |
| D-1997 | 4999 | 3 | 4 | 22-span | Adipose | GalNAc | −5.5 ± 40.6 | −7.8 ± 43.4 | −7.4 ± 46.8 |
| D-1705 | 5043 | 3 | 4 | 22-span | Adipose | GalNAc | 6.6 ± 93.3 | 2.5 ± 81 | −2.2 ± 89.2 |
| D-1886 | 5043 | 20 | 4 | 22-span | Adipose | C22 | −91.4 ± 5.8 | −90.7 ± 6 | −90.82 ± 7.3 |
| D-1980 | 5043 | 3 | 4 | 22-span | Adipose | GalNAc | −1.1 ± 45.8 | −12.7 ± 40.1 | 1.1 ± 48 |
| D-1984 | 5043 | 3 | 4 | 22-span | Adipose | GalNAc | −30.8 ± 60.8 | −28.8 ± 60.2 | −34.7 ± 51.4 |
| D-2004 | 5043 | 20 | 3 | 22-span | Adipose | C22 | −90.9 ± 7.3 | −91 ± 6 | −92.8 ± 5.6 |
| D-1989 | 5043 | 3 | 4 | 22-span | Adipose | GalNAc | −29.2 ± 64 | −36 ± 54.6 | −18.9 ± 74.2 |
| D-1994 | 5043 | 3 | 4 | 22-span | Adipose | GalNAc | 160.7 ± 277.8 | 170.5 ± 276.8 | 144.6 ± 227.8 |
| D-1999 | 5043 | 3 | 4 | 22-span | Adipose | GalNAc | 43.8 ± 119.1 | 49.5 ± 123.3 | 50.5 ± 127.9 |
| D-1704 | 5045 | 3 | 4 | 22-span | Adipose | GalNAc | −45.7 ± 33.2 | −45.7 ± 35.2 | −46 ± 29.9 |
| D-1885 | 5045 | 20 | 4 | 22-span | Adipose | C22 | −97 ± 2.2 | −97 ± 1.9 | −96.7 ± 3 |
| D-1979 | 5045 | 3 | 4 | 22-span | Adipose | GalNAc | −60.8 ± 31.4 | −58.4 ± 37 | −65.6 ± 25.6 |
| D-1983 | 5045 | 3 | 4 | 22-span | Adipose | GalNAc | −31.4 ± 68.4 | −24.2 ± 78.3 | −34.1 ± 65.3 |
| D-2003 | 5045 | 20 | 4 | 22-span | Adipose | C22 | −92.5 ± 5.1 | −92.8 ± 4.5 | −94.6 ± 3.6 |
| D-1988 | 5045 | 3 | 4 | 22-span | Adipose | GalNAc | 125 ± 249.1 | 108.8 ± 235.9 | 162.6 ± 294.1 |
| D-1993 | 5045 | 3 | 4 | 22-span | Adipose | GalNAc | −27 ± 97 | −31.1 ± 85.3 | −24.5 ± 101.3 |
| D-1998 | 5045 | 3 | 4 | 22-span | Adipose | GalNAc | 17.7 ± 133.4 | 6.3 ± 116.3 | 14.3 ± 134.1 |
| D-1623 | 5080 | 3 | 4 | 22-span | Adipose | GalNAc | −25.3 ± 71.6 | −29.6 ± 65.9 | −23.2 ± 76.9 |
| D-1862 | 5080 | 20 | 4 | 22-span | Adipose | C22 | −92.1 ± 4.3 | −91.8 ± 4.8 | −94.6 ± 3.7 |
| D-1981 | 5080 | 3 | 4 | 22-span | Adipose | GalNAc | −1.8 ± 67.6 | −8.1 ± 63.5 | 0.5 ± 72.2 |
| D-1985 | 5080 | 3 | 4 | 22-span | Adipose | GalNAc | −21.9 ± 83 | −25.4 ± 79 | −24.5 ± 86.3 |
| D-2005 | 5080 | 20 | 4 | 22-span | Adipose | C22 | −85.9 ± 9.7 | −86.8 ± 8.5 | −92.3 ± 5.5 |
| D-1990 | 5080 | 3 | 4 | 22-span | Adipose | GalNAc | −4.1 ± 104 | −5.1 ± 100.6 | −2 ± 107.4 |
| D-1995 | 5080 | 3 | 4 | 22-span | Adipose | GalNAc | −23.7 ± 41.3 | −29.9 ± 39.2 | −28.1 ± 41.4 |
| D-2000 | 5080 | 3 | 4 | 22-span | Adipose | GalNAc | −71.9 ± 15.2 | −73.3 ± 14.4 | −69.7 ± 18.6 |
| D-1955 | 4412 | 3 | 4 | 22-span | Adipose | GalNAc | −36.7 ± 62.3 | −36.3 ± 61.6 | −34.6 ± 68.6 |
| D-1970 | 5249 | 3 | 4 | 22-span | Adipose | GalNAc | −51.1 ± 48.8 | −48.6 ± 52.8 | −54.4 ± 47.3 |
| D-1972 | 5274 | 3 | 4 | 22-span | Adipose | GalNAc | −37 ± 22.6 | −28.7 ± 27.9 | −37.3 ± 21 |
| D-1774 | 5276 | 3 | 3 | 22-span | Adipose | GalNAc | 171 ± 226.8 | 165.7 ± 214.9 | 168.2 ± 242.3 |
| D-1976 | 5276 | 20 | 4 | 22-span | Adipose | C22 | −82.7 ± 15.8 | −82.9 ± 15 | −87.8 ± 11.5 |
| D-1977 | 5276 | 3 | 4 | 22-span | Adipose | GalNAc | −21.3 ± 82.3 | −14.2 ± 87.6 | −23.4 ± 78.2 |
| D-1982 | 5276 | 3 | 4 | 22-span | Adipose | GalNAc | 164.9 ± 233.4 | 167.4 ± 228.3 | 157.3 ± 227.8 |
| D-2001 | 5276 | 20 | 4 | 22-span | Adipose | C22 | −92.3 ± 2.3 | −91.7 ± 2.6 | −95.2 ± 1.4 |
| D-1986 | 5276 | 3 | 4 | 22-span | Adipose | GalNAc | 80.1 ± 94 | 67.7 ± 71.1 | 77.4 ± 85.6 |
| D-1991 | 5276 | 3 | 4 | 22-span | Adipose | GalNAc | −47.7 ± 10.6 | −47.9 ± 11.6 | −47.2 ± 11.1 |
| D-1996 | 5276 | 3 | 4 | 22-span | Adipose | GalNAc | 251.3 ± 448.9 | 228.4 ± 404.2 | 294.1 ± 491.1 |
| D-2017 | 1333 | 3 | 4 | 22-span | Adipose | GalNAc | 51.1 ± 20.6 | −49.3 ± 23 | −48.6 ± 24.1 |
| D-1597 | 1333 | 3 | 4 | 22-span | Adipose | GalNAc | 58.2 ± 158.7 | 46.4 ± 139.6 | 44.4 ± 138.8 |
| D-1853 | 1333 | 20 | 4 | 22-span | Adipose | C22 | −50.4 ± 2.9 | −51.3 ± 2 | −65.9 ± 7.5 |
| D-1667 | 1309 | 3 | 4 | 22-span | Adipose | GalNAc | 154.6 ± 218.9 | 143.5 ± 199.5 | 163 ± 206.2 |
| D-1849 | 1309 | 20 | 4 | 22-span | Adipose | C22 | −71.6 ± 27.5 | −70 ± 30 | −79.5 ± 23.8 |
| D-1636 | 2144 | 3 | 4 | 22-span | Adipose | GalNAc | −69.5 ± 25.7 | −69.3 ± 23.4 | −77.5 ± 19.5 |
| D-1858 | 2144 | 20 | 4 | 22-span | Adipose | C22 | −66.2 ± 18.2 | −63.6 ± 22.3 | −81.7 ± 10.5 |
| D-1650 | 3000 | 3 | 4 | 22-span | Adipose | GalNAc | 121 ± 235.1 | 104.5 ± 202.1 | 179.8 ± 319.6 |
| D-2035 | 3000 | 20 | 4 | 22-span | Adipose | C22 | −65 ± 28.1 | −62.1 ± 29.7 | −66.4 ± 31.9 |
| D-1557 | 3000 | 3 | 4 | 22-span | Adipose | GalNAc | 67.1 ± 166.8 | 93.7 ± 184 | 74.4 ± 166.2 |
| D-1861 | 3000 | 20 | 4 | 22-span | Adipose | C22 | −96 ± 5.4 | −95.3 ± 6.4 | −97 ± 3.6 |

TABLE 15
siRNA Efficacy in Liver 4 Weeks Following siRNA Injection

| SIRNA | Trigger | Dose | | | | Avg. % Change in mRNA ± STD | | |
|---|---|---|---|---|---|---|---|---|
| Duplex | Family | (mg/kg) | N | AAV No. | Carrier | eGFP-1 | eGFP-2 | BGHpA |
| D-1614 | 4999 | 1 | 4 | 3 | GalNAc | −40.7 ± 17.9 | −39.6 ± 15.7 | −41.1 ± 19.3 |
| D-1611 | 5043 | 1 | 4 | 3 | GalNAc | −31.7 ± 28 | −32.7 ± 26 | −48.1 ± 20.5 |
| D-1742 | 4484 | 1 | 4 | 3 | GalNAc | −3.9 ± 22.4 | −0.9 ± 17.6 | −23.5 ± 16.7 |
| D-1743 | 4485 | 1 | 4 | 3 | GalNAc | −35.7 ± 39.2 | −33.8 ± 43.6 | −45.2 ± 26.3 |
| D-1744 | 4717 | 1 | 4 | 3 | GalNAc | −44.9 ± 30.1 | −49.8 ± 23.9 | −45.9 ± 12.8 |
| D-1745 | 4799 | 1 | 4 | 3 | GalNAc | −15 ± 25.8 | −21.9 ± 24 | −4.6 ± 31 |
| D-1746 | 4801 | 1 | 4 | 3 | GalNAc | −29.3 ± 49.2 | −32.5 ± 46.5 | −31.7 ± 38.3 |
| D-1747 | 4802 | 1 | 4 | 3 | GalNAc | 14.7 ± 53.5 | 16 ± 55.1 | 14.9 ± 46 |
| D-1748 | 4806 | 1 | 4 | 3 | GalNAc | 33.3 ± 51.8 | 21.9 ± 46.2 | −11.2 ± 21.5 |
| D-1749 | 4950 | 1 | 4 | 3 | GalNAc | −26.3 ± 7.9 | −22.5 ± 7.1 | −32.4 ± 10.3 |
| D-1750 | 4951 | 1 | 4 | 3 | GalNAc | −15.7 ± 33.3 | −9.4 ± 36.4 | −33.6 ± 22.4 |
| D-1751 | 4953 | 1 | 4 | 3 | GalNAc | 7.4 ± 34.9 | 12.5 ± 35.5 | −15.3 ± 33.1 |
| D-1752 | 4954 | 1 | 4 | 3 | GalNAc | −5.1 ± 35.9 | −2.1 ± 35.1 | −25.5 ± 23.3 |
| D-1753 | 4955 | 1 | 4 | 3 | GalNAc | 6.2 ± 38.7 | 13.5 ± 38.1 | 14.8 ± 55.2 |
| D-1754 | 4958 | 1 | 4 | 3 | GalNAc | −21 ± 18.5 | −19.8 ± 13.2 | −35.3 ± 4.6 |
| D-1755 | 4965 | 1 | 4 | 3 | GalNAc | 12.9 ± 31.8 | 7.1 ± 27.4 | 7.3 ± 24.2 |
| D-1756 | 4970 | 1 | 4 | 3 | GalNAc | 23.6 ± 25.4 | 25.9 ± 26 | 17.3 ± 23.6 |
| D-1757 | 4996 | 1 | 4 | 3 | GalNAc | −15.5 ± 17.8 | −10.8 ± 22 | −27.5 ± 9.6 |
| D-1758 | 4997 | 1 | 4 | 3 | GalNAc | 6.9 ± 13.4 | 8.3 ± 13.3 | −24.1 ± 13.7 |
| D-1759 | 5008 | 1 | 4 | 3 | GalNAc | 70.4 ± 77 | 79.3 ± 88.9 | 14.4 ± 36.1 |
| D-1760 | 5056 | 1 | 4 | 3 | GalNAc | −5.1 ± 19.6 | −2 ± 18.9 | −42.6 ± 7 |
| D-1761 | 5080 | 1 | 4 | 3 | GalNAc | 9.2 ± 33.4 | 15.1 ± 33 | −37.6 ± 4.3 |
| D-1762 | 5114 | 1 | 4 | 3 | GalNAc | −27.6 ± 26.5 | −21.7 ± 27 | −47.8 ± 13.6 |
| D-1763 | 5115 | 1 | 4 | 3 | GalNAc | 24.8 ± 64.5 | 25.4 ± 60.1 | −2.1 ± 28.1 |
| D-1764 | 5154 | 1 | 4 | 3 | GalNAc | 8.7 ± 50.5 | 15.7 ± 57.8 | −4.1 ± 32.8 |
| D-1765 | 5155 | 1 | 4 | 3 | GalNAc | 28.9 ± 64.5 | 40.4 ± 72.9 | 3.6 ± 35.5 |
| D-1766 | 5195 | 1 | 4 | 3 | GalNAc | 28.3 ± 40.2 | 28.8 ± 32.9 | 52.2 ± 56.6 |
| D-1767 | 5200 | 1 | 4 | 3 | GalNAc | 77.3 ± 52.2 | 82.7 ± 68.3 | 46.4 ± 48.2 |
| D-1614 | 4999 | 1 | 4 | 3 | GalNAc | −83.8 ± 2.8 | −82.2 ± 2.6 | −73.4 ± 3.8 |
| D-1611 | 5043 | 1 | 4 | 3 | GalNAc | −50.5 ± 5.6 | −50 ± 9 | −50 ± 3.7 |
| D-1768 | 5247 | 1 | 4 | 3 | GalNAc | −55.7 ± 3.3 | −53.3 ± 4.1 | −49.9 ± 4.3 |
| D-1769 | 5249 | 1 | 4 | 3 | GalNAc | −56.7 ± 10 | −53.6 ± 9.5 | −52.2 ± 2.4 |
| D-1770 | 5251 | 1 | 4 | 3 | GalNAc | −33.4 ± 10 | −31.6 ± 10.2 | −38.9 ± 8.5 |
| D-1771 | 5254 | 1 | 4 | 3 | GalNAc | −32.5 ± 16.7 | −28.3 ± 20.6 | −30.7 ± 8.4 |
| D-1772 | 5259 | 1 | 4 | 3 | GalNAc | −42.9 ± 22.8 | −39.9 ± 23.9 | −44.1 ± 18.5 |
| D-1773 | 5274 | 1 | 4 | 3 | GalNAc | −52.4 ± 16.3 | −46.6 ± 21.8 | −62.1 ± 8.1 |
| D-1774 | 5276 | 1 | 4 | 3 | GalNAc | −51.9 ± 23.5 | −50.2 ± 23.8 | −44 ± 37.8 |
| D-1775 | 5344 | 1 | 4 | 3 | GalNAc | 1.8 ± 46.5 | 8.3 ± 63.6 | 8.3 ± 38.4 |
| D-1776 | 5402 | 1 | 4 | 3 | GalNAc | −33.2 ± 37 | −21.4 ± 44.9 | −26.9 ± 44.4 |
| D-1777 | 4412 | 1 | 4 | 3 | GalNAc | −51.3 ± 10.8 | −46.8 ± 9.3 | −51 ± 7.5 |
| D-1778 | 4777 | 1 | 4 | 3 | GalNAc | −28.5 ± 43.7 | −17.5 ± 55.4 | −33.7 ± 35.1 |
| D-1779 | 4780 | 1 | 4 | 3 | GalNAc | −19 ± 26.9 | −4.3 ± 26.2 | −16 ± 21.4 |
| D-1780 | 4819 | 1 | 4 | 3 | GalNAc | −18.8 ± 39.4 | −4.3 ± 46.1 | −1.9 ± 45.2 |
| D-1781 | 4834 | 1 | 4 | 3 | GalNAc | −63.4 ± 8.6 | −58.4 ± 12.7 | −60 ± 12.2 |
| D-1782 | 4931 | 1 | 4 | 3 | GalNAc | 41.3 ± 12.2 | −34.9 ± 12.7 | −23.6 ± 10.1 |
| D-1783 | 4932 | 1 | 4 | 3 | GalNAc | −59.1 ± 10.8 | −57.2 ± 11.4 | −52.7 ± 8.6 |
| D-1784 | 4933 | 1 | 4 | 3 | GalNAc | −33.6 ± 7.4 | −31.2 ± 9.6 | −17.7 ± 9.5 |
| D-1785 | 4935 | 1 | 4 | 3 | GalNAc | −27 ± 31.3 | −14.9 ± 37.2 | −22.9 ± 27.1 |
| D-1786 | 4939 | 1 | 4 | 3 | GalNAc | −34.6 ± 17.4 | −34.6 ± 17.1 | −32.2 ± 23.8 |
| D-1787 | 4940 | 1 | 4 | 3 | GalNAc | 0.6 ± 31.7 | −12 ± 20.3 | −5.1 ± 13.9 |
| D-1788 | 4989 | 1 | 4 | 3 | GalNAc | −42.4 ± 25.6 | −38.8 ± 23.8 | −30.5 ± 18.8 |
| D-1789 | 4991 | 1 | 4 | 3 | GalNAc | −37.1 ± 8.7 | −30.7 ± 8.1 | −17.2 ± 6.8 |
| D-1790 | 5201 | 1 | 4 | 3 | GalNAc | −22.6 ± 50.6 | −17.1 ± 54.8 | −11.9 ± 52.9 |
| D-1791 | 5203 | 1 | 4 | 3 | GalNAc | −19 ± 34.6 | −17.9 ± 35.4 | −2.2 ± 51.2 |
| D-1792 | 5204 | 1 | 4 | 3 | GalNAc | −55.4 ± 6.5 | −49.7 ± 6.7 | −31 ± 12.8 |
| D-1793 | 5207 | 1 | 4 | 3 | GalNAc | −46 ± 13.6 | −42.6 ± 18.7 | −26.8 ± 22.9 |
| D-1597 | 1333 | 1 | 4 | 1 | GalNAc | −7.1 ± 14.9 | −3.7 ± 15.6 | −10.5 ± 13.8 |
| D-1544 | 2144 | 1 | 4 | 1 | GalNAc | −26.1 ± 20.1 | −22.6 ± 14 | −32.8 ± 16.4 |
| D-1794 | 1305 | 1 | 4 | 1 | GalNAc | −9.6 ± 21.6 | −2.6 ± 18.8 | −19.9 ± 17.5 |
| D-1795 | 1306 | 1 | 4 | 1 | GalNAc | 2.7 ± 27.1 | 7.9 ± 32.2 | 4.2 ± 26.5 |
| D-1796 | 1308 | 1 | 4 | 1 | GalNAc | −8.8 ± 26.4 | −5.8 ± 23.5 | −20.8 ± 18.6 |
| D-1797 | 1472 | 1 | 4 | 1 | GalNAc | 0.6 ± 49.8 | 0.3 ± 54.3 | −20.3 ± 38.7 |
| D-1798 | 1500 | 1 | 4 | 1 | GalNAc | −4.6 ± 8.9 | −3.9 ± 9.5 | −19.3 ± 18 |
| D-1809 | 2296 | 1 | 4 | 1 | GalNAc | 12.4 ± 65.3 | 15 ± 64.7 | 21.4 ± 78.9 |
| D-1810 | 2343 | 1 | 4 | 1 | GalNAc | −30 ± 20.2 | −28.8 ± 20.4 | −38.3 ± 13.1 |
| D-1811 | 2355 | 1 | 4 | 1 | GalNAc | −20.1 ± 39.6 | −20.6 ± 32.7 | −18.9 ± 30.3 |
| D-1812 | 2417 | 1 | 4 | 1 | GalNAc | −42.3 ± 12.1 | −38.8 ± 12.2 | −45.6 ± 4.9 |
| D-1813 | 2432 | 1 | 4 | 1 | GalNAc | −28.6 ± 22.1 | −22.1 ± 27.3 | −36.7 ± 14.5 |
| D-1814 | 2688 | 1 | 4 | 1 | GalNAc | 21.5 ± 13 | 23.6 ± 14.6 | −6.8 ± 10.2 |
| D-1815 | 2690 | 1 | 4 | 1 | GalNAc | −21.3 ± 19.1 | −18.3 ± 17.1 | −34.5 ± 9.8 |
| D-1816 | 2886 | 1 | 4 | 1 | GalNAc | −23.6 ± 42.2 | −18.2 ± 45.8 | −49.4 ± 24.2 |
| D-1817 | 1326 | 1 | 4 | 1 | GalNAc | −14.2 ± 21 | −13.1 ± 16 | −36.1 ± 9.9 |
| D-1818 | 1331 | 1 | 4 | 1 | GalNAc | 11.5 ± 72 | 12.7 ± 62.6 | −21.3 ± 35.1 |
| D-1819 | 1407 | 1 | 4 | 1 | GalNAc | −20.5 ± 12.3 | −17.4 ± 20.2 | −35.4 ± 5.6 |
| D-1597 | 1333 | 1 | 4 | 1 | GalNAc | −29.9 ± 43.6 | −36.7 ± 36 | −40.4 ± 31.5 |
| D-1544 | 2144 | 1 | 4 | 1 | GalNAc | 16.9 ± 24.5 | 10.7 ± 23.7 | 1.5 ± 30 |
| D-1820 | 1514 | 1 | 4 | 1 | GalNAc | −26 ± 41.7 | −23.6 ± 41.8 | −26.4 ± 37.4 |
| D-1821 | 1564 | 1 | 4 | 1 | GalNAc | 16.7 ± 67 | 12.9 ± 68.8 | 105.3 ± 51.2 |
| D-1822 | 1611 | 1 | 4 | 1 | GalNAc | 58.7 ± 72 | 57.7 ± 80 | 45.4 ± 67.1 |
| D-1823 | 1615 | 1 | 4 | 1 | GalNAc | 27.2 ± 21.2 | 38.5 ± 23.9 | 24.7 ± 30.4 |
| D-1824 | 1616 | 1 | 4 | 1 | GalNAc | −17.8 ± 56.8 | −13.7 ± 59.5 | −12.9 ± 59.4 |
| D-1825 | 1618 | 1 | 4 | 1 | GalNAc | −20.6 ± 35.1 | −21.3 ± 35 | −31.1 ± 22 |
| D-1826 | 1693 | 1 | 4 | 1 | GalNAc | −28.1 ± 32.7 | −13.8 ± 32.7 | −34.2 ± 30.4 |
| D-1827 | 1697 | 1 | 4 | 1 | GalNAc | 0.8 ± 18.9 | −1.8 ± 19.2 | 4.9 ± 41.3 |
| D-1828 | 1700 | 1 | 4 | 1 | GalNAc | 32.6 ± 24.6 | 33 ± 14 | 15.6 ± 19.4 |
| D-1829 | 1701 | 1 | 4 | 1 | GalNAc | 17.8 ± 50.2 | 11.2 ± 46.5 | 2.7 ± 40.7 |
| D-1830 | 1703 | 1 | 4 | 1 | GalNAc | 51.5 ± 39 | 44.7 ± 43.9 | 32.3 ± 35.8 |
| D-1831 | 1704 | 1 | 4 | 1 | GalNAc | 9.4 ± 36.6 | 9.7 ± 36.7 | −5.9 ± 23.2 |
| D-1832 | 1716 | 1 | 4 | 1 | GalNAc | 21.8 ± 29.4 | 29.7 ± 25.6 | 25.8 ± 28.6 |
| D-1833 | 1717 | 1 | 4 | 1 | GalNAc | 24.7 ± 45.7 | 33.3 ± 42.4 | 27.2 ± 34.9 |
| D-1834 | 1832 | 1 | 4 | 1 | GalNAc | 91.8 ± 94.3 | 125.8 ± 122.3 | 80.5 ± 91.5 |
| D-1835 | 1833 | 1 | 4 | 1 | GalNAc | 72.7 ± 75.1 | 98.8 ± 91.9 | 72.8 ± 71.2 |
| D-1836 | 1834 | 1 | 4 | 1 | GalNAc | 4 ± 25.5 | 3.4 ± 27.5 | 7.3 ± 26.1 |
| D-1837 | 1856 | 1 | 4 | 1 | GalNAc | −8.2 ± 8.8 | −10.2 ± 9.3 | −16.2 ± 15.9 |
| D-1838 | 1900 | 1 | 4 | 1 | GalNAc | 35.2 ± 35.2 | 42.4 ± 32.3 | 13.6 ± 28.3 |
| D-1839 | 2275 | 1 | 4 | 1 | GalNAc | 54.8 ± 62.1 | 57.1 ± 67.4 | 9.3 ± 30.7 |
| D-1840 | 2437 | 1 | 4 | 1 | GalNAc | 80 ± 18.3 | 79.2 ± 26.8 | 79.4 ± 22.8 |
| D-1841 | 2439 | 1 | 4 | 1 | GalNAc | 49.2 ± 23.8 | 43.4 ± 12.9 | 42.3 ± 19.9 |
| D-1842 | 2534 | 1 | 4 | 1 | GalNAc | 24.1 ± 22.7 | 12.5 ± 17.4 | 16.7 ± 10.3 |
| D-1843 | 2693 | 1 | 4 | 1 | GalNAc | 87.7 ± 40.7 | 83.7 ± 39.7 | 62.3 ± 32.7 |
| D-1844 | 2719 | 1 | 4 | 1 | GalNAc | 24.4 ± 44.4 | 26.8 ± 38.5 | 19.9 ± 22.8 |
| D-1845 | 2726 | 1 | 4 | 1 | GalNAc | 26.8 ± 46.2 | 29.3 ± 49.8 | 25.5 ± 43.9 |

TABLE 16
siRNA Efficacy in Liver 4 Weeks Following siRNA Injection

| SIRNA | Trigger | Dose | | | | Avg. % Change in mRNA ± STD | | |
|---|---|---|---|---|---|---|---|---|
| Duplex | Family | (mg/kg) | N | AAV No. | Carrier | eGFP-1 | eGFP-2 | BGHpA |
| D-1744 | 4717 | 3 | 4 | 3 | GalNAc | −62.6 ± 19.9 | −61.8 ± 21.1 | −63 ± 14 |
| D-1896 | 4717 | 3 | 4 | 3 | GalNAc | −81.8 ± 6.7 | −81.3 ± 7.2 | −75.2 ± 9.1 |
| D-1902 | 4717 | 3 | 4 | 3 | GalNAc | −59.6 ± 14.4 | −59.4 ± 15.3 | −62.5 ± 8.5 |
| D-1908 | 4717 | 3 | 4 | 3 | GalNAc | −67.3 ± 9.2 | −65.2 ± 11.5 | −60.4 ± 15 |
| D-1781 | 4834 | 3 | 4 | 3 | GalNAc | −64.9 ± 8.5 | −65 ± 6.9 | −60.9 ± 3.9 |
| D-1894 | 4834 | 3 | 4 | 3 | GalNAc | −66.9 ± 10.7 | −65.6 ± 10.5 | −61 ± 8 |
| D-1900 | 4834 | 3 | 4 | 3 | GalNAc | −54.2 ± 25.6 | −56.4 ± 26 | −57 ± 13.9 |
| D-1906 | 4834 | 3 | 4 | 3 | GalNAc | −64.2 ± 10.8 | −62.9 ± 11 | −64.9 ± 7.8 |
| D-1783 | 4932 | 3 | 4 | 3 | GalNAc | −79.1 ± 8.3 | −79.3 ± 7.9 | −74.5 ± 4.7 |
| D-1895 | 4932 | 3 | 4 | 3 | GalNAc | −75.3 ± 17.4 | −74.1 ± 17.5 | −64.9 ± 13.2 |
| D-1901 | 4932 | 3 | 4 | 3 | GalNAc | −50.8 ± 7.8 | −47.6 ± 9.7 | −57.1 ± 6.7 |
| D-1907 | 4932 | 3 | 4 | 3 | GalNAc | −74.6 ± 11.3 | −73.9 ± 12.4 | −70.6 ± 15 |
| D-1614 | 4999 | 3 | 4 | 3 | GalNAc | −72.2 ± 10.1 | −71.9 ± 10.2 | −63.3 ± 10.6 |
| D-1611 | 5043 | 3 | 4 | 3 | GalNAc | −66.7 ± 24.4 | −67.8 ± 22.8 | −70.6 ± 17.9 |
| D-1792 | 5204 | 3 | 4 | 3 | GalNAc | −55.6 ± 9.1 | −52.9 ± 7.6 | −62.9 ± 6.5 |
| D-1892 | 5204 | 3 | 4 | 3 | GalNAc | −54.2 ± 12.8 | −53.1 ± 14.4 | −56.1 ± 11.1 |
| D-1898 | 5204 | 3 | 4 | 3 | GalNAc | −69.1 ± 3.8 | −68.5 ± 3.2 | −66.8 ± 2.3 |
| D-1904 | 5204 | 3 | 4 | 3 | GalNAc | −39.5 ± 13 | −39.3 ± 14.2 | −49.1 ± 11 |
| D-1768 | 5247 | 3 | 4 | 3 | GalNAc | −67.2 ± 7.6 | −69.4 ± 7 | −69.4 ± 8.7 |
| D-1891 | 5247 | 3 | 4 | 3 | GalNAc | −37.2 ± 16 | −39.3 ± 15.8 | −57.1 ± 11.9 |
| D-1897 | 5247 | 3 | 4 | 3 | GalNAc | −61.7 ± 9.1 | −60.4 ± 9.6 | −67.2 ± 6.4 |
| D-1903 | 5247 | 3 | 4 | 3 | GalNAc | −39.3 ± 28.3 | −38.4 ± 30.2 | −56.1 ± 16.5 |
| D-1774 | 5276 | 3 | 4 | 3 | GalNAc | −70 ± 20.2 | −70.5 ± 18.8 | −68.2 ± 21.8 |
| D-1893 | 5276 | 3 | 4 | 3 | GalNAc | −59.4 ± 11.7 | −56.8 ± 12.6 | −61.5 ± 7.3 |
| D-1899 | 5276 | 3 | 4 | 3 | GalNAc | −73.8 ± 6.3 | −72.9 ± 7.9 | −72.4 ± 5.3 |
| D-1905 | 5276 | 3 | 4 | 3 | GalNAc | −59.7 ± 8.2 | −59.1 ± 8.2 | −61.8 ± 3.6 |
| D-1914 | 4717 | 3 | 4 | 3 | GalNAc | −55.9 ± 5.1 | −51 ± 6 | −64.9 ± 12.9 |
| D-1920 | 4717 | 3 | 4 | 3 | GalNAc | −61.8 ± 10.6 | −58.2 ± 13 | −59.2 ± 7.6 |
| D-1926 | 4717 | 3 | 4 | 3 | GalNAc | −40.6 ± 25 | −39.3 ± 19.9 | −52.3 ± 11.1 |
| D-1932 | 4717 | 3 | 4 | 3 | GalNAc | −69.5 ± 8.3 | −68.7 ± 6.7 | −67.6 ± 7.4 |
| D-1912 | 4834 | 3 | 4 | 3 | GalNAc | −25.9 ± 30.2 | −7.6 ± 46.6 | −43.6 ± 20.4 |
| D-1918 | 4834 | 3 | 4 | 3 | GalNAc | −71.9 ± 40.5 | −71.3 ± 41 | −69.3 ± 41.3 |
| D-1924 | 4834 | 3 | 4 | 3 | GalNAc | −27.1 ± 27 | −29.6 ± 21.3 | −36.2 ± 18.8 |
| D-1930 | 4834 | 3 | 4 | 3 | GalNAc | −62.5 ± 17.3 | −64.9 ± 17.4 | −64.3 ± 15.7 |
| D-1913 | 4932 | 3 | 4 | 3 | GalNAc | −55.9 ± 12.3 | −47.5 ± 11.4 | −53.1 ± 12.5 |
| D-1919 | 4932 | 3 | 4 | 3 | GalNAc | −38 ± 37.3 | −34.7 ± 44.3 | −55.1 ± 22.1 |
| D-1925 | 4932 | 3 | 4 | 3 | GalNAc | −56.9 ± 9.8 | −51.5 ± 11.8 | −58.3 ± 8.8 |
| D-1931 | 4932 | 3 | 4 | 3 | GalNAc | −62.1 ± 14.1 | −63.2 ± 10.2 | −58.1 ± 14.8 |
| D-1614 | 4999 | 3 | 4 | 3 | GalNAc | −76.1 ± 7 | −78.2 ± 5.6 | −73.3 ± 9.8 |
| D-1611 | 5043 | 3 | 4 | 3 | GalNAc | −46.6 ± 8 | −47.3 ± 7.3 | −52.1 ± 11.5 |
| D-1910 | 5204 | 3 | 4 | 3 | GalNAc | −30.7 ± 40.6 | −33.5 ± 39.2 | −41.1 ± 35.3 |
| D-1916 | 5204 | 3 | 4 | 3 | GalNAc | −59.5 ± 8.3 | −59.6 ± 10.9 | −59.3 ± 4.4 |
| D-1922 | 5204 | 3 | 4 | 3 | GalNAc | −16.2 ± 25.6 | −20 ± 22.8 | −24.5 ± 22.7 |
| D-1928 | 5204 | 3 | 4 | 3 | GalNAc | −61.8 ± 6.1 | −58.5 ± 8 | −56.4 ± 15.8 |
| D-1909 | 5247 | 3 | 4 | 3 | GalNAc | −59 ± 20.7 | −60.6 ± 16.4 | −65.7 ± 12.5 |
| D-1915 | 5247 | 3 | 4 | 3 | GalNAc | −52.8 ± 20.8 | −37.6 ± 29.6 | −66 ± 11.9 |
| D-1921 | 5247 | 3 | 4 | 3 | GalNAc | −54.4 ± 13.4 | −48.2 ± 19.7 | −66 ± 12.1 |
| D-1927 | 5247 | 3 | 4 | 3 | GalNAc | −47.4 ± 21.1 | −35.6 ± 31.6 | −57.9 ± 17.4 |
| D-1911 | 5276 | 3 | 4 | 3 | GalNAc | −49.6 ± 22.2 | −47.1 ± 24.8 | −60.6 ± 18.2 |
| D-1917 | 5276 | 3 | 4 | 3 | GalNAc | −79.6 ± 17.6 | −77.6 ± 19.8 | −76.4 ± 17.7 |
| D-1923 | 5276 | 3 | 4 | 3 | GalNAc | −63.8 ± 17.3 | −61.4 ± 19.7 | −59.5 ± 21.4 |
| D-1929 | 5276 | 3 | 4 | 3 | GalNAc | −69 ± 10.5 | −68.8 ± 10 | −59 ± 11.2 |

TABLE 17
siRNA Efficacy in Liver 4 Weeks Following siRNA Injection

| SIRNA | Trigger | Dose | | AAV | | Avg. % Change in mRNA ± STD | | |
|---|---|---|---|---|---|---|---|---|
| Duplex | Family | (mg/kg) | N | No. | Carrier | eGFP-1 | eGFP-2 | BGHpA |
| D-1709 | 4999 | 3 | 4 | 22span | GalNAc | −83.2 ± 8.6 | −82.9 ± 9.3 | −83.7 ± 7.5 |
| D-2008 | 4999 | 3 | 4 | 22span | GalNAc | −62.7 ± 8.7 | −65.6 ± 11.3 | −76.2 ± 7.4 |
| D-2017 | 4999 | 3 | 4 | 22span | GalNAc | −66.4 ± 15.5 | −66.4 ± 16.1 | −72.6 ± 11.5 |
| D-2049 | 4999 | 3 | 4 | 22span | GalNAc | −78.6 ± 11.1 | −79.7 ± 10.1 | −85.5 ± 5.7 |
| D-2054 | 4999 | 3 | 4 | 22span | GalNAc | −82.6 ± 6.2 | −82.4 ± 5.9 | −87.3 ± 3.8 |
| D-1704 | 5045 | 3 | 4 | 22span | GalNAc | −81.1 ± 8.4 | −81.8 ± 6.9 | −83.3 ± 6 |
| D-2012 | 5045 | 3 | 4 | 22span | GalNAc | −91.6 ± 1.1 | −91.3 ± 0.8 | −90.4 ± 1.2 |
| D-2021 | 5045 | 3 | 4 | 22span | GalNAc | −84.5 ± 6.7 | −85.3 ± 5.2 | −84.8 ± 6.3 |
| D-2043 | 5045 | 3 | 4 | 22span | GalNAc | −74 ± 12.9 | −74.7 ± 11.1 | −79.2 ± 8.6 |
| D-2047 | 5045 | 3 | 4 | 22span | GalNAc | −80.1 ± 13.4 | −81 ± 11.1 | −81.6 ± 11.4 |
| D-2052 | 5045 | 3 | 4 | 22span | GalNAc | −84.2 ± 5.9 | −84.1 ± 6.5 | −83.3 ± 4.9 |
| D-1623 | 5080 | 3 | 4 | 22span | GalNAc | −59.6 ± 11.3 | −62.9 ± 11.1 | −62.9 ± 11 |
| D-2014 | 5080 | 3 | 4 | 22span | GalNAc | −89.1 ± 10.6 | −88.8 ± 9.1 | −88.5 ± 9.8 |
| D-2023 | 5080 | 3 | 4 | 22span | GalNAc | −89.1 ± 6.5 | −88.2 ± 6.2 | −87.6 ± 5.3 |
| D-2036 | 4995 | 3 | 4 | 22span | GalNAc | −59.3 ± 40.7 | −60.4 ± 39.2 | −66 ± 29.9 |
| D-2037 | 4996 | 3 | 4 | 22span | GalNAc | −42.8 ± 23.2 | −48.5 ± 16.9 | −42.5 ± 20.2 |
| D-2038 | 4997 | 3 | 4 | 22span | GalNAc | 76.6 ± 62.9 | 105.4 ± 74.5 | 88.1 ± 60.4 |
| D-2039 | 4998 | 3 | 4 | 22span | GalNAc | −43.5 ± 27.1 | −43.6 ± 30.2 | −56.6 ± 19 |
| D-2040 | 5042 | 3 | 4 | 22span | GalNAc | −83.5 ± 10.5 | −83.8 ± 9.5 | −84.3 ± 7.2 |
| D-1705 | 5043 | 3 | 4 | 22span | GalNAc | −86.5 ± 5.5 | −86.9 ± 5.4 | −90.5 ± 4.1 |
| D-2013 | 5043 | 3 | 4 | 22span | GalNAc | −77.8 ± 15.6 | −78.8 ± 15.2 | −85.1 ± 10.8 |
| D-2022 | 5043 | 3 | 4 | 22span | GalNAc | −79.2 ± 5.7 | −80.3 ± 5 | −87.6 ± 3.7 |
| D-2044 | 5043 | 3 | 4 | 22span | GalNAc | −72.4 ± 32.6 | −70.3 ± 37.2 | −82 ± 21.7 |
| D-2048 | 5043 | 3 | 4 | 22span | GalNAc | −64.1 ± 18.9 | −66.2 ± 18.5 | −76.3 ± 10.2 |
| D-2053 | 5043 | 3 | 4 | 22span | GalNAc | −83.7 ± 5 | −84.9 ± 4.9 | −89.8 ± 3.2 |
| D-2042 | 5274 | 3 | 4 | 22span | GalNAc | −58.7 ± 23.5 | −62.9 ± 21.2 | −74.4 ± 14.3 |
| D-2046 | 5274 | 3 | 4 | 22span | GalNAc | −51.9 ± 37.4 | −55.9 ± 32.1 | −70.4 ± 18.9 |
| D-2051 | 5274 | 3 | 4 | 22span | GalNAc | −71.2 ± 14.4 | −72.6 ± 13 | −82.1 ± 8.2 |
| D-2079 | 5274 | 3 | 4 | 22span | GalNAc | −83.4 ± 6.6 | −84.7 ± 5.8 | −89.1 ± 5.1 |
| D-2080 | 5274 | 3 | 4 | 22span | GalNAc | −84.7 ± 4.6 | −85.2 ± 4.4 | −90.1 ± 2.6 |
| D-2081 | 5274 | 3 | 4 | 22span | GalNAc | −83.1 ± 11 | −83.7 ± 10.8 | −88.5 ± 8.3 |
| D-2082 | 5274 | 3 | 4 | 22span | GalNAc | −60.4 ± 18.6 | −61.8 ± 18 | −75 ± 9.5 |
| D-2083 | 5274 | 3 | 4 | 22span | GalNAc | −72.3 ± 21.8 | −73.2 ± 20.8 | −82.2 ± 11.4 |
| D-2059 | 4412 | 3 | 4 | 22span | GalNAc | −53.8 ± 19 | −52.8 ± 26 | −68.3 ± 10.9 |
| D-2058 | 4412 | 3 | 4 | 22span | GalNAc | −76.9 ± 18.2 | −76.6 ± 20.2 | −83.9 ± 11.5 |
| D-2060 | 4412 | 3 | 4 | 22span | GalNAc | −62.4 ± 17.8 | −60.8 ± 18.1 | −74.6 ± 11.4 |
| D-1774 | 5276 | 3 | 4 | 22span | GalNAc | −59.9 ± 14.9 | −59.8 ± 15.9 | −74 ± 6.8 |
| D-2084 | 5276 | 3 | 4 | 22span | GalNAc | −46.3 ± 25.2 | −47.5 ± 23.2 | −65.6 ± 13.1 |
| D-1955 | 4412 | 3 | 4 | 22span | GalNAc | −88.2 ± 5.2 | −87.8 ± 5.5 | −88.1 ± 4.7 |
| D-2091 | 4412 | 3 | 4 | 22span | GalNAc | −89.2 ± 2.4 | −89.4 ± 2.9 | −90.6 ± 1 |
| D-2061 | 4412 | 3 | 4 | 22span | GalNAc | −93.3 ± 3 | −93.5 ± 2.9 | −93.6 ± 3.1 |
| D-2041 | 4412 | 3 | 4 | 22span | GalNAc | −60.5 ± 18.9 | −60.7 ± 18.9 | −69 ± 13.4 |
| D-2045 | 4412 | 3 | 4 | 22span | GalNAc | −80 ± 12 | −80.2 ± 10 | −82.6 ± 10.5 |
| D-2050 | 4412 | 3 | 4 | 22span | GalNAc | −56.1 ± 55.5 | −58.3 ± 50.9 | −62.3 ± 44.7 |
| D-2057 | 4412 | 3 | 4 | 22span | GalNAc | −76.7 ± 14.4 | −78.8 ± 11.8 | −76.9 ± 12.4 |
| D-1970 | 5249 | 3 | 4 | 22span | GalNAc | −82.4 ± 13 | −83.5 ± 11.9 | −82.9 ± 13 |
| D-2076 | 5249 | 3 | 4 | 22span | GalNAc | −60.6 ± 11.6 | −65 ± 9.7 | −66.8 ± 10.1 |
| D-2078 | 5249 | 3 | 4 | 22span | GalNAc | −72.6 ± 17.4 | −74 ± 16.1 | −70.8 ± 20.2 |
| D-1768 | 5247 | 3 | 4 | 22span | GalNAc | −83.6 ± 10.3 | −83.8 ± 10 | −83.3 ± 9.6 |
| D-2075 | 5247 | 3 | 4 | 22span | GalNAc | −88.6 ± 10 | −89.2 ± 9.1 | −87.8 ± 11 |
| D-2077 | 5247 | 3 | 4 | 22span | GalNAc | −93 ± 2.2 | −92.7 ± 2.6 | −94.5 ± 1 |
| D-1597 | 1333 | 3 | 4 | 22span | GalNAc | −83.4 ± 9.3 | −83.3 ± 9.2 | −86.8 ± 7.4 |
| D-2006 | 1333 | 3 | 4 | 22span | GalNAc | −79.3 ± 9.7 | −79.2 ± 9.6 | −79.7 ± 9.9 |
| D-2015 | 1333 | 3 | 4 | 22span | GalNAc | −65.3 ± 12.8 | −66 ± 12.1 | −72 ± 8.2 |
| D-2090 | 4999 | 3 | 4 | 22span | GalNAc | −94.5 ± 1.8 | −94 ± 2.1 | −94.6 ± 1.6 |
| D-2093 | 5274 | 3 | 4 | 22span | GalNAc | −81.6 ± 13 | −79.6 ± 15.1 | −86.9 ± 7.1 |
| D-1899 | 5276 | 3 | 4 | 22span | GalNAc | −78.5 ± 10.1 | −78.2 ± 9.9 | −83.4 ± 5.9 |

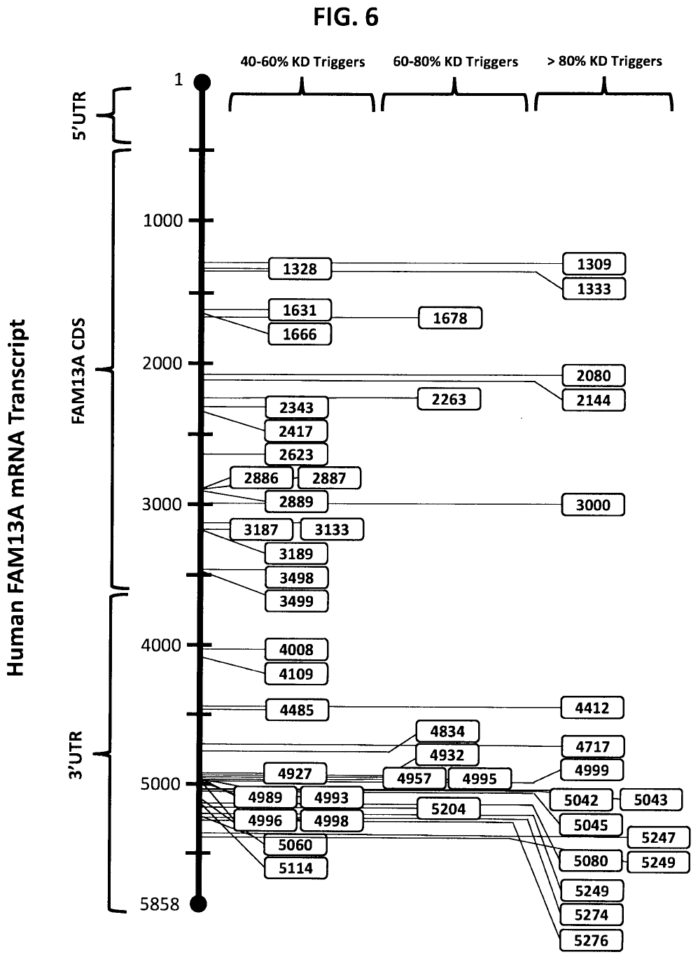

Testing of FAM13A-directed siRNA molecules within the AAV human FAM13A mouse model showed that a variety of different regions within FAM13A mRNA can be targeted to effectively reduce FAM13A expression. As shown in FIG. 6, the effective siRNA triggers targeted regions throughout the FAM13A mRNA transcript (SEQ ID NO: 1). In the above tables, the region targeted by the siRNA is specified by the trigger family, which refers to the first nucleotide in the range of nucleotides of SEQ ID NO: 1 that is targeted by a given siRNA molecule.

Trigger families that achieved a maximum knockdown of between 40-60% relative to vehicle control (for at least one probe set with at least one duplex) were T-1328, T-1631, T-1666, T-2343, T-2417, T-2623, T-2886, T-2887, T-2889, T-3133, T-3187, T-3189, T-3498, T-3499, T-4008, T-4109, T-4485, T-4927, T-4989, T-4993, T-4996, T-4998, T-5060, and T-5114.

Trigger families that achieved a maximum knockdown of between 60-80% relative to vehicle control (for at least one probe set with at least one duplex) were T-1678, T-2263, T-4834, T-4932, T-4957, T-4995, and T-5204. Exemplary duplexes within these families that proved effective in reducing FAM13A expression by 60-80% included D-1615, D-1695, and D-1867 from trigger family T-1678; D-1573 from trigger family T-2263; D-1781, D-1894, D-1906, D-1918, and D-1930 from trigger family T-4834; D-1783, D-1895, D-1907, and D-1931 from trigger family T-4932; D-1631, D-1696, D-1703, D-1717, D-1724, and D-1731 from trigger family T-4957; D-2036 from T-4995; and D-1792, D-1898, and D-1928 from trigger family T-5204.

Trigger families that achieved greater than 80% knockdown relative to vehicle control (for at least one probe set with at least one duplex) were T-1309, T-1333, T-2080, T-2144, T-3000, T-4412, T-4717, T-4999, T-5042, T-5043, T-5045, T-5080, T-5247, T-5249, T-5274, and T-5276. Exemplary duplexes within these families that proved effective in reducing FAM13A expression by greater than 80% included D-1667, D-1686, and D-1849 from trigger family T-1309; D-1597, D-1853, and D-2017 from trigger family T-1333; D-1680, D-1685, and D-1690 from trigger family T-2080; D-1682 and D-1858 from trigger family T-2144; D-1557, D-1650, and D-1861 from trigger family T-3000; D-1955 from trigger family T-4412; D-1896 from trigger family T-4717; D-1614, D-1697, D-1702, D-1709, D-1856, D-1863, D-1865, D-1866, D-1869, D-1873, D-1877, D-1878, D-1879, D-1880, D-1881, D-1884, D-1887, D-1987, D-1992, D-1997, and D-2002 from trigger family T-4999; D-2040 from trigger family T-5042; D-1698, D-1705, D-1864, D-1870, D-1875, D-1883, D-1886, D-1980, D-1984, D-1989, D-1994, and D-2004 from trigger family T-5043; D-1699, D-1612, D-1704, D-1868, D-1871, D-1876, D-1885, D-1888, D-1979, D-1983, D-1988, D-1993, D-1998, and D-2003 from trigger family T-5045; D-1623, D-1846, D-1862, D-1981, D-1985, D-1990, D-1995, D-2000, and D-2005 from trigger family T-5080; D-1768, D-2075, and D-2077 from trigger family T-5247; D-1970 from trigger family T-5249; D-1972 from trigger family T-5274; and D-1975, D-1991, D-1976, D-1977, D-1982, D-1996, and D-2001 from trigger family T-5276.

In testing a range of different modification patterns for some trigger families, it was found that some triggers consistently facilitated high levels of knockdown of FAM13A knockdown. For example, 31 different modification patterns were tested in the above AAV-based experiments using the T-4999 trigger family sequence (D-1614, D-1697, D-1702, D-1709, D-1716, D-1723, D-1730, D-1737, D-1856, D-1863, D-1865, D-1866, D-1869, D-1872, D-1877, D-1878, D-1879, D-1880, D-1881, D-1884, D-1887, D-1978, D-1987, D-1992, D-1997, D-2002, D-2008, D-2017, D-2049, D-2054, and D-2090; see Table 2 for sense and antisense sequences, and modification patterns used, in these duplexes). Each of these duplexes utilized a different modification pattern in the context of the same sense and antisense sequences (SEQ ID NOs: 498 and 1042). Of these duplexes, 25 modification patterns were observed to facilitate greater than 80% knockdown of FAM13A mRNA in at least one assay, and the remaining 6 were observed to facilitate between 60% and 80% knockdown in at least one assay. These data indicate that the T-4999 trigger family is a particularly effective and reliable trigger for reducing FAM13A expression.

Another effective and reliable trigger family is the T-5043 trigger family. For this family, 25 different modification patterns were tested in the above AAV-based experiments (D-1611, D-1698, D-1705, D-1712, D-1719, D-1726, D-1733, D-1740, D-1855, D-1864, D-1870, D-1875, D-1883, D-1886, D-1980, D-1984, D-1989, D-1994, D-1999, D-2004, D-2013, D-2022, D-2044, D-2048, and D-2053; see Table 2 for sense and antisense sequences, and modification patterns used, in these duplexes). Each of these duplexes utilized a different modification pattern in the context of the same sense and antisense sequences (SEQ ID NOs: 503 and 1047). Of these duplexes, 16 modification patterns were observed to facilitate greater than 80% knockdown of FAM13A mRNA in at least one assay, 8 were observed to facilitate between 60% and 80% knockdown in at least one assay, and 1 was observed to facilitate between 40% and 60% knockdown of FAM13A mRNA in at least one assay.

A third particularly effective trigger family is the T-5045 trigger family (whose target sequence largely overlaps with the T-5043 trigger family). For this family, 25 different modification patterns were tested in the above AAV-based experiments (D-1612, D-1699, D-1704, D-1711, D-1718, D-1725, D-1732, D-1739, D-1868, D-1871, D-1876, D-1882, D-1885, D-1888, D-1979, D-1983, D-1988, D-1993, D-1998, D-2003, D-2012, D-2021, D-2043, D-2047, and D-2052; see Table 2 for sense and antisense sequences, and modification patterns used, in these duplexes). Each of these duplexes utilized a different modification pattern in the context of the same sense and antisense sequences (SEQ ID NOs: 504 and 1048). Of these duplexes, 18 modification patterns were observed to facilitate greater than 80% knockdown of FAM13A mRNA in at least one assay, and 7 were observed to facilitate between 60% and 80% knockdown in at least one assay.

Other trigger families that were able to show effective knockdown with multiple modification patterns included the T-1309, T-1333, T-2144, T-3000, T-5080, and T-5226 trigger families.

From a broad perspective, testing a wide range of triggers across the FAM13A transcript revealed that which regions of the transcript were susceptible to RNAi-mediated knockdown. FIG. 6 is a diagram compiling the locations of where different effective trigger families target the FAM13A mRNA transcript (as provided in SEQ ID NO: 1), along with categorizing the maximal degree to which those trigger families were able to knock down FAM13A expression in the above AAV-based assays. The triggers were divided according to whether the maximum observed knockdown for that trigger fell within the range of 40-60% knockdown, 60-80% knockdown, or greater than 80% knockdown.

As shown in FIG. 6, one region of the human FAM13A mRNA transcript that is particularly susceptive to RNAi-based knockdown is the portion between nucleotides 4900 and 5300 of the FAM13A mRNA transcript. Within this small region, 24 distinct trigger families were identified that facilitated knockdown of FAM13A, most of which were validated with multiple different duplexes having different modification patterns. These families included 12 trigger families that facilitated greater than 80% knockdown, 5 families that facilitated between 60% and 80% knockdown, and 7 families that facilitated between 40% and 60% knockdown. This unexpected concentration of successful targets indicates that targeting between nucleotides 4900 and 5300 is a particularly useful strategy for knocking down FAM13A expression.

Other regions that also were susceptible at multiple target locations included nucleotides 1300-1375, nucleotides 1625-1700, and nucleotides 2075-2175. Therefore, these data also indicate that targeting any of these regions is a useful strategy for knocking down FAM13A expression.

These AAV-based experiments also tested the effectiveness of conjugating different ligands to siRNA duplexes in facilitating knockdown in different tissues. FIGS. 8A-8D and Table 14 show the results of testing FAM13A siRNA from the T-4999 and T-5043 families, when the duplexes had been conjugated to either GalNAc (Formula VII) or the fatty acid C22. In these figures, the “*” denotes those duplexes that were conjugated to C22, while those without an asterisk were conjugated to GalNAc. Knockdown data was gathered both the liver and adipose tissue, after systemic administration. GalNAc-conjugated duplexes were administered at 3 mg/kg, while C22 conjugated triggers were administered at 20 mg/kg. All of the tested T-4999 and T-5043 duplexes were able to reduce expression of FAM13A in the liver. In the adipose tissue, the GalNAc-conjugated triggers were less effective in reducing FAM13A expression, with some having no detectable effect. In contrast, the C22-conjugated triggers consistently facilitated reduction of FAM13A expression in adipose tissue to a similar degree as they facilitated in the liver. Examination of these data in combination with studies of weight, fat mass, and metabolic characterization (see Examples 2, 6, and 7) indicates that GalNAc targeting is surprisingly able to achieve similar results to C22 targeting, despite having less effect on FAM13A expression in biologically significant adipose tissue.

The above data also allows for comparison of linkages used to attach C22 to siRNA duplexes. The two tested linkages are through a phosphodiester bond (PO) and through a phosphorothioate bond (PS). Unexpectedly, linking C22 with PS led to significantly better knockdown than linking with PO. This was observed through comparison of pairs of duplexes, which only differ in their conjugation method. For example, one T-4999 trigger family pair showed an increase of 43% in knockdown when switching from PO to PS linkage (compare D-1697 (PO; 37% KD) and D-1856 (PS; 80% KD)). Another T-4999 trigger family pair showed a more modest increase of 6% in knockdown when switching from PO to PS linkage (compare D-1869 (PO; 74% KD) and D-1887 (PS; 80% KD)). A T-5080 trigger family pair showed an increase of 25% in knockdown when switching from PO to PS linkage (compare D-1846 (PO; 44% KD) and D-1862 (PS; 69% KD)). A T-5043 trigger family pair showed an increase of 45% in knockdown when switching from PO to PS linkage (compare D-1698 (PO; 13% KD) and D-1855 (PS; 58% KD)). Another T-5043 trigger family pair showed an increase of 30% in knockdown when switching from PO to PS linkage (compare D-1875 (PO; 40% KD) and D-1886 (PS; 70% KD)). And a T-5045 trigger family pair showed an increase of 38% in knockdown when switching from PO to PS linkage (compare D-1871 (PO; 27% KD) and (D-1882 (PS; 65% KD)). These and other data in Tables 4-17 above show that linking C22 to a siRNA duplex with PS unexpectedly and consistently led to significantly better knockdown than linking to that same siRNA duplex with PO.

### Example 6: In Vivo Knockdown of Endogenous Murine Fam13a in Obesity Model

To determine which human siRNA duplexes (see Examples 4 and 5) would be suitable for testing with endogenous murine Fam13a knockdown experiments, effective trigger families (see Examples 4 and 5 above) were reviewed for cross-reactivity with murine Fam13a mRNA. This review found that the T-4999 trigger family aligned with the murine Fam13a sequence for all except one base of its sequence. Hypothesizing that this might be sufficient to still show knockdown activity, experiments were undertaken to assess the efficacy of T-4999 FAM13A siRNA molecules on endogenous murine Fam13a mRNA in the diet-induced obesity (DIO) model in C57BL/6 mice.

Three duplexes from the T-4999 trigger family were chosen for this assay: D-1709 (GalNAc conjugated via PS), D-1869 (C22 conjugated via PO), and D-1887 (C22 conjugated via PS). Duplexes D-2086 (GalNAc conjugated via PS) and D-2087 (C22 conjugated via PS), which target human FAM13A but were not predicted to bind mouse FAM13A, were used as negative controls. D-2086 (GalNAc conjugated via PS) and D-2087 (C22 conjugated via PS), two duplexes that fully match the murine Fam13a mRNA sequence, were also tested.

Male C57BL6 mice were fed a diet containing high fat content (Research Diets D12492, 60% kcal derived from fat) beginning at 5 weeks of age. When the mice reached 19 weeks of age (14 weeks on the high-fat diet), the mice received a subcutaneous injection of buffer (PBS) or the FAM13A siRNA molecule at a dose of 3 mg/kg body mass or 20 mg/kg body mass in PBS (n=8 mice per group). Body mass was measured continuously throughout the study. Body composition was measured by NMR (EchoMRI 3n1 Body Composition Analyzer) at baseline (2 days prior to injection) and on day 25 post-injection. Liver and subcutaneous white adipose tissue (ScWAT) were collected 4 weeks following siRNA administration and analyzed.

RNA from harvested animal tissues was processed for qPCR analysis. RNA was isolated from 50-100 mg tissue using RNeasy 96 universal tissue kit RNA isolation protocol following manufacturer's instructions (Qiagen). Real-time PCR was performed using TaqMan® RNA-to-Ct™ 1-Step Kit following manufacturer's instructions (ThermoFisher) with 50 ng RNA per reaction and a primer probe set complementary to the murine Fam13a mRNA. A percentage change in murine Fam13a mRNA in liver or ScWAT for each animal was calculated relative to the level of murine Fam13a mRNA in the liver or ScWAT of animals administered PBS buffer control.

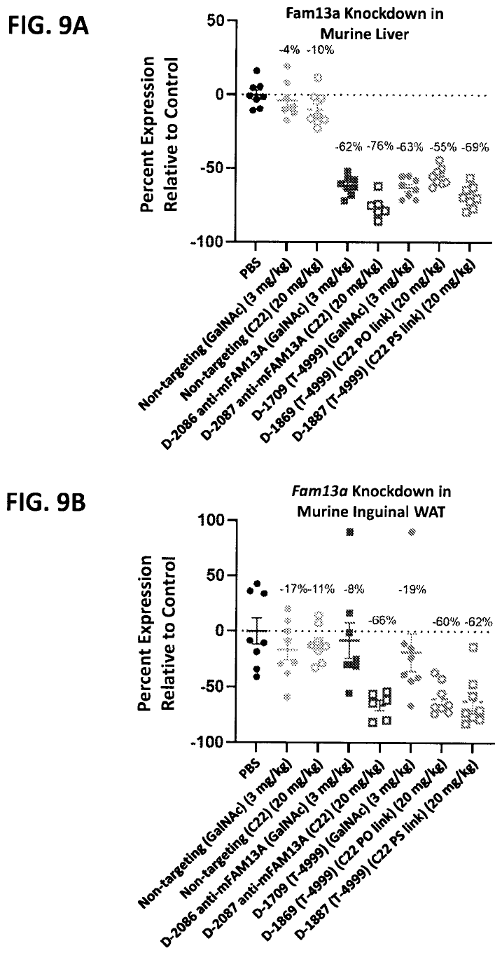

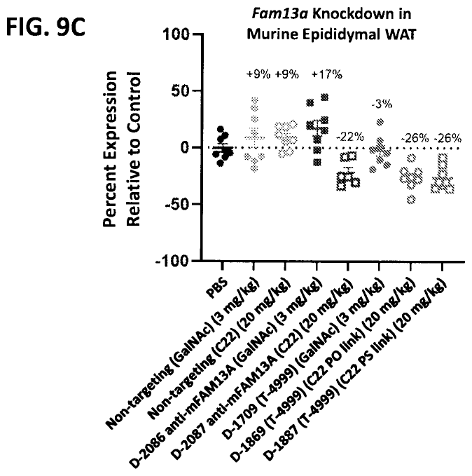

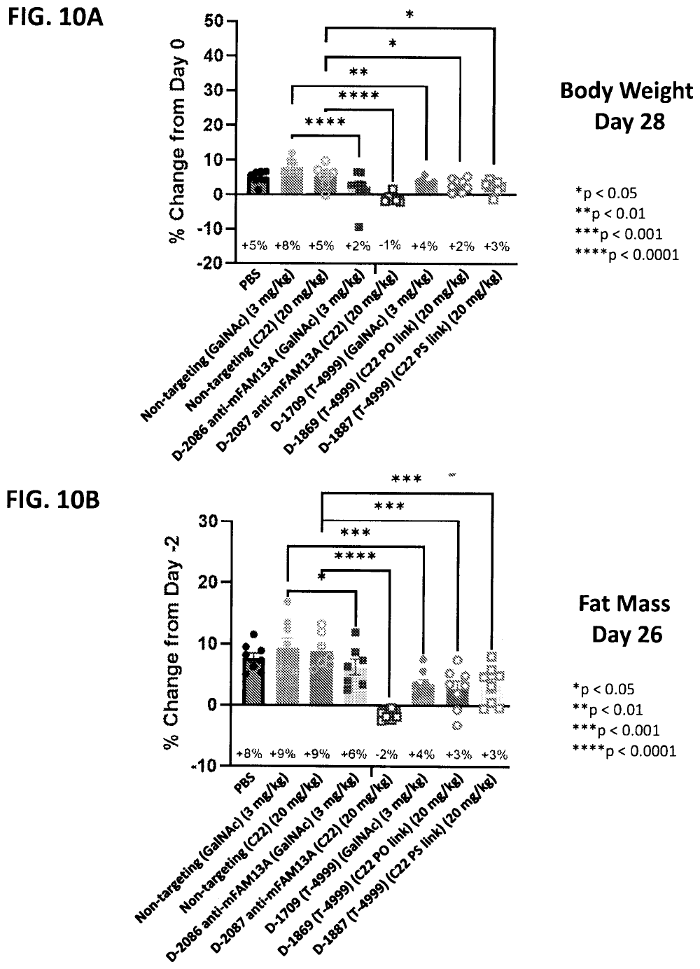

Results of these studies are shown in FIGS. 9A-9C and FIGS. 10A-10B. These figures show the level of knockdown achieved in each mouse's liver, inguinal WAT, and epididymal WAT. Each of the non-targeting control siRNA duplexes displayed expression levels the same as buffer-only control mice (in all three tissues).

In the liver, all the Fam13a-directed duplexes effectively reduced expression of murine Fam13a (FIG. 9A). The GalNAc-linked duplexes, D-2086 and D-1709, reduced Fam13a expression in the liver equivalently (by 62% and 63%, respectively). This showed that the T-4999 duplex (D-1709) was effective despite having one mismatch with the target sequence. Each of the C22-linked duplexes also facilitated Fam13a knockdown in the liver, with D-2087 resulting in 76% knockdown, D-1869 resulting in 55% knockdown, and D-1887 resulting in 69% knockdown.

In inguinal WAT, the C22-linked duplexes were more effective than the GalNAc-linked duplexes in reducing murine Fam13a expression. The GalNAc-linked duplexes, D-2086 and D-1709, reduced expression in the liver by 8% and 19%, respectively. In contrast, the C22-linked duplexes resulted in Fam13a knockdown at similar levels to that achieved in the liver: D-2087 resulted in 66% knockdown, D-1869 resulted in 60% knockdown, and D-1887 resulted in 62% knockdown.

In epididymal WAT, less knockdown was observed than in the other two tissue types. Neither of the GalNAc-linked duplexes resulted in significant knockdown of murine Fam13a. In contrast, the C22-linked duplexes resulted in some Fam13a knockdown: D-2087 resulted in 22% knockdown, D-1869 resulted in 26% knockdown, and D-1887 resulted in 26% knockdown.

FIG. 10A shows the effects of siRNA treatment on the body weight of the DIO mice. Untreated and control treated mice had a 5-8% increase in body weight over the course of the experiment. Treatment with any of the Fam13a duplexes decreased or prevented that weight gain. The GalNAc-linked duplexes, D-2086 and D-1709, limited the weight gain to 2% and 4%, respectively. The C22-linked duplexes also limited the weight gain, with D-2087 actually resulting in a 1% weight loss for the mice, D-1869 limiting the gain to 2%, and D-1887 limiting the gain to 3%.

FIG. 10B shows the effects of siRNA treatment on the fat mass of the DIO mice. Untreated and control treated mice had an 8-9% increase in fat mass over the course of the experiment. Treatment with any of the Fam13a duplexes decreased or prevented that weight gain. The GalNAc-linked duplexes, D-2086 and D-1709, limited the weight gain to 6% and 4%, respectively. The C22-linked duplexes also limited the weight gain, with D-2087 actually resulting in a 2% weight loss for the mice, D-1869 limiting the gain to 3%, and D-1887 limiting the gain to 3%.

These data provide further support for FAM13A siRNA (and the T-4999 trigger family specifically) being used for a variety of purposes, such as reducing abdominal adiposity, reducing body weight, reducing fat mass, improving metabolic parameters including insulin resistance and non-alcoholic steatohepatitis (NASH), and reducing risk of myocardial infarction.

### Example 7: FAM13A siRNA in Nonhuman Primates

To assess the efficacy of the FAM13A siRNA molecules in a nonhuman primate model, top performing FAM13A siRNA molecules from the in vitro and in vivo activity assays described in Examples 4 and 5 were evaluated for in vivo efficacy using cynomolgus monkeys. In particular, triggers from the T-4999 and T-5043 families were selected. Because the selected triggers target a sequence present in both human and cynomolgus FAM13A mRNA, it was expected that they would also be effective in knocking down endogenous cynomolgus FAM13A.

For these experiments, the sense strand in each tested siRNA molecule was conjugated to the trivalent GalNAc moiety shown in Formula VII or to docosanoic acid (C22), using the methods described in Example 3. Accordingly, the experiment used the T-4999 duplexes T-1709 (GalNAc conjugated via PS) and D-1887 (C22 conjugated via PS), and the T-5043 duplexes D-1705 (GalNAc conjugated via PS) and D-1886 (C22 conjugated via PS).

The study design is provided in Table 18 below. Briefly, there were N=3 animals per treatment group (naïve and non-naïve, female, lean cynomolgus monkeys, Cambodian origin, 3 years old). A single subcutaneous dose was administered in the mid-scapular region to each animal. Liver tissue biopsies were collected pre-dose on day −14 or day −11, and post-dose on days 14, 30, and 45 (relative to dosing on day 0). Adipose tissue biopsies were collected pre-dose on day −14 or day −11 (omental fat), and post-dose on days 14 (falciform fat), 30 (omental fat), and 45 (omental and falciform fat). Blood for clinical chemistry analysis was collected via femoral vein on days −14 (prior to biopsy), −7, 7, 14 (prior to biopsy), 20, 25, 30 (prior to biopsy), 35, and 45 (prior to necropsy). Animals were fasted on days −14, 14, and 30 due to the tissue biopsy collection procedures.

TABLE 18
NHP Study Design

| | | | | | | Dose | Dose |
|---|---|---|---|---|---|---|---|
| | | SIRNA | Trigger | | Dose | Level | Conc. |
| Group | Animals | Duplex | Family | Carrier | Route | (mg/kg) | (mg/mL) |
| 1 | 3 | D-1709 | 4999 | GalNAc | SC | 3 | 0.6 |
| 2 | 3 | D-1887 | 4999 | C22 | SC | 20 | 4 |
| 3 | 3 | D-1705 | 5043 | GalNAc | SC | 3 | 0.6 |
| 4 | 3 | D-1886 | 5043 | C22 | SC | 20 | 4 |

For analysis of FAM13A knockdown level in liver and adipose tissue, the total RNA was isolated from 10 to 20 mg tissue for each tissue sample from each time point. A cDNA sample was then prepared from each total RNA sample and diluted 1:10 for ddPCR analysis. A cynomolgus FAM13A primer/probe set and a cynomolgus PPIB primer/probe set was used in the analysis. The percent mRNA knockdown was calculated relative to the pre-dose FAM13A expression level for each individual animal and then averaged across timepoints.

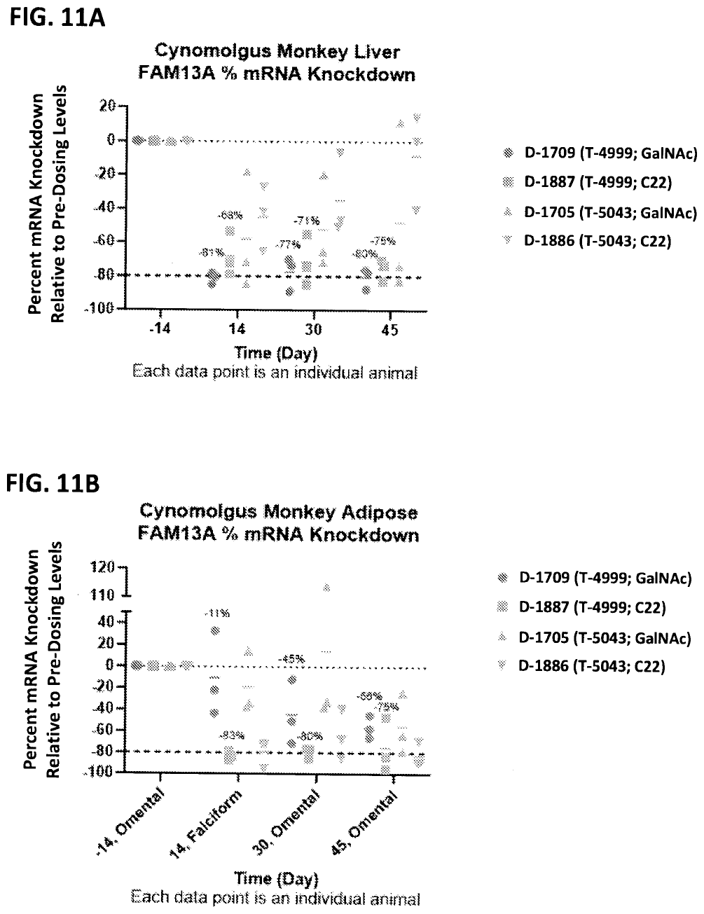

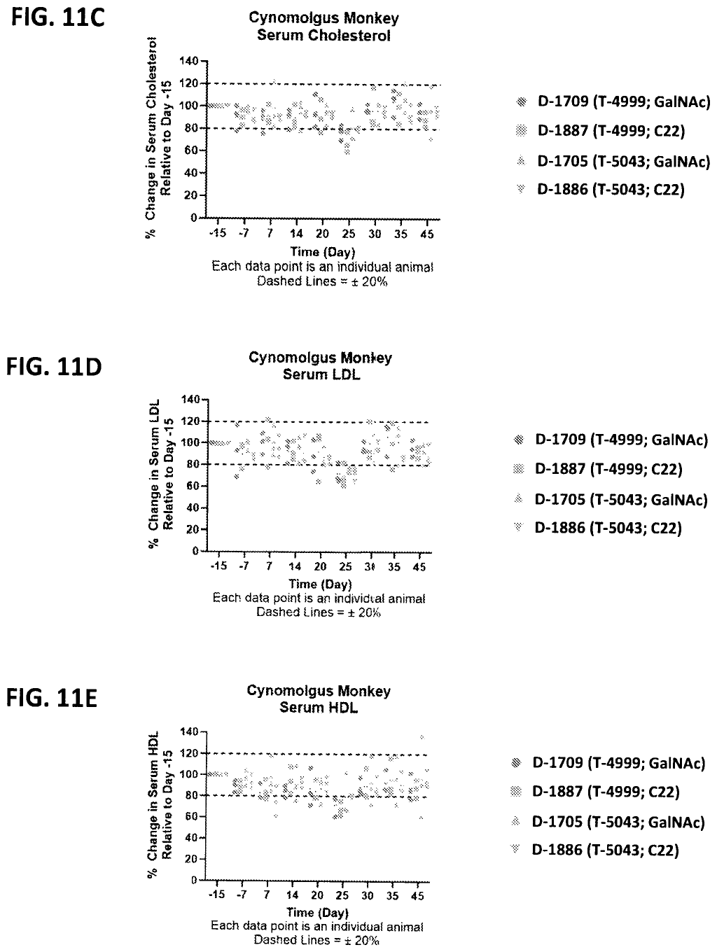

The data on knockdown of FAM13A mRNA levels in the liver is shown in FIG. 11A. The most effective duplex in the liver was D-1709, the GalNAc-conjugated siRNA from the T-4999 trigger family. In the liver, the single dose of D-1709 reduced FAM13A mRNA levels by an average of 81% by day 14, and the knockdown was maintained at day 30 (77%) and day 45 (80%) without any subsequent treatment. The duplex D-1887, which is identical to D-1709 aside from being C22-conjugated, was almost as effective as D-1709 (albeit at a higher dose). The single dose of D-1887 reduced FAM13A mRNA levels by an average of 68% by day 14, and the knockdown was increased on day 30 (71%) and day 45 (75%) without any subsequent treatment.

FIG. 11A also shows the liver knockdown achieved by two duplexes from the T-5043 trigger family. The single dose of D-1705 (GalNAc) reduced FAM13A mRNA levels by an average of 58% by day 14, and the knockdown was maintained at day 30 (52%) and day 45 (48%) without any subsequent treatment. However, the knockdown was much higher in two of the animals, as one of the three treated animals was a possible outlier that exhibited minimal knockdown. The other duplex in the T-5043 family, D-1886 (C22), reduced FAM13A mRNA levels by an average of 45% by day 14, but the knockdown levels decreased by day 30 (35%) and day 45 (8.4%).

FIG. 11B shows the data on knockdown of FAM13A mRNA in the adipose tissue. The most effective duplexes in the adipose tissue were D-1887 (T-4999; C22) and D-1886 (T-5043; C22). The single dose of D-1887 reduced FAM13A mRNA levels by an average of 83% by day 14, and the knockdown was maintained on day 30 (80%) and day 45 (75%) without any subsequent treatment. Similarly, the single dose of D-1886 reduced FAM13A mRNA levels by an average of 79% by day 14, and the knockdown was maintained on day 30 (64%) and day 45 (83%) without any subsequent treatment. The two GalNAc conjugated duplexes demonstrated a lag time in silencing activity but were also effective in knocking down FAM13A. The single dose of D-1709 reduced FAM13A mRNA levels by an average of 11% by day 14, and the knockdown increased at day 30 (45%) and day 45 (56%) without any subsequent treatment. The single dose of D-1705 had minimal effects on FAM13A mRNA levels at day 14 (decreased 19%) and day 30 (increased 15%), but an average knockdown of 55% was observed on day 45.

FIGS. 11C-11E show results of the clinical chemistry analysis performed on blood serum samples from the treated animals. For all of the tested duplexes, there was a consistent approximately 20% or greater decrease in serum cholesterol (FIG. 11C), serum LDL (FIG. 11D), and serum HDL (FIG. 11E) between 20 and 30 days after siRNA treatment. These decreases are consistent with the effects of FAM13A-targeted siRNA on the blood chemistry of mice (see Example 2 and FIG. 5). Accordingly, these data provide further support for FAM13A siRNA (and the T-4999 and T-5043 trigger families specifically) being used for a variety of purposes, such as reducing abdominal adiposity, reducing body weight, reducing fat mass, improving metabolic parameters including insulin resistance and non-alcoholic steatohepatitis (NASH), and reducing risk of myocardial infarction.

Further evidence for the efficacy of FAM13A siRNA in treatment of such conditions will be gathered through the use of obese cynomolgus monkeys. These animals will be monitored after the administration of the T-4999 duplexes T-1709 (GalNAc conjugated via PS) and D-1887 (C22 conjugated via PS), and the T-5043 duplexes D-1705 (GalNAc conjugated via PS) and D-1886 (C22 conjugated via PS). The weight, fat mass, blood chemistry, and other metabolic parameters will be monitored and correlated with the knockdown of FAM13A expression in both the liver and adipose tissue.

1. An RNAi construct comprising a sense strand and an antisense strand, wherein the sense strand comprises a sequence that is sufficiently complementary to the sequence of the antisense strand to form a duplex region, and
wherein the antisense strand comprises:
(a) a region that has substantial identity to at least 15 contiguous nucleotides within nucleotides 1300-1375 or 4900-5300 of the FAM13A mRNA sequence set forth in SEQ ID NO: 1, such that there are no more than 2 mismatches between the antisense strand's region of substantial identity and the contiguous nucleotides; or
(b) a region that has substantial identity to at least 15 contiguous nucleotides from an antisense sequence listed in Table 1 or Table 2, such that there are no more than 2 mismatches between the antisense strand's region of substantial identity and the contiguous nucleotides.

2-5. (canceled)

6. The RNAi construct of claim 1, wherein the sense strand and antisense strand form a duplex region of about 15 to about 30 base pairs in length.

7. (canceled)

8. The RNAi construct of claim 6, wherein the duplex region is about 19 to about 21 base pairs in length.

9. (canceled)

10. The RNAi construct of claim 6, wherein the sense strand and the antisense strand are each independently about 19 to about 23 nucleotides in length.

11. (canceled)

12. The RNAi construct of claim 1, wherein the RNAi construct comprises one or two nucleotide overhangs of 1 to 4 unpaired nucleotides.

13-14. (canceled)

15. The RNAi construct of claim 1, wherein the RNAi construct comprises one or more modified nucleotides.

16-17. (canceled)

18. The RNAi construct of claim 15, wherein all the nucleotides in the sense and antisense strands are modified nucleotides.

19. The RNAi construct of claim 18, wherein the modified nucleotides are 2′-O-methyl modified nucleotides, 2′-fluoro modified nucleotides, or a combination thereof.

20. The RNAi construct of claim 1, wherein the sense strand comprises an abasic nucleotide as the terminal nucleotide at its 3′ end, its 5′ end, or both its 3′ and 5′ ends.

21. (canceled)

22. The RNAi construct of claim 1, wherein the sense strand, the antisense strand, or both the sense and antisense strands comprise one or more phosphorothioate internucleotide linkages.

23-25. (canceled)

26. The RNAi construct of claim 1, wherein the antisense strand comprises or consists of a sequence selected from the antisense sequences listed in Table 1 or Table 2.

27. The RNAi construct of claim 1, wherein the sense strand comprises a sequence selected from the sense sequences listed in Table 1 or Table 2.

28. The RNAi construct of claim 1, wherein the sense and antisense strands, respectively, comprise SEQ ID NOs: 15 and 559, SEQ ID NOs: 24 and 568, SEQ ID NOs: 125 and 669, SEQ ID NOs: 127 and 671, SEQ ID NOs: 222 and 766, SEQ ID NOs: 406 and 950, SEQ ID NOs: 448 and 992, SEQ ID NOs: 498 and 1042, SEQ ID NOs: 502 and 1046, SEQ ID NOs: 503 and 1047, SEQ ID NOs: 504 and 1048, SEQ ID NOs: 513 and 1057, SEQ ID NOs: 526 and 1070, SEQ ID NOs: 527 and 1071, SEQ ID NOs: 533 and 1077, or SEQ ID NOs: 534 and 1078.

29. (canceled)

30. The RNAi construct of claim 28, wherein the sense and antisense strands, respectively, comprise SEQ ID NOs: 498 and 1042.

31. The RNAi construct of claim 1, wherein the RNAi construct is D-1557, D-1597, D-1612, D-1614, D-1623, D-1650, D-1667, D-1680, D-1682, D-1685, D-1686, D-1690, D-1697, D-1698, D-1699, D-1702, D-1704, D-1705, D-1709, D-1768, D-1846, D-1849, D-1853, D-1856, D-1858, D-1861, D-1862, D-1863, D-1864, D-1865, D-1866, D-1868, D-1869, D-1870, D-1871, D-1873, D-1875, D-1876, D-1877, D-1878, D-1879, D-1880, D-1881, D-1883, D-1884, D-1885, D-1886, D-1887, D-1888, D-1899, D-1896, D-1955, D-1970, D-1972, D-1975, D-1976, D-1977, D-1979, D-1980, D-1981, D-1982, D-1983, D-1984, D-1985, D-1987, D-1988, D-1989, D-1990, D-1991, D-1992, D-1993, D-1994, D-1995, D-1996, D-1997, D-1998, D-2000, D-2001, D-2002, D-2003, D-2004, D-2005, D-2012, D-2013, D-2014, D-2017, D-2021, D-2022, D-2023, D-2040, D-2044, D-2045, D-2047, D-2049, D-2051, D-2052, D-2053, D-2054, D-2058, D-2061, D-2075, D-2077, D-2079, D-2080, D-2081, D-2083, D-2090, D-2091, or D-2093.

32. (canceled)

33. The RNAi construct of claim 1, wherein the sense and antisense strands, respectively, comprise SEQ ID NOs: 1800 and 2648 (D-1709) or SEQ ID NOs: 2861 and 3115 (D-1887).

34. The RNAi construct of claim 1, wherein the RNAi construct further comprises a ligand.

35-36. (canceled)

37. The RNAi construct of claim 34, wherein the ligand comprises a multivalent galactose moiety or multivalent N-acetyl-galactosamine moiety.

38. The RNAi construct of claim 37, wherein the multivalent galactose moiety or multivalent N-acetyl-galactosamine moiety is trivalent or tetravalent.

39. The RNAi construct of claim 34, wherein the ligand is a long-chain fatty acid.

40. (canceled)

41. The RNAi construct of claim 39, wherein the long-chain fatty acid is docosanoic acid (C22).

42-43. (canceled)

44. The RNAi construct of claim 34, wherein the ligand is attached through a phosphodiester or phosphorothioate linkage.

45. A pharmaceutical composition comprising the RNAi construct of claim 1 and a pharmaceutically acceptable carrier or excipient.

46. A method for reducing the expression of FAM13A protein in a patient in need thereof comprising administering to the patient the RNAi construct of claim 1.

47-48. (canceled)

49. The method of claim 46, wherein the patient is diagnosed with or at risk for obesity, abdominal obesity, NASH, hepatosteatosis, insulin resistance, type 2 diabetes, hypertriglyceridemia, or hypercholesterolemia.

50. A method for reducing the body weight or fat mass of a patient comprising administering to the patient the RNAi construct of claim 1.

51. (canceled)

52. The method of claim 50, wherein the waist to hip ratio is greater than 1.0.

53. The method of claim 50, wherein the patient has been diagnosed with abdominal obesity.

54. The method of claim 46, wherein the RNAi construct or pharmaceutical composition is administered to the patient via a parenteral route of administration.

55-57. (canceled)

58. A method of reducing body weight or fat mass by administering an RNAi construct comprising a sense strand, an antisense strand, and a ligand that targets delivery to hepatocytes, wherein the antisense sense strand has a sequence that is complementary to a FAM13 mRNA sequence.

59. (canceled)

60. The method of claim 58, wherein the antisense strand comprises a region comprising a sequence that is substantially complementary to at least 15 contiguous nucleotides within nucleotides 1300-1375 or 4900-5300 of the FAM13A mRNA sequence set forth in SEQ ID NO: 1.

61. The method of claim 58, wherein the antisense strand comprises a region comprising a sequence that is substantially complementary to a FAM13A mRNA sequence, and wherein said region comprises at least 15 contiguous nucleotides from an antisense sequence listed in Table 1 or Table 2.

62-84. (canceled)

85. The method of claim 58, wherein the antisense strand or sense strand comprises a sequence selected from the antisense sequences listed in Table 1 or Table 2.

86. (canceled)

87. The method of claim 58, wherein the sense and antisense strands, respectively, comprise SEQ ID NOs: 15 and 559, SEQ ID NOs: 24 and 568, SEQ ID NOs: 125 and 669, SEQ ID NOs: 127 and 671, SEQ ID NOs: 222 and 766, SEQ ID NOs: 406 and 950, SEQ ID NOs: 448 and 992, SEQ ID NOs: 498 and 1042, SEQ ID NOs: 502 and 1046, SEQ ID NOs: 503 and 1047, SEQ ID NOs: 504 and 1048, SEQ ID NOs: 513 and 1057, SEQ ID NOs: 526 and 1070, SEQ ID NOs: 527 and 1071, SEQ ID NOs: 533 and 1077, or SEQ ID NOs: 534 and 1078.

88. (canceled)

89. The method of claim 87, wherein the sense and antisense strands, respectively, comprise SEQ ID NOs: 498 and 1042.

90. The method of any claim 58, wherein the RNAi construct is D-1557, D-1597, D-1612, D-1614, D-1623, D-1650, D-1667, D-1680, D-1682, D-1685, D-1686, D-1690, D-1697, D-1698, D-1699, D-1702, D-1704, D-1705, D-1709, D-1768, D-1846, D-1849, D-1853, D-1856, D-1858, D-1861, D-1862, D-1863, D-1864, D-1865, D-1866, D-1868, D-1869, D-1870, D-1871, D-1873, D-1875, D-1876, D-1877, D-1878, D-1879, D-1880, D-1881, D-1883, D-1884, D-1885, D-1886, D-1887, D-1888, D-1899, D-1896, D-1955, D-1970, D-1972, D-1975, D-1976, D-1977, D-1979, D-1980, D-1981, D-1982, D-1983, D-1984, D-1985, D-1987, D-1988, D-1989, D-1990, D-1991, D-1992, D-1993, D-1994, D-1995, D-1996, D-1997, D-1998, D-2000, D-2001, D-2002, D-2003, D-2004, D-2005, D-2012, D-2013, D-2014, D-2017, D-2021, D-2022, D-2023, D-2040, D-2044, D-2045, D-2047, D-2049, D-2051, D-2052, D-2053, D-2054, D-2058, D-2061, D-2075, D-2077, D-2079, D-2080, D-2081, D-2083, D-2090, D-2091, or D-2093.

91. (canceled)

92. The method of claim 58, wherein the sense and antisense strands, respectively, comprise SEQ ID NOs: 1800 and 2648 (D-1709) or SEQ ID NOs: 2861 and 3115 (D-1887).

93-97. (canceled)
